(19) United States
(12) Patent Application Publication
(10) Pub. No.: US 2022/0041647 A1
(43) Pub. Date: Feb. 10, 2022

(54) ESTRA-1,3,5(10)-TRIENE COMPOUNDS CONDENSED IN POSITION 16(17) WITH A PYRAZOLE RING AS INHIBITORS OF 17-HSD1

(71) Applicant: FORENDO PHARMA LTD, Turku (FI)

(72) Inventors: Leena HIRVELÄ, Turku (FI); Marjo HAKOLA, Turku (FI); Tero LINNANEN, Turku (FI); Pasi KOSKIMIES, Turku (FI); Camilla STJERNSCHANTZ, Turku (FI)

(21) Appl. No.: 17/299,584

(22) PCT Filed: Dec. 5, 2019
(86) PCT No.: PCT/FI2019/050874

§ 371 Date: Jun. 3, 2021

(30) Foreign Application Priority Data
Dec. 5, 2018 (FI) 20186056

(51) Int. Cl. C07J 71/00; C07J 41/00; C07J 43/00; A61K 45/06; A61K 31/58

(52) U.S. Cl. CPC C07J 71/0068; C07J 41/0072; A61K 31/58; A61K 45/06; C07J 43/003

(57) ABSTRACT

The invention relates to compounds of formula (I) and pharmaceutically acceptable salts thereof

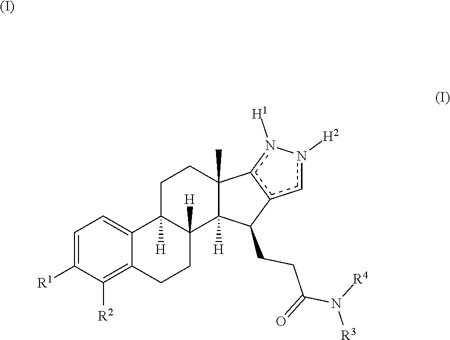

wherein R1 to R4 are as defined in the claims. The invention further relates to their use as inhibitors of 17β-HSD1 and in treatment or prevention of steroid hormone dependent diseases or disorders, such as steroid hormone dependent diseases or disorders requiring the inhibition of the 17β-HSD1 enzyme and/or requiring the lowering of the endogenous estradiol concentration. The present invention also relates to the preparation of the aforementioned compounds and to pharmaceutical compositions comprising as an active ingredient(s) one or more of the aforementioned compounds or pharmaceutically acceptable salts thereof.

### FIELD OF THE INVENTION

The present invention relates to novel steroidal C-15 derivatives, to their pharmaceutically acceptable salts, and their use in therapy. The invention further relates to pharmaceutical compositions comprising these compounds as active ingredients and to methods for their preparation.

### BACKGROUND OF THE INVENTION

17β-hydroxysteroid dehydrogenases (17β-HSDs), also known as 17-ketosteroid reductases (17-KSR) are NAD(H)- and/or NAPD(H)-dependent alcohol oxidoreductase enzymes which catalyse the last and key step in formation of all estrogens and androgens. More specifically 17β-HSDs catalyse the dehydrogenation (oxidation) of 17-hydroxysteroids into corresponding 17-ketosteroids or hydrogenation (reduction) of inactive 17-ketosteroids into corresponding active 17-hydroxysteroids.

As both estrogens and androgens have the highest affinity for their receptors in the 17β-hydroxy form, the 17β-HSD/KSRs regulate the biological activity of the sex hormones. At present, 15 human members of 17β-HSDs have been described (type 1-15). Different types of 17β-HSD/KSRs differ in their substrate and cofactor specificities. The 17KSR activities convert low-activity precursors to more potent forms while 17β-HSD activities decrease the potency of estrogens and androgens and consequently may protect tissues from excessive hormone action.

Each type of 17β-HSD has a selective substrate affinity and a distinctive, although in some cases overlapping, tissue distribution.

Type 1 17β-hydroxysteroid dehydrogenase (17β-HSD1) is most abundantly expressed in the ovarian granulosa cells of the developing follicles in ovaries and in human placenta, both being estrogen biosynthetic tissues. In addition, 17β-HSD1 is expressed in estrogen target tissues, including breast, endometrium and bone. The human 17β-HSD1 is specific to estrogenic substrates and in vivo catalyzes the reduction of estrone to estradiol.

Type 2 17β-hydroxysteroid dehydrogenase (17β-HSD2) on the other hand converts estradiol, testosterone and 5a-dihydrotestrosterone to their less active forms estrone, androstenedione and 5a-androstanedione, respectively. Due to its wide and abundant expression in number of various estrogen and androgen target tissues, such as uterus, placenta, liver and the gastrointestinal and urinary tracts, it has been suggested that type 2 enzyme protects tissues from excessive steroid actions.

Estradiol (E2) is about 10 times as potent as estrone (E1) and about 80 times as potent as estratriol (E3) in its estrogenic effect. In contrast to certain other estrogens, estradiol binds well to both estrogen receptors ERα and ERβ, and thus regulates the expression of a variety of genes.

Although both 17β-HSD1 and 17β-HSD2 are present in healthy premenopausal humans, increased ratio of 17β-HSD1 to 17-HSD2 in the tumors of postmenopausal patients with hormone-dependent breast cancer has been shown in several studies. 17HSD1 gene amplification and loss of heterozygosity of 17HSD2 allele are potential mechanisms involved to increased reductive estrogen synthesis pathway in breast tumors. Increased ratio of type 1 enzyme to type 2 enzyme results in an increased level of estradiol that then promotes the proliferation of the cancerous tissue via the estrogen receptors (ER). High levels of estrogen thus support certain cancers such as breast cancer and cancer of the uterine lining i.e. endometrial cancer and uterine cancer.

Similarly it has been suggested that 17β-HSD2 is down-regulated in endometriosis while both aromatase and 17β-HSD1 are expressed or up-regulated in comparison with normal endometrium. This again results in the presence of high concentration of estradiol (E2) which drives the proliferation of the tissue. Similar mechanism has been elucidated in uterine leiomyoma (uterine fibroids) and endometrial hyperplasia.

Reduction of the endogenous estradiol concentration in affected tissues will result in reduced or impaired proliferation of 17β-estradiol cells in said tissues and may thus be utilized in prevention and treatment of malign and benign estradiol dependent pathologies. Due to the proposed involvement of 17β-estradiol in a number of malign and benign pathologies, inhibitors of 17β-hydroxysteroid dehydrogenases, that can be used to impair endogenous production of estradiol from estrone, can have therapeutic value in the prevention or the treatment of such disorders or diseases are in great demand.

Some small-molecule inhibitors of 17β-HSD1 enzyme have been identified and reviewed in Poirier D. (2003) Curr Med Chem 10: 453-77 and Poirier D. (2010) Expert Opin. Ther. Patents 20(9): 1123-1145. Further, small molecule inhibitors of 17β-HSD's have been disclosed in WO 2001/42181, WO 2003/022835, WO2003/033487, WO2004/046111, WO2004/060488, WO2004/110459, WO 2005/032527, and WO 2005/084295.

WO2004/085457 discloses steroidal compounds capable of inhibiting 17β-hydroxysteroid dehydrogenase. WO2006/003012 discloses 2-substituted D-homo-estriene derivatives suitable for the treatment of estrogen-dependent diseases that can be influenced by the inhibition of the 17β-hydroxysteroid dehydrogenase type 1. Similarly WO2006/003013 presents 2-substituted estratrienones usable for preventing and treating estrogen-dependent diseases influenced by inhibiting 17β-hydroxysteroid dehydrogenase type 1.

15-substituted estradiol analogues acting as locally active estrogens are presented in WO2004/085345. WO2006/027347 discloses 15 β-substituted estradiol derivatives having selective estrogenic activity for the treatment or prevention of estrogen receptor-related diseases and physiological conditions. Further, WO2005/047303 discloses 3, 15 substituted estrone derivatives capable of inhibiting the 17β-hydroxysteroid dehydrogenase type 1.

International application WO2008/034796 relates to estratrien triazoles suitable for use in treatment and prevention of steroid hormone dependent diseases or disorders requiring the inhibition of a 17β-hydroxysteroid dehydrogenases such as 17β-HSD type 1, type 2 or type 3 enzyme. Inhibitors of 17β-HSD type 3 enzyme have been disclosed in WO99/46279.

International applications WO2014/207309, WO2014/207310 and WO2014/207311 relate to estrone C-15 thiazole derivatives, estrone C-17 ketimine C-15 thiazole derivatives and estradiol C-15 thiazole derivatives, respectively, as well as their use in therapy.

### BRIEF DESCRIPTION OF THE INVENTION

An object of the present invention is to provide compounds useful in treating disorders and diseases associated with increased level of estradiol and/or treatable by inhibition of 17β-HSD1 enzyme. It is further an object of the present invention to provide compounds that show little or no inhibitory effect on 17β-HSD2 enzyme.

One of the problems associated with the known 17β-HSD1 inhibitors is the disposition, in particular the metabolic stability, of the compounds. It is therefore yet a further object of the present invention to provide compounds with improved metabolic stability.

Some further problems associated with the known 17β3-HSD1 inhibitors are the formation of conjugative metabolites and species selectivity of the compounds. It is therefore yet a further object of the present invention to provide compounds with improved properties in these parameters.

One further problem associated with the known 17β-HSD1 inhibitors is that while some inhibitors may show 17β-HSD1 inhibition, said inhibitors may not exhibit properties of low 17β-HSD2 inhibition, metabolic stability and/or inhibition in other species. It is therefore yet a further object of the present invention to provide compounds with improved one or more of said property (properties), including inhibition of 17β-HSD1.

The present invention provides novel compounds of formula (I)

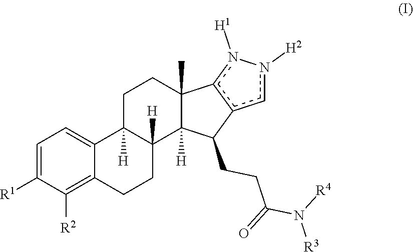

wherein

R1 and R2 are each independently selected from the group consisting of H and halogen;

(i) R3 is selected from the group consisting of H and C1-4-alkyl and

R4 is selected from the group consisting of
C1-6-alkyl optionally substituted with OH;
—(CH₂)ₙ—R5, where n is 1 to 3 and R5 is a 3 to 7 membered alicycle;
4 to 7 membered unsubstituted saturated alicycle or unsubstituted heterocycle comprising one heteroatom selected from the group consisting of nitrogen, sulfur, and oxygen;
5 membered partially unsaturated heterocycle or aromatic heterocycle comprising 1 to 3 heteroatom(s) selected from the group consisting of nitrogen, sulfur, and oxygen, and being optionally substituted with one or two substituents selected from the group consisting of halogen, CN, C1-4-alkyl, C1-3-(per)haloalkyl, OH, C1-3-alkoxy, C(O)N(C1-3-alkyl)₂, and 6 membered saturated heterocycle comprising 1 to 3 heteroatom(s) independently selected from the group consisting of nitrogen, oxygen and sulfur and being optionally substituted with one or two substituent(s) independently selected from the group consisting of halogen, CN, C1-4-alkyl, C1-3-(per)haloalkyl, OH, and C1-3-alkoxy;
5 membered unsubstituted unsaturated or aromatic heterocycle comprising 1 to 3 heteroatom(s) independently selected from the group consisting of nitrogen, sulfur, and oxygen;
5 membered unsaturated or aromatic heterocycle comprising 1 to 3 heteroatom(s) independently selected from the group consisting of nitrogen, and oxygen, and being optionally substituted with one or two substituent(s) independently selected from the group consisting of halogen, CN, C1-4-alkyl, C1-3-(per)haloalkyl, OH, C1-3-alkoxy, C(O)N(C1-3-alkyl)₂, and 6 membered saturated heterocycle containing 1 to 3 heteroatom(s) independently selected from the group consisting of nitrogen, oxygen and sulfur and being optionally substituted with one or two substituent(s) independently selected from the group consisting of halogen, CN, C1-4 alkyl, C1-3-(per)haloalkyl, OH, and C1-3-alkoxy;
phenyl, 6 membered unsaturated, or aromatic heterocycle comprising 1 to 3 heteroatom(s) independently selected from the group consisting of nitrogen, sulfur, and oxygen, and being optionally substituted with one to five substituent(s) independently selected from the group consisting of halogen, CN, C1-4 alkyl, C1-3-(per)haloalkyl, OH, oxo, C1-3-alkoxy, morpholino, C(O)N(C1-3-alkyl)₂ and a 6 membered saturated heterocycle with 1 to 3 heteroatom(s) selected from the group consisting of nitrogen, oxygen and sulfur, optionally substituted with C1-4 alkyl; and
6 membered saturated heterocycle comprising 1 to 3 heteroatom(s) independently selected from the group consisting of nitrogen, oxygen and sulfur and being optionally substituted with one to three substituent(s) independently selected from the group consisting of halogen, CN, C1-4-alkyl, C1-3-(per)haloalkyl, OH, oxo, and C1-3-alkoxy, or two adjacent substituents may form a 5 or 6 membered saturated fused ring;

or

(ii) R3 and R4 form together with the nitrogen atom they are attached to a group selected from a 4 to 7 membered saturated heterocycle comprising said nitrogen atom and optionally one additional heteroatom selected from the group consisting of nitrogen, oxygen and sulfur including sulfonyl, and being optionally substituted with a substituent selected from the group consisting of halogen, CN, methyl, C1-3-(per)haloalkyl, OH, oxo, C1-3-alkoxy and a 4 to 7 membered alicycle or saturated heterocycle with 1 to 3 heteroatoms selected from the group consisting of nitrogen, oxygen and sulfur, optionally substituted with C1-4-alkyl;

or

(iii) R3 and R4 form together with the nitrogen atom they are attached to a group selected from a 4 to 7 membered saturated heterocycle comprising said nitrogen atom and said saturated heterocycle forms a fused or spirocycle ring with a 4 to 7 membered saturated or unsaturated alicycle or heterocycle with 1 to 3 heteroatoms selected from the group nitrogen, oxygen and sulfur;

provided that only one of hydrogens H¹ and H² is present at the same time, and the position of the double bonds in the pyrazole ring to which the hydrogens H¹ and H² are attached is determined based on which of the hydrogen H¹ and H² is present

or a pharmaceutically acceptable salt thereof.

The invention also relates to pharmaceutical composition comprising an effective amount on one or more compound(s) of formula (I).

Further the invention relates to a compound of formula (I) or a pharmaceutical acceptable salt thereof for use as a medicament.

Still further the invention relates to a compound of formula (I) or pharmaceutically acceptable salt thereof for use in the treatment of estradiol dependent malign or benign diseases or disorders.

Finally the invention provides a method for the preparation of compounds according to formula (I) and its intermediate compound according to formula (II).

### DETAILED DESCRIPTION OF THE INVENTION

Compounds of the present disclosure contain steroidal core structure having a defined stereochemistry that is the natural configuration of estrogens.

Compounds of the present disclosure bear a side chain at C15, which, together with the specific substitution pattern of the A ring and C16-C17 fused pyrazole ring provides the inventive properties of the compounds of the present disclosure. These three modifications of native steroidal enhance the metabolic and/or inhibitory properties of the compounds of the present disclosure. Furthermore, metabolic and/or inhibitory properties are enhanced on other species, like in rabbit. The rabbit is by far the most common non-rodent species used for evaluation of reprotoxicity of small molecules. Target inhibition in the rabbit can therefore be considered an important and/or desirable feature for new compounds.

Compounds of the present disclosure show inhibition selectivity between 17β-HSD1 and 17β-HSD2. It is to be understood that compounds of the present disclosure show a higher inhibition of 17β-HSD1 than 17β-HSD2.

Compounds of the present disclosure show a 17β-HSD1 inhibition of at least 40%, preferably at least 60%, more preferably at least 70%, even more preferably at least 80%, most preferably at least 90%. The term “17β-HSD1 inhibition” as used herein and hereafter refers to inhibition of 17β-HSD1 by a compound (with a concentration of 100 nM) of the present disclosure determined with a method disclosed in chapter “Pharmacological tests” of the present disclosure.

In addition, or alternatively, compounds of the present disclosure show a 17β-HSD2 inhibition equal to or less than 40%, preferably equal to or less than 20%, more preferably equal to or less than 10%. The term “17β-HSD2 inhibition” as used herein and hereafter refers to inhibition of 17β-HSD2 by a compound (with a concentration of 1 μM) of the present disclosure determined with a method disclosed in chapter “Pharmacological tests” of the present disclosure.

In addition, or alternatively, compounds of the present disclosure show a metabolic stability corresponding to a T1/2 of at least 5 min, preferably at least 10 min, more preferably at least 20 min, even more preferably at least 40 min, still even more preferably at least 80 min, even more preferably at least 100 min, most preferably at least 140 min. The term “metabolic stability” as used herein and hereafter refers to susceptibility of compounds of the present disclosure to biotransformation. Example of metabolic stability include, but is not limited to, in vitro metabolic stability determined by using human hepatocyte incubation of a compound (with a concentration of 1 μM) of the present disclosure and expressed by the half life (T1/2, min), determined with a method disclosed in chapter “Pharmacological tests” of the present disclosure.

In addition, or alternatively, compounds of the present disclosure show inhibition in other species, wherein the inhibition is at least 10%, more preferably at least 20%, even more preferably at least 40%, most preferably at least 50%. The term “inhibition in other species” as used herein and hereafter refers to 17β-HSD1 inhibition in other species than human by a compound of the present disclosure. Examples of other species include, but is not limited to, rabbit, rat, mouse, pig, and dog. Example of inhibition in other species include, but is not limited to, the inhibition of E1 to E2 conversion in rabbit placenta tissue by a compound (with a concentration of 100 nM) of the present disclosure determined with a method disclosed in chapter “Pharmacological tests” of the present disclosure.

It is to be understood that the combination of the features of 17β-HSD1 inhibition, 17β-HSD2 inhibition, metabolic stability and/or inhibition in other species may be desirable. Therefore, the novel and inventive compounds of the present disclosure may exhibit a superior combination of said features.

The term “halogen” as used herein and hereafter by itself or as part of other groups refers to the Group VIIa elements and includes F, Cl, Br and I groups.

The term “alkyl” as used herein and hereafter is an aliphatic linear, branched or cyclic, especially linear or branched, hydrocarbon group having the indicated number of carbon atoms, for example C₁₋₆-alkyl has 1 to 6 carbon atoms in the alkyl moiety and thus, for example, C₁₋₄-alkyl includes methyl, ethyl, n-propyl, isopropyl, n-butyl, sec-butyl, isobutyl, tert-butyl and C₁₋₆-alkyl additionally includes branched and straight chain pentyl and hexyl.

The term “alicycle” as used herein and hereafter refers to a cyclic aliphatic hydrocarbon, which is a hydrocarbon group with a ring structure with only carbon atoms forming the ring structure. The alicycle can be saturated, partially unsaturated or unsaturated.

The term “(per)haloalkyl” as used herein and hereafter refers to any of the above alkyl groups where one or more hydrogen atoms are replaced by halogen(s): in particular I, Br, F or Cl. Examples of haloalkyl groups include without limitation chloromethyl, fluoromethyl and —CH₂CF₃. The term “perhaloalkyl” is understood to refer to an alkyl group, in which all the hydrogen atoms are replaced by halogen atoms. Preferred examples include trifluoromethyl (—CF₃) and trichloromethyl (—CCl₃).

The term “C₁₋₃-alkoxy” as used herein and hereafter refers to a —O—(C₁₋₃-alkyl) group where the “C₁₋₃-alkyl” has the above-defined meaning. Examples of preferred alkoxy groups include, but are not limited to, methoxy, ethoxy, and isopropyloxy.

The term “sulfonyl” as used herein and hereafter refers to a sulfonyl group having the general structure —S(═O)₂— or —SO₂— where the sulfur (S) is attached to two separate carbon atoms and the sulfur is substituted with two oxogroups. The sulfonyl group can also be part of a ring structure with the carbon atoms to which it is attached. The ring structure can include only carbon atoms in addition to the sulfonyl group or other heteroatoms such as but not limited to nitrogen, oxygen and sulfur.

The term “6 membered saturated heterocycle containing 1 to 3 heteroatom(s) independently selected from the group consisting of nitrogen, oxygen and sulfur”, refers to a monocyclic ring, which is saturated and has 4 to 6 ring atoms, and comprises 1 heteroatom selected from N, S and O while the remaining ring atoms are carbon atoms. It may be substituted with one or two substituent(s) as denoted, in particular one, at any suitable ring atom, including N. Preferred substituent groups include, but are not limited to halogen, in particular fluoro, CN, methoxy, and methyl.

The term “4 to 6 membered unsubstituted saturated heterocycle containing 1 heteroatom selected from the group consisting of nitrogen, sulfur, and oxygen”, refers to a monocyclic ring, which is saturated and has 4 to 6 ring atoms, and comprises 1 heteroatom selected from N, S and O while the remaining ring atoms are carbon atoms. Representing groups include oxetanyl, pyrrolidinyl, piperidinyl, and tetrahydropyranyl, in particular oxetanyl and tetrahydropyranyl.

The term “5 membered partially unsaturated heterocycle comprising 1 to 3 heteroatom(s) selected from the group consisting of nitrogen, sulfur, and oxygen” refers to a monocyclic ring which is partially unsaturated with 5 ring atoms comprising at least one double bond between the ring atoms and contains 1 to 3 heteroatom(s) selected from the group consisting of N, S and O, while the remaining ring atoms are carbon atoms. It may be substituted with one or two substituents as denoted, in particular one, at any suitable ring atom, including N. Preferred substituent groups include, but are not limited to halogen, in particular fluoro, CN, methoxy, and methyl.

The term “5 membered unsubstituted unsaturated or aromatic heterocycle containing 1 to 3 heteroatom(s) independently selected from the group consisting of nitrogen, sulfur, and oxygen” refers to a monocyclic ring with 5 ring atoms and which may be aromatic or unsaturated and which contains 1 to 3 heteroatom(s) independently selected from N, S and O, while the remaining ring atoms are carbon atoms.

The term “5 membered unsaturated or aromatic heterocycle” refers to a monocyclic ring with 5 ring atoms and which may be aromatic or unsaturated and comprises 1 to 3 heteroatom(s) independently selected from the group consisting of N, and O, while the remaining ring atoms are carbon atoms. It may be substituted with one or two substituents as denoted, in particular one, at any suitable ring atom, including N. Preferred substituent groups include, but are not limited to halogen, in particular fluoro, CN, methoxy, and methyl. Representing groups include oxazolyl and methyloxazolyl.

The term “6 membered unsaturated or aromatic heterocycle comprising 1 to 3 further heteroatom(s) independently selected from the group consisting of nitrogen, and oxygen” refers to a monocyclic ring with 6 ring atoms and which may be aromatic or unsaturated containing 1 to 3 heteroatom(s) independently selected from the group consisting of N, S, and O, while the remaining ring atoms are carbon atoms. It may be substituted with one or two, preferably one, substituents as denoted, in particular one, at any suitable ring atom, including N. Preferred substituent groups include, but are not limited to halogen, in particular fluoro, CN, methoxy, and methyl. Advantageously the substituent is at the para- and meta positions of the ring. Representing groups include pyridinyl, fluoropyridinyl, cyanopyridinyl, methylpyridinyl, dimethylpyridinyl, isopropylpyridinyl, hydroxypyridinyl, methoxypyridinyl, morpholinopyridinyl, methylpiperazinylpyridinyl, pyrazinyl, methylpyridazinyl, and methoxypyridazinyl; in particular fluoropyridinyl, methoxypyridinyl, methylpyridazinyl, and methoxypyridazinyl.

The term “a 5 to 6 membered saturated heterocycle comprising nitrogen atom”, refers to a saturated monocyclic ring with 6 ring atoms and contains 1 nitrogen atom while the remaining ring atoms are carbon atoms. It may be substituted with one or two substituent(s) as denoted, in particular one, at any suitable ring atom, including N. Preferred substituent groups include, but are not limited to halogen, in particular fluoro, CN, methoxy, and methyl. Representing groups include pyrrolidinyl, and methoxymethylpyrrolidinyl.

The term “an unsubstituted bicyclic spirocyclic or fused heterocycle comprising said nitrogen atom and optionally 1 or 2 further heteroatom(s) selected from a group consisting of nitrogen, oxygen and sulfur” refers to a bicyclic ring system where the rings may be joined together as a spirocyclic system or as a fused system, preferably as a spirocyclic system, and contains a nitrogen atom and optionally 1 or 2 further heteroatom(s) selected from N, O and S as indicated while the remaining ring atoms are carbon atoms. Representing groups include oxaazaspiro[4.5]decanyl.

The term “a 5 or 6 membered saturated fused ring” refers to a fused ring, which is saturated or partly unsaturated and adds 3 to 4, accordingly, additional ring atoms to the original ring into which is fused and optionally comprises 1 to 3 heteroatoms each independently selected from N, S and O while the remaining ring atoms are carbon atoms.

The term “optionally substituted” as used herein and hereafter in context of a phenyl group denotes phenyl that is either unsubstituted or substituted independently with one or more, in particular 1, 2, or 3, substituent(s) attached at any available atom to produce a stable compound, e.g. pyridinyl may be substituted once with a denoted substituent attached to any suitably position of the pyridinyl ring. In general “substituted” refers to a substituent group as defined herein in which one or more bonds to a hydrogen atom contained therein are replaced by a bond to a non-hydrogen atom unless otherwise denoted. In particular the substituent groups are each independently selected from the group consisting of halogen, in particular F; C1-4-alkyl, in particular methyl; OH; C1-4-alkoxy, in particular methoxy; and CN.

“Optional” or “optionally” denotes that the subsequently described event or circumstance may but need not occur, and that the description includes instances where the event or circumstance occurs and instances in which it does not. “Comprises” or “comprising” denotes that the subsequently described set may but need not include other elements.

The expression “pharmaceutically acceptable” represents being useful in the preparation a pharmaceutical composition that is generally safe, non-toxic, and neither biologically nor otherwise undesirable, and includes being useful for both veterinary use as well as human pharmaceutical use.

The expression “acid addition salt” includes any non-toxic organic and inorganic acid addition salts that compounds of formula (I) can form. Illustrative inorganic acids, which form suitable salts, include, but are not limited to, hydrogen chloride, hydrogen bromide, sulphuric and phosphoric acids. Illustrative organic acids, which form suitable salts, include, but are not limited to, acetic acid, lactic acid, malonic acid, succinic acid, glutaric acid, fumaric acid, malic acid, tartaric acid, citric acid, ascorbic acid, maleic acid, benzoic acid, phenylacetic acid, cinnamic acid, methane sulfonic acid, salicylic acid, and the like. The term “acid addition salt” as used herein also comprises solvates which the compounds and salts thereof are able to form, such as, for example, hydrates, alcoholates, and the like. These salts also include salts useful for the chiral resolution of racemates.

The expression “base addition salt” includes any non-toxic base addition salts that the compound of formula (I) can form. Suitable base salts include, but are not limited to, those derived from inorganic bases such as aluminum, ammonium, calcium, copper, iron, lithium, magnesium, manganese, potassium, sodium, and zinc salts, in particular sodium and ammonium salts. Further examples of organic base addition salt include salts of trialkylamines, such as triethyl amine and trimethyl amine, and choline salts.

The present invention relates to novel compounds of formula (I)

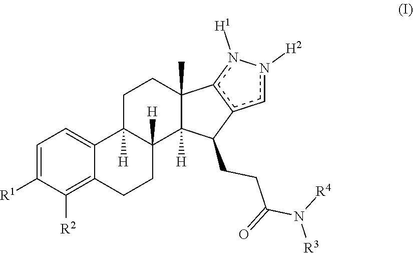

wherein

R1 and R2 are each independently selected from the group consisting of H and halogen;

(i) R3 is selected from the group consisting of H and C1-4 alkyl and

R4 is selected from the group consisting of
C1-6-alkyl optionally substituted with OH;
—(CH₂)ₙ—R5, where n is 1 to 3 and R5 is a 3 to 7 membered alicycle;
4 to 7 membered unsubstituted saturated alicycle or unsubstituted heterocycle comprising one heteroatom selected from the group consisting of nitrogen, sulfur, and oxygen;
5 membered partially unsaturated heterocycle or aromatic heterocycle comprising 1 to 3 heteroatom(s) selected from the group consisting of nitrogen, sulfur, and oxygen, and being optionally substituted with one or two substituents selected from the group consisting of halogen, CN, C1-4-alkyl, C1-3-(per)haloalkyl, OH, C1-3-alkoxy, C(O)N(C1-3-alkyl)₂, and 6 membered saturated heterocycle comprising 1 to 3 heteroatom(s) independently selected from the group consisting of nitrogen, oxygen and sulfur and being optionally substituted with one or two substituent(s) independently selected from the group consisting of halogen, CN, C1-4-alkyl, C1-3-(per)haloalkyl, OH, and C1-3-alkoxy;
5 membered unsubstituted unsaturated or aromatic heterocycle comprising 1 to 3 heteroatom(s) independently selected from the group consisting of nitrogen, sulfur, and oxygen;
5 membered unsaturated or aromatic heterocycle comprising 1 to 3 heteroatom(s) independently selected from the group consisting of nitrogen, and oxygen, and being optionally substituted with one or two substituent(s) independently selected from the group consisting of halogen, CN, C1-4-alkyl, C1-3-(per)haloalkyl, OH, C1-3-alkoxy, C(O)N(C1-3-alkyl)₂, and 6 membered saturated heterocycle containing 1 to 3 heteroatom(s) independently selected from the group consisting of nitrogen, oxygen and sulfur and being optionally substituted with one or two substituent(s) independently selected from the group consisting of halogen, CN, C1-4 alkyl, C1-3-(per)haloalkyl, OH, and C1-3-alkoxy;
phenyl, 6 membered unsaturated, or aromatic heterocycle comprising 1 to 3 heteroatom(s) independently selected from the group consisting of nitrogen, sulfur, and oxygen, and being optionally substituted with one to five substituent(s) independently selected from the group consisting of halogen, CN, C1-4 alkyl, C1-3-(per)haloalkyl, OH, oxo, C1-3-alkoxy, morpholino, C(O)N(C1-3-alkyl)₂ and a 6 membered saturated heterocycle with 1 to 3 heteroatom(s) selected from the group consisting of nitrogen, oxygen and sulfur, optionally substituted with C1-4 alkyl; and
6 membered saturated heterocycle comprising 1 to 3 heteroatom(s) independently selected from the group consisting of nitrogen, oxygen and sulfur and being optionally substituted with one to three substituent(s) independently selected from the group consisting of halogen, CN, C1-4-alkyl, C1-3-(per)haloalkyl, OH, oxo, and C1-3-alkoxy, or two adjacent substituents may form a 5 or 6 membered saturated fused ring;

or

(ii) R3 and R4 form together with the nitrogen atom they are attached to a group selected from a 4 to 7 membered saturated heterocycle comprising said nitrogen atom and optionally one additional heteroatom selected from the group consisting of nitrogen, oxygen and sulfur including SO₂, and being optionally substituted with a substituent selected from the group consisting of halogen, CN, methyl, C1-3-(per)haloalkyl, OH, oxo, C1-3-alkoxy and a 4 to 7 membered alicycle or saturated heterocycle with 1 to 3 heteroatoms selected from the group consisting of nitrogen, oxygen and sulfur, optionally substituted with C1-4-alkyl; or

(iii) R3 and R4 form together with the nitrogen atom they are attached to a group selected from a 4 to 7 membered saturated heterocycle comprising said nitrogen atom and said saturated heterocycle forms a fused or spirocycle ring with a 4 to 7 membered saturated or unsaturated alicycle or heterocycle with 1 to 3 heteroatoms selected from the group nitrogen, oxygen and sulfur;

provided that only one of hydrogens H¹ and H² is present at the same time, and the position of the double bonds in the pyrazole ring to which the hydrogens H¹ and H² are attached is determined based on which of the hydrogen H¹ and H² is present

or a pharmaceutically acceptable salt thereof.

In one embodiment of the invention the pyrazole ring of the compound is in the form shown in formula (Ia),

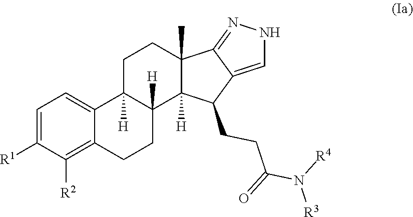

in which formula (Ia) R1, R2, R3, R4 and R5 are as defined above.

In one embodiment of the invention substituent R1 is selected from the group consisting of H, F and Cl, substituent R2 is selected from the group consisting of H, F and Cl, and substituents R3, R4 and R5 are as defined above.

In one embodiment of the invention substituent R1 is selected from the group consisting of H, F and Cl, substituent R2 is H or F, and substituents R3, R4 and R5 are as defined above.

In one embodiment of the invention substituent R1 is H, substituent R2 is F and substituents R3, R4 and R5 are as defined above.

In one embodiment of the invention substituent R3 is H and substituent R4 is a 6 membered unsaturated or aromatic heterocycle with 1 to 3 heteroatoms selected from the group consisting of nitrogen, oxygen and sulfur, optionally substituted with one or two substituent(s) selected from the group consisting of CN, C1-4 alkyl, C1-3-alkoxy, halogen and C(O)N(C1-3-alkyl)₂ or alternatively R4 is a substituent with formula:

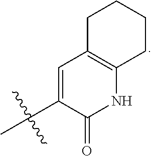 [SMILES: *c1cc2c([nH]c1=O)CCCC2]

In one embodiment of the invention substituent R3 is H and substituent R4 is a 5 membered unsaturated or aromatic heterocycle with 1 to 3 heteroatoms selected from the group consisting of nitrogen, oxygen and sulfur, optionally substituted with one or two substituent(s) selected from the group consisting of CN, C1-4-alkyl, C1-3-alkoxy, halogen and C(O)N(C1-3-alkyl)₂.

In one embodiment of the invention substituent R4 is a 5 membered unsubstituted unsaturated or aromatic heterocycle comprising 1 to 3 heteroatom(s), wherein the 1 to 3 heteroatoms of the 5 membered unsubstituted unsaturated or aromatic heterocycle are independently selected from nitrogen and oxygen, the 1 to 3 heteroatoms of the 5 membered unsubstituted unsaturated or aromatic heterocycle are independently selected from 2 nitrogens and 1 sulphur, or the 1 to 3 heteroatoms of the 5 membered unsubstituted unsaturated or aromatic heterocycle are independently selected from 2 nitrogens and 1 oxygen, and substituents R1, R2, and R3 are as defined above, or a pharmaceutically acceptable salt thereof.

In one embodiment of the invention substituent R1 is selected from the group consisting of H, Cl or F, substituent R2 is selected from the group consisting of H, Cl or F, substituent R3 is H, and substituent R4 is a 6 membered aromatic heterocycle with 1 to 3 heteroatoms selected from the group consisting of nitrogen, oxygen and sulfur, optionally substituted with one or two substituent(s) selected from the group consisting of CN, C1-4 alkyl, C1-3-alkoxy, halogen and C(O)N(C1-3-alkyl)₂, or alternatively substituent R4 is a substituent with formula:

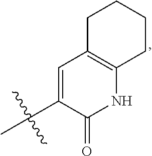 [SMILES: *c1cc2c([nH]c1=O)CCCC2]

or a pharmaceutically acceptable salt thereof.

In one embodiment of the invention substituent R1 is H or F, substituent R2 is H or F, substituent R3 is H, and substituent R4 is a 6 membered aromatic heterocycle with 1 to 3 heteroatoms selected from the group consisting of nitrogen, oxygen and sulfur, optionally substituted with one or two substituent(s) selected from the group consisting of CN, C1-4 alkyl, C1-3-alkoxy, halogen and C(O)N(C1-3-alkyl)₂, or alternatively substituent R4 is a substituent with formula:

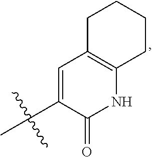 [SMILES: *c1cc2c([nH]c1=O)CCCC2]

or a pharmaceutically acceptable salt thereof.

In one embodiment of the invention substituent R1 is H, substituent R2 is H or F, substituent R3 is H, substituent R4 is a 6 membered aromatic heterocycle with 1 to 3 heteroatoms selected from the group consisting of nitrogen, oxygen and sulfur, optionally substituted with one or two substituent(s) selected from the group consisting of CN, C1-4-alkyl, C1-3-alkoxy, halogen and C(O)N(C1-3-alkyl)₂, or a pharmaceutically acceptable salt thereof.

In one embodiment of the invention substituent R1 is H, substituent R2 is F, substituent R3 is H, substituent R4 is a 6 membered aromatic heterocycle with 1 to 3 heteroatoms selected from the group consisting of nitrogen, oxygen and sulfur, optionally substituted with one or two substituent(s) selected from the group consisting of CN, methyl, methoxy, F and C(O)N(methyl)₂, or alternatively substituent R4 is a substituent with formula:

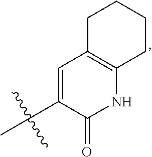 [SMILES: *c1cc2c([nH]c1=O)CCCC2]

or a pharmaceutically acceptable salt thereof.

In one embodiment of the invention the compound of formula (I) is a compound selected from the list:
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(6-methoxypyridazin-3-yl)-propanamide,
N-(5-Cyanopyridin-2-yl)-3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(4-fluoropyridin-2-yl)propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(5-methoxypyridin-2-yl)propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(4-methylpyridin-2-yl)propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(2-oxo-1,2,5,6,7,8-hexahydroquinolin-3-yl)propanamide,
6-(3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamido)-N,N-dimethylnicotinamide,
N-(6-methoxypyridazin-3-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide,
N,N-dimethyl-6-(3-((8aS,12S)-8a-methyl-1,2,6b,7,8,8a,10,12,12a,12b-decahydronaphtho[2′,1′:4,5]indeno[1,2-c]pyrazol-12-yl)propanamido)nicotinamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(1-methyl-1H-pyrazol-3-yl)-propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(1-methyl-1H-pyrazol-4-yl)-propanamide,
N-(5-(tert-butyl)isoxazol-3-yl)-3-((8aS,12S)-4-fluoro-8a-methyl-1,2,6b,7,8,8a,10,12,12a,12b-decahydronaphtho[2′,1′:4,5]indeno[1,2-c]pyrazol-12-yl)propanamide,
N-(5-fluoropyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide,
N-(4-fluoropyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide and
3-((8aS,12S)-8a-methyl-1,2,6b,7,8,8a,10,12,12a,12b-decahydronaphtho[2′,1′:4,5]indeno[1,2-c]pyrazol-12-yl)-N-(1-methyl-1H-pyrazol-3-yl)propanamide, or a pharmaceutically acceptable salt thereof.

The invention also relates to a method for the preparation of a compound according to formula (I) in which method a compound of formula (II) is reacted with hydrazine hydrate to form a compound according to formula (I).

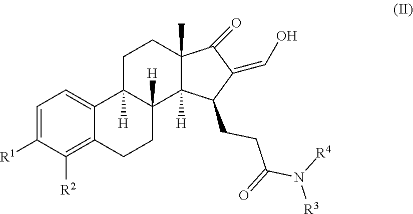

The method for preparing the compound according to the invention having formula (I) involves more specifically a method where the compound of formula (II) is dissolved in methanol (1.5 mL). Hydrazine hydrate (200 mol %) is added and stirred at +50° C. under nitrogen 30 minutes. The solvent is evaporated. Evaporation residue is dissolved in ethyl acetate, washed trice with 1N hydrochloric acid. The aqueous layers are combined and then is washed with ethyl acetate, finally the aqueous layer is neutralized (pH˜8) and the product is extracted with ethyl acetate. The product is purified by chromatography or by crystallization. More details of the various methods of preparation for the compound according to the invention can be found in the Examples.

The current invention also relates to the intermediate according to formula (II).

Further the invention relates to compounds of formula (I) for use as a medicament. Specifically, the medicament can be for use in treatment or prevention of a disease selected from a group consisting of breast cancer, prostate carcinoma, ovarian cancer, uterine cancer, endometrial cancer, endometrial hyperplasia, endometriosis, uterine fibroids, adenomyosis, polycystic ovarian syndrome, dysmenorrhea, menorrhagia, metrorrhagia, contraception, prostadynia, benign prostatic hyperplasia, urinary dysfunction, lower urinary tract symptoms, chronic prostatitis/chronic pelvic pain syndrome (CP/CPPS), systemic lupus erythematosus (SLE), multiple sclerosis, obesity, rheumatoid arthritis, chronic obstructive pulmonary disease (COPD), lung cancer, colon cancer, tissue wounds, skin wrinkles and cataracts.

Still further the invention relates to a pharmaceutical composition comprising an effective amount of one or more compound(s) of formula (I), together with one or more pharmaceutically acceptable excipient(s). The pharmaceutical composition can also comprise one or more other active ingredients.

Representative examples of compounds of formula (I) are shown in Table 1.

TABLE 1

| | |
|---|---|
| 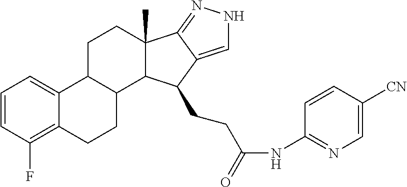 [SMILES: C[C@]12CCC3c4cccc(F)c4CCC3C1[C@H](CCC(=O)Nc1ccc(C#N)cn1)c1c[nH]nc12] | 3 |
| 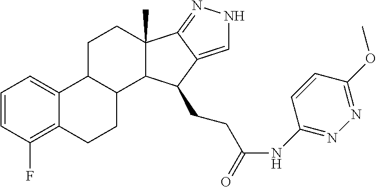 [SMILES: COc1ccc(NC(=O)CC[C@@H]2c3c[nH]nc3[C@@]3(C)CCC4c5cccc(F)c5CCC4C23)nn1] | 6 |
| 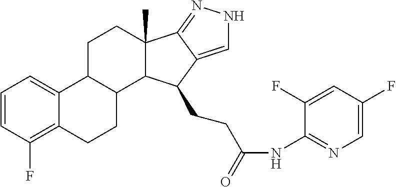 [SMILES: C[C@]12CCC3c4cccc(F)c4CCC3C1[C@H](CCC(=O)Nc1ncc(F)cc1F)c1c[nH]nc12] | 9 |
| 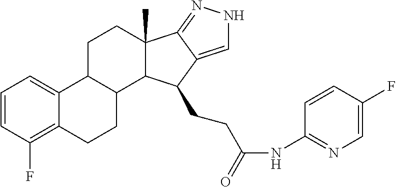 [SMILES: C[C@]12CCC3c4cccc(F)c4CCC3C1[C@H](CCC(=O)Nc1ccc(F)cn1)c1c[nH]nc12] | 12 |
| 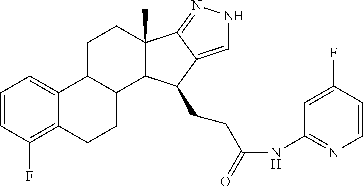 [SMILES: C[C@]12CCC3c4cccc(F)c4CCC3C1[C@H](CCC(=O)Nc1cc(F)ccn1)c1c[nH]nc12] | 15 |
| 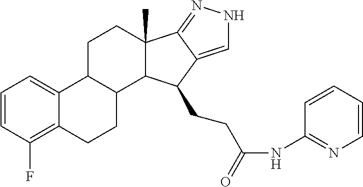 [SMILES: C[C@]12CCC3c4cccc(F)c4CCC3C1[C@H](CCC(=O)Nc1ccccn1)c1c[nH]nc12] | 18 |
| 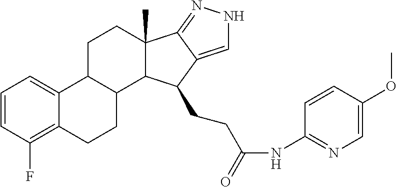 [SMILES: COc1ccc(NC(=O)CC[C@@H]2c3c[nH]nc3[C@@]3(C)CCC4c5cccc(F)c5CCC4C23)nc1] | 21 |
| 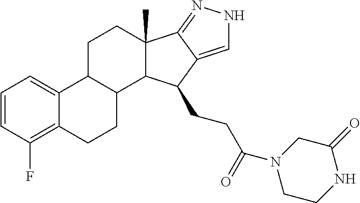 [SMILES: C[C@]12CCC3c4cccc(F)c4CCC3C1[C@H](CCC(=O)N1CCNC(=O)C1)c1c[nH]nc12] | 24 |
| 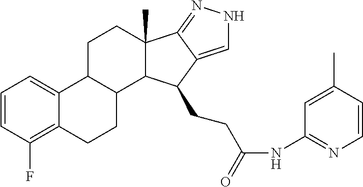 [SMILES: Cc1ccnc(NC(=O)CC[C@@H]2c3c[nH]nc3[C@@]3(C)CCC4c5cccc(F)c5CCC4C23)c1] | 27 |
| 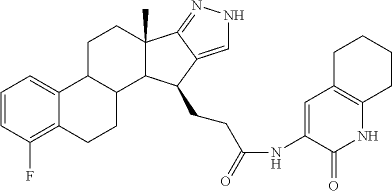 [SMILES: C[C@]12CCC3c4cccc(F)c4CCC3C1[C@H](CCC(=O)Nc1cc3c([nH]c1=O)CCCC3)c1c[nH]nc12] | 30 |
| 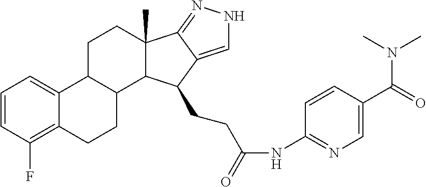 [SMILES: CN(C)C(=O)c1ccc(NC(=O)CC[C@@H]2c3c[nH]nc3[C@@]3(C)CCC4c5cccc(F)c5CCC4C23)nc1] | 33 |
| 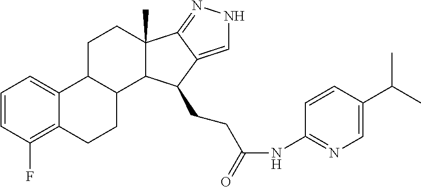 [SMILES: CC(C)c1ccc(NC(=O)CC[C@@H]2c3c[nH]nc3[C@@]3(C)CCC4c5cccc(F)c5CCC4C23)nc1] | 36 |
| 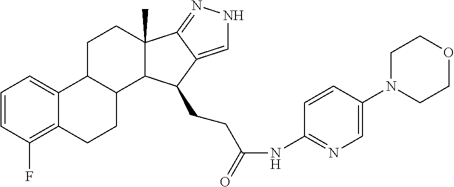 [SMILES: C[C@]12CCC3c4cccc(F)c4CCC3C1[C@H](CCC(=O)Nc1ccc(N3CCOCC3)cn1)c1c[nH]nc12] | 39 |
| 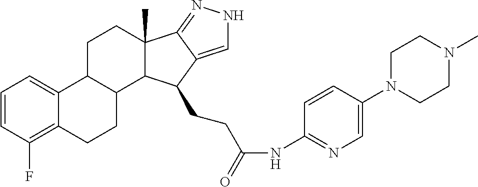 [SMILES: CN1CCN(c2ccc(NC(=O)CC[C@@H]3c4c[nH]nc4[C@@]4(C)CCC5c6cccc(F)c6CCC5C34)nc2)CC1] | 42 |
| 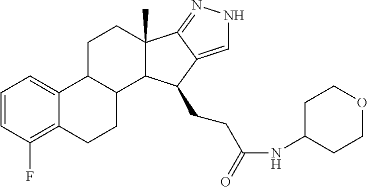 [SMILES: C[C@]12CCC3c4cccc(F)c4CCC3C1[C@H](CCC(=O)NC1CCOCC1)c1c[nH]nc12] | 45 |
| 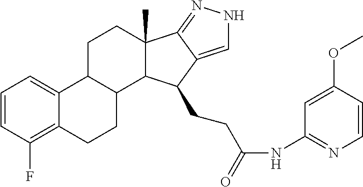 [SMILES: COc1ccnc(NC(=O)CC[C@@H]2c3c[nH]nc3[C@@]3(C)CCC4c5cccc(F)c5CCC4C23)c1] | 48 |
| 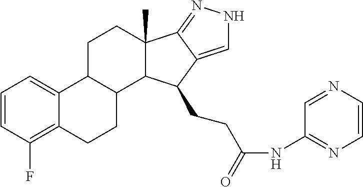 [SMILES: C[C@]12CCC3c4cccc(F)c4CCC3C1[C@H](CCC(=O)Nc1cnccn1)c1c[nH]nc12] | 51 |
| 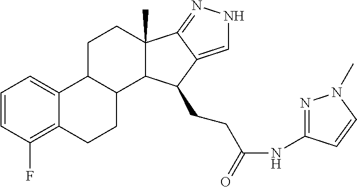 [SMILES: Cn1ccc(NC(=O)CC[C@@H]2c3c[nH]nc3[C@@]3(C)CCC4c5cccc(F)c5CCC4C23)n1] | 54 |
| 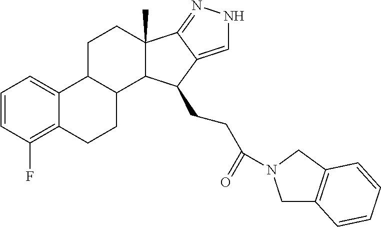 [SMILES: C[C@]12CCC3c4cccc(F)c4CCC3C1[C@H](CCC(=O)N1Cc3ccccc3C1)c1c[nH]nc12] | 57 |
| 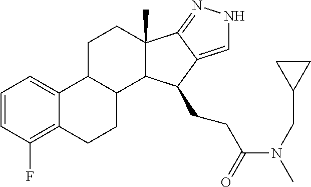 [SMILES: CN(CC1CC1)C(=O)CC[C@@H]1c2c[nH]nc2[C@@]2(C)CCC3c4cccc(F)c4CCC3C12] | 60 |
| 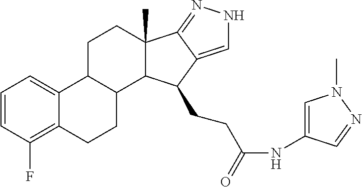 [SMILES: Cn1cc(NC(=O)CC[C@@H]2c3c[nH]nc3[C@@]3(C)CCC4c5cccc(F)c5CCC4C23)cn1] | 63 |
| 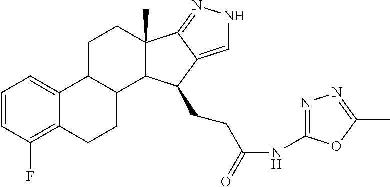 [SMILES: Cc1nnc(NC(=O)CC[C@@H]2c3c[nH]nc3[C@@]3(C)CCC4c5cccc(F)c5CCC4C23)o1] | 66 |
| 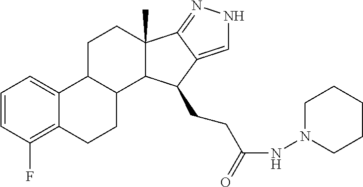 [SMILES: C[C@]12CCC3c4cccc(F)c4CCC3C1[C@H](CCC(=O)NN1CCCCC1)c1c[nH]nc12] | 69 |
| 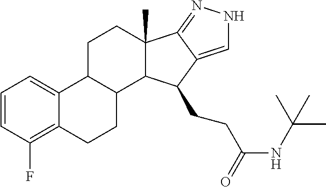 [SMILES: CC(C)(C)NC(=O)CC[C@@H]1c2c[nH]nc2[C@@]2(C)CCC3c4cccc(F)c4CCC3C12] | 72 |
| 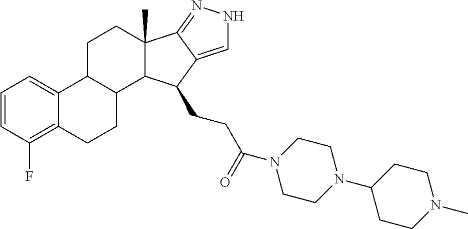 [SMILES: CN1CCC(N2CCN(C(=O)CC[C@@H]3c4c[nH]nc4[C@@]4(C)CCC5c6cccc(F)c6CCC5C34)CC2)CC1] | 75 |
| 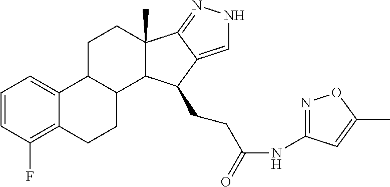 [SMILES: Cc1cc(NC(=O)CC[C@@H]2c3c[nH]nc3[C@@]3(C)CCC4c5cccc(F)c5CCC4C23)no1] | 78 |
| 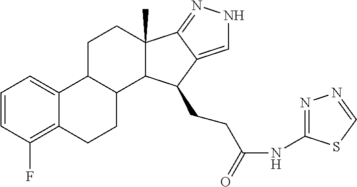 [SMILES: C[C@]12CCC3c4cccc(F)c4CCC3C1[C@H](CCC(=O)Nc1nncs1)c1c[nH]nc12] | 81 |
| 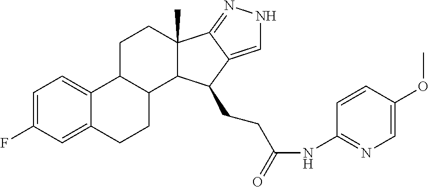 [SMILES: COc1ccc(NC(=O)CC[C@@H]2c3c[nH]nc3[C@@]3(C)CCC4c5ccc(F)cc5CCC4C23)nc1] | 84 |
| 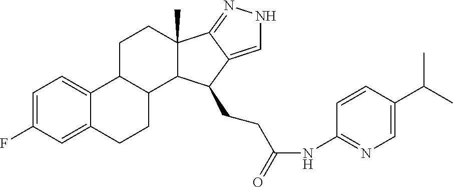 [SMILES: CC(C)c1ccc(NC(=O)CC[C@@H]2c3c[nH]nc3[C@@]3(C)CCC4c5ccc(F)cc5CCC4C23)nc1] | 87 |
| 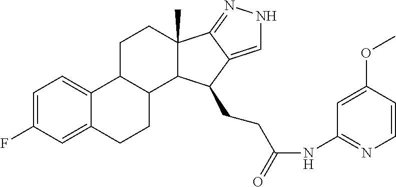 [SMILES: COc1ccnc(NC(=O)CC[C@@H]2c3c[nH]nc3[C@@]3(C)CCC4c5ccc(F)cc5CCC4C23)c1] | 90 |
| 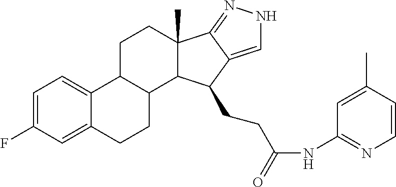 [SMILES: Cc1ccnc(NC(=O)CC[C@@H]2c3c[nH]nc3[C@@]3(C)CCC4c5ccc(F)cc5CCC4C23)c1] | 93 |
| 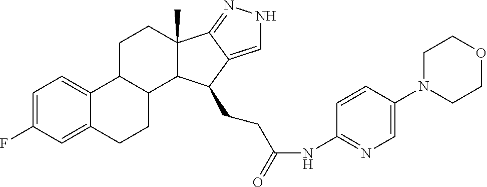 [SMILES: C[C@]12CCC3c4ccc(F)cc4CCC3C1[C@H](CCC(=O)Nc1ccc(N3CCOCC3)cn1)c1c[nH]nc12] | 96 |
| 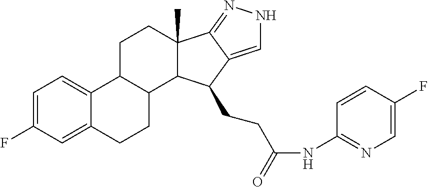 [SMILES: C[C@]12CCC3c4ccc(F)cc4CCC3C1[C@H](CCC(=O)Nc1ccc(F)cn1)c1c[nH]nc12] | 99 |
| 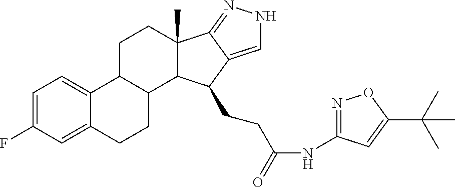 [SMILES: CC(C)(C)c1cc(NC(=O)CC[C@@H]2c3c[nH]nc3[C@@]3(C)CCC4c5ccc(F)cc5CCC4C23)no1] | 102 |
| 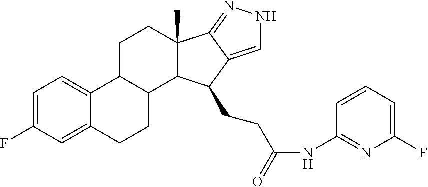 [SMILES: C[C@]12CCC3c4ccc(F)cc4CCC3C1[C@H](CCC(=O)Nc1cccc(F)n1)c1c[nH]nc12] | 105 |
| 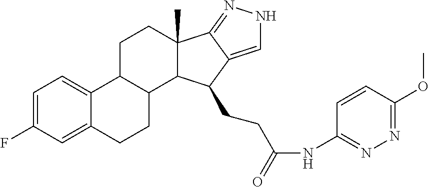 [SMILES: COc1ccc(NC(=O)CC[C@@H]2c3c[nH]nc3[C@@]3(C)CCC4c5ccc(F)cc5CCC4C23)nn1] | 108 |
| 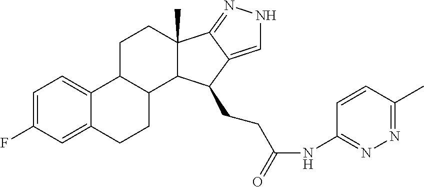 [SMILES: Cc1ccc(NC(=O)CC[C@@H]2c3c[nH]nc3[C@@]3(C)CCC4c5ccc(F)cc5CCC4C23)nn1] | 111 |
| 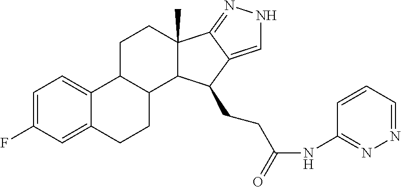 [SMILES: C[C@]12CCC3c4ccc(F)cc4CCC3C1[C@H](CCC(=O)Nc1cccnn1)c1c[nH]nc12] | 114 |
| 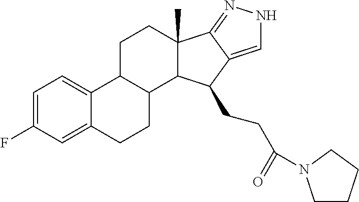 [SMILES: C[C@]12CCC3c4ccc(F)cc4CCC3C1[C@H](CCC(=O)N1CCCC1)c1c[nH]nc12] | 117 |
| 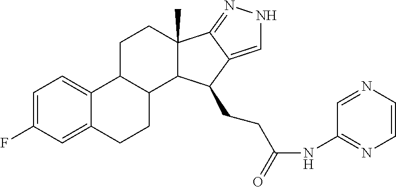 [SMILES: C[C@]12CCC3c4ccc(F)cc4CCC3C1[C@H](CCC(=O)Nc1cnccn1)c1c[nH]nc12] | 120 |
| 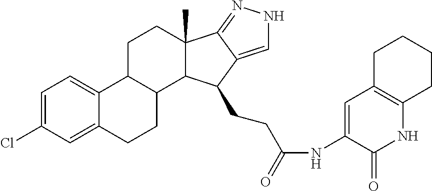 [SMILES: C[C@]12CCC3c4ccc(Cl)cc4CCC3C1[C@H](CCC(=O)Nc1cc3c([nH]c1=O)CCCC3)c1c[nH]nc12] | 123 |
| 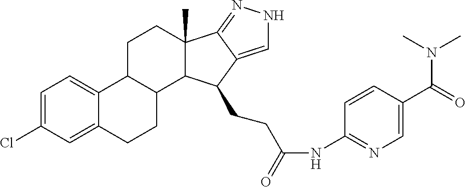 [SMILES: CN(C)C(=O)c1ccc(NC(=O)CC[C@@H]2c3c[nH]nc3[C@@]3(C)CCC4c5ccc(Cl)cc5CCC4C23)nc1] | 126 |
| 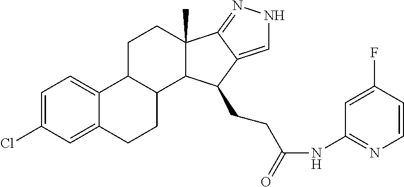 [SMILES: C[C@]12CCC3c4ccc(Cl)cc4CCC3C1[C@H](CCC(=O)Nc1cc(F)ccn1)c1c[nH]nc12] | 129 |
| 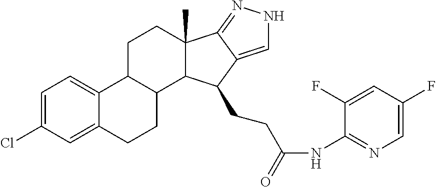 [SMILES: C[C@]12CCC3c4ccc(Cl)cc4CCC3C1[C@H](CCC(=O)Nc1ncc(F)cc1F)c1c[nH]nc12] | 132 |
| 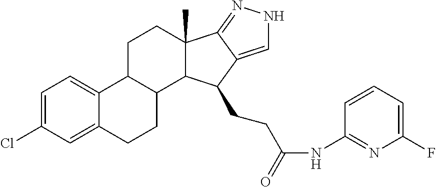 [SMILES: C[C@]12CCC3c4ccc(Cl)cc4CCC3C1[C@H](CCC(=O)Nc1cccc(F)n1)c1c[nH]nc12] | 135 |
| 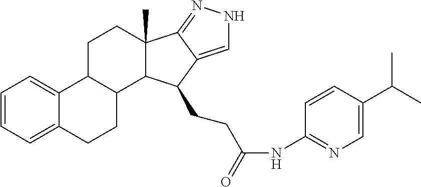 [SMILES: CC(C)c1ccc(NC(=O)CC[C@@H]2c3c[nH]nc3[C@@]3(C)CCC4c5ccccc5CCC4C23)nc1] | 138 |
| 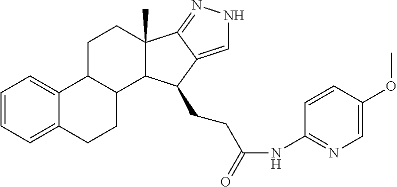 [SMILES: COc1ccc(NC(=O)CC[C@@H]2c3c[nH]nc3[C@@]3(C)CCC4c5ccccc5CCC4C23)nc1] | 141 |
| 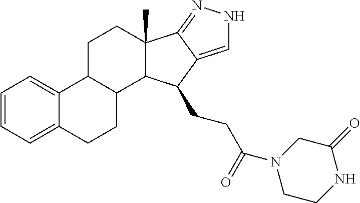 [SMILES: C[C@]12CCC3c4ccccc4CCC3C1[C@H](CCC(=O)N1CCNC(=O)C1)c1c[nH]nc12] | 144 |
| 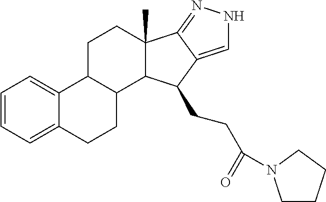 [SMILES: C[C@]12CCC3c4ccccc4CCC3C1[C@H](CCC(=O)N1CCCC1)c1c[nH]nc12] | 147 |
| 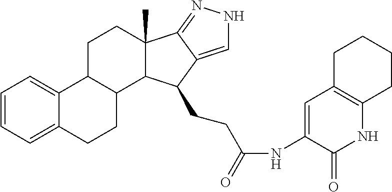 [SMILES: C[C@]12CCC3c4ccccc4CCC3C1[C@H](CCC(=O)Nc1cc3c([nH]c1=O)CCCC3)c1c[nH]nc12] | 150 |
| 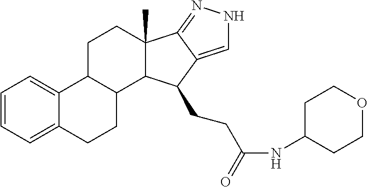 [SMILES: C[C@]12CCC3c4ccccc4CCC3C1[C@H](CCC(=O)NC1CCOCC1)c1c[nH]nc12] | 153 |
| 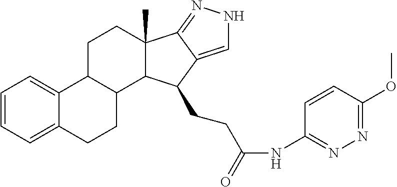 [SMILES: COc1ccc(NC(=O)CC[C@@H]2c3c[nH]nc3[C@@]3(C)CCC4c5ccccc5CCC4C23)nn1] | 156 |
| 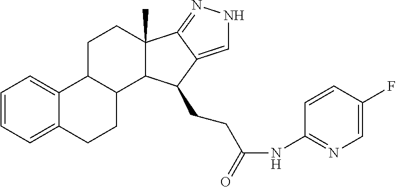 [SMILES: C[C@]12CCC3c4ccccc4CCC3C1[C@H](CCC(=O)Nc1ccc(F)cn1)c1c[nH]nc12] | 159 |
| 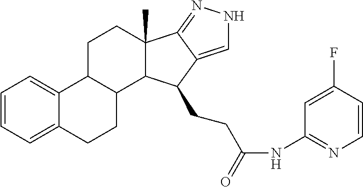 [SMILES: C[C@]12CCC3c4ccccc4CCC3C1[C@H](CCC(=O)Nc1cc(F)ccn1)c1c[nH]nc12] | 162 |
| 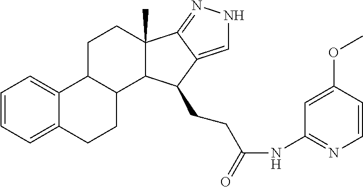 [SMILES: COc1ccnc(NC(=O)CC[C@@H]2c3c[nH]nc3[C@@]3(C)CCC4c5ccccc5CCC4C23)c1] | 165 |
| 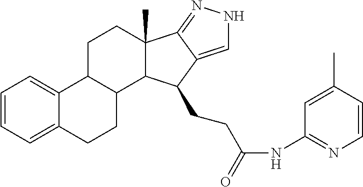 [SMILES: Cc1ccnc(NC(=O)CC[C@@H]2c3c[nH]nc3[C@@]3(C)CCC4c5ccccc5CCC4C23)c1] | 168 |
| 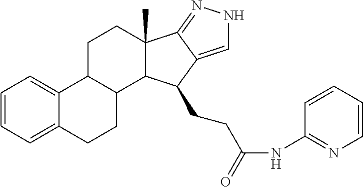 [SMILES: C[C@]12CCC3c4ccccc4CCC3C1[C@H](CCC(=O)Nc1ccccn1)c1c[nH]nc12] | 171 |
| 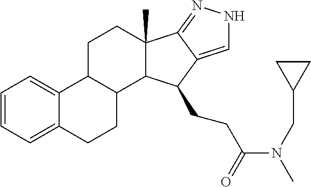 [SMILES: CN(CC1CC1)C(=O)CC[C@@H]1c2c[nH]nc2[C@@]2(C)CCC3c4ccccc4CCC3C12] | 174 |
| 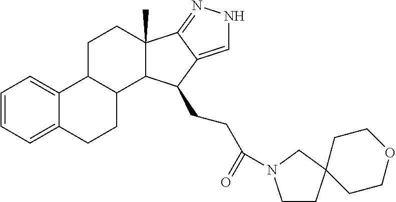 [SMILES: C[C@]12CCC3c4ccccc4CCC3C1[C@H](CCC(=O)N1CCC3(CCOCC3)C1)c1c[nH]nc12] | 177 |
| 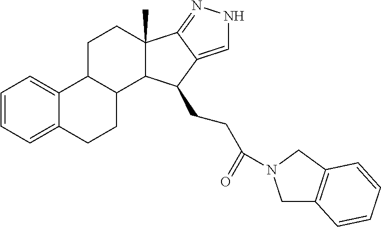 [SMILES: C[C@]12CCC3c4ccccc4CCC3C1[C@H](CCC(=O)N1Cc3ccccc3C1)c1c[nH]nc12] | 180 |
| 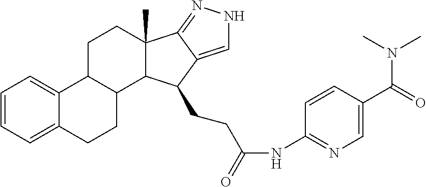 [SMILES: CN(C)C(=O)c1ccc(NC(=O)CC[C@@H]2c3c[nH]nc3[C@@]3(C)CCC4c5ccccc5CCC4C23)nc1] | 183 |
| 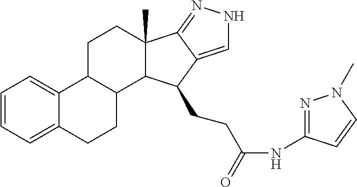 [SMILES: Cn1ccc(NC(=O)CC[C@@H]2c3c[nH]nc3[C@@]3(C)CCC4c5ccccc5CCC4C23)n1] | 186 |
| 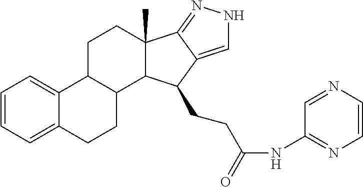 [SMILES: C[C@]12CCC3c4ccccc4CCC3C1[C@H](CCC(=O)Nc1cnccn1)c1c[nH]nc12] | 189 |
| 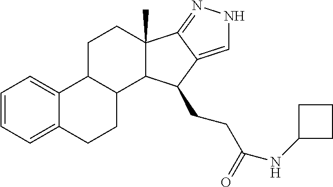 [SMILES: C[C@]12CCC3c4ccccc4CCC3C1[C@H](CCC(=O)NC1CCC1)c1c[nH]nc12] | 192 |
| 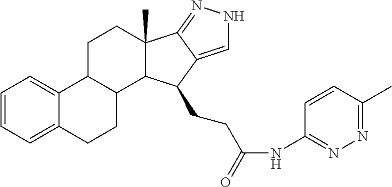 [SMILES: Cc1ccc(NC(=O)CC[C@@H]2c3c[nH]nc3[C@@]3(C)CCC4c5ccccc5CCC4C23)nn1] | 195 |
| 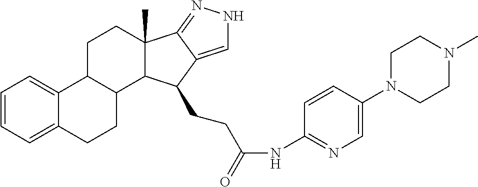 [SMILES: CN1CCN(c2ccc(NC(=O)CC[C@@H]3c4c[nH]nc4[C@@]4(C)CCC5c6ccccc6CCC5C34)nc2)CC1] | 198 |
| 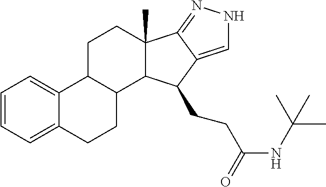 [SMILES: CC(C)(C)NC(=O)CC[C@@H]1c2c[nH]nc2[C@@]2(C)CCC3c4ccccc4CCC3C12] | 201 |
| 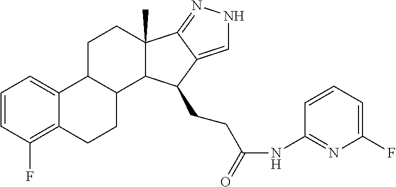 [SMILES: C[C@]12CCC3c4cccc(F)c4CCC3C1[C@H](CCC(=O)Nc1cccc(F)n1)c1c[nH]nc12] | 204 |
| 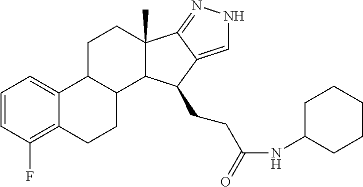 [SMILES: C[C@]12CCC3c4cccc(F)c4CCC3C1[C@H](CCC(=O)NC1CCCCC1)c1c[nH]nc12] | 207 |
| 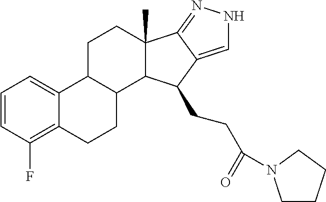 [SMILES: C[C@]12CCC3c4cccc(F)c4CCC3C1[C@H](CCC(=O)N1CCCC1)c1c[nH]nc12] | 210 |
| 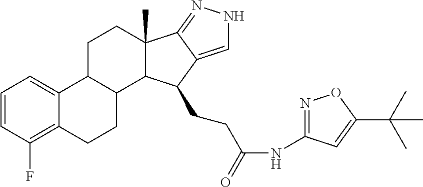 [SMILES: CC(C)(C)c1cc(NC(=O)CC[C@@H]2c3c[nH]nc3[C@@]3(C)CCC4c5cccc(F)c5CCC4C23)no1] | 213 |
| 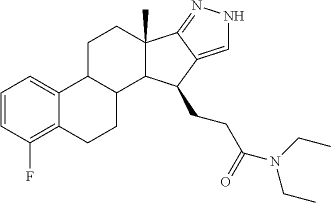 [SMILES: CCN(CC)C(=O)CC[C@@H]1c2c[nH]nc2[C@@]2(C)CCC3c4cccc(F)c4CCC3C12] | 216 |
| 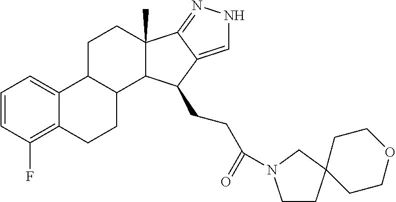 [SMILES: C[C@]12CCC3c4cccc(F)c4CCC3C1[C@H](CCC(=O)N1CCC3(CCOCC3)C1)c1c[nH]nc12] | 219 |
| 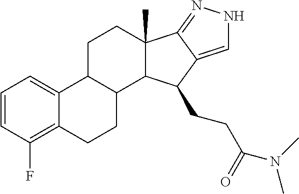 [SMILES: CN(C)C(=O)CC[C@@H]1c2c[nH]nc2[C@@]2(C)CCC3c4cccc(F)c4CCC3C12] | 222 |
| 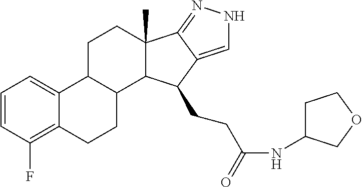 [SMILES: C[C@]12CCC3c4cccc(F)c4CCC3C1[C@H](CCC(=O)NC1CCOC1)c1c[nH]nc12] | 225 |
| 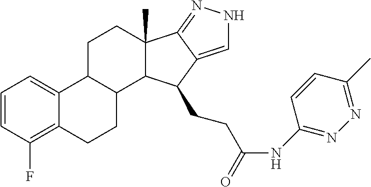 [SMILES: Cc1ccc(NC(=O)CC[C@@H]2c3c[nH]nc3[C@@]3(C)CCC4c5cccc(F)c5CCC4C23)nn1] | 228 |
| 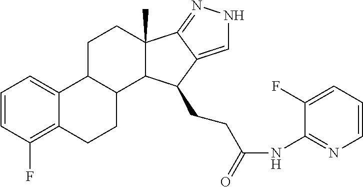 [SMILES: C[C@]12CCC3c4cccc(F)c4CCC3C1[C@H](CCC(=O)Nc1ncccc1F)c1c[nH]nc12] | 231 |
| 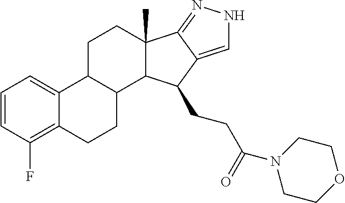 [SMILES: C[C@]12CCC3c4cccc(F)c4CCC3C1[C@H](CCC(=O)N1CCOCC1)c1c[nH]nc12] | 234 |
| 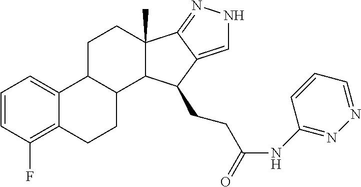 [SMILES: C[C@]12CCC3c4cccc(F)c4CCC3C1[C@H](CCC(=O)Nc1cccnn1)c1c[nH]nc12] | 237 |
| 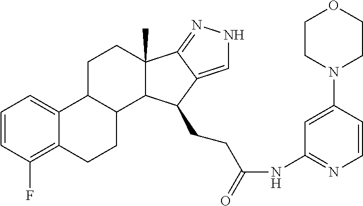 [SMILES: C[C@]12CCC3c4cccc(F)c4CCC3C1[C@H](CCC(=O)Nc1cc(N3CCOCC3)ccn1)c1c[nH]nc12] | 240 |
| 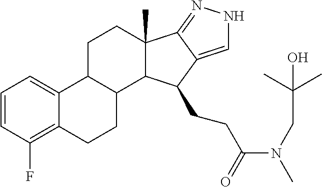 [SMILES: CN(CC(C)(C)O)C(=O)CC[C@@H]1c2c[nH]nc2[C@@]2(C)CCC3c4cccc(F)c4CCC3C12] | 243 |
| 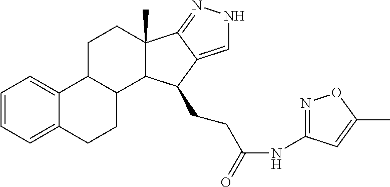 [SMILES: Cc1cc(NC(=O)CC[C@@H]2c3c[nH]nc3[C@@]3(C)CCC4c5ccccc5CCC4C23)no1] | 246 |
| 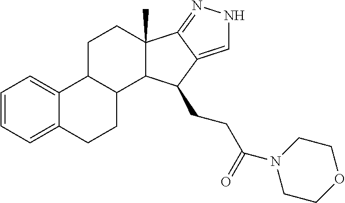 [SMILES: C[C@]12CCC3c4ccccc4CCC3C1[C@H](CCC(=O)N1CCOCC1)c1c[nH]nc12] | 249 |
| 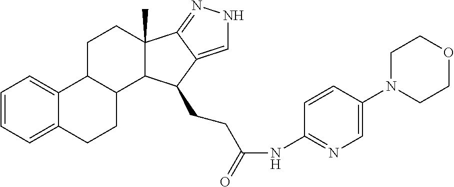 [SMILES: C[C@]12CCC3c4ccccc4CCC3C1[C@H](CCC(=O)Nc1ccc(N3CCOCC3)cn1)c1c[nH]nc12] | 252 |
| 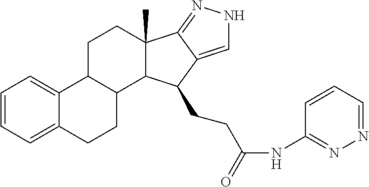 [SMILES: C[C@]12CCC3c4ccccc4CCC3C1[C@H](CCC(=O)Nc1cccnn1)c1c[nH]nc12] | 255 |
| 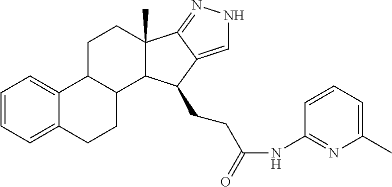 [SMILES: Cc1cccc(NC(=O)CC[C@@H]2c3c[nH]nc3[C@@]3(C)CCC4c5ccccc5CCC4C23)n1] | 258 |
| 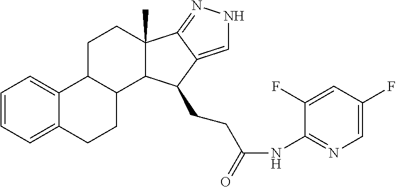 [SMILES: C[C@]12CCC3c4ccccc4CCC3C1[C@H](CCC(=O)Nc1ncc(F)cc1F)c1c[nH]nc12] | 261 |
| 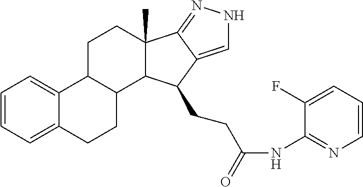 [SMILES: C[C@]12CCC3c4ccccc4CCC3C1[C@H](CCC(=O)Nc1ncccc1F)c1c[nH]nc12] | 264 |
| 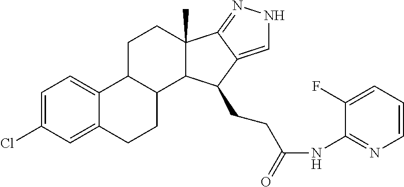 [SMILES: C[C@]12CCC3c4ccc(Cl)cc4CCC3C1[C@H](CCC(=O)Nc1ncccc1F)c1c[nH]nc12] | 267 |

### Examples of the Invention

### Preparation of Synthesis Starting Materials and Precursors

### Preparation of the Starting Material Acid IX

Compound SM-IX was synthesized from Estrone (Scheme 1). Methods of Horwitz et al U. Med. Chem., 1986, 29 (5), 692-698) yielded amine SM-III which was fluorinated using conditions of Labrie et al. WO2008124922. The fluoride SM-IV was converted to enone SM-VI by silylation/oxidation method of Kobayashi et (Tetrahedron, 71(35), 5918-5924; 2015). The allylation, hydroboration and oxidation of SM-VI to SM-IX was performed as in patents WO2005/047303 and WO2006/125800.

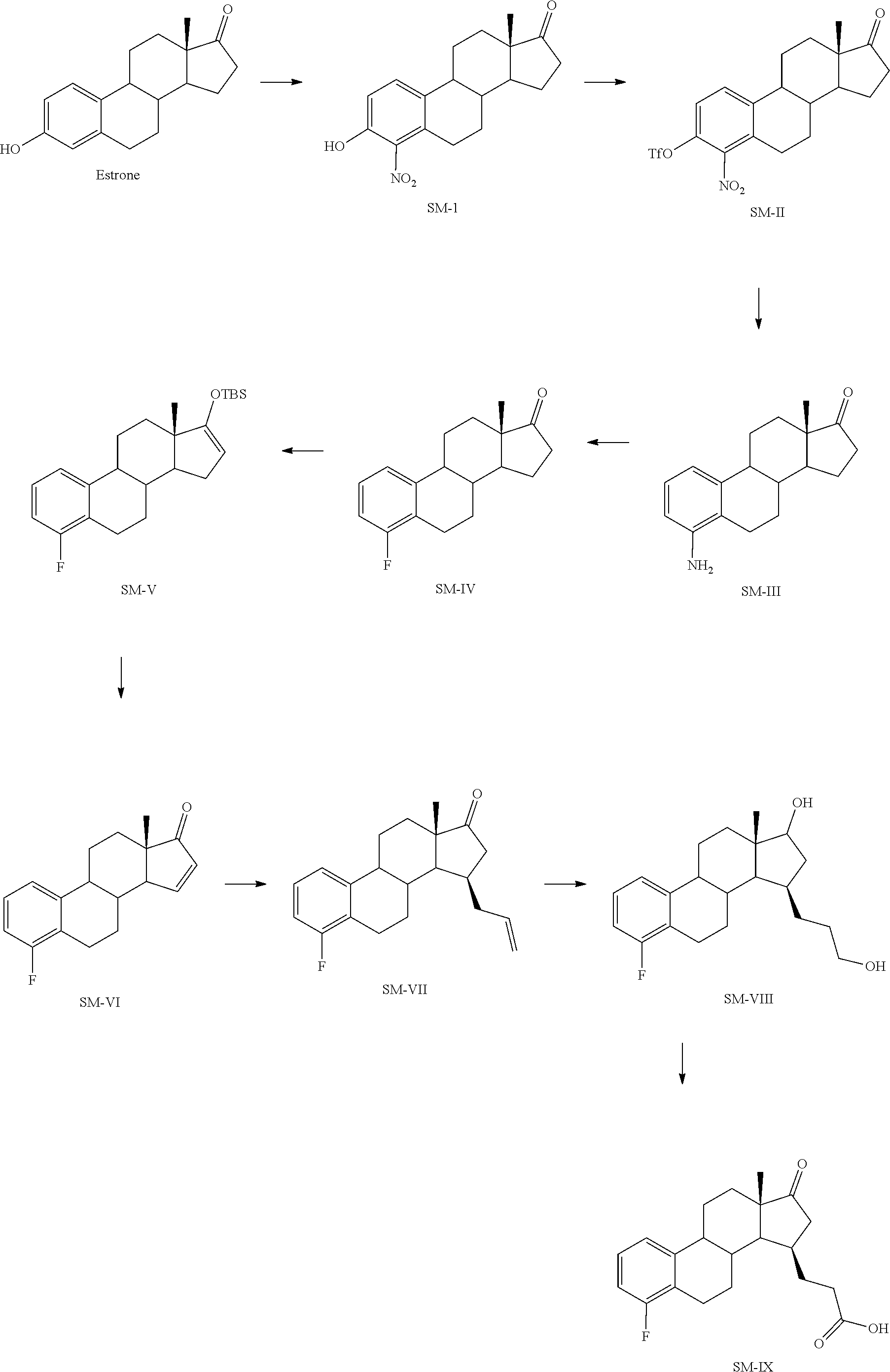

### Compound SM-IV

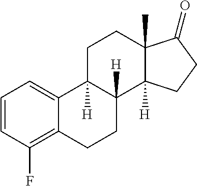

A solution of Compound SM-III (11.00 g, 40.8 mmol, 100 mol-%) in dichloromethane (430 mL) was added to neat boron trifluoride diethyl etherate (7.9 mL, 64.20 mmol, 157 mol-%) while stirring at −15° C. under nitrogen (approx 10-15 mins addition time). The reaction mixture was stirred for 15 min. at −15° C. before a solution of tert-butyl nitrite (5.9 mL, 49.80 mmol, 122 mol-%) in dichloromethane (50 mL) was added to it dropwise over a period of 10 min. The reaction mixture was stirred for another 15 min. at −15° C., and afterwards at 0-5° C. for 30 min.

The solution was added to n-pentane (2.25 L) on order to give a beige precipitate. The liquors were decanted and the residue was washed with more n-pentane (400 mL). The beige solid (12.00 g) was dried in vacuo at room temperature overnight.

The crude material was purified by flash column chromatography using n-hexanes and ethyl acetate (10-30%) as solvent system. The yield of Compound SM-IV as a cream solid was 70% (7.82 g).

¹H NMR (400 MHz, CDCl₃) δ ppm 0.91 (s, 3H, —CH₃), 1.34-1.70 (m, 6H), 1.93-1.99 (m, 1H), 2.04-2.21 (m, 3H), 2.27-2.46 (m, 2H), 2.48-2.56 (m, 1H), 2.66-2.77 (m, 1H), 2.95-3.03 (m, 1H), 6.84-6.90 (m, 1H, —ArH), 7.06-7.16 (m, 2H, 2×-ArH).

### Compound SM-V

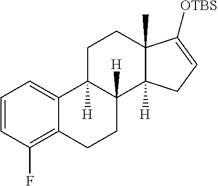

tert-Butyldimethylsilyl triflate (7.1 mL, 31.10 mmol, 110 mol-%) was added dropwise, over a period of 20 min., to a stirred solution of Compound SM-IV (7.70 g, 28.27 mmol, 100 mol-%) and triethylamine (6.0 mL, 42.72 mmol, 151 mol-%) in dichloromethane (75 mL) at room temperature under nitrogen and stirred for 2 h.

The reaction mixture was diluted with dichloromethane (95 mL) and it was washed with a saturated aqueous solution of sodium bicarbonate (2×70 mL) and brine (70 mL). The organic layer was dried over sodium sulfate, filtered and concentrated. The yield of Compound SM-V as a cream solid was quantitative (11.42 g) and used in the next reaction without further purification.

¹H NMR (400 MHz, CDCl₃) δ ppm 0.14-0.19 (m, 6H, 2×-CH₃), 0.86 (s, 3H, —CH₃), 0.94 (s, 9H, 3×-CH₃), 1.21-1.62 (m, 5H), 1.78-2.06 (m, 3H), 2.08-2.16 (m, 1H), 2.25-2.38 (m, 2H), 2.64-2.88 (m, 1H), 2.90-2.99 (m, 1H), 4.48 (dd, 1H, J=3.1, 1.5 Hz), 6.82-6.88 (m, 1H, —ArH), 7.05-7.13 (m, 2H, 2×-ArH).

### Compound SM-VI

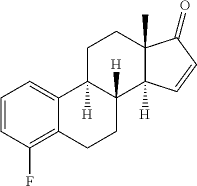

A mixture of Compound SM-V (11.42 g, 28.27 mmol, 100 mol-%) and palladium acetate (0.63 g, 2.83 mmol, 10 mol-%) in dimethylsulfoxide (75 mL) and dichloromethane (50 mL) was stirred at 35° C. under an oxygen atmosphere (balloon) for 16 h, palladium acetate (126 mg, 0.56 mmol, 2 mol-%) was added to the mixture and it was stirred for another 7 h at 35° C.

The reaction mixture was cooled to room temperature and it was poured into a saturated aqueous solution of sodium bicarbonate (300 mL). The mixture was extracted with ethyl acetate (400 mL). The organic layer was washed with water (300 mL) and brine (200 mL) and dried over sodium sulfate, filtered and concentrated to afford an orange/brown solid.

The crude material was purified by flash column chromatography using n-hexanes and ethyl acetate (0-30%) as solvent system. The yield of Compound SM-VI as a pinkish/white solid was 72% (5.50 g).

¹H NMR (400 MHz, CDCl₃) δ ppm 1.11 (s, 3H, —CH₃), 1.46-1.58 (m, 1H), 1.66-1.88 (m, 3H), 1.97-2.07 (m, 1H), 2.23-2.31 (m, 1H), 2.35-2.54 (m, 3H), 2.72-2.84 (m, 1H), 3.03 (dd, 1H, J=17.9, 6.4 Hz), 6.11 (dd, 1H, J=6.0, 3.2 Hz), 6.83-6.92 (m, 1H, —ArH), 7.05-7.18 (m, 2H, 2×-ArH), 7.63-7.66 (m, 1H).

MS m/z (ES⁺): 271 (M+H).

### Compound SM-VII

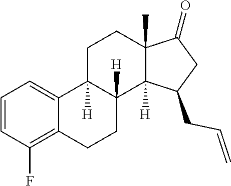

A dry three-neck flask was charged under a nitrogen atmosphere with copper iodide (7.90 g, 41.48 mmol, 350 mol-%), lithium chloride (1.76 g, 41.48 mmol, 350 mol-%) and anhydrous tetrahydrofuran (60 mL). The mixture was stirred for 20 min. at room temperature and it was cooled to −70° C. Allyl magnesium bromide (41.5 mL, 41.48 mmol, 350 mol-%) was then added dropwise, keeping the temperature under −70° C. Chlorotrimethylsilane (5.3 mL, 41.48 mmol, 350 mol-%) was added dropwise to the reaction mixture, keeping the temperature at −70° C., followed by the addition of a solution of Compound SM-VI (3.20 g, 11.85 mmol, 350 mol-%) in anhydrous tetrahydrofuran (60 mL), which was added dropwise keeping the temperature below −65° C. The reaction mixture was allowed to warm slowly to room temperature and stirred overnight.

The mixture was poured into a saturated aqueous solution of ammonium chloride (75 mL) and extracted with ethyl acetate (3×70 mL). The combined extracts were washed with 1M HCl (2×50 mL), water (2×50 mL) and diluted aqueous ammonia solution (5×25 mL) (until the solution was colourless). The organic layer was dried over sodium sulfate, filtered and concentrated. The crude material was purified by flash column chromatography using n-hexanes and ethyl acetate (10%) as solvent system. The yield of Compound SM-VII was 77% (2.85 g).

¹H NMR (400 MHz, CDCl₃) δ ppm 1.05 (s, 3H, —CH₃), 1.40-1.57 (m, 3H), 1.71-1.82 (m, 2H), 1.89-1.96 (m, 1H), 2.04-2.20 (m, 2H), 2.31-2.50 (m, 6H), 2.72-2.84 (m, 1H), 2.94-3.03 (m, 1H), 5.02-5.08 (m, 2H, CH═CH₂), 5.69-5.81 (m, 1H, CH═CH₂), 6.88 (t, 1H, ArH, J=8.7 Hz), 7.05-7.16 (m, 2H, 2×ArH).

### Compound SM-VIII

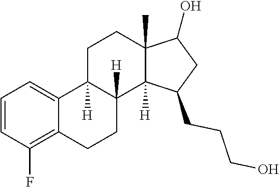

A dry, nitrogen flushed, flask was charged with Compound SM-VII (2.85 g, 9.13 mmol, 100 mol-%) and anhydrous tetrahydrofuran (70 mL). A 1 M solution of borane THE complex (18.3 mL, 18.30 mmol, 200 mol-%) was added dropwise to the previous solution. The resulting reaction mixture was refluxed for 1 h, cooled in an ice-bath to −5° C. and a 3M aqueous solution of sodium hydroxide (28 mL) was added to it very cautiously. After the addition was complete and the effervescence ceased, hydrogen peroxide 30% (28 mL) was added and the mixture was gently refluxed for 2 h.

The reaction mixture was cooled to room temperature and was extracted with ethyl acetate (3×70 mL). The combined extracts were washed with water (2×50 mL) and brine (50 mL), dried over sodium sulfate, filtered and concentrated. The yield of Compound SM-VIII was quantitative (3.09 g).

¹H NMR (400 MHz, CDCl₃) δ ppm 0.82 (s, 3H, —CH₃), 1.13-1.64 (m, 9H), 1.81-1.88 (m, 1H), 1.91-2.06 (m, 2H), 2.16-2.27 (m, 2H), 2.30-2.39 (m, 1H), 2.63-2.74 (m, 1H), 2.81-2.89 (m, 1H), 3.54-3.69 (m, 3H), 6.76-6.82 (m, 1H, —ArH), 6.98-7.08 (m, 2H, 2×-ArH).

Acid SM-IX: [3-((13S,15R)-4-fluoro-13-methyl-17-oxo-7,8,9,11,12,13,14,15,16,17-decahydro-6H-cyclopenta[a]phenanthren-15-yl)propanoic acid]

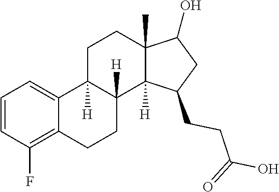

Periodic acid (5.15 g, 22.60 mmol, 500 mol-%) and chromium trioxide (23 mg, 0.23 mmol, 5.0 mol-%) were dissolved in a mixture of acetonitrile (36 mL) and water (12 mL). The solution was cooled to 0° C. in an ice/salt bath. A slurry of Compound SM-VIII (1.5 g, 4.52 mmol, 100 mol-%) in acetonitrile (30 mL) was added to the previous solution over a period of 40 min. maintaining the temperature at or below 0° C. The reaction mixture was stirred for 1 h at 0° C., then the mixture was slowly warmed to room temperature and stirred for 3.5 h.

The reaction mixture was poured into aqueous sodium phosphate dibasic (˜5 g in 100 mL) and extracted with ethyl acetate (3×60 mL). The organic extracts were combined and washed with a 5% aqueous solution of sodium bisulfite (2×40 mL), water (50 mL) and brine (50 mL), dried over sodium sulfate, filtered and concentrated. The crude material was purified by flash column chromatography using n-hexanes, ethyl acetate (10-30%) and acetic acid (1%) as solvent system. The product was dissolved in toluene (50 mL) and stirred for 15 min. Solvent was removed in vacuo and the solid was dried under vacuum at 50° C. The crude yield of Acid SM-IX as a white solid was 71% (1.11 g).

¹H NMR (400 MHz, CDCl₃) δ ppm 0.99 (s, 3H, —CH₃), 1.31-1.53 (m, 3H), 1.55-1.78 (m, 3H), 1.83-2.00 (m, 2H), 2.09-2.17 (m, 1H), 2.23-2.47 (m, 7H), 2.68-2.80 (m, 1H), 2.88-2.97 (m, 1H), 6.81 (t, 1H, —ArH, J=8.6 Hz), 6.98-7.10 (m, 2H, 2×-ArH).

MS m/z (ES−): 343 (M−H).

### Preparation of the Starting Material Acid SM-XV

C-3 Fluoro SM-XV was synthesized from Estrone (Scheme 2) via the Compound SM-X, which may be synthesized as disclosed in Messinger et al. Mol Cell Endocrinol. 2009 (301) 216-224. The detailed synthesis of compound X starting from estrone has been described in WO2008065100, WO2005/047303 and WO2006/125800. The acid SM-X was methylated by heating in methanol in the presence of sulphuric acid followed by triflation. Bistributyltin derivative SM-XIII was prepared from the corresponding triflate SM-XII followed by fluorination to XIV in 75% yield, (ref. WO 2010059943 and Furuya et al JACS 2009, 13 (15),1662). Several estrone deoxyfluorination methods are available (Labrie, Fernand et al. PCT Int. Appl., 9946279, 16 Sep. 1999; Labrie, Fernand et al. PCT Int. Appl., 2004089971, 21 Oct. 2004).

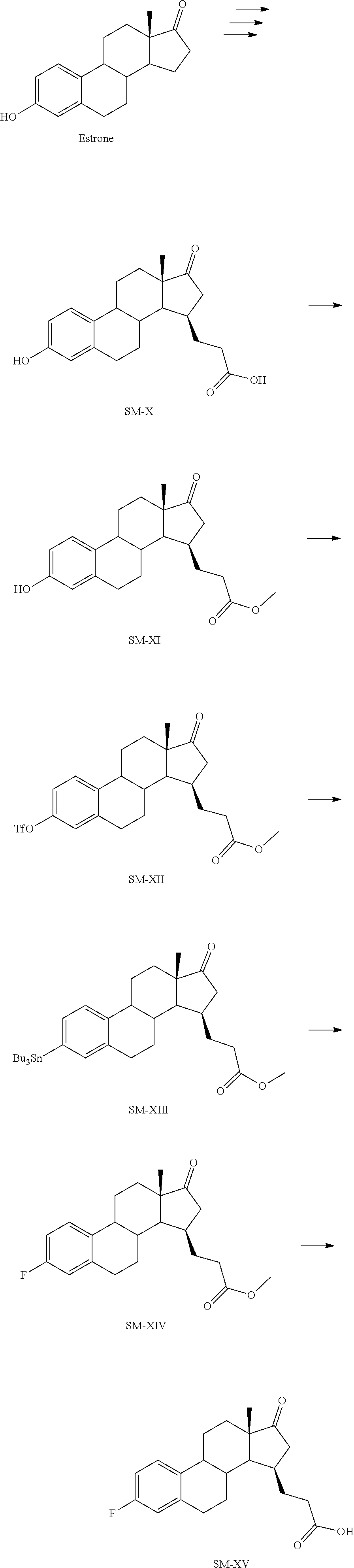

### Compound XIII

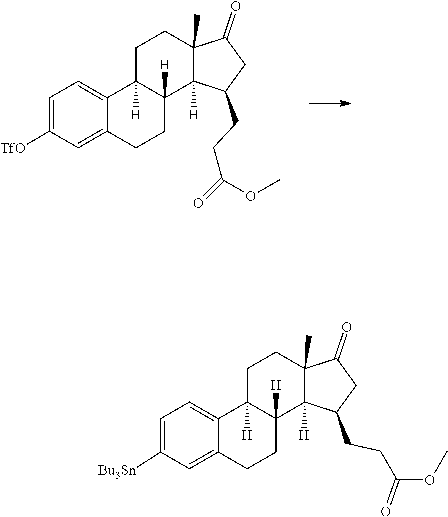

To a screw-cap sealed tube was added Compound SM-XII (10.0 g, 20.47 mmol, 100 mol-%) and 1,4-dioxane (120 mL). Bistributyltin (230.7 mL, 40.99 mmol, 200 mol-%) and LiCl (4.2 g, 102.3 mmol, 500 mol-%) were added to reaction mixture. The reaction mixture was degassed with argon gas for 10 min then added Pd(PPh₃)4 (1.41 g, 1.22 mmol, 6 mol-%) to it. The tube was sealed under nitrogen and the mixture was stirred and heated at 100° C. in a preheated oil bath for 4 hours. The mixture was cooled to room temperature and quenched with water (100 mL), extracted with ethyl acetate (2×200 mL), then filtered through celite, washing well with ethyl acetate. The solvents were concentrated to brown viscous oil. The crude product was purified by flash chromatography eluting with a gradient of 0 to 10% ethyl acetate in hexanes to give the Compound SM-XIII.

¹H NMR (400 MHz, CDCl₃) δ ppm: 7.29-7.19 (m, 3H), 3.69 (s, 3H), 2.95 (bs, 2H), 2.42-0.87 (m, 46H). MS m/z (ES+): poor ionization.

### Compound SM-XIV

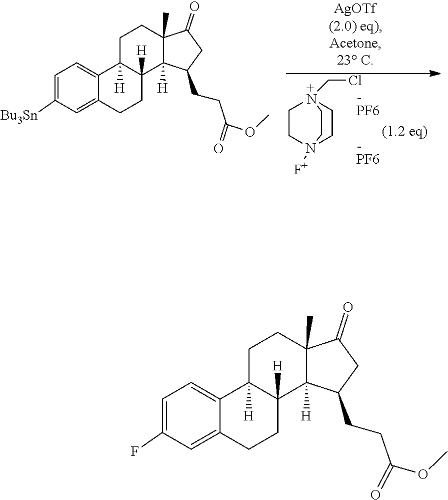

To a stirred solution of Compound SM-XIII (14.0 g, 22.2 mmol, 1.0 eq) in acetone (140 mL) was added AgOTf (11.41 g, 44.4 mmol, 2.0 eq) at room temperature. The reaction mixture was cooled to 0° C. and added 1-Chloromethyl-4-fluoro-1,4-diazoniabicyclo[2.2.2]octane bis(hexafluorophosphate) (12.53 g, 26.6 mmol, 1.2 eq) and the reaction mixture was stirred for 40 min. The reaction was quenched with water (100 mL) and extracted with ethyl acetate (2×150 mL). The organic layer was dried over sodium sulphate, filtered and concentrated. The crude compound was purified by flash chromatography eluted with 0-20% ethyl acetate in hexane. Compound SM-XIV (6.0 g, 75.9%) was afforded as a white solid.

¹H NMR (400 MHz, DMSO-d6) δ ppm: 7.30-7.27 (m, 1H), 7.10-7.08 (d, 1H, J=8 Hz), 6.94-6.89 (m, 1H), 3.59 (s, 3H), 2.87 (bs, 2H), 2.45-2.07 (m, 8H), 1.86-1.32 (m, 8H), 0.95 (s, 3H). MS m/z (ES+): poor ionization.

### Acid SM-XV

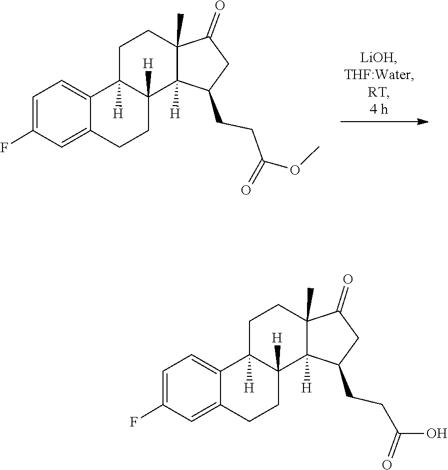

To a stirred solution of compound SM-XIV (6.0 g, 16.7 mmol, 1.0 eq) in THE (60 mL), water (10.5 mL) and was added LiOH.H₂O (1.41 g, 33.5, 2.0 eq) and stirred for 4 h at RT. The reaction mixture was cooled to 10° C., and neutralized with 1 N HCl (pH=6) and extracted with ethyl acetate (2×50 mL). The organic layer was dried over sodium sulphate, filtered and concentrated. The crude compound was triturated with n-pentane (2×10 mL) followed by prep HPLC purification to afford Acid SM-XV (2.2 g, 38.19%) as a white solid.

¹H NMR (400 MHz, DMSO-d6) δ ppm: 12.06 (s, 1H), 7.29-7.27 (d, 1H, J=8 Hz), 7.16-7.14 (d, 2H, J=8 Hz), 2.87 (bs, 2H), 2.37-2.12 (m, 8H), 1.82-1.67 (m, 4H), 1.55-1.38 (m, 4H), 0.84 (s, 3H). MS m/z (ES+): 343.23 (M−H).

### Acid SM-XVII

The Triflate SM-XII in scheme 3 was prepared followed by methods of Messinger et al, WO2008065100. SM-XII was converted to chloro derivative SM-XVI by using t-BuBrettPhos in the presence of tris(dibenzylidene-acetone)dipalladium(0) (Pan et al., Organic Letters, 13(18), 4974-4976; 2011) followed by LiOH treatment in THF:water affording the desired Acid SM-XVII.

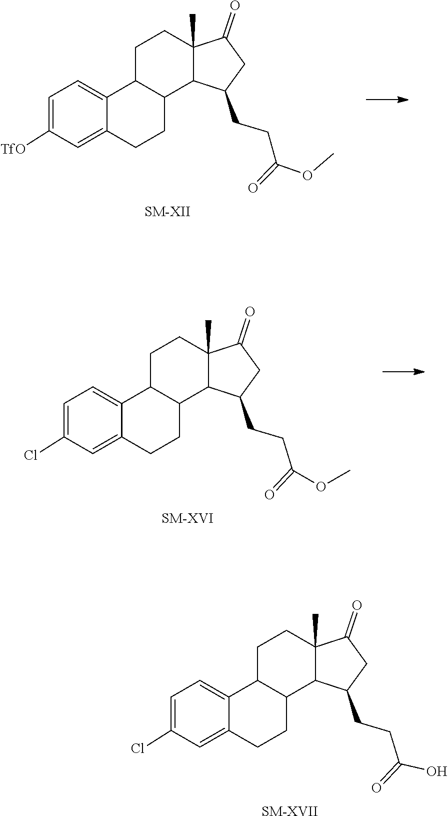

### Compound SM-XVI

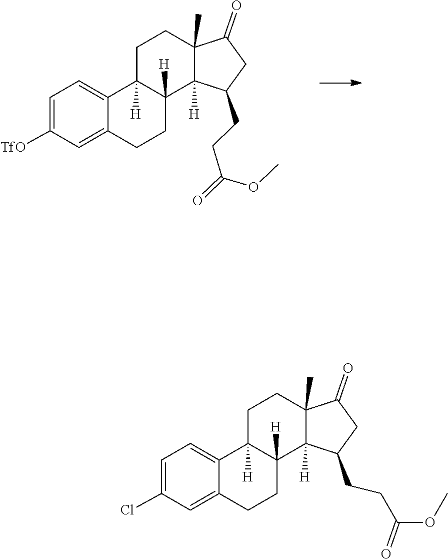

To a screw-cap sealed tube was added tris(dibenzylideneacetone)dipalladium(0) (0.084 g, 0.092 mmol, 3 mol-%) and t-BuBrettPhos (0.133 g, 0.27 mmol, 9 mol-%) and 1,4-dioxane (10 mL) and the tube was sealed under nitrogen. The mixture was stirred and heated at 130° C. in a preheated oil bath for 3 minutes. The catalyst mixture was cooled to room temperature and this mixture was added to a solution of the Compound SM-XII (1.5 g, 3.04 mmol, 100 mol-%) in 1,4-dioxane (11 mL), potassium chloride (0.908 g, 12.28 mmol, 400 mol-%) and potassium fluoride (0.178 g, 3.0 mmol, 100 mol-%). The mixture was stirred and heated at 130° C. in a preheated oil bath for 3 hours. The mixture was cooled to room temperature and then filtered through celite, washing with ethyl acetate. The solvents were concentrated to leave brown viscous oil. The crude product was purified by flash chromatography eluting with a gradient of 0 to 20% the SM-Compound XVI.

¹H NMR (400 MHz, DMSO-d6) δ ppm: 7.29-7.27 (d, 1H, J=8 Hz), 7.16-7.14 (d, 2H, J=8 Hz), 3.59 (s, 3H), 2.87 (bs, 2H), 2.41-2.07 (m, 8H), 1.85-1.38 (m, 8H), 0.95 (s, 3H). MS m/z (ES+): poor ionization.

### Acid SM-XVII

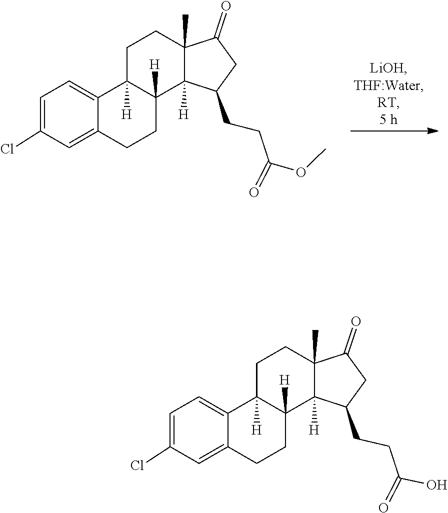

To a stirred solution of Compound SM-XVI (1.7 g, 4.54 mmol, 1.0 eq) in THF:MeOH:Water (12.5 mL, 2:2:1) and was added LiOH.H₂O (0.572 g, 13.6, 3.0 eq) at RT. The reaction mixture was heated at 80° C. for 1.5 h. The reaction progress was monitored by TLC and LC-MS. The reaction mixture was cooled to RT, diluted with water 10 mL and washed with ethyl acetate 3×3 mL. The aqueous layer was neutralized with 1 N HCl (pH=6) and extracted with ethyl acetate (2×50 mL). The organic layer was dried over sodium sulphate, filtered and concentrated. The crude product was triturated with n-pentane (2×10 mL) to afford Acid SM-XVII (1.3 g, 79%) as a white solid.

¹H NMR (400 MHz, DMSO-d6) δ ppm: 12.06 (s, 1H), 7.29-7.27 (d, 1H, J=8 Hz), 7.16-7.14 (d, 2H, J=8 Hz), 2.87 (bs, 2H), 2.37-2.12 (m, 8H), 1.82-1.67 (m, 4H), 1.55-1.38 (m, 4H), 0.84 (s, 3H). MS m/z (ES+): 358.9 (M−H).

### Preparation of the Starting Material Acid SM-XXVI

Compound SM-XXVI was synthesized from Estrone via the triflate SM-XVIII, which was prepared by methods of Messinger et al, WO2008065100. The C15-C16 SM-XXIII was prepared according to methods described in WO2008065100. The allylation, hydroboration and oxidation of SM-XXIII to SM-XXVI was performed as in patents WO2005/047303 and WO2006/125800.

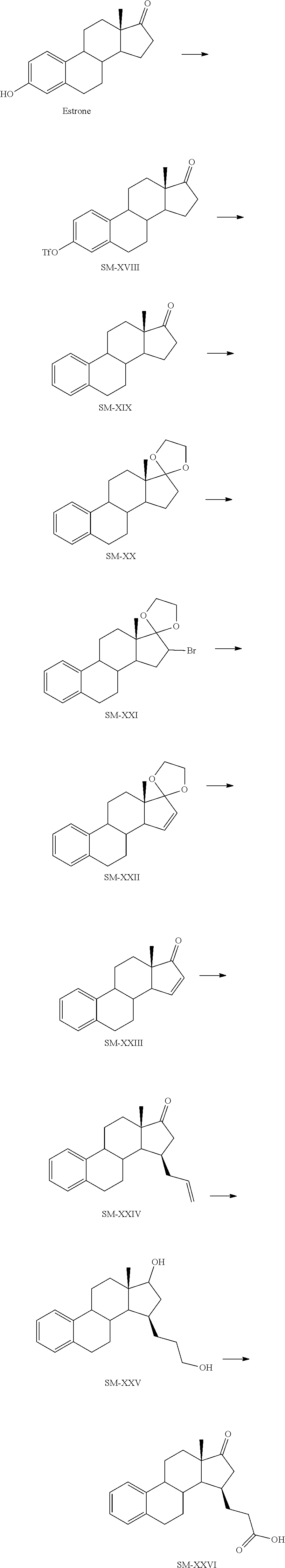

### Acid SM-XXVI

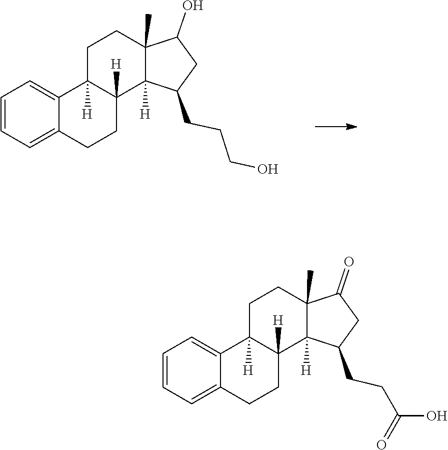

A stirred solution of (8R,9S,13S,14S,15R)-15-(3-hydroxypropyl)-13-methyl-7,8,9,11,12,13,14,15,16,17-decahydro-6H-cyclopenta[a]phenanthren-17-ol (44.0 g 0.140 mol) in acetone (875 mL) was cooled to 0° C. In another RBF, Jones reagent was prepared by dissolving the chromic acid (35 g, 0.350 mol) in water (350 mL) and con. Sulphuric acid (41.14 g, 0.420 mol). The Jones reagent prepared was added in 45 minutes to the starting material solution maintaining the temperature at 0-2° C. for 2-3 h. The reaction mass was quenched using ice cold water (875 mL), the sticky material was filtered and dissolved in 3N NaOH solution (200 mL). The mixture was extracted with ethyl acetate (3×200 mL). The aqueous layer was neutralized with aqueous 2N HCl (pH=6) and extracted with ethyl acetate (3×200 ml). The combined organic layer was washed with brine (200 mL), dried over anhydrous sodium sulphate and solvent was evaporated to obtain solid Acid SM-XXVI 3-((8R,9S,13S,14S,15R)-13-methyl-17-oxo-7,8,9,11,12,13,14,15,16,17-decahydro-6H-cyclopenta[a]phenanthren-15-yl)propanoic acid (24 g, 52%) as a white solid.

¹H NMR (400 MHz, DMSO-d6) δ ppm: 12.0 (s, 1H), 7.27-7.25 (d, 1H, J=8 Hz), 7.13-7.05 (m, 3H), 2.87 (bs, 2H), 2.41-2.10 (m, 8H), 1.87-1.36 (m, 8H), 0.95 (s, 3H).

MS m/z (ES+): 325.23 (M−H).

### General Information

Commercial grade reagents and solvents were used without further purification. Thin-layer chromatography (TLC) was performed on Merck-plates; precoated aluminium sheets. Visualization of plates was done the following techniques: 1) ultraviolet illumination (254 nm), 2) dipping the plate into anisaldehyde or vanilline solution followed by heating. ¹H-NMR spectra were measured with a Bruker DPX (200 MHz) or Avance III 400 (400 MHz) spectrometer with the solvent as indicated.

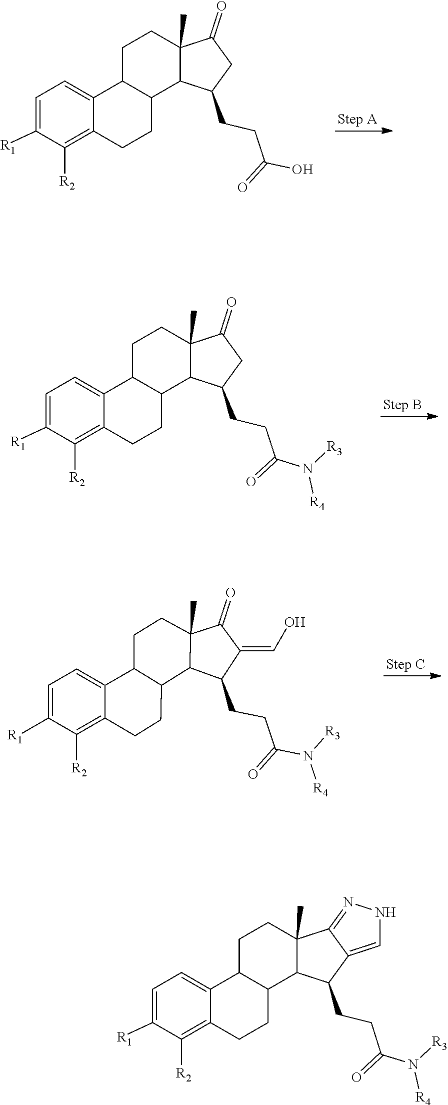

Synthesis Methods for Step A

### Method A1: General Procedure for Amide Preparation by the T3P-Method

The acid (100 mg, 100 mol %) was dissolved in dry THF (3 mL). The corresponding amine (200 mol %) and pyridine (300 mol %) were added. T3P (200 mol %) was added dropwise to the reaction mixture. Stirred at room temperature or at +50° C. until the reaction was completed. The evaporation residue was dissolved in EtOAc and 10% NaHCO₃ was carefully added. The aqueous layer was extracted with ethyl acetate. The organic layers were combined, washed with dilute HCl, water and brine, and dried with sodium sulphate. The crude product was generally purified by chromatography.

### Method A2: General Procedure for Amide Preparation by the EDCI-Method

The acid (150 mg, 100 mol %) was dissolved in dry DMF or DCM (4 mL). HOBt (220 mol %) and EDCI (220 mol %) and the amine (200 mol %) were added to the reaction mixture and stirring was continued at +50° C. until the reaction was completed. Water (4 ml) was added to the reaction mixture when the product precipitates by water addition, followed by washing with water several times.

### Method A3: Modified Procedure for Amide Preparation by the EDCI-Method

The acid (200 mg, 100 mol %) was dissolved in dry dichloromethane (4 mL). The amine (150 mol %), N-methylmorpholine (300 mol %) and 1-hydroxy-1H-benzotriazole (220 mol-%) was added to the reaction mixture. After stirring for 5 minutes, the reaction mixture was cooled to 0-5° C. EDCI (220 mol %) was added to the reaction mixture. Stirred at room temperature until the reaction was completed. The reaction mixture was diluted with DCM (˜5 ml), washed with 0.5 N HCl solution (2×10 ml), water (3×10 ml) and brine (2×10 ml). The organic layer was dried over sodium sulfate. The crude product was purified if needed.

Synthesis Method for Step B

### Method B: General Procedure for Preparation of the Hydroxymethylenes by the Ethylformate/NaH-Method

The steroidal C-17 carbonyl intermediate containing suitable amide unit at the C-15 position (95 mg, 100 mol %) was co-evaporated with toluene (3×10 mL), then dissolved in dry THE (400 μl), To the reaction mixture was added under nitrogen dry toluene (1000 μl), ethyl formate (600 mol %) and NaH (450 mol %) and then stirred at room temperature until the reaction was completed. The solvent was evaporated, and the residue was dissolved in EtOAc and washed with dilute hydrochloric acid, water and brine, and dried with sodium sulfate.

Synthesis Method for Step C

### Method C: General Procedure for Pyrazole Preparation by the Hydrazine Hydrate-Method

The hydroxymethylene derivative (90-100 mg, 100 mol %) was dissolved in methanol (1.5 ml). Hydrazine hydrate (200 mol %) was added and stirred at +50° C. under nitrogen 30 minutes. The solvent was evaporated. Evaporation residue was dissolved in ethyl acetate, washed trice with 1N hydrochloric acid. The aqueous layers were combined and then was washed with ethyl acetate, finally the aqueous layer was neutralized (pH ˜8) and the product was extracted with ethyl acetate. The product was purified by chromatography or by crystallization or trituration.

### Compound 1

### N-(5-Cyanopyridin-2-yl)-3-((13S,15R)-4-fluoro-13-methyl-17-oxo-7,8,9,11,12,13,14,15,16,17-decahydro-6H-cyclopenta[a]phenanthren-15-yl)propanamide

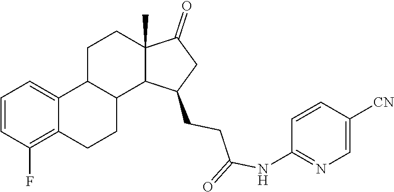 [SMILES: C[C@]12CCC3c4cccc(F)c4CCC3C1[C@H](CCC(=O)Nc1ccc(C#N)cn1)CC2=O]

The compound 1 was prepared Method A1 from Acid SM-IX and 5-cyano-2-aminopyridine by stirring overnight at room temperature. The yield was 83%.

¹H-NMR (400 MHz, DMSO-d₆): 0.98 (s, 3H), 1.33-2.48 (m, 15H), 2.57 (m, 1H), 2.68-2.90 (m, 2H), 6.97 (dd, 1H), 7.14 (m, 2H), 8.25 (s, 2H), 8.78 (s, 1H), 11.04 (s, 1H).

### Compound 2

### N-(5-Cyanopyridin-2-yl)-3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)propanamide

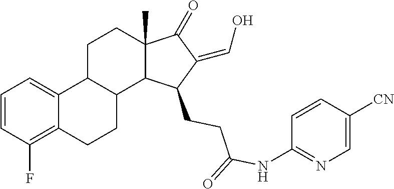 [SMILES: C[C@]12CCC3c4cccc(F)c4CCC3C1[C@H](CCC(=O)Nc1ccc(C#N)cn1)/C(=C/O)C2=O]

The compound 2 was prepared from the compound 1 by Method B stirring overnight at room temperature in 51% yield.

¹H NMR (400 MHz, DMSO-d₆): 1.00 (s, 3H), 1.38-2.37 (m, 12H), 2.64 (m, 2H), 2.74-2.96 (m, 3H), 6.96 (dd, 1H), 7.14 (m, 2H), 7.57 (s, 1H), 8.23 (m, 2H), 8.76 (s, 1H), 11.03 (s, 1H).

### Compound 3

### N-(5-Cyanopyridin-2-yl)-3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamide

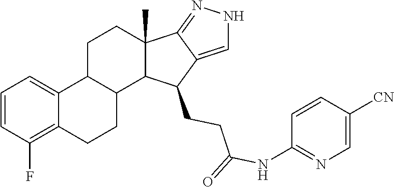 [SMILES: C[C@]12CCC3c4cccc(F)c4CCC3C1[C@H](CCC(=O)Nc1ccc(C#N)cn1)c1c[nH]nc12]

The compound 3 was prepared in 80% yield from the compound 2 by the Method C and purified by chromatography.

¹H NMR (400 MHz, DMSO-d₆): 1.11 (s, 3H), 1.43-2.45 (m, 11H), 2.56 (m, 2H), 2.76-2.93 (m, 3H), 6.97 (dd, 1H), 7.16 (m, 2H), 7.39 (s, 1H), 8.25 (m, 2H), 8.79 (d, 1H), 11.08 (s, 1H), 12.13 (br s, 1H).

### Compound 4

### 3-((13S,15R)-4-fluoro-13-methyl-17-oxo-7,8,9,11,12,13,14,15,16,17-decahydro-6H-cyclopenta[a]phenanthren-15-yl)-N-(6-methoxypyridazin-3-yl)propanamide

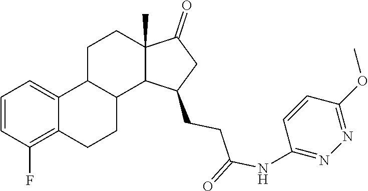 [SMILES: COc1ccc(NC(=O)CC[C@@H]2CC(=O)[C@@]3(C)CCC4c5cccc(F)c5CCC4C23)nn1]

The compound 4 was prepared Method A2 from Acid SM-IX and 3-amino-6-methoxypyridazine stirring four hours at room temperature. The yield was 95%.

¹H-NMR (200 MHz, DMSO-d₆): 0.98 (s, 3H), 1.20-2.47 (m, 16H), 2.60-2.97 (m, 2H), 3.98 (s, 3H), 6.89-7.06 (m, 1H), 7.08-7.21 (m, 2H), 7.25 (d, 1H), 8.26 (d, 1H), 10.94 (br s, 1H).

### Compound 5

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(6-methoxypyridazin-3-yl)propanamide

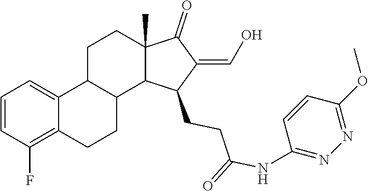 [SMILES: COc1ccc(NC(=O)CC[C@@H]2/C(=C/O)C(=O)[C@@]3(C)CCC4c5cccc(F)c5CCC4C23)nn1]

The compound 5 was prepared from the compound 4 by Method B in 89% yield.

¹H NMR (400 MHz, DMSO-d₆): 1.00 (s, 3H), 1.38-2.37 (m, 11H), 2.68-2.98 (m, 5H), 3.98 (s, 3H), 6.97 (dd, 1H), 7.14 (m, 2H), 7.23 (d, 1H), 7.55 (s, 1H), 8.24 (d, 2H), 10.87 (s, 1H).

### Compound 6

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(6-methoxypyridazin-3-yl)propanamide

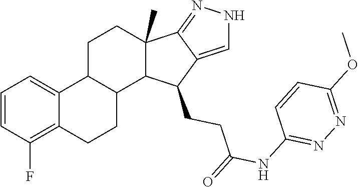 [SMILES: COc1ccc(NC(=O)CC[C@@H]2c3c[nH]nc3[C@@]3(C)CCC4c5cccc(F)c5CCC4C23)nn1]

The compound 6 was prepared in 55% yield from compound 5 by Method C purifying the crude product by trituration with heptane-ethanol 1:1 mixture.

¹H NMR (400 MHz, DMSO-d₆): 1.11 (s, 3H), 1.41-2.44 (m, 11H), 2.53-2.92 (m, 5H), 3.99 (s, 3H), 6.97 (dd, 1H), 7.15 (m, 2H), 7.24 (d, 1H), 7.42 (s, 1H), 8.26 (d, 1H), 10.97 (s, 1H), 12.13 (br s, 1H).

### Compound 7

### N-(3,5-difluoropyridin-2-yl)-3-((13S,15R)-4-fluoro-13-methyl-17-oxo-7,8,9,11,12,13,14,15,16,17-decahydro-6H-cyclopenta[a]phenanthren-15-yl)propanamide

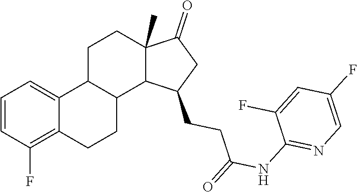 [SMILES: C[C@]12CCC3c4cccc(F)c4CCC3C1[C@H](CCC(=O)Nc1ncc(F)cc1F)CC2=O]

The compound 7 was prepared by Method A1 from Acid SM-IX using 2-amino-3,5-difluoropyridine as an amine stirring two hours at room temperature. The yield was 91%.

¹H-NMR (400 MHz, DMSO-d₆): 0.98 (s, 3H), 1.34-2.55 (m, 16H), 2.69-2.90 (m, 2H), 6.97 (dd, 1H), 7.14 (m, 2H), 8.01 (dd, 1H), 8.34 (d, 1H), 10.31 (s, 1H).

### Compound 8

### N-(3,5-difluoropyridin-2-yl)-3-((13S,15S,Z)-4-fluoro-16-(hydroxylmethylene)-13-methyl-17-oxo-7,8,9,11,12,13,14,15,16,17-decahydro-6H-cyclopenta[a]phenanthren-15-yl)propanamide

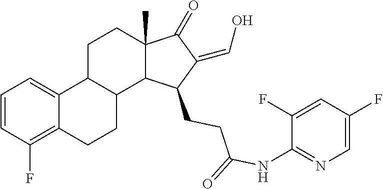 [SMILES: C[C@]12CCC3c4cccc(F)c4CCC3C1[C@H](CCC(=O)Nc1ncc(F)cc1F)/C(=C/O)C2=O]

The compound 8 was prepared from the compound 7 by Method B stirring three hours at room temperature in 96% yield.

¹H NMR (400 MHz, DMSO-d₆): 0.99 (s, 3H), 1.39-2.45 (m, 12H), 2.67-2.96 (m, 5H), 6.97 (dd, 1H), 7.14 (m, 2H), 7.57 (s, 1H), 8.00 (m, 1H), 8.33 (d, 1H), 10.31 (s, 1H).

### Compound 9

### N-(3,5-difluoropyridin-2-yl)-3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamide

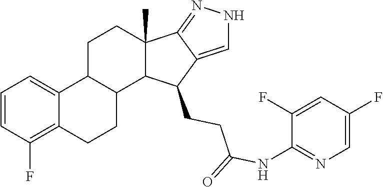 [SMILES: C[C@]12CCC3c4cccc(F)c4CCC3C1[C@H](CCC(=O)Nc1ncc(F)cc1F)c1c[nH]nc12]

The compound 9 was prepared from the compound 8 by Method C in 84% yield.

¹H NMR (400 MHz, DMSO-d₆): 1.11 (s, 3H), 1.37-2.48 (m, 13H), 2.71-2.92 (m, 3H), 6.97 (dd, 1H), 7.16 (m, 2H), 7.44 (s, 1H), 8.00 (m, 1H), 8.34 (d, 1H), 10.36 (s, 1H), 12.14 (br s, 1H).

### Compound 10

### 3-((13S,15R)-4-fluoro-13-methyl-17-oxo-7,8,9,11,12,13,14,15,16,17-decahydro-6H-cyclopenta[a]phenanthren-15-yl)-N-(5-fluoropyridin-2-yl)propanamide

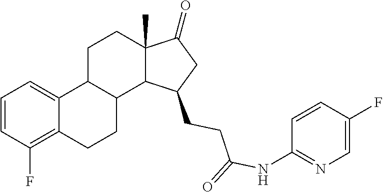 [SMILES: C[C@]12CCC3c4cccc(F)c4CCC3C1[C@H](CCC(=O)Nc1ccc(F)cn1)CC2=O]

The compound 10 was prepared in 99% yield by Method A1 from Acid SM-IX using 2-amino-5-fluoropyridine as an amine stirring 3 hours at room temperature.

¹H NMR (200 MHz, DMSO-d₆): 0.98 (s, 3H), 1.24-2.46 (m, 16H), 2.59-3.03 (m, 2H), 6.90-7.05 (m, 1H), 7.06-7.22 (m, 2H), 7.73 (td, 1H), 8.15 (dd, 1H), 8.32 (d, 1H), 10.63 (s, 1H). MS m/z (TOF ES⁺): 439 (M+1)

### Compound 11

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(5-fluoropyridin-2-yl)propanamide

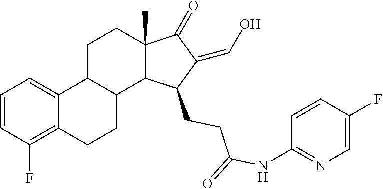 [SMILES: C[C@]12CCC3c4cccc(F)c4CCC3C1[C@H](CCC(=O)Nc1ccc(F)cn1)/C(=C/O)C2=O]

The compound 11 was prepared from the compound 10 by Method B in 99% yield.

¹H NMR (400 MHz, DMSO-d₆): 0.99 (s, 3H), 1.39-2.37 (m, 11H), 2.58 (m, 2H), 2.70-2.97 (m, 3H), 6.96 (dd, 1H), 7.14 (m, 2H), 7.54 (s, 1H), 7.71 (m, 1H), 8.14 (dd, 1H), 8.30 (d, 1H), 10.55 (s, 1H), 10.99 (br s, 1H).

### Compound 12

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(5-fluoropyridin-2-yl)propanamide

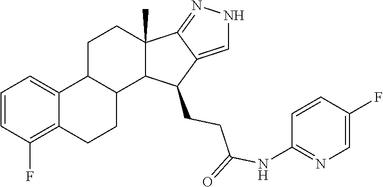 [SMILES: C[C@]12CCC3c4cccc(F)c4CCC3C1[C@H](CCC(=O)Nc1ccc(F)cn1)c1c[nH]nc12]

The compound 12 was prepared from the compound 11 by Method C in 90% yield.

¹H NMR (400 MHz, DMSO-d₆): 1.11 (s, 3H), 1.48-2.42 (m, 13H), 2.72-2.93 (m, 3H), 6.97 (dd, 1H), 7.16 (m, 2H), 7.40 (s, 1H), 7.72 (m, 1H), 8.15 (m, 1H), 8.31 (d, 1H), 10.66 (s, 1H), 12.15 (br s, 1H).

### Compound 13

### 3-((13S,15R)-4-fluoro-13-methyl-17-oxo-7,8,9,11,12,13,14,15,16,17-decahydro-6H-cyclopenta[a]phenanthren-15-yl)-N-(4-fluoropyridin-2-yl)propanamide

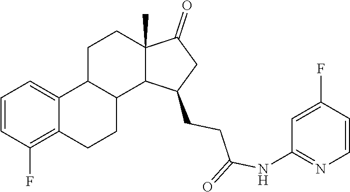 [SMILES: C[C@]12CCC3c4cccc(F)c4CCC3C1[C@H](CCC(=O)Nc1cc(F)ccn1)CC2=O]

The compound 13 was synthesized in 83% yield by the Method A1 in THE by using acid SM-IX and 2-amino-4-fluoropyridine as starting materials in overnight reaction time.

¹H-NMR (400 MHz, DMSO-d₆): 0.98 (s, 3H), 1.36-1.46 (m, 3H), 1.58-1.74 (m, 4H), 1.89-1.94 (m, 1H), 2.16-2.43 (m, 7H), 2.68-2.91 (m, 3H), 6.95-7.04 (m, 2H), 7.05-7.20 (m, 2H), 7.93 (dd, 1H), 8.34 (dd, 1H), 10.83 (s, 1H).

### Compound 14

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(4-fluoropyridin-2-yl)propanamide

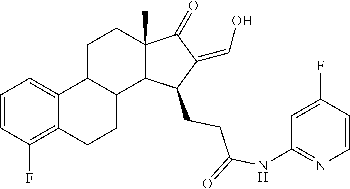 [SMILES: C[C@]12CCC3c4cccc(F)c4CCC3C1[C@H](CCC(=O)Nc1cc(F)ccn1)/C(=C/O)C2=O]

The compound 14 was prepared from the compound 13 by the Method B in 44% yield.

¹H NMR (400 MHz, DMSO-d₆): 1.00 (s, 3H), 1.32-2.30 (m, 12H), 2.55-2.96 (m, 5H), 6.96 (dd, 1H), 7.02 (m, 1H), 7.14 (m, 2H), 7.56 (s, 1H), 7.92 (dd, 1H), 8.34 (m, 1H), 10.80 (s, 1H).

### Compound 15

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(4-fluoropyridin-2-yl)propanamide

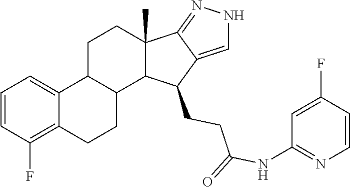 [SMILES: C[C@]12CCC3c4cccc(F)c4CCC3C1[C@H](CCC(=O)Nc1cc(F)ccn1)c1c[nH]nc12]

The compound 15 was prepared from the compound 14 by Method C in 57% yield.

¹H NMR (400 MHz, DMSO-d₆): 1.11 (s, 3H), 1.40-2.42 (m, 13H), 2.76-2.93 (m, 3H), 6.97 (dd, 1H), 7.03 (m, 1H), 7.16 (m, 2H), 7.40 (s, 1H), 7.93 (dd, 1H), 8.36 (dd, 1H), 10.87 (s, 1H), 12.16 (br s, 1H).

### Compound 16

### 3-((13S,15R)-4-fluoro-13-methyl-17-oxo-7,8,9,11,12,13,14,15,16,17-decahydro-6H-cyclopenta[a]phenanthren-15-yl)-N-(pyridin-2-yl)propanamide

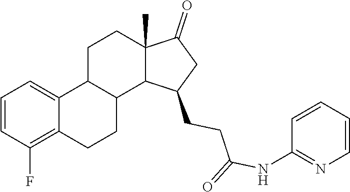 [SMILES: C[C@]12CCC3c4cccc(F)c4CCC3C1[C@H](CCC(=O)Nc1ccccn1)CC2=O]

The compound 16 was synthesized in 51% yield by the Method A2 in DMF by using acid SM-IX and 2-aminopyridine as starting materials in overnight reaction time.

¹H-NMR (400 MHz, DMSO-d₆): 0.98 (s, 3H), 1.34-1.47 (m, 3H), 1.59-1.68 (m, 4H), 1.78-1.90 (m, 1H), 2.17-2.46 (m, 8H), 2.68-2.82 (m, 2H), 6.95-6.99 (m, 1H), 7.07-7.13 (m, 1H), 7.14-7.20 (m, 2H), 7.76 (dd, 1H), 8.10 (d, 1H), 8.30 (dd, 1H), 10.50 (s, 1H).

### Compound 17

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(pyridin-2-yl)propanamide

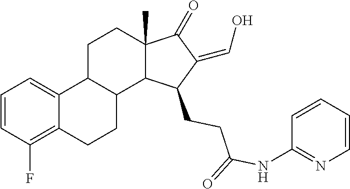 [SMILES: C[C@]12CCC3c4cccc(F)c4CCC3C1[C@H](CCC(=O)Nc1ccccn1)/C(=C/O)C2=O]

The compound 17 was prepared from the compound 16 by the Method B in 97% yield.

¹H NMR (400 MHz, DMSO-d₆): 0.99 (s, 3H), 1.35-2.23 (m, 12H), 2.68-2.98 (m, 5H), 6.96 (dd, 1H), 7.05 (m, 1H), 7.14 (m, 2H), 7.70 (s, 1H), 7.75 (dd, 1H), 8.09 (d, 1H), 8.30 (m, 1H), 10.70 (s, 1H).

### Compound 18

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(pyridin-2-yl)propanamide

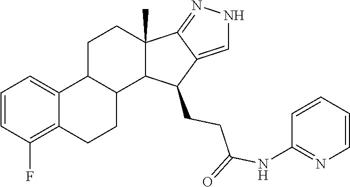 [SMILES: C[C@]12CCC3c4cccc(F)c4CCC3C1[C@H](CCC(=O)Nc1ccccn1)c1c[nH]nc12]

The compound 18 was prepared from the compound 17 by the Method C in 91% yield.

¹H NMR (400 MHz, DMSO-d₆): 1.11 (s, 3H), 1.40-2.45 (m, 13H), 2.67-2.99 (m, 3H), 6.97 (dd, 1H), 7.07 (m, 1H), 7.16 (m, 2H), 7.41 (s, 1H), 7.76 (dd, 1H), 8.10 (d, 1H), 8.30 (d, 1H), 10.54 (s, 1H), 12.13 (br s, 1H).

### Compound 19

### 3-((13S,15R)-4-fluoro-13-methyl-17-oxo-7,8,9,11,12,13,14,15,16,17-decahydro-6H-cyclopenta[a]phenanthren-15-yl)-N-(5-methoxypyridin-2-yl)propanamide

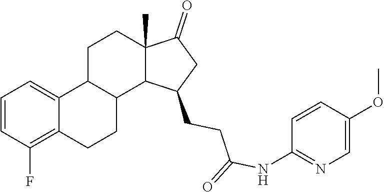 [SMILES: COc1ccc(NC(=O)CC[C@@H]2CC(=O)[C@@]3(C)CCC4c5cccc(F)c5CCC4C23)nc1]

The compound 19 was synthesized in 80% yield by the Method A2 in DMF stirring at +50° C. for two hours by using acid SM-IX and 5-methoxy-2-aminopyridine as starting materials in overnight reaction time.

¹H-NMR (200 MHz, CDCl₃): 1.07 (s, 3H), 1.35-2.53 (m, 16H), 2.72-3.03 (m, 2H), 3.85 (s, 3H), 6.83-6.92 (m, 1H), 7.05-7.18 (m, 2H), 7.24-7.30 (m, 1H), 7.92-8.01 (m, 2H), 8.15 (d, 1H).

### Compound 20

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(5-methoxypyridin-2-yl)propanamide

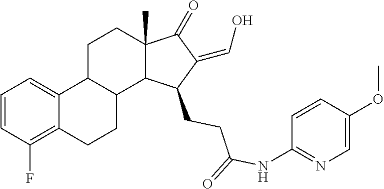 [SMILES: COc1ccc(NC(=O)CC[C@@H]2/C(=C/O)C(=O)[C@@]3(C)CCC4c5cccc(F)c5CCC4C23)nc1]

The compound 20 was prepared from the compound 19 by the Method B in quantitative yield.

¹H NMR (400 MHz, DMSO-d₆): 0.99 (s, 3H), 1.41-2.35 (m, 12H), 2.68-2.96 (m, 4H), 3.80 (s, 3H), 6.96 (m, 1H), 7.14 (m, 2H), 7.40 (d, 1H), 7.55 (s, 1H), 8.01 (s, 2H), 10.32 (s, 1H), 11.04 (br s, 1H).

### Compound 21

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(5-methoxypyridin-2-yl)propanamide

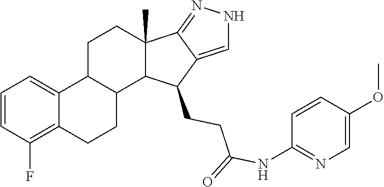 [SMILES: COc1ccc(NC(=O)CC[C@@H]2c3c[nH]nc3[C@@]3(C)CCC4c5cccc(F)c5CCC4C23)nc1]

The compound 21 was prepared from the compound 20 by the Method C in 57% yield.

¹H NMR (400 MHz, DMSO-d₆): 1.10 (s, 3H), 1.40-2.47 (m, 13H), 2.70-2.99 (m, 3H), 3.80 (s, 3H), 6.97 (m, 1H), 7.16 (m, 2H), 7.41 (s, 2H), 8.03 (m, 2H), 10.41 (s, 1H), 12.12 (br s, 1H).

### Compound 22

### 4-(3-((13S,15R)-4-fluoro-13-methyl-17-oxo-7,8,9,11,12,13,14,15,16,17-decahydro-6H-cyclopenta[a]phenanthren-15-yl)propanoyl)piperazin-2-one

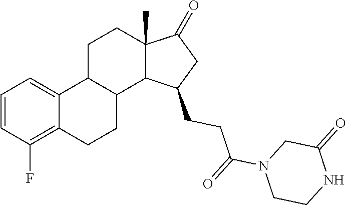 [SMILES: C[C@]12CCC3c4cccc(F)c4CCC3C1[C@H](CCC(=O)N1CCNC(=O)C1)CC2=O]

The compound 22 was synthesized in 81% yield by the Method A1 in THE using acid SM-IX and piperazin-2-one as starting materials in three hours reaction time.

¹H-NMR (400 MHz, DMSO-d₆): 0.97 (s, 3H), 1.37-2.45 (m, 17H), 2.68-2.90 (m, 2H), 3.57-3.64 (m, 4H), 3.93 (dd, 1H), 6.97 (m, 1H), 7.13-7.20 (m, 2H), 8.09 (d, 1H).

### Compound 23

### 4-(3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)propanoyl)piperazin-2-one

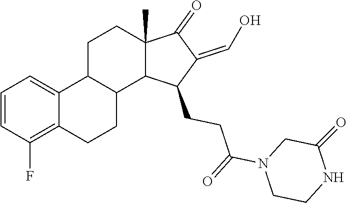 [SMILES: C[C@]12CCC3c4cccc(F)c4CCC3C1[C@H](CCC(=O)N1CCNC(=O)C1)/C(=C/O)C2=O]

The compound 23 was prepared from the compound 22 by the Method B in 71% yield.

¹H NMR (400 MHz, DMSO-d₆): 0.97 (s, 3H), 1.35-2.37 (m, 14H), 2.67-2.92 (m, 3H), 3.16-3.25 (m, 3H), 3.58-3.67 (m, 2H), 3.93 (d, 1H), 6.97 (dd, 1H), 7.14 (m, 2H), 7.53 (s, 1H), 8.11 (d, 1H).

### Compound 24

### 4-(3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanoyl)piperazin-2-one

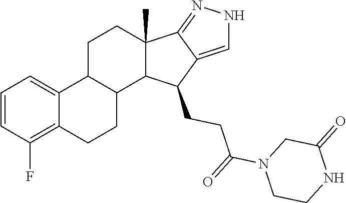 [SMILES: C[C@]12CCC3c4cccc(F)c4CCC3C1[C@H](CCC(=O)N1CCNC(=O)C1)c1c[nH]nc12]

The compound 24 was prepared from the compound 23 by the Method C in 36% yield.

¹H NMR (400 MHz, DMSO-d₆): 1.09 (s, 3H), 1.30-2.47 (m, 13H), 2.67-2.92 (m, 3H), 3.15-3.25 (m, 2H), 3.51-3.68 (m, 2H), 3.92 (d, 1H), 4.02-4.08 (dd, 1H), 6.98 (m, 1H), 7.16 (m, 2H), 7.45 (d, 1H), 8.10 (d, 1H), 12.12 (br s, 1H).

### Compound 25

### 3-((13S,15R)-4-fluoro-13-methyl-17-oxo-7,8,9,11,12,13,14,15,16,17-decahydro-6H-cyclopenta[a]phenanthren-15-yl)-N-(4-methylpyridin-2-yl)propanamide

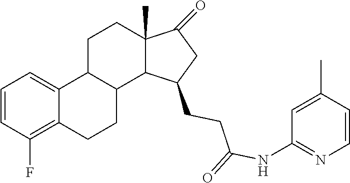 [SMILES: Cc1ccnc(NC(=O)CC[C@@H]2CC(=O)[C@@]3(C)CCC4c5cccc(F)c5CCC4C23)c1]

The compound 25 was synthesized in 37% yield after chromatographic purification by the Method A2 in THE by using 200 mol-% of EDCI and HOBT from acid SM-IX and 2-amino-4-methylpyridine as starting materials in 4.5 hours reaction time.

¹H-NMR (400 MHz, DMSO-d₆): 0.98 (s, 3H), 1.30-2.48 (m, 16H), 2.30 (s, 3H), 2.65-2.78 (m, 1H), 2.80-2.92 (m, 1H), 6.90-6.93 (m, 1H), 6.94-7.00 (m, 1H), 7.10-7.21 (m, 2H), 7.95 (s, 1H), 8.13-8.17 (m, 1H), 10.42 (s, 1H).

### Compound 26

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(4-methylpyridin-2-yl)propanamide

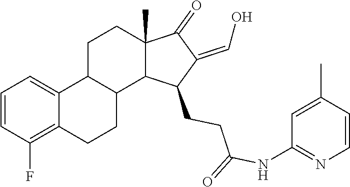 [SMILES: Cc1ccnc(NC(=O)CC[C@@H]2/C(=C/O)C(=O)[C@@]3(C)CCC4c5cccc(F)c5CCC4C23)c1]

The compound 26 was synthesized in quantitative yield from the compound 25 by the Method B in 5 hours reaction time by using 500 mol-% of ethyl formate and 300 mol-% of NaH.

¹H-NMR (400 MHz, DMSO-d₆): 0.99 (s, 3H), 1.33-2.60 (m, 13H), 2.30 (s, 3H), 2.65-3.00 (m, 3H), 6.92 (d, 1H), 6.94-7.00 (m, 1H), 7.10-7.21 (m, 2H), 7.55 (s, 1H), 7.94 (s, 1H), 8.14 (d, 1H), 10.35 (s, 1H), 11.00 (br s, 1H).

### Compound 27

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(4-methylpyridin-2-yl)propanamide

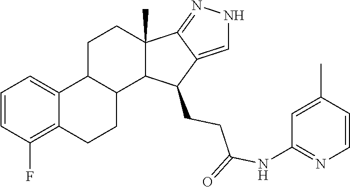 [SMILES: Cc1ccnc(NC(=O)CC[C@@H]2c3c[nH]nc3[C@@]3(C)CCC4c5cccc(F)c5CCC4C23)c1]

The compound 27 was synthesized in 51% yield from the compound 26 by the Method C in 0.5 hours reaction time.

¹H-NMR (400 MHz, DMSO-d₆): 1.10 (s, 3H), 1.33-2.60 (m, 13H), 2.30 (s, 3H), 2.69-2.95 (m, 3H), 6.92 (d, 1H), 6.94-7.00 (m, 1H), 7.10-7.21 (m, 2H), 7.42 (s, 1H), 7.96 (s, 1H), 8.16 (d, 1H), 10.47 (s, 1H), 12.16 (br s, 1H).

### Compound 28

### 3-((13S,15R)-4-fluoro-13-methyl-17-oxo-7,8,9,11,12,13,14,15,16,17-decahydro-6H-cyclopenta[a]phenanthren-15-yl)-N-(2-oxo-1,2,5,6,7,8-hexahydroquinolin-3-yl)propanamide

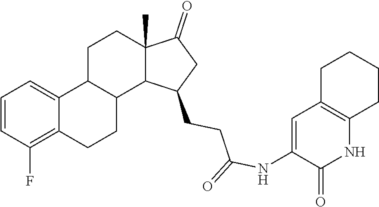 [SMILES: C[C@]12CCC3c4cccc(F)c4CCC3C1[C@H](CCC(=O)Nc1cc3c([nH]c1=O)CCCC3)CC2=O]

The compound 28 was synthesized in 86% yield by the Method A1 in THE by using acid SM-IX and 3-amino-1,2,5,6,7,8-hexahydroquinolin-2-one as starting materials in overnight reaction time.

¹H-NMR (400 MHz, DMSO-d₆): 0.97 (s, 3H), 1.30-2.47 (m, 24H), 2.65-2.93 (m, 2H), 6.94-7.00 (m, 1H), 7.12-7.21 (m, 2H), 8.01 (s, 1H), 9.15 (s, 1H), 11.68 (br s, 1H).

### Compound 29

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(2-oxo-1,2,5,6,7,8-hexahydroquinolin-3-yl)propanamide

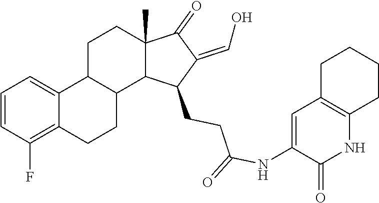 [SMILES: C[C@]12CCC3c4cccc(F)c4CCC3C1[C@H](CCC(=O)Nc1cc3c([nH]c1=O)CCCC3)/C(=C/O)C2=O]

The compound 29 was synthesized in 73% yield from the compound 28 by the Method B in overnight reaction time.

¹H-NMR (400 MHz, DMSO-d₆): 0.98 (s, 3H), 1.30-2.65 (m, 21H), 2.65-2.97 (m, 3H), 6.94-7.00 (m, 1H), 7.11-7.21 (m, 2H), 7.55 (s, 1H), 8.00 (s, 1H), 9.00 (s, 1H), 11.20 (br s, 1H), 11.68 (s, 1H).

### Compound 30

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(2-oxo-1,2,5,6,7,8-hexahydroquinolin-3-yl)propanamide

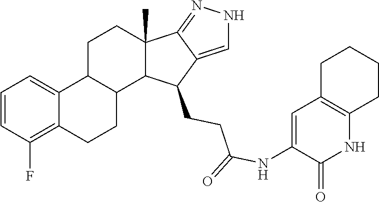 [SMILES: C[C@]12CCC3c4cccc(F)c4CCC3C1[C@H](CCC(=O)Nc1cc3c([nH]c1=O)CCCC3)c1c[nH]nc12]

The compound 30 was synthesized in 72% yield from the compound 29 by the Method C in 2 hours reaction time.

¹H-NMR (400 MHz, DMSO-d₆): 1.10 (s, 3H), 1.30-2.65 (m, 21H), 2.69-2.97 (m, 3H), 6.94-7.00 (m, 1H), 7.11-7.21 (m, 2H), 7.42 (s, 1H), 8.00 (s, 1H), 9.15 (s, 1H), 11.68 (s, 1H), 12.12 (br s, 1H).

### Compound 31

### 6-(3-((13S,15R)-4-fluoro-13-methyl-17-oxo-7,8,9,11,12,13,14,15,16,17-decahydro-6H-cyclopenta[a]phenanthren-15-yl)propanamido)-N,N-dimethylnicotinamide

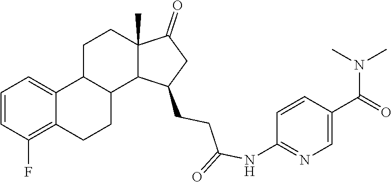 [SMILES: CN(C)C(=O)c1ccc(NC(=O)CC[C@@H]2CC(=O)[C@@]3(C)CCC4c5cccc(F)c5CCC4C23)nc1]

The compound 31 was synthesized in quantitative yield by the Method A1 in THE by using acid SM-IX and 6-amino-N,N-dimethylpyridine-3-carboxamide as starting materials in overnight reaction time.

¹H-NMR (400 MHz, DMSO-d₆): 0.98 (s, 3H), 1.30-2.47 (m, 16H), 2.66-2.94 (m, 2H), 2.98 (s, 6H), 6.94-7.00 (m, 1H), 7.12-7.21 (m, 2H), 7.85 (dd, 1H), 8.14 (d, 1H), 8.38 (d, 1H), 10.72 (s, 1H).

### Compound 32

### 6-(3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)propanamido)-N,N-dimethylnicotinamide

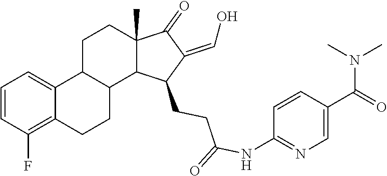 [SMILES: CN(C)C(=O)c1ccc(NC(=O)CC[C@@H]2/C(=C/O)C(=O)[C@@]3(C)CCC4c5cccc(F)c5CCC4C23)nc1]

The compound 32 was synthesized in 33% yield from the compound 31 by the Method B in overnight reaction time.

¹H-NMR (400 MHz, DMSO-d₆): 1.00 (s, 3H), 1.30-2.65 (m, 13H), 2.66-2.94 (m, 3H), 2.97 (s, 6H), 6.93-7.00 (m, 1H), 7.11-7.21 (m, 2H), 7.55 (s, 1H), 7.84 (dd, 1H), 8.14 (d, 1H), 8.37 (d, 1H), 10.66 (s, 1H), 10.98 (br s, 1H).

### Compound 33

### 6-(3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamido)-N,N-dimethylnicotinamide

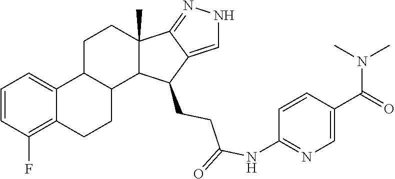 [SMILES: CN(C)C(=O)c1ccc(NC(=O)CC[C@@H]2c3c[nH]nc3[C@@]3(C)CCC4c5cccc(F)c5CCC4C23)nc1]

The compound 33 was synthesized in 59% yield from the compound 32 by the Method C in one hour reaction time.

¹H-NMR (400 MHz, DMSO-d₆): 1.11 (s, 3H), 1.35-2.60 (m, 13H), 2.69-2.94 (m, 3H), 2.98 (s, 6H), 6.93-7.02 (m, 1H), 7.11-7.21 (m, 2H), 7.41 (s, 1H), 7.85 (dd, 1H), 8.15 (d, 1H), 8.39 (d, 1H), 10.76 (s, 1H), 12.14 (br s, 1H).

### Compound 34

### 3-((13S,15R)-4-fluoro-13-methyl-17-oxo-7,8,9,11,12,13,14,15,16,17-decahydro-6H-cyclopenta al phenanthren-15-yl)-N-(5-isopropylpyridin-2-yl)propanamide

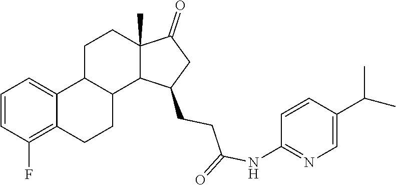 [SMILES: CC(C)c1ccc(NC(=O)CC[C@@H]2CC(=O)[C@@]3(C)CCC4c5cccc(F)c5CCC4C23)nc1]

The compound 34 was synthesized in 20% yield after chromatographic purification by the Method A1 in THE by using acid SM-IX and 2-amino-5-isopropylpyridine as starting materials in 4 hours reaction time.

¹H-NMR (200 MHz, CDCl₃): 1.07 (s, 3H), 1.25 (s, 3H), 1.28 (s, 3H), 1.34-2.60 (m, 17H), 2.72-3.05 (m, 2H), 6.83-6.92 (m, 1H), 7.05-7.18 (m, 2H), 7.57-7.63 (m, 1H), 8.09-8.17 (m, 2H), 8.49 (br s, 1H).

### Compound 35

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(5-isopropylpyridin-2-yl)propanamide

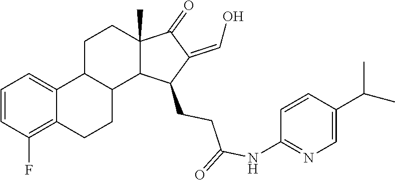 [SMILES: CC(C)c1ccc(NC(=O)CC[C@@H]2/C(=C/O)C(=O)[C@@]3(C)CCC4c5cccc(F)c5CCC4C23)nc1]

The compound 35 was synthesized in 51% yield after chromatographic purification from the compound 34 by the Method B in overnight reaction time by using 1000 mol-% of ethyl formate and 600 mol-% of NaH.

¹H-NMR (400 MHz, DMSO-d₆): 0.99 (s, 3H), 1.21 (s, 6H), 1.30-2.63 (m, 14H), 2.64-3.05 (m, 3H), 6.93-7.00 (m, 1H), 7.11-7.21 (m, 2H), 7.56 (s, 1H), 7.63-7.67 (m, 1H), 7.99-8.02 (m, 1H), 8.18 (s, 1H), 10.41 (s, 1H), 11.03 (br s, 1H).

### Compound 36

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(5-isopropylpyridin-2-yl)propanamide

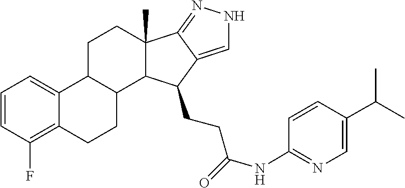 [SMILES: CC(C)c1ccc(NC(=O)CC[C@@H]2c3c[nH]nc3[C@@]3(C)CCC4c5cccc(F)c5CCC4C23)nc1]

The compound 36 was synthesized in 88% yield from the compound 35 by the Method C at 60° C. in one-hour reaction time.

¹H-NMR (400 MHz, DMSO-d₆): 1.10 (s, 3H), 1.20 (s, 3H), 1.21 (s, 3H), 1.35-2.49 (m, 14H), 2.64-2.95 (m, 3H), 6.93-7.00 (m, 1H), 7.11-7.21 (m, 2H), 7.41 (s, 1H), 7.66 (dd, 1H), 8.03 (d, 1H), 8.19 (d, 1H), 10.48 (s, 1H), 12.13 (br s, 1H).

### Compound 37

### 3-((13S,15R)-4-fluoro-13-methyl-17-oxo-7,8,9,11,12,13,14,15,16,17-decahydro-6H-cyclopenta[a]phenanthren-15-yl)-N-(5-morpholinopyridin-2-yl)propanamide

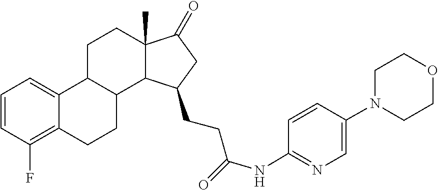 [SMILES: C[C@]12CCC3c4cccc(F)c4CCC3C1[C@H](CCC(=O)Nc1ccc(N3CCOCC3)cn1)CC2=O]

The compound 37 was synthesized in 82% yield after chromatographic purification by the Method A1 in DCM by using acid SM-IX and 5-morpholinopyridin-2-amine as starting materials and triethylamine as base in 2 hours reaction time.

¹H-NMR (400 MHz, DMSO-d₆): 0.98 (s, 3H), 1.30-2.46 (m, 16H), 2.63-2.80 (m, 1H), 2.81-2.96 (m, 1H), 3.03-3.15 (m, 4H), 3.68-3.80 (m, 4H), 6.90-7.03 (m, 1H), 7.10-7.22 (m, 2H), 7.40 (dd, 1H), 7.95-8.01 (m, 2H), 10.29 (s, 1H).

### Compound 38

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(5-morpholinopyridin-2-yl)propanamide

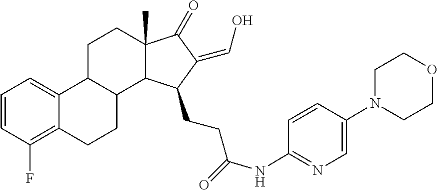 [SMILES: C[C@]12CCC3c4cccc(F)c4CCC3C1[C@H](CCC(=O)Nc1ccc(N3CCOCC3)cn1)/C(=C/O)C2=O]

The compound 38 was synthesized in 57% yield after chromatographic purification from the compound 37 by the Method B in 2 days reaction time by using 1500 mol-% of ethyl formate and 1050 mol-% of NaH.

¹H-NMR (400 MHz, DMSO-d₆): 0.99 (s, 3H), 1.30-2.60 (m, 13H), 2.63-2.99 (m, 3H), 3.03-3.15 (m, 4H), 3.68-3.80 (m, 4H), 6.90-7.03 (m, 1H), 7.10-7.22 (m, 2H), 7.39 (dd, 1H), 7.56 (s, 1H), 7.95 (d, 1H), 7.99 (d, 1H), 10.28 (s, 1H), 11.04 (br s, 1H).

### Compound 39

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(5-morpholinopyridin-2-yl)propanamide

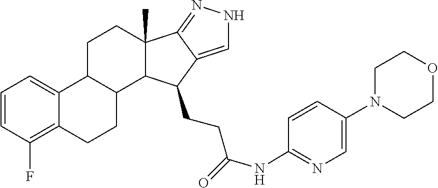 [SMILES: C[C@]12CCC3c4cccc(F)c4CCC3C1[C@H](CCC(=O)Nc1ccc(N3CCOCC3)cn1)c1c[nH]nc12]

The compound 39 was synthesized in 71% yield after chromatographic purification from the compound 38 by the Method C in one-hour reaction time.

¹H-NMR (400 MHz, DMSO-d₆): 1.10 (s, 3H), 1.35-2.49 (m, 13H), 2.67-2.95 (m, 3H), 3.05-3.14 (m, 4H), 3.69-3.78 (m, 4H), 6.92-7.03 (m, 1H), 7.12-7.22 (m, 2H), 7.39 (dd, 1H), 7.41 (s, 1H), 7.97 (d, 1H), 8.00 (d, 1H), 10.32 (s, 1H), 12.11 (br s, 1H).

### Compound 40

### 3-((13S,15R)-4-fluoro-13-methyl-17-oxo-7,8,9,11,12,13,14,15,16,17-decahydro-6H-cyclopenta[a]phenanthren-15-yl)-N-(5-(4-methylpiperazin-1-yl)pyridin-2-yl)propanamide

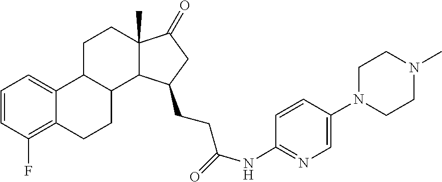 [SMILES: CN1CCN(c2ccc(NC(=O)CC[C@@H]3CC(=O)[C@@]4(C)CCC5c6cccc(F)c6CCC5C34)nc2)CC1]

The compound 40 was synthesized in 83% yield after chromatographic purification by the Method A1 in DCM by using acid SM-IX and 1-methyl-4-(6-aminopyridin-3-yl)piperazine as starting materials and triethylamine as base in 2 hours reaction time.

¹H-NMR (400 MHz, DMSO-d₆): 0.98 (s, 3H), 1.30-2.42 (m, 16H), 2.21 (s, 3H), 2.43-2.48 (m, 4H), 2.63-2.80 (m, 1H), 2.81-2.96 (m, 1H), 3.05-3.15 (m, 4H), 6.93-7.03 (m, 1H), 7.10-7.22 (m, 2H), 7.39 (dd, 1H), 7.92-8.00 (m, 2H), 10.27 (s, 1H).

### Compound 41

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(5-(4-methylpiperazin-1-yl pyridin-2-yl)propanamide

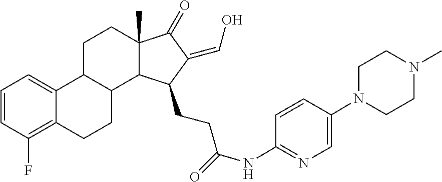 [SMILES: CN1CCN(c2ccc(NC(=O)CC[C@@H]3/C(=C/O)C(=O)[C@@]4(C)CCC5c6cccc(F)c6CCC5C34)nc2)CC1]

The compound 41 was synthesized in 57% yield from the compound 40 by the Method B in overnight reaction time.

¹H-NMR (400 MHz, DMSO-d₆): 0.99 (s, 3H), 1.30-2.49 (m, 13H), 2.28 (s, 3H), 2.51-2.60 (m, 4H), 2.63-2.96 (m, 3H), 3.10-3.20 (m, 4H), 6.93-7.03 (m, 1H), 7.09-7.22 (m, 2H), 7.39 (dd, 1H), 7.57 (s, 1H), 7.93 (d, 1H), 7.99 (d, 1H), 10.28 (s, 1H).

### Compound 42

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(5-(4-methylpiperazin-1-yl)pyridin-2-yl)propanamide

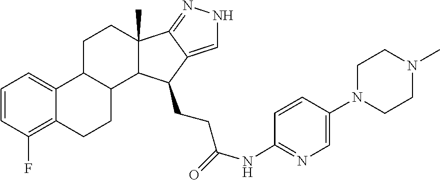 [SMILES: CN1CCN(c2ccc(NC(=O)CC[C@@H]3c4c[nH]nc4[C@@]4(C)CCC5c6cccc(F)c6CCC5C34)nc2)CC1]

The compound 42 was synthesized in 53% yield from the compound 41 by the Method C in 2.5 hours reaction time.

¹H-NMR (400 MHz, DMSO-d₆): 1.10 (s, 3H), 1.30-2.49 (m, 13H), 2.21 (s, 3H), 2.42-2.59 (m, 4H), 2.65-2.96 (m, 3H), 3.05-3.20 (m, 4H), 6.93-7.03 (m, 1H), 7.09-7.22 (m, 2H), 7.36-7.39 (m, 1H), 7.40 (s, 1H), 7.95 (d, 1H), 7.99 (d, 1H), 10.31 (s, 1H), 12.12 (br s, 1H).

### Compound 43

### 3-((13S,15R)-4-fluoro-13-methyl-17-oxo-7,8,9,11,12,13,14,15,16,17-decahydro-6H-cyclopenta[a]phenanthren-15-yl)-N-(tetrahydro-2H-pyran-4-yl)propanamide

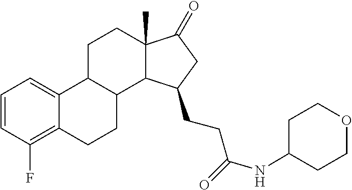 [SMILES: C[C@]12CCC3c4cccc(F)c4CCC3C1[C@H](CCC(=O)NC1CCOCC1)CC2=O]

The compound 43 was synthesized in 56% yield after chromatographic purification by the Method A3 in DCM by using acid SM-IX and 4-aminotetrahydropyran as starting materials in 5 hours reaction time.

¹H-NMR (400 MHz, DMSO-d₆): 0.96 (s, 3H), 1.30-2.41 (m, 20H), 2.67-2.76 (m, 1H), 2.85-2.90 (m, 1H), 3.29-3.30 (m, 2H), 3.70-3.77 (m, 1H), 3.80-3.83 (m, 2H), 6.94-7.00 (m, 1H), 7.10-7.22 (m, 2H), 7.84 (d, 1H).

### Compound 44

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(tetrahydro-2H-pyran-4-yl)propanamide

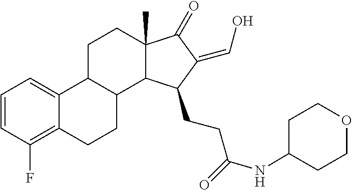 [SMILES: C[C@]12CCC3c4cccc(F)c4CCC3C1[C@H](CCC(=O)NC1CCOCC1)/C(=C/O)C2=O]

The compound 44 was synthesized in quantitative yield from the compound 43 by the Method B in 6 hours reaction time by using 1000 mol-% of ethyl formate and 600 mol-% of NaH.

¹H-NMR (400 MHz, DMSO-d₆): 0.97 (s, 3H), 1.30-2.45 (m, 17H), 2.65-2.95 (m, 3H), 3.25-3.45 (m, 2H), 3.70-3.95 (m, 3H), 6.94-7.00 (m, 1H), 7.10-7.22 (m, 2H), 7.49 (s, 1H), 8.14 (d, 1H), 11.59 (br s, 1H).

### Compound 45

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(tetrahydro-2H-pyran-4-yl)propanamide

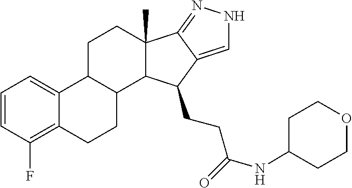 [SMILES: C[C@]12CCC3c4cccc(F)c4CCC3C1[C@H](CCC(=O)NC1CCOCC1)c1c[nH]nc12]

The compound 45 was synthesized in 76% yield from the compound 44 by the Method C in 1.5 hours reaction time.

¹H-NMR (400 MHz, DMSO-d₆): 1.09 (s, 3H), 1.30-2.49 (m, 17H), 2.65-2.95 (m, 3H), 3.25-3.45 (m, 2H), 3.70-3.90 (m, 3H), 6.94-7.01 (m, 1H), 7.10-7.22 (m, 2H), 7.40 (s, 1H), 7.86 (d, 1H), 12.12 (br s, 1H).

### Compound 46

### 3-((13S,15R)-4-fluoro-13-methyl-17-oxo-7,8,9,11,12,13,14,15,16,17-decahydro-6H-cyclopenta[a]phenanthren-15-yl)-N-(4-methoxypyridin-2-yl)propanamide

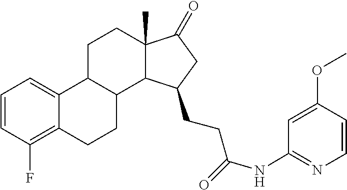 [SMILES: COc1ccnc(NC(=O)CC[C@@H]2CC(=O)[C@@]3(C)CCC4c5cccc(F)c5CCC4C23)c1]

The compound 46 was synthesized in 47% yield after chromatographic purification by the Method A2 in THE by using acid SM-IX and 2-amino-4-methoxypyridine as starting materials in 10 hours and overnight at room temperature. Reaction needed 250 mol-% of amine, EDCI and HOBT.

¹H-NMR (400 MHz, DMSO-d₆): 0.98 (s, 3H), 1.30-2.48 (m, 16H), 2.65-2.78 (m, 1H), 2.80-2.92 (m, 1H), 3.81 (s, 3H), 6.68-6.72 (m, 1H), 6.94-7.00 (m, 1H), 7.10-7.21 (m, 2H), 7.73 (s, 1H), 8.10-8.13 (m, 1H), 10.47 (s, 1H).

### Compound 47

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(4-methoxypyridin-2-yl)propanamide

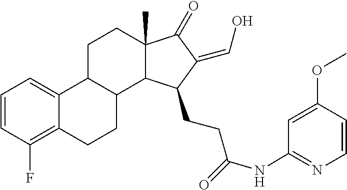 [SMILES: COc1ccnc(NC(=O)CC[C@@H]2/C(=C/O)C(=O)[C@@]3(C)CCC4c5cccc(F)c5CCC4C23)c1]

The compound 47 was synthesized in 86% yield from the compound 46 by the Method B in 2.5 hours reaction time.

¹H-NMR (400 MHz, DMSO-d₆): 0.99 (s, 3H), 1.30-2.65 (m, 13H), 2.62-2.97 (m, 3H), 3.80 (s, 3H), 6.65-6.70 (dd, 1H), 6.93-7.00 (m, 1H), 7.10-7.21 (m, 2H), 7.58 (s, 1H), 7.73 (d, 1H), 8.10-8.13 (d, 1H), 10.46 (s, 1H), 11.04 (br s).

### Compound 48

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(4-methoxypyridin-2-yl)propanamide

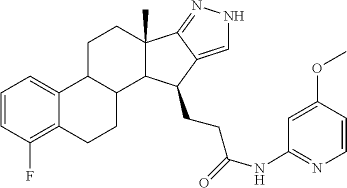 [SMILES: COc1ccnc(NC(=O)CC[C@@H]2c3c[nH]nc3[C@@]3(C)CCC4c5cccc(F)c5CCC4C23)c1]

The compound 48 was synthesized in 47% yield from the compound 47 by the Method C in 3 hours reaction time.

¹H-NMR (400 MHz, DMSO-d₆): 1.11 (s, 3H), 1.30-2.49 (m, 13H), 2.70-2.97 (m, 3H), 3.80 (s, 3H), 6.68-6.72 (dd, 1H), 6.93-7.01 (m, 1H), 7.10-7.21 (m, 2H), 7.39 (s, 1H), 7.73 (d, 1H), 8.10-8.14 (d, 1H), 10.50 (s, 1H), 12.12 (br s).

### Compound 49

### 3-((13S,15R)-4-fluoro-13-methyl-17-oxo-7,8,9,11,12,13,14,15,16,17-decahydro-6H-cyclopenta[a]phenanthren-15-yl)-N-(pyrazin-2-yl)propanamide

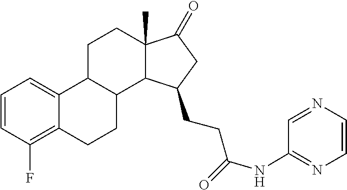 [SMILES: C[C@]12CCC3c4cccc(F)c4CCC3C1[C@H](CCC(=O)Nc1cnccn1)CC2=O]

The compound 49 was synthesized in 53% yield after chromatographic purification by the Method A1 in DCM by using acid SM-IX and aminopyrazine as starting materials in 5 hours reaction time.

¹H-NMR (400 MHz, DMSO-d₆): 0.99 (s, 3H), 1.30-2.60 (m, 16H), 2.69-2.78 (m, 1H), 2.84-2.92 (m, 1H), 6.94-7.00 (m, 1H), 7.12-7.20 (m, 2H), 8.33-8.40 (m, 2H), 9.35 (s, 1H), 10.81 (s, 1H).

### Compound 50

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(pyrazin-2-yl)propanamide

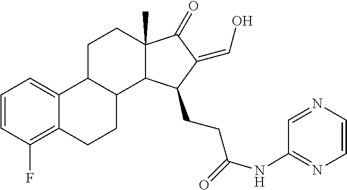 [SMILES: C[C@]12CCC3c4cccc(F)c4CCC3C1[C@H](CCC(=O)Nc1cnccn1)/C(=C/O)C2=O]

The compound 50 was synthesized in quantitative yield from the compound 49 by the Method B in overnight reaction time by using 750 mol-% of ethyl formate and 450 mol-% of NaH.

¹H-NMR (400 MHz, DMSO-d₆): 1.00 (s, 3H), 1.30-2.49 (m, 13H), 2.55-2.99 (m, 3H), 6.94-7.00 (m, 1H), 7.11-7.20 (m, 2H), 7.54 (s, 1H), 8.33-8.40 (m, 2H), 9.34 (s, 1H), 10.75 (s, 1H), 10.98 (br s, 1H).

### Compound 51

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(pyrazin-2-yl)propanamide

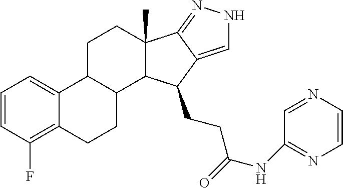 [SMILES: C[C@]12CCC3c4cccc(F)c4CCC3C1[C@H](CCC(=O)Nc1cnccn1)c1c[nH]nc12]

The compound 51 was synthesized in 30% yield after chromatographic purification from the compound 50 by the Method C in one-hour reaction time.

¹H-NMR (400 MHz, DMSO-d₆): 1.11 (s, 3H), 1.35-2.60 (m, 13H), 2.65-2.99 (m, 3H), 6.94-7.01 (m, 1H), 7.11-7.20 (m, 2H), 7.40 (s, 1H), 8.33-8.41 (m, 2H), 9.35 (s, 1H), 10.85 (s, 1H), 12.16 (br s, 1H).

### Compound 52

### 3-((13S,15R)-4-fluoro-13-methyl-17-oxo-7,8,9,11,12,13,14,15,16,17-decahydro-6H-cyclopenta[a]phenanthren-15-yl)-N-(1-methyl-1H-pyrazol-3-yl)propanamide

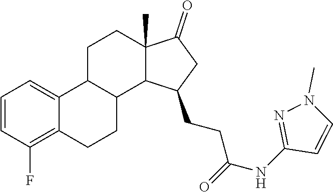 [SMILES: Cn1ccc(NC(=O)CC[C@@H]2CC(=O)[C@@]3(C)CCC4c5cccc(F)c5CCC4C23)n1]

The compound 52 was synthesized in 80% yield by the Method A1 in THE by using acid SM-IX and 1-methyl-1H-pyrazol-3-amine as starting materials and 300 mol-% of T3P in overnight reaction time.

¹H-NMR (400 MHz, DMSO-d₆): 0.97 (s, 3H), 1.30-2.49 (m, 16H), 2.65-2.92 (m, 2H), 3.72 (s, 3H), 6.43 (d, 1H), 6.94-7.00 (m, 1H), 7.12-7.20 (m, 2H), 7.52 (d, 1H), 10.36 (s, 1H).

### Compound 53

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(1-methyl-1H-pyrazol-3-yl)propanamide

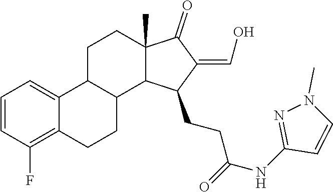 [SMILES: Cn1ccc(NC(=O)CC[C@@H]2/C(=C/O)C(=O)[C@@]3(C)CCC4c5cccc(F)c5CCC4C23)n1]

The compound 53 was synthesized in quantitative yield from the compound 52 by the Method B in 4 hours reaction time.

¹H-NMR (400 MHz, DMSO-d₆): 0.99 (s, 3H), 1.30-2.49 (m, 13H), 2.65-2.96 (m, 3H), 3.72 (s, 3H), 6.42 (d, 1H), 6.93-7.02 (m, 1H), 7.11-7.23 (m, 2H), 7.51 (d, 1H), 7.54 (s, 1H), 10.35 (s, 1H), 11.02 (br s, 1H).

### Compound 54

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(1-methyl-1H-pyrazol-3-yl)propanamide

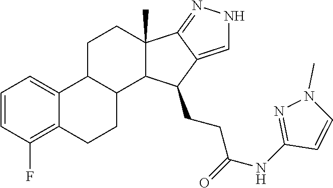 [SMILES: Cn1ccc(NC(=O)CC[C@@H]2c3c[nH]nc3[C@@]3(C)CCC4c5cccc(F)c5CCC4C23)n1]

The compound 54 was synthesized in 69% yield from the compound 53 by the Method C in one-hour reaction time.

¹H-NMR (400 MHz, DMSO-d₆): 1.10 (s, 3H), 1.30-2.49 (m, 13H), 2.65-2.96 (m, 3H), 3.73 (s, 3H), 6.44 (d, 1H), 6.93-7.02 (m, 1H), 7.11-7.23 (m, 2H), 7.40 (s, 1H), 7.52 (d, 1H), 10.41 (s, 1H), 12.12 (br s, 1H).

### Compound 55

### (13S,15R)-4-fluoro-15-(3-(isoindolin-2-yl)-3-oxopropyl)-13-methyl-6,7,8,9,11,12,13,14,15,16-decahydro-17H-cyclopenta[a]phenanthren-17-one

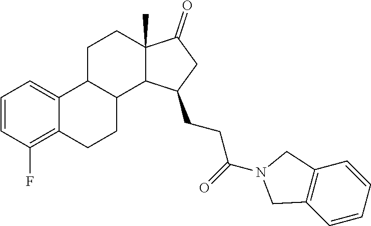 [SMILES: C[C@]12CCC3c4cccc(F)c4CCC3C1[C@H](CCC(=O)N1Cc3ccccc3C1)CC2=O]

The compound 55 was synthesized in quantitative yield by the Method A1 in THE by using acid SM-IX and isoindoline as starting materials in overnight reaction time.

¹H-NMR (400 MHz, CDCl₃): 1.09 (s, 3H), 1.34-2.60 (m, 16H), 2.72-3.05 (m, 2H), 4.82 (s, 4H), 6.83-6.92 (m, 1H), 7.05-7.18 (m, 2H), 7.25-7.34 (m, 4H).

### Compound 56

### (13S,15S,Z)-4-fluoro-16-(hydroxymethylene)-15-(3-(isoindolin-2-yl)-3-oxopropyl)-13-methyl-6,7,8,9,11,12,13,14,15,16-decahydro-17H-cyclopenta[a]phenanthren-17-one

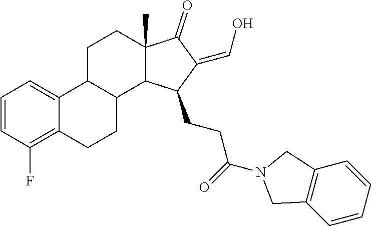 [SMILES: C[C@]12CCC3c4cccc(F)c4CCC3C1[C@H](CCC(=O)N1Cc3ccccc3C1)/C(=C/O)C2=O]

The compound 56 was synthesized in 34% yield from the compound 55 by the Method B in overnight reaction time.

¹H-NMR (400 MHz, DMSO-d₆): 1.00 (s, 3H), 1.30-2.62 (m, 13H), 2.63-3.05 (m, 3H), 4.65 (s, 2H), 4.75-4.88 (m, 2H), 6.93-7.02 (m, 1H), 7.11-7.23 (m, 2H), 7.25-7.40 (m, 4H), 7.55 (s, 1H), 11.09 (br s, 1H).

### Compound 57

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-1-(isoindolin-2-yl)propan-1-one

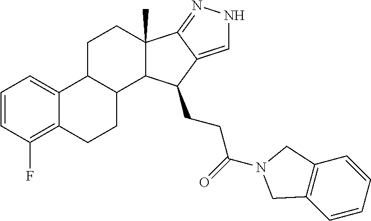 [SMILES: C[C@]12CCC3c4cccc(F)c4CCC3C1[C@H](CCC(=O)N1Cc3ccccc3C1)c1c[nH]nc12]

The compound 57 was synthesized in 93% yield from the compound 56 by the Method C in one-hour reaction time.

¹H-NMR (400 MHz, DMSO-d₆): 1.12 (s, 3H), 1.30-2.49 (m, 13H), 2.63-3.00 (m, 3H), 4.64 (s, 2H), 4.80-4.99 (m, 2H), 6.91-7.05 (m, 1H), 7.11-7.23 (m, 2H), 7.25-7.40 (m, 4H), 7.48 (s, 1H), 12.13 (br s, 1H).

### Compound 58

### N-(cyclopropylmethyl)-3-((13S,15R)-4-fluoro-13-methyl-17-oxo-7,8,9,11,12,13,14,15,16,17-decahydro-6H-cyclopenta[a]phenanthren-15-yl)-N-methylpropanamide

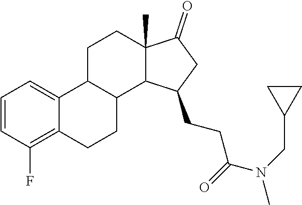 [SMILES: CN(CC1CC1)C(=O)CC[C@@H]1CC(=O)[C@@]2(C)CCC3c4cccc(F)c4CCC3C12]

The compound 58 was synthesized in 34% yield by the Method A1 in THE by using acid SM-IX and 400 mol-% of (cyclopropylmethyl)(methyl)amine, 600 mol-% of pyridine and 400 mol-% of T3P in overnight reaction time.

¹H-NMR (400 MHz, CDCl₃): 0.21-0.27 (m, 2H), 0.45-0.55 (m, 1H), 0.55-0.65 (m, 1H), 0.87-1.02 (m, 1H), 1.08 (s, 3H), 1.30-2.65 (m, 16H), 2.75-3.00 (m, 2H), 3.01/3.07 (2×s, 3H, isomers), 3.13-3.36 (m, 2H), 6.83-6.92 (m, 1H), 7.04-7.18 (m, 2H).

### Compound 59

### N-(cyclopropylmethyl)-3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-methylpropanamide

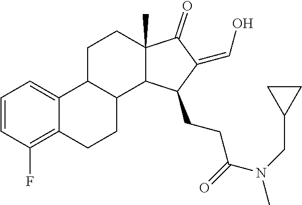 [SMILES: CN(CC1CC1)C(=O)CC[C@@H]1/C(=C/O)C(=O)[C@@]2(C)CCC3c4cccc(F)c4CCC3C12]

The compound 59 was synthesized in quantitative yield from the compound 58 by the Method B in overnight reaction time by using 1200 mol-% of ethyl formate and 800 mol-% of NaH.

¹H-NMR (400 MHz, DMSO-d₆): 0.15-0.27 (m, 2H), 0.35-0.55 (m, 2H), 0.90-1.02 (m, 1H), 1.17 (s, 3H), 1.30-2.49 (m, 13H), 2.59-3.00 (m, 3H), 2.88/3.04 (2×s, 3H, isomers), 3.05-3.30 (m, 2H), 6.93-7.02 (m, 1H), 7.11-7.23 (m, 2H), 7.48-7.51 (br s, 1H), 11.33/11.50 (2×br s, 1H, isomers).

### Compound 60

### N-(cyclopropylmethyl)-3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-methylpropanamide

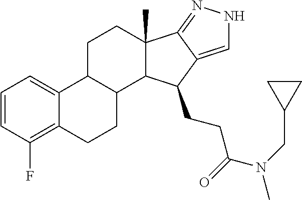 [SMILES: CN(CC1CC1)C(=O)CC[C@@H]1c2c[nH]nc2[C@@]2(C)CCC3c4cccc(F)c4CCC3C12]

The compound 60 was synthesized in 30% yield after chromatographic purification from the compound 59 by the Method C in one-hour reaction time.

¹H-NMR (400 MHz, DMSO-d₆): 0.15-0.27 (m, 2H), 0.35-0.55 (m, 2H), 0.90-1.02 (m, 1H), 1.10 (s, 3H), 1.30-2.49 (m, 13H), 2.68-3.00 (m, 3H), 2.86/3.04 (2×s, 3H, isomers), 3.05-3.30 (m, 2H), 6.93-7.02 (m, 1H), 7.11-7.23 (m, 2H), 7.38-7.45 (2×br s, 1H, isomers), 12.12 (br s, 1H).

### Compound 61

### 3-((13S,15R)-4-fluoro-13-methyl-17-oxo-7,8,9,11,12,13,14,15,16,17-decahydro-6H-cyclopenta[a]phenanthren-15-yl)-N-(1-methyl-1H-pyrazol-4-yl)propanamide

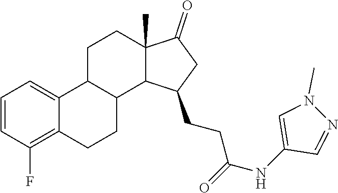 [SMILES: Cn1cc(NC(=O)CC[C@@H]2CC(=O)[C@@]3(C)CCC4c5cccc(F)c5CCC4C23)cn1]

The compound 61 was synthesized in 70% yield by the Method A2 in DMF by using acid SM-IX and 1-methyl-1H-pyrazol-4-amine as starting materials.

¹H-NMR (400 MHz, CDCl₃): 1.07 (s, 3H), 1.39-2.48 (m, 16H), 2.76-3.02 (m, 2H), 3.49 (s, 3H), 6.88 (dd, 1H), 7.06-7.15 (m, 2H), 7.36 (m, 2H), 7.91 (s, 1H).

### Compound 62

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(1-methyl-1H-pyrazol-4-yl)propanamide

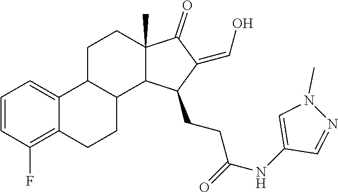 [SMILES: Cn1cc(NC(=O)CC[C@@H]2/C(=C/O)C(=O)[C@@]3(C)CCC4c5cccc(F)c5CCC4C23)cn1]

The compound 62 was synthesized in 76% yield from the compound 61 by the Method B in overnight reaction time.

¹H-NMR (400 MHz, DMSO-d₆): 0.99 (s, 3H), 1.36-2.45 (m, 13H), 2.69-2.93 (m, 3H), 3.77 (s, 3H), 6.97 (m, 1H), 7.09-7.22 (m, 2H), 7.36 (s, 1H), 7.55 (s, 1H), 7.84 (s, 1H), 9.98 (s, 1H), 11.10 (br s, 1H).

### Compound 63

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(1-methyl-1H-pyrazol-4-yl)propanamide

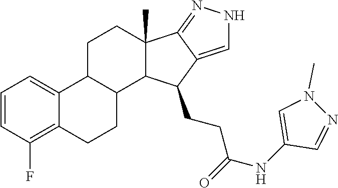 [SMILES: Cn1cc(NC(=O)CC[C@@H]2c3c[nH]nc3[C@@]3(C)CCC4c5cccc(F)c5CCC4C23)cn1]

The compound 63 was synthesized in 84% yield from the compound 62 by the Method C in one-hour reaction time.

¹H-NMR (400 MHz, DMSO-d₆): 1.10 (s, 3H), 1.47-2.29 (m, 13H), 2.67-2.92 (m, 3H), 3.77 (s, 3H), 6.98 (m, 1H), 7.16 (m, 2H), 7.37 (d, 2H), 7.87 (s, 1H), 9.95 (s, 1H), 12.15 (br s, 1H).

### Compound 64

### 3-((13S,15R)-4-fluoro-13-methyl-17-oxo-7,8,9,11,12,13,14,15,16,17-decahydro-6H-cyclopenta[a]phenanthren-15-yl)-N-(5-methyl-1,3,4-oxadiazol-2-yl)propanamide

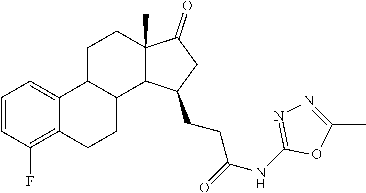 [SMILES: Cc1nnc(NC(=O)CC[C@@H]2CC(=O)[C@@]3(C)CCC4c5cccc(F)c5CCC4C23)o1]

The compound 64 was synthesized in 45% yield by the Method A2 in DMF by using acid SM-IX and 5-methyl-1,3,4-oxadiazol-2-ylamine as starting materials in 5 hours reaction time.

¹H-NMR (400 MHz, DMSO-d₆): 0.97 (s, 3H), 1.30-2.43 (m, 16H), 2.44 (s, 3H), 2.66-2.98 (m, 2H), 6.92-7.03 (m, 1H), 7.10-7.22 (m, 2H), 11.50 (br s, 1H).

### Compound 65

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(5-methyl-1,3,4-oxadiazol-2-yl)propanamide

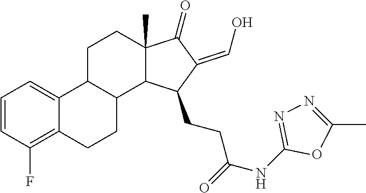 [SMILES: Cc1nnc(NC(=O)CC[C@@H]2/C(=C/O)C(=O)[C@@]3(C)CCC4c5cccc(F)c5CCC4C23)o1]

The compound 65 was synthesized in 97% yield from the compound 64 by the Method B in overnight reaction time by using 900 mol-% of ethyl formate and 600 mol-% of NaH.

¹H-NMR (400 MHz, DMSO-d₆): 0.99 (s, 3H), 1.30-2.65 (m, 13H), 2.43 (s, 3H), 2.66-2.99 (m, 3H), 6.92-7.03 (m, 1H), 7.10-7.22 (m, 2H), 7.56 (s, 1H), 10.96 (br s, 1H), 11.52 (br s, 1H).

### Compound 66

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(5-methyl-1,3,4-oxadiazol-2-yl)propanamide

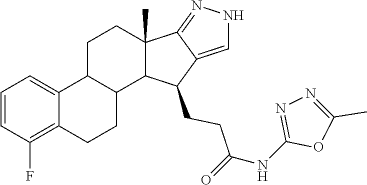 [SMILES: Cc1nnc(NC(=O)CC[C@@H]2c3c[nH]nc3[C@@]3(C)CCC4c5cccc(F)c5CCC4C23)o1]

The compound 66 was synthesized in 3% yield from the compound 65 by the Method C in 2.5 hours reaction time.

¹H-NMR (400 MHz, CDCl₃): 1.18 (s, 3H), 1.40-2.50 (m, 13H), 2.57 (s, 3H), 2.65-3.05 (m, 3H), 6.84-6.95 (m, 1H), 7.03-7.17 (m, 2H).

### Compound 67

### 3-((13S,15R)-4-fluoro-13-methyl-17-oxo-7,8,9,11,12,13,14,15,16,17-decahydro-6H-cyclopenta[a]phenanthren-15-yl)-N-(piperidin-1-yl)propanamide

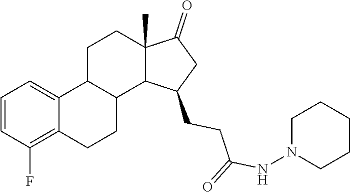 [SMILES: C[C@]12CCC3c4cccc(F)c4CCC3C1[C@H](CCC(=O)NN1CCCCC1)CC2=O]

The compound 67 was synthesized in 45% yield after chromatographic purification by the Method A3 in DCM by using acid SM-IX and piperidin-1-amine as starting materials in 4 hours reaction time.

¹H-NMR (400 MHz, DMSO-d₆): 0.95/0.96 (2×s, 3H, isomers), 1.30-2.45 (m, 23H), 2.60-2.98 (m, 5H), 6.92-7.03 (m, 1H), 7.10-7.22 (m, 2H), 8.33/8.75 (2×s, 1H, isomers).

### Compound 68

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-piperidin-1-yl)propanamide

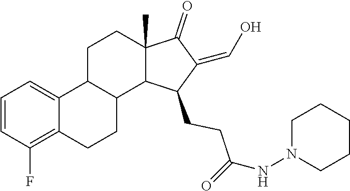 [SMILES: C[C@]12CCC3c4cccc(F)c4CCC3C1[C@H](CCC(=O)NN1CCCCC1)/C(=C/O)C2=O]

The compound 68 was synthesized in 77% yield from the compound 67 by the Method B in overnight reaction time by using 1200 mol-% of ethyl formate and 800 mol-% of NaH.

¹H-NMR (400 MHz, DMSO-d₆): 0.97 (s, 3H), 1.30-2.45 (m, 20H), 2.56-2.95 (m, 6H), 6.92-7.03 (m, 1H), 7.10-7.22 (m, 2H), 7.50/7.53 (2×s, 1H, isomers), 8.49/9.05 (2×s, 1H, isomers), 11.37 (br s, 1H).

### Compound 69

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(piperidin-1-yl)propanamide

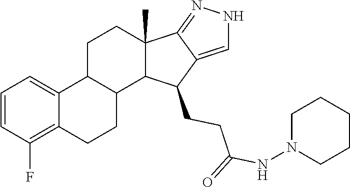 [SMILES: C[C@]12CCC3c4cccc(F)c4CCC3C1[C@H](CCC(=O)NN1CCCCC1)c1c[nH]nc12]

The compound 69 was synthesized in 72% yield from the compound 68 by the Method C in one hour reaction time.

¹H-NMR (400 MHz, DMSO-d₆): 1.08/1.09 (2×s, 3H, isomers), 1.29-2.49 (m, 20H), 2.59-2.95 (m, 6H), 6.92-7.03 (m, 1H), 7.10-7.22 (m, 2H), 7.32/7.41 (2×s, 1H, isomers), 8.31/8.79 (2×s, 1H, isomers), 12.11 (br s, 1H).

### Compound 70

### N-(tert-butyl)-3-((13S,15R)-4-fluoro-13-methyl-17-oxo-7,8,9,11,12,13,14,15,16,17-decahydro-6H-cyclopenta[a]phenanthren-15-yl)propanamide

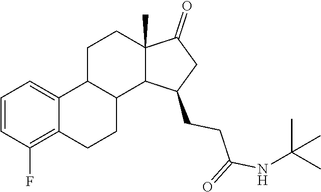 [SMILES: CC(C)(C)NC(=O)CC[C@@H]1CC(=O)[C@@]2(C)CCC3c4cccc(F)c4CCC3C12]

The compound 70 was synthesized in 81% yield by the Method A1 in DCM by using acid SM-IX, 300 mol-% of tert-butylamine, 450 mol-% of pyridine and 300 mol-% of T3P in overnight reaction time and then warming at 40° C. for 5 hours.

¹H-NMR (400 MHz, DMSO-d₆): 0.95 (s, 3H), 1.25 (s, 9H), 1.30-2.43 (m, 16H), 2.66-2.95 (m, 2H), 6.92-7.02 (m, 1H), 7.10-7.22 (m, 2H), 7.46 (s, 1H).

### Compound 71

### N-(tert-butyl)-3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-_y)propanamide

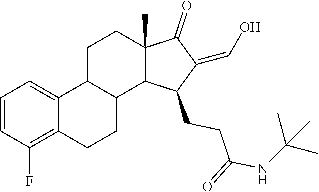 [SMILES: CC(C)(C)NC(=O)CC[C@@H]1/C(=C/O)C(=O)[C@@]2(C)CCC3c4cccc(F)c4CCC3C12]

The compound 71 was synthesized in 77% yield from the compound 70 by the Method B in 2.5 hours reaction time.

¹H-NMR (400 MHz, DMSO-d₆): 0.98 (s, 3H), 1.28 (s, 9H), 1.30-2.43 (m, 13H), 2.66-2.95 (m, 3H), 6.92-7.02 (m, 1H), 7.10-7.22 (m, 2H), 7.50 (s, 1H), 7.93 (s, 1H), 11.91 (br s, 1H).

### Compound 72

### N-(tert-butyl)-3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamide

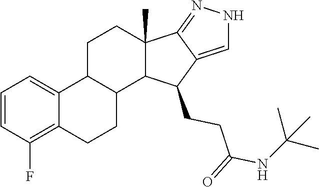 [SMILES: CC(C)(C)NC(=O)CC[C@@H]1c2c[nH]nc2[C@@]2(C)CCC3c4cccc(F)c4CCC3C12]

The compound 72 was synthesized in 89% yield from the compound 71 by the Method C in 30 minutes reaction time.

¹H-NMR (400 MHz, DMSO-d₆): 1.09 (s, 3H), 1.25 (s, 9H), 1.30-2.45 (m, 13H), 2.66-2.95 (m, 3H), 6.92-7.02 (m, 1H), 7.10-7.22 (m, 2H), 7.38 (s, 1H), 7.45 (s, 1H), 12.12 (br s, 1H).

### Compound 73

### (13S,15R)-4-fluoro-13-methyl-15-(3-(4-(1-methylpiperidin-4-yl)piperazin-1-yl)-3-oxopropyl)-6,7,8,9,11,12,13,14,15,16-decahydro-17H-cyclopenta[a]phenanthren-17-one

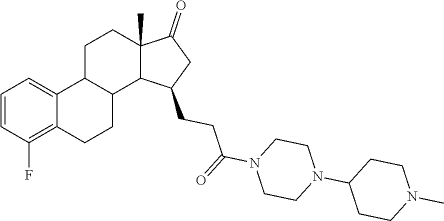 [SMILES: CN1CCC(N2CCN(C(=O)CC[C@@H]3CC(=O)[C@@]4(C)CCC5c6cccc(F)c6CCC5C34)CC2)CC1]

The compound 73 was synthesized in 70% yield after chromatographic purification by the Method A3 in DCM by using acid SM-IX and 1-(1-Methyl-4-piperidinyl)piperazine as starting materials in 5 hours reaction time.

¹H-NMR (400 MHz, DMSO-d₆): 0.96 (s, 3H), 1.30-2.49 (m, 28H), 2.12 (s, 3H), 2.65-2.95 (m, 3H), 3.35-3.50 (m, 4H), 6.92-7.02 (m, 1H), 7.10-7.22 (m, 2H).

### Compound 74

### (13S,15S,Z)-4-fluoro-16-(hydroxymethylene)-13-methyl-15-(3-(4-(1-methylpiperidin-4-yl)piperazin-1-yl)-3-oxopropyl)-6,7,8,9,11,12,13,14,15,16-decahydro-17H-cyclopenta[a]phenanthren-17-one

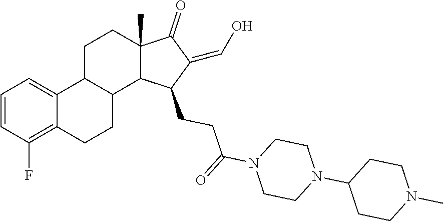 [SMILES: CN1CCC(N2CCN(C(=O)CC[C@@H]3/C(=C/O)C(=O)[C@@]4(C)CCC5c6cccc(F)c6CCC5C34)CC2)CC1]

The compound 74 was synthesized in 89% yield from the compound 73 by the Method B in 6 hours reaction time.

¹H-NMR (400 MHz, CDCl₃): 1.08 (s, 3H), 1.35-3.10 (m, 29H), 2.29 (s, 3H), 3.40-3.70 (m, 4H), 6.83-6.92 (m, 1H), 7.02-7.22 (m, 2H), 7.66 (s, 1H).

### Compound 75

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-1-(4-(1-methylpiperidin-4-yl)piperazin-1-yl)propan-1-one

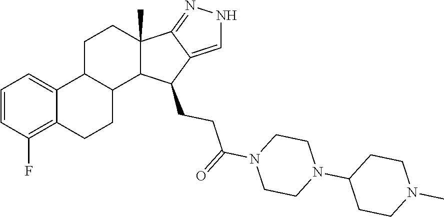 [SMILES: CN1CCC(N2CCN(C(=O)CC[C@@H]3c4c[nH]nc4[C@@]4(C)CCC5c6cccc(F)c6CCC5C34)CC2)CC1]

The compound 75 was synthesized in 85% yield from the compound 74 by the Method C in one hour reaction time.

¹H-NMR (400 MHz, DMSO-d₆): 1.09 (s, 3H), 1.30-2.49 (m, 24H), 2.12 (s, 3H), 2.65-2.95 (m, 5H), 3.35-3.50 (m, 4H), 6.92-7.02 (m, 1H), 7.10-7.22 (m, 2H), 7.41 (s, 1H), 12.11 (br s, 1H).

### Compound 76

### 3-((13S,15R)-4-fluoro-13-methyl-17-oxo-7,8,9,11,12,13,14,15,16,17-decahydro-6H-cyclopenta[a]phenanthren-15-yl)-N-(5-methylisoxazol-3-yl)propanamide

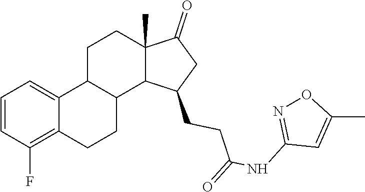 [SMILES: Cc1cc(NC(=O)CC[C@@H]2CC(=O)[C@@]3(C)CCC4c5cccc(F)c5CCC4C23)no1]

The compound 76 was synthesized in 95% yield by the Method A1 in DCM by using acid SM-IX and 3-amino-5-methylisoxazole as starting materials in 4 hours reaction time.

¹H-NMR (200 MHz, DMSO-d₆): 0.97 (s, 3H), 1.24-2.46 (m, 16H), 2.37 (s, 3H), 2.58-3.01 (m, 2H), 6.64 (s, 1H), 6.88-7.06 (m, 1H), 7.07-7.25 (m, 2H), 10.88 (s, 1H).

### Compound 77

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(5-methylisoxazol-3-yl)propanamide

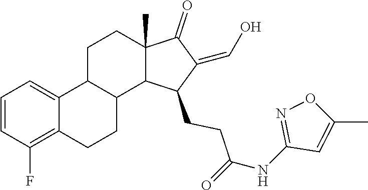 [SMILES: Cc1cc(NC(=O)CC[C@@H]2/C(=C/O)C(=O)[C@@]3(C)CCC4c5cccc(F)c5CCC4C23)no1]

The compound 77 was synthesized in quantitative yield from the compound 76 by the Method B in one hour reaction time.

¹H-NMR (400 MHz, DMSO-d₆): 0.99 (s, 3H), 1.30-2.60 (m, 13H), 2.36 (s, 3H), 2.65-3.10 (m, 3H), 6.63 (s, 1H), 6.91-7.06 (m, 1H), 7.07-7.25 (m, 2H), 7.53 (s, 1H), 10.82 (s, 1H), 11.00 (br s, 1H).

### Compound 78

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(5-methylisoxazol-3-yl)propanamide

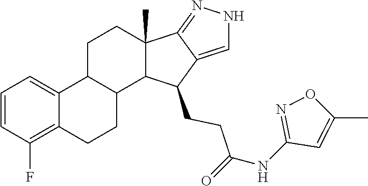 [SMILES: Cc1cc(NC(=O)CC[C@@H]2c3c[nH]nc3[C@@]3(C)CCC4c5cccc(F)c5CCC4C23)no1]

The compound 78 was synthesized in 59% yield from the compound 77 by the Method C in 0.5 hours reaction time.

¹H-NMR (400 MHz, DMSO-d₆): 1.10 (s, 3H), 1.32-2.49 (m, 13H), 2.36 (s, 3H), 2.65-3.10 (m, 3H), 6.65 (s, 1H), 6.93-7.04 (m, 1H), 7.09-7.25 (m, 2H), 7.38 (s, 1H), 10.92 (s, 1H), 12.14 (br s, 1H).

### Compound 79

### 3-((13S,15R)-4-fluoro-13-methyl-17-oxo-7,8,9,11,12,13,14,15,16,17-decahydro-6H-cyclopenta[a]phenanthren-15-yl)-N-(1,3,4-thiadiazol-2-yl)propanamide

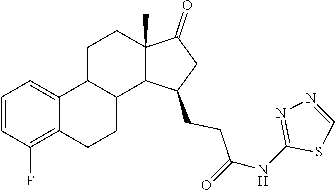 [SMILES: C[C@]12CCC3c4cccc(F)c4CCC3C1[C@H](CCC(=O)Nc1nncs1)CC2=O]

The compound 79 was synthesized in 61% yield after chromatographic purification by the Method A3 in DCM by using acid SM-IX and 2-amino-1,3,4-thiadiazole as starting materials in 5.5 hours reaction time.

¹H-NMR (200 MHz, CDCl₃): 1.03 (s, 3H), 1.20-3.05 (m, 18H), 6.80-6.95 (m, 1H), 7.03-7.18 (m, 2H), 8.82 (s, 1H), 13.67 (br s, 1H).

### Compound 80

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(1,3,4-thiadiazol-2-yl)propanamide

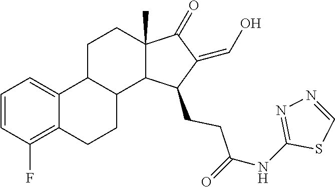 [SMILES: C[C@]12CCC3c4cccc(F)c4CCC3C1[C@H](CCC(=O)Nc1nncs1)/C(=C/O)C2=O]

The compound 80 was synthesized in 98% yield from the compound 81 by the Method B in overnight reaction time.

¹H-NMR (400 MHz, DMSO-d₆): 1.00 (s, 3H), 1.30-2.49 (m, 13H), 2.65-3.05 (m, 3H), 6.91-7.04 (m, 1H), 7.10-7.25 (m, 2H), 7.57 (s, 1H), 9.13 (s, 1H), 10.98 (br s, 1H), 12.63 (br s, 1H).

### Compound 81

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(1,3,4-thiadiazol-2-yl)propanamide

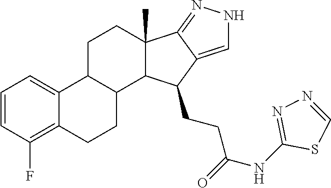 [SMILES: C[C@]12CCC3c4cccc(F)c4CCC3C1[C@H](CCC(=O)Nc1nncs1)c1c[nH]nc12]

The compound 81 was synthesized in 98% yield from the compound 80 by the Method C in 30 minutes reaction time.

¹H-NMR (400 MHz, DMSO-d₆): 1.11 (s, 3H), 1.35-2.49 (m, 11H), 2.51-3.01 (m, 5H), 6.91-7.04 (m, 1H), 7.10-7.25 (m, 2H), 7.37 (s, 1H), 9.15 (s, 1H), 12.15 (br s, 1H), 12.61 (br s, 1H).

### Compound 82

### 3-((13S,15R)-3-fluoro-13-methyl-17-oxo-7,8,9,11,12,13,14,15,16,17-decahydro-6H-cyclopenta[a]phenanthren-15-yl)-N-(5-methoxypyridin-2-yl)propanamide

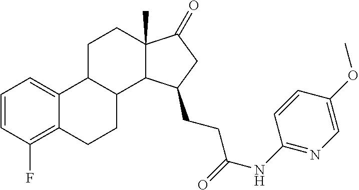 [SMILES: COc1ccc(NC(=O)CC[C@@H]2CC(=O)[C@@]3(C)CCC4c5cccc(F)c5CCC4C23)nc1]

The compound 82 was synthesized in 62% yield by the Method A2 in DMF by using acid SM-IX and 5-methoxypyridine-2-amine as starting materials in 2 hours reaction time.

¹H-NMR (200 MHz, CDCl₃): 1.07 (s, 3H), 1.39-2.50 (m, 16H), 2.94 (m, 2H), 3.85 (s, 3H), 6.79-6.88 (m, 2H), 7.19-7.30 (m, 2H), 7.90 (br s, 1H), 7.95 (d, 1H), 8.14 (d, 1H).

### Compound 83

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(5-methoxypyridin-2-yl propanamide

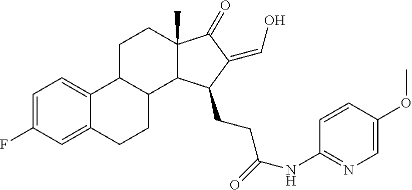 [SMILES: COc1ccc(NC(=O)CC[C@@H]2/C(=C/O)C(=O)[C@@]3(C)CCC4c5ccc(F)cc5CCC4C23)nc1]

The compound 83 was prepared from the compound 82 by the Method B in quantitative yield.

¹H NMR (400 MHz, DMSO-d₆): 0.99 (s, 3H), 1.48-2.46 (m, 12H), 2.83-2.97 (m, 3H), 3.79 (s, 3H), 6.89-6.95 (m, 2H), 7.27 (m, 1H), 7.40 (dd, 1H), 7.54 (s, 1H), 8.01 (dd, 2H), 10.31 (s, 1H), 11.05 (br s, 1H).

### Compound 84

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(5-methoxypyridin-2-yl)propanamide

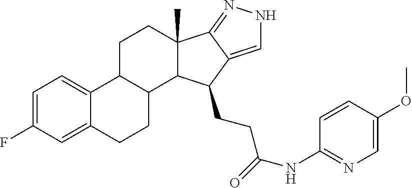 [SMILES: COc1ccc(NC(=O)CC[C@@H]2c3c[nH]nc3[C@@]3(C)CCC4c5ccc(F)cc5CCC4C23)nc1]

The compound 84 was prepared from the compound 83 by the Method C in 71% yield.

¹H NMR (400 MHz, DMSO-d₆): 1.10 (s, 3H), 1.40-1.74 (m, 5H), 2.02-2.48 (m, 8H), 2.78-2.98 (m, 3H), 3.80 (s, 3H), 6.90-7.09 (m, 2H), 7.30 (m, 1H), 7.41 (dd, 2H), 8.02 (d, 1H), 8.05 (d, 1H), 10.41 (s, 1H), 12.13 (br s, 1H).

### Compound 85

### 3-((13S,15R)-3-fluoro-13-methyl-17-oxo-7,8,9,11,12,13,14,15,16,17-decahydro-6H-cyclopenta[a]phenanthren-15-yl)-N-(5-isopropylpyridin-2-yl)propanamide

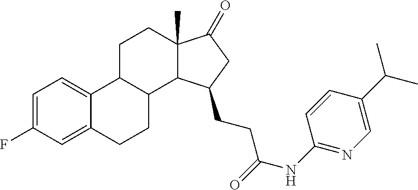 [SMILES: CC(C)c1ccc(NC(=O)CC[C@@H]2CC(=O)[C@@]3(C)CCC4c5ccc(F)cc5CCC4C23)nc1]

The compound 85 was synthesized in 60% yield by the Method A1 using Acid SM-XV and 2-amino-5-isopropylpyridine as starting materials in overnight reaction time.

¹H NMR (200 MHz, DMSO-d₆): 0.98 (s, 3H), 1.20 (d, 6H), 1.28-2.49 (m, 16H), 2.74-3.02 (m, 3H), 6.79-7.03 (m, 2H), 7.19-7.39 (m, 1H), 7.66 (d, 1H), 8.02 (d, 1H), 8.19 (s, 1H), 10.43 (s, 1H).

### Compound 86

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(5-isopropylpyridin-2-yl)-propanamide

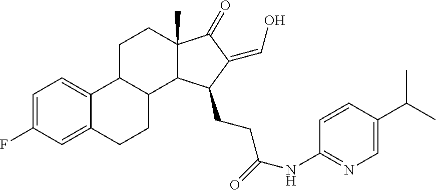 [SMILES: CC(C)c1ccc(NC(=O)CC[C@@H]2/C(=C/O)C(=O)[C@@]3(C)CCC4c5ccc(F)cc5CCC4C23)nc1]

The compound 86 was prepared from the compound 85 by the Method B in 98% yield.

¹H NMR (400 MHz, DMSO-d₆): 0.99 (s, 3H), 1.19 &1.21 (2×s, 6H), 1.38-2.35 (m, 12H), 2.55 (m, 2H), 2.84-2.97 (m, 4H), 6.89-6.95 (m, 2H), 7.28 (m, 1H), 7.55 (s, 1H), 7.65 (dd, 1H), 8.00 (d, 1H), 8.17 (d, 1H), 10.37 (s, 1H).

### Compound 87

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(5-isopropylpyridin-2-yl)propanamide

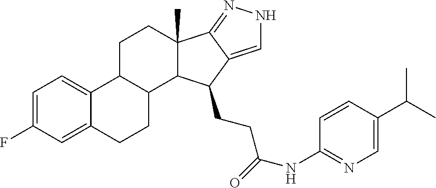 [SMILES: CC(C)c1ccc(NC(=O)CC[C@@H]2c3c[nH]nc3[C@@]3(C)CCC4c5ccc(F)cc5CCC4C23)nc1]

The compound 87 was prepared from the compound 86 by the Method C in 71% yield.

¹H NMR (400 MHz, DMSO-d₆): 1.18 (s, 3H), 1.21 & 1.23 (2×s, 6H), 1.46-2.31 (m, 11H), 2.58-2.63 (m, 3H), 2.91-3.02 (m, 4H), 6.91-6.96 (m, 2H), 7.31 (m, 1H), 7.91 (dd, 2H), 8.05 (d, 1H), 8.22 (s, 1H), 11.67 (s, 1H).

### Compound 88

### 3-((13S,15R)-3-fluoro-13-methyl-17-oxo-7,8,9,11,12,13,14,15,16,17-decahydro-6H-cyclopenta a phenanthren-15-yl)-N-(4-methoxypyridin-2-yl)propanamide

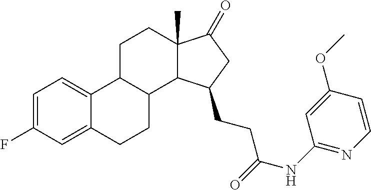 [SMILES: COc1ccnc(NC(=O)CC[C@@H]2CC(=O)[C@@]3(C)CCC4c5ccc(F)cc5CCC4C23)c1]

The compound 88 was prepared in 84% yield by the Method A2 from Acid SM-XV and 4-methoxy-2-aminopyridine stirring first at +50° C. for five hours and then overnight at room temperature.

¹H-NMR (400 MHz, DMSO-d₆): 0.98 (s, 3H), 1.37-2.45 (m, 16H), 2.94 (m, 2H), 3.80 (s, 3H), 6.69 (dd, 1H), 6.92 (m, 2H), 7.29 (dd, 1H), 7.73 (d, 1H), 8.11 (d, 1H), 10.46 (s, 1H).

### Compound 89

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(4-methoxypyridin-2-yl)-propanamide

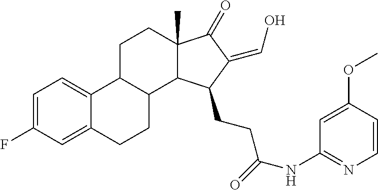 [SMILES: COc1ccnc(NC(=O)CC[C@@H]2/C(=C/O)C(=O)[C@@]3(C)CCC4c5ccc(F)cc5CCC4C23)c1]

The compound 89 was prepared from the compound 88 by the Method B in quantitative yield.

¹H NMR (400 MHz, DMSO-d₆): 0.99 (s, 3H), 1.45-2.32 (m, 12H), 2.55 (m, 1H), 2.83-2.97 (m, 3H), 3.80 (s, 3H), 6.68 (dd, 1H), 6.92 (m, 2H), 7.28 (dd, 1H), 7.53 (s, 1H), 7.72 (s, 1H), 8.10 (d, 1H), 10.38 (s, 1H), 10.98 (br s, 1H).

### Compound 90

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(4-methoxypyridin-2-yl)propanamide

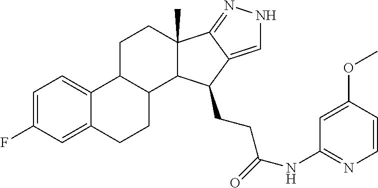 [SMILES: COc1ccnc(NC(=O)CC[C@@H]2c3c[nH]nc3[C@@]3(C)CCC4c5ccc(F)cc5CCC4C23)c1]

The compound 90 was prepared from the compound 89 by the Method C in 71% yield.

¹H NMR (400 MHz, DMSO-d₆): 1.10 (s, 3H), 1.39-2.46 (m, 13H), 2.81 (m, 1H), 2.92 (m, 2H), 3.80 (s, 3H), 6.70 (dd, 1H), 6.93 (m, 2H), 7.30 (dd, 1H), 7.39 (s, 1H), 7.74 (d, 1H), 8.12 (d, 1H), 10.50 (s, 1H), 12.13 (br s, 1H). MS m/z 475 (M+1)

### Compound 91

### 3-((13S,15R)-3-fluoro-13-methyl-17-oxo-7,8,9,11,12,13,14,15,16,17-decahydro-6H-cyclopenta[a]phenanthren-15-yl)-N-(4-methylpyridin-2-yl)propanamide

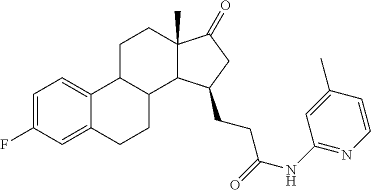 [SMILES: Cc1ccnc(NC(=O)CC[C@@H]2CC(=O)[C@@]3(C)CCC4c5ccc(F)cc5CCC4C23)c1]

The compound 91 was prepared in 41% yield by the Method A2 from Acid SM-XV and 4-methyl-2-aminopyridine stirring at +50° C. for seven hours and then at room temperature for overnight.

¹H-NMR (400 MHz, DMSO-d₆): 0.98 (s, 3H), 1.33-2.48 (m, 18H), 2.89 (m, 2H), 6.92 (m, 3H), 7.29 (dd, 2H), 7.95 (s, 1H), 8.15 (d, 1H), 10.41 (s, 1H).

### Compound 92

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-4-methylpyridin-2-yl)-propanamide

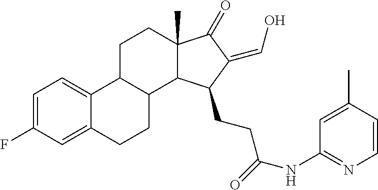 [SMILES: Cc1ccnc(NC(=O)CC[C@@H]2/C(=C/O)C(=O)[C@@]3(C)CCC4c5ccc(F)cc5CCC4C23)c1]

The compound 92 was prepared from the compound 91 by the Method B in quantitative yield.

¹H NMR (400 MHz, DMSO-d₆): 0.99 (s, 3H), 1.35-2.45 (m, 12H), 2.55 (m, 1H), 2.88 (m, 3H), 3.80 (s, 3H), 6.92 (m, 3H), 7.28 (m, 1H), 7.54 (s, 1H), 7.94 (s, 1H), 8.14 (s, 1H), 10.33 (s, 1H), 11.00 (br s, 1H).

### Compound 93

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(4-methylpyridin-2-yl)-propanamide

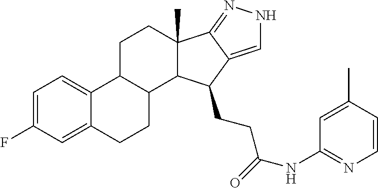 [SMILES: Cc1ccnc(NC(=O)CC[C@@H]2c3c[nH]nc3[C@@]3(C)CCC4c5ccc(F)cc5CCC4C23)c1]

The compound 93 was prepared from the compound 92 by the Method C in 55% yield.

¹H NMR (400 MHz, DMSO-d₆): 1.10 (s, 3H), 1.41-2.38 (m, 16H), 2.81 (m, 1H), 2.92 (m, 2H), 6.93 (m, 3H), 7.30 (m, 1H), 7.40 (s, 1H), 7.96 (s, 1H), 8.16 (d, 1H), 10.45 (s, 1H), 12.12 (br s, 1H). MS m/z 459 (M+1)

### Compound 94

### 3-((13S,15R)-3-fluoro-13-methyl-17-oxo-7,8,9,11,12,13,14,15,16,17-decahydro-6H-cyclopenta[a]phenanthren-15-yl)-N-(5-morpholinopyridin-2-yl)propanamide

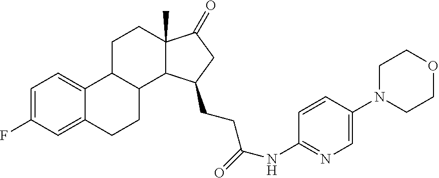 [SMILES: C[C@]12CCC3c4ccc(F)cc4CCC3C1[C@H](CCC(=O)Nc1ccc(N3CCOCC3)cn1)CC2=O]

The compound 94 was synthesized in 63% yield after chromatographic purification by the Method A1 in DCM by using acid SM-XV and 5-morpholinopyridin-2-amine as starting materials and triethylamine as base in 2 hours reaction time.

¹H-NMR (400 MHz, DMSO-d₆): 0.98 (s, 3H), 1.30-2.47 (m, 16H), 2.81-2.96 (m, 2H), 3.06-3.12 (m, 4H), 3.70-3.78 (m, 4H), 6.90-6.95 (m, 2H), 7.25-7.32 (t, 1H), 7.40 (dd, 1H), 7.95-8.01 (m, 2H), 10.28 (s, 1H).

### Compound 95

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(5-morpholinopyridin-2-yl)propanamide

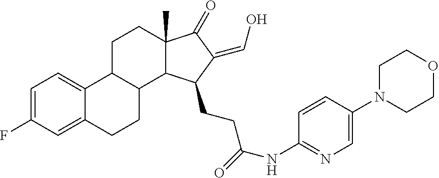 [SMILES: C[C@]12CCC3c4ccc(F)cc4CCC3C1[C@H](CCC(=O)Nc1ccc(N3CCOCC3)cn1)/C(=C/O)C2=O]

The compound 95 was synthesized in 94% yield from the compound 94 by the Method B in overnight reaction time.

¹H-NMR (400 MHz, DMSO-d₆): 0.99 (s, 3H), 1.30-2.60 (m, 13H), 2.81-3.00 (m, 3H), 3.06-3.12 (m, 4H), 3.70-3.78 (m, 4H), 6.88-6.95 (m, 2H), 7.25-7.32 (t, 1H), 7.40 (dd, 1H), 7.54 (s, 1H), 7.92-8.01 (m, 2H), 10.24 (s, 1H), 11.02 (br s, 1H).

### Compound 96

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(5-morpholinopyridin-2-yl)propanamide

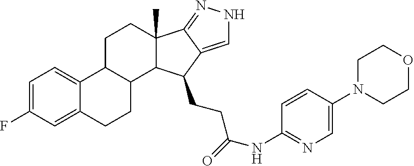 [SMILES: C[C@]12CCC3c4ccc(F)cc4CCC3C1[C@H](CCC(=O)Nc1ccc(N3CCOCC3)cn1)c1c[nH]nc12]

The compound 96 was synthesized in 73% yield from the compound 95 by the Method C in one hour reaction time.

¹H-NMR (400 MHz, DMSO-d₆): 1.10 (s, 3H), 1.30-2.50 (m, 13H), 2.75-2.95 (m, 3H), 3.06-3.12 (m, 4H), 3.70-3.78 (m, 4H), 6.88-6.96 (m, 2H), 7.26-7.33 (t, 1H), 7.38-7.43 (m, 2H), 7.94-8.01 (m, 2H), 10.32 (s, 1H), 12.11 (br s, 1H).

### Compound 97

### 3-((13S,15R)-3-fluoro-13-methyl-17-oxo-7,8,9,11,12,13,14,15,16,17-decahydro-6H-cyclopenta[a]phenanthren-15-yl)-N-(5-fluoropyridin-2-yl)propanamide

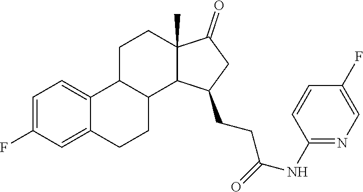 [SMILES: C[C@]12CCC3c4ccc(F)cc4CCC3C1[C@H](CCC(=O)Nc1ccc(F)cn1)CC2=O]

The compound 97 was synthesized in 93% yield by the Method A1 in DCM by using acid SM-XV and 2-amino-5-fluoropyridine as starting materials in 2 hours reaction time.

¹H-NMR (200 MHz, DMSO-d₆): 0.98 (s, 3H), 1.22-2.45 (m, 16H), 2.80-2.95 (m, 2H), 6.83-7.03 (m, 2H), 7.20-7.39 (m, 1H), 7.73 (td, 1H), 8.14 (dd, 1H), 8.31 (d, 1H), 10.62 (s, 1H).

### Compound 98

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(5-fluoropyridin-2-yl)propanamide

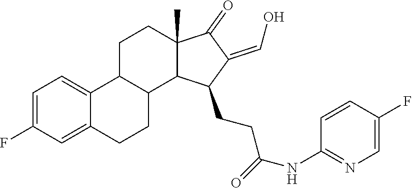 [SMILES: C[C@]12CCC3c4ccc(F)cc4CCC3C1[C@H](CCC(=O)Nc1ccc(F)cn1)/C(=C/O)C2=O]

The compound 98 was synthesized in 81% yield from the compound 97 by the Method B in overnight reaction time by using 500 mol-% of ethyl formate and 300 mol-% of NaH.

¹H-NMR (400 MHz, DMSO-d₆): 0.99 (s, 3H), 1.30-2.60 (m, 13H), 2.80-2.99 (m, 3H), 6.85-7.03 (m, 2H), 7.20-7.39 (m, 1H), 7.58 (s, 1H), 7.71 (td, 1H), 8.13 (dd, 1H), 8.30 (d, 1H), 10.62 (s, 1H).

### Compound 99

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(5-fluoropyridin-2-yl)propanamide

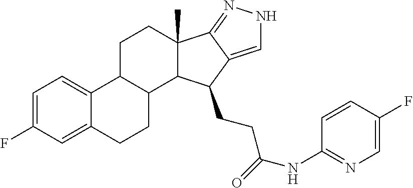 [SMILES: C[C@]12CCC3c4ccc(F)cc4CCC3C1[C@H](CCC(=O)Nc1ccc(F)cn1)c1c[nH]nc12]

The compound 99 was synthesized in 45% yield from the compound 98 by the Method C in one hour reaction time.

¹H-NMR (400 MHz, DMSO-d₆): 1.10 (s, 3H), 1.35-2.49 (m, 13H), 2.79-2.99 (m, 3H), 6.85-7.00 (m, 2H), 7.25-7.35 (m, 1H), 7.40 (s, 1H), 7.73 (td, 1H), 8.14 (dd, 1H), 8.31 (d, 1H), 10.66 (s, 1H), 12.12 (s, 1H).

### Compound 100

### N-(5-(tert-butyl)isoxazol-3-yl)-3-((13 S,15R)-3-fluoro-13-methyl-17-oxo-7,8,9,11,12,13,14,15,16,17-decahydro-6H-cyclopenta[a]phenanthren-15-yl)propanamide

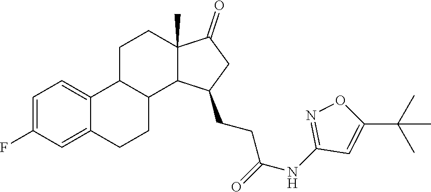 [SMILES: CC(C)(C)c1cc(NC(=O)CC[C@@H]2CC(=O)[C@@]3(C)CCC4c5ccc(F)cc5CCC4C23)no1]

The compound 100 was prepared in 39% yield by the Method A1 from Acid SM-XV and 3-amino-5-tert-butylisoxazole stirring at room temperature for five hours.

The product was used directly to the next step.

### Compound 101

### N-(5-(tert-butyl)isoxazol-3-yl)-3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)propanamide

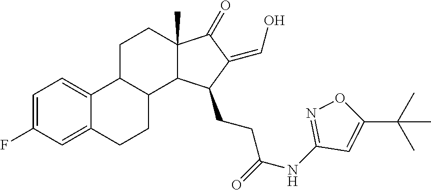 [SMILES: CC(C)(C)c1cc(NC(=O)CC[C@@H]2/C(=C/O)C(=O)[C@@]3(C)CCC4c5ccc(F)cc5CCC4C23)no1]

The compound 101 was prepared from the compound 100 by the Method B in quantitative yield.

¹H NMR (400 MHz, CDCl₃): 1.16 (s, 3H), 1.26 (s, 9H), 1.32-2.84 (m, 17H), 6.68-6.85 (m, 3H), 7.19-7.23 (m, 1H), 7.31 (s, 1H), 9.47 (s, 1H)

### Compound 102

### N-(5-(tert-butyl)isoxazol-3-yl)-3-((8aS,12 S)-4-fluoro-8a-methyl-1,2,6b,7,8,8a,10,12,12a,12b-decahydronaphtho[2′,1′:4,5]indeno[1,2-c]pyrazol-12-yl)propanamide

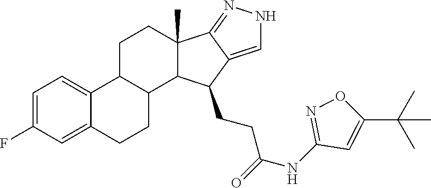 [SMILES: CC(C)(C)c1cc(NC(=O)CC[C@@H]2c3c[nH]nc3[C@@]3(C)CCC4c5ccc(F)cc5CCC4C23)no1]

The compound 102 was prepared from the compound 101 by the Method C in 46% yield.

¹H NMR (400 MHz, DMSO-d₆): 1.10 (s, 3H), 1.29 (s, 9H), 1.41-2.43 (m, 13H), 2.80-2.92 (m, 3H), 6.61 (m, 1H), 6.93 (m, 2H), 7.30 (m, 1H), 7.39 (s, 1H), 10.97 (s, 1H), 12.13 (s, 1H).

### Compound 103

### 3-((13S,15R)-3-fluoro-13-methyl-17-oxo-7,8,9,11,12,13,14,15,16,17-decahydro-6H-cyclopenta[a]phenanthren-15-yl)-N-(6-fluoropyridin-2-yl)propanamide

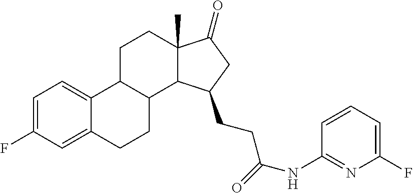 [SMILES: C[C@]12CCC3c4ccc(F)cc4CCC3C1[C@H](CCC(=O)Nc1cccc(F)n1)CC2=O]

The compound 103 was synthesized in 82% yield by the Method A1 in DCM by using acid SM-XV and 2-amino-6-fluoropyridine as starting materials in 3 hours reaction time.

¹H-NMR (200 MHz, DMSO-d₆): 0.98 (s, 3H), 1.33-2.47 (m, 16H), 2.89 (m, 2H), 6.84 (dd, 1H), 6.92 (m, 2H), 7.29 (m, 1H), 7.94 (dd, 1H), 8.01 (m, 1H), 10.67 (s, 1H).

### Compound 104

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(6-fluoropyridin-2-yl)propanamide

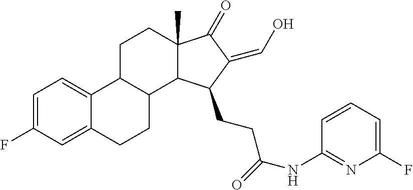 [SMILES: C[C@]12CCC3c4ccc(F)cc4CCC3C1[C@H](CCC(=O)Nc1cccc(F)n1)/C(=C/O)C2=O]

The compound 104 was synthesized in 92% yield from the compound 103 by the Method B in two hours reaction time.

¹H-NMR (400 MHz, DMSO-d₆): 0.99 (s, 3H), 1.36-2.33 (m, 12H), 2.57 (m, 2H), 2.89 (m, 3H), 6.81 (dd, 1H), 6.92 (m, 2H), 7.28 (m, 1H), 7.57 (s, 1H), 7.93 (dd, 1H), 8.00 (m, 1H), 10.66 (s, 1H).

### Compound 105

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(6-fluoropyridin-2-yl)propanamide

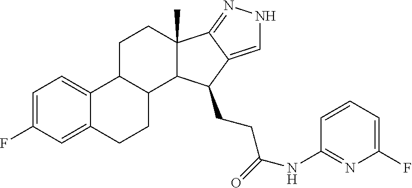 [SMILES: C[C@]12CCC3c4ccc(F)cc4CCC3C1[C@H](CCC(=O)Nc1cccc(F)n1)c1c[nH]nc12]

The compound 105 was synthesized in 50% yield from the compound 104 by the Method C in 30 minutes reaction time.

¹H-NMR (400 MHz, DMSO-d₆): 1.10 (s, 3H), 1.42-2.38 (m, 14H), 2.78-2.95 (m, 3H), 6.83 (m, 1H), 6.93 (m, 2H), 7.30 (m, 1H), 7.39 (s, 1H), 7.94 (m, 1H), 8.00 (m, 1H), 10.72 (s, 1H).

### Compound 106

### 3-((13S,15R)-3-fluoro-13-methyl-17-oxo-7,8,9,11,12,13,14,15,16,17-decahydro-6H-cyclopenta[a]phenanthren-15-yl)-N-(6-methoxypyridazin-3-yl)propanamide

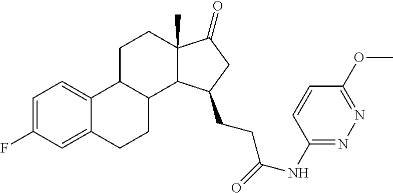 [SMILES: COc1ccc(NC(=O)CC[C@@H]2CC(=O)[C@@]3(C)CCC4c5ccc(F)cc5CCC4C23)nn1]

The compound 106 was synthesized in 79% yield by the Method A2 in THE by using acid SM-XV and 3-amino-6-methoxypyridazine as starting materials in 3 hours reaction time.

¹H-NMR (400 MHz, DMSO-d₆): 0.98 (s, 3H), 1.30-2.47 (m, 16H), 2.79-3.02 (m, 2H), 3.98 (s, 3H), 6.85-7.03 (m, 2H), 7.24 (d, 1H), 7.26-7.35 (m, 1H), 8.25 (d, 1H), 10.93 (br s, 1H).

### Compound 107

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(6-methoxypyridazin-3-yl)propanamide

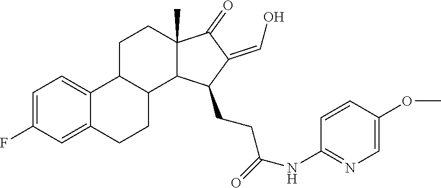 [SMILES: COc1ccc(NC(=O)CC[C@@H]2/C(=C/O)C(=O)[C@@]3(C)CCC4c5ccc(F)cc5CCC4C23)nc1]

The compound 107 was synthesized in quantitative yield from the compound 106 by the Method B in overnight reaction time.

¹H-NMR (400 MHz, DMSO-d₆): 0.99 (s, 3H), 1.30-2.47 (m, 13H), 2.55-3.00 (m, 3H), 3.98 (s, 3H), 6.85-7.03 (m, 2H), 7.23 (d, 1H), 7.25-7.33 (m, 1H), 7.55 (s, 1H), 8.23 (d, 1H), 10.88 (s, 1H), 10.96 (br s, 1H).

### Compound 108

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(6-methoxypyridazin-3-yl)propanamide

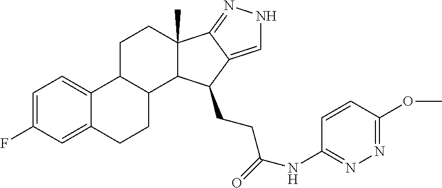 [SMILES: COc1ccc(NC(=O)CC[C@@H]2c3c[nH]nc3[C@@]3(C)CCC4c5ccc(F)cc5CCC4C23)nn1]

The compound 108 was synthesized in 92% yield from the compound 107 by the Method C in one hour reaction time.

¹H-NMR (400 MHz, DMSO-d₆): 1.11 (s, 3H), 1.30-2.47 (m, 13H), 2.75-3.00 (m, 3H), 3.98 (s, 3H), 6.85-7.03 (m, 2H), 7.23 (d, 1H), 7.25-7.33 (m, 1H), 7.41 (s, 1H), 8.25 (d, 1H), 10.96 (s, 1H), 12.12 (br s, 1H).

### Compound 109

### 3-((13S,15R)-3-fluoro-13-methyl-17-oxo-7,8,9,11,12,13,14,15,16,17-decahydro-6H-cyclopenta[a]phenanthren-15-yl)-N-(6-methylpyridazin-3-yl)propanamide

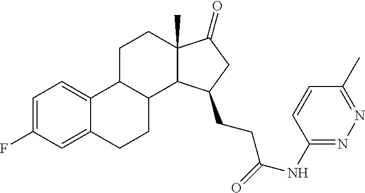 [SMILES: Cc1ccc(NC(=O)CC[C@@H]2CC(=O)[C@@]3(C)CCC4c5ccc(F)cc5CCC4C23)nn1]

The compound 109 was synthesized in 79% yield by the Method A2 in THE by using acid SM-XV and 3-amino-6-methylpyridazine as starting materials in 3 hours reaction time.

¹H-NMR (400 MHz, DMSO-d₆): 0.98 (s, 3H), 1.30-2.46 (m, 16H), 2.55 (s, 3H), 2.80-3.00 (m, 2H), 6.85-7.02 (m, 2H), 7.23-7.35 (m, 1H), 7.54 (d, 1H), 8.22 (d, 1H), 11.03 (br s, 1H).

### Compound 110

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(6-methylpyridazin-3-yl)propanamide

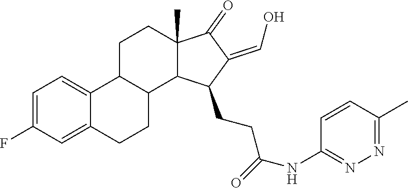 [SMILES: Cc1ccc(NC(=O)CC[C@@H]2/C(=C/O)C(=O)[C@@]3(C)CCC4c5ccc(F)cc5CCC4C23)nn1]

The compound 110 was synthesized in quantitative yield from the compound 109 by the Method B in overnight reaction time.

¹H-NMR (400 MHz, DMSO-d₆): 0.99 (s, 3H), 1.30-2.70 (m, 13H), 2.55 (s, 3H), 2.80-3.00 (m, 3H), 6.85-7.02 (m, 2H), 7.21-7.33 (m, 1H), 7.52 (d, 1H), 7.53 (s, 1H), 8.21 (d, 1H), 10.96 (br s, 2×1H).

### Compound 111

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(6-methylpyridazin-3-yl)propanamide

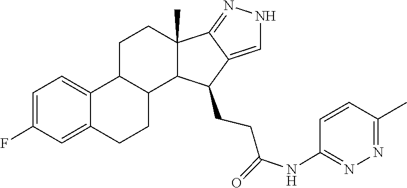 [SMILES: Cc1ccc(NC(=O)CC[C@@H]2c3c[nH]nc3[C@@]3(C)CCC4c5ccc(F)cc5CCC4C23)nn1]

The compound 111 was synthesized in 24% yield after chromatographic purification from the compound 110 by the Method C in one hour reaction time.

¹H-NMR (400 MHz, DMSO-d₆): 1.11 (s, 3H), 1.30-2.59 (m, 13H), 2.55 (s, 3H), 2.77-3.00 (m, 3H), 6.85-7.02 (m, 2H), 7.23-7.33 (m, 1H), 7.41 (s, 1H), 7.53 (d, 1H), 8.22 (d, 1H), 11.07 (s, 1H), 12.13 (br s, 1H).

### Compound 112

### 3-((13S,15R)-3-fluoro-13-methyl-17-oxo-7,8,9,11,12,13,14,15,16,17-decahydro-6H-cyclopenta[a]phenanthren-15-yl)-N-(pyridazin-3-yl)propanamide

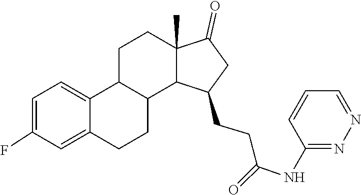 [SMILES: C[C@]12CCC3c4ccc(F)cc4CCC3C1[C@H](CCC(=O)Nc1cccnn1)CC2=O]

The compound 112 was synthesized in 79% yield by the Method A2 in THE by using acid SM-XV and 3-aminopyridazine as starting materials in 3.5 hours reaction time.

¹H-NMR (400 MHz, DMSO-d₆): 0.98 (s, 3H), 1.30-2.62 (m, 16H), 2.80-2.95 (m, 2H), 6.85-7.03 (m, 2H), 7.20-7.31 (m, 1H), 7.67 (dd, 1H), 8.32 (d, 1H), 8.95 (d, 1H), 11.13 (s, 1H).

### Compound 113

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(pyridazin-3-yl)propanamide

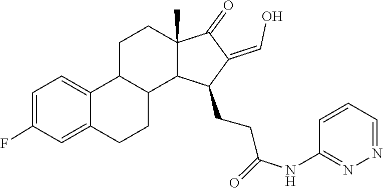 [SMILES: C[C@]12CCC3c4ccc(F)cc4CCC3C1[C@H](CCC(=O)Nc1cccnn1)/C(=C/O)C2=O]

The compound 113 was synthesized in quantitative yield from the compound 112 by the Method B in overnight reaction time.

¹H-NMR (400 MHz, DMSO-d₆): 1.00 (s, 3H), 1.30-2.70 (m, 13H), 2.80-2.99 (m, 3H), 6.85-7.03 (m, 2H), 7.20-7.33 (m, 1H), 7.55 (s, 1H), 7.65 (dd, 1H), 8.31 (d, 1H), 8.93 (d, 1H), 11.06 (s, 1H), 11.07 (br s, 1H).

### Compound 114

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(pyridazin-3-yl)propanamide

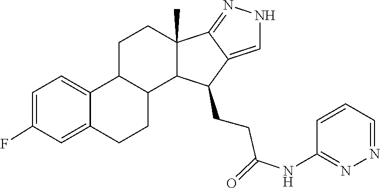 [SMILES: C[C@]12CCC3c4ccc(F)cc4CCC3C1[C@H](CCC(=O)Nc1cccnn1)c1c[nH]nc12]

The compound 114 was synthesized in 45% yield after chromatographic purification from the compound 113 by the Method C in one hour reaction time.

¹H-NMR (400 MHz, DMSO-d₆): 1.11 (s, 3H), 1.30-2.65 (m, 13H), 2.80-2.99 (m, 3H), 6.85-7.02 (m, 2H), 7.25-7.35 (m, 1H), 7.42 (s, 1H), 7.66 (dd, 1H), 8.33 (d, 1H), 8.95 (d, 1H), 11.17 (s, 1H), 12.14 (br s, 1H).

### Compound 115

### (13S,15R)-3-fluoro-13-methyl-15-(3-oxo-3-(pyrrolidin-1-yl)propyl)-6,7,8,9,11,12,13,14,15,16-decahydro-17H-cyclopenta[a]phenanthren-17-one

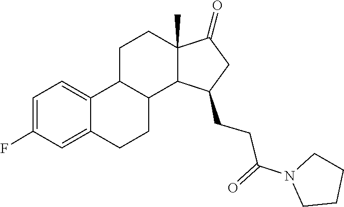 [SMILES: C[C@]12CCC3c4ccc(F)cc4CCC3C1[C@H](CCC(=O)N1CCCC1)CC2=O]

The compound 115 was synthesized in 46% yield after chromatographic purification by the Method A1 in DCM by using acid SM-XV and pyrrolidine as starting materials in 4 hours reaction time.

¹H-NMR (200 MHz, CDCl₃): 1.07 (s, 3H), 1.33-2.50 (m, 20H), 2.79-3.09 (m, 2H), 3.35-3.55 (m, 4H), 6.70-6.90 (m, 2H), 7.17-7.26 (m, 1H).

### Compound 116

### (13S,15S,Z)-3-fluoro-16-(hydroxymethylene)-13-methyl-15-(3-oxo-3-(pyrrolidin-1-yl)propyl)-6,7,8,9,11,12,13,14,15,16-decahydro-17H-cyclopenta[a]phenanthren-17-one

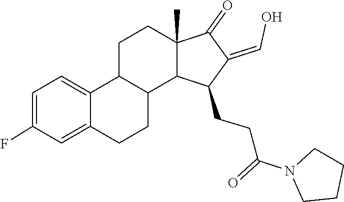 [SMILES: C[C@]12CCC3c4ccc(F)cc4CCC3C1[C@H](CCC(=O)N1CCCC1)/C(=C/O)C2=O]

The compound 116 was synthesized in 98% yield from the compound 115 by the Method B in 2 hours reaction time.

¹H-NMR (400 MHz, DMSO-d₆): 0.97 (s, 3H), 1.30-2.46 (m, 17H), 2.80-3.00 (m, 3H), 3.25-3.45 (m, 4H), 6.86-6.98 (m, 2H), 7.25-7.35 (m, 1H), 7.52 (s, 1H).

### Compound 117

### 3-((8aS,12S)-4-fluoro-8a-methyl-1,2,6b,7,8,8a,10,12,12a,12b-decahydronaphtho[2′,1′:4,S]indeno[1,2-c]pyrazol-12-yl)-1-(pyrrolidin-1-yl)propan-1-one

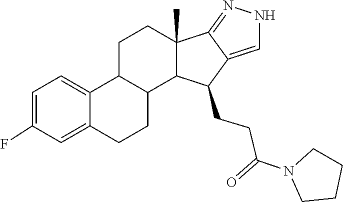 [SMILES: C[C@]12CCC3c4ccc(F)cc4CCC3C1[C@H](CCC(=O)N1CCCC1)c1c[nH]nc12]

The compound 117 was synthesized in 88% yield from the compound 116 by the Method C in 0.5 hours reaction time.

¹H-NMR (400 MHz, DMSO-d₆): 1.09 (s, 3H), 1.30-2.44 (m, 17H), 2.80-3.00 (m, 3H), 3.21-3.49 (m, 4H), 6.87-6.98 (m, 2H), 7.27-7.35 (m, 1H), 7.40 (s, 1H), 12.10 (br s, 1H).

### Compound 118

### 3-((13S,15R)-3-fluoro-13-methyl-17-oxo-7,8,9,11,12,13,14,15,16,17-decahydro-6H-cyclopenta[a]phenanthren-15-yl)-N-(pyrazin-2-yl)propanamide

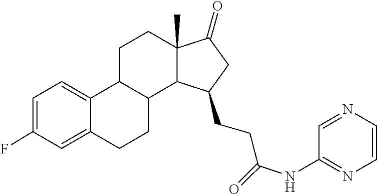 [SMILES: C[C@]12CCC3c4ccc(F)cc4CCC3C1[C@H](CCC(=O)Nc1cnccn1)CC2=O]

The compound 118 was synthesized in 44% yield by the Method A1 in DCM by using acid SM-XV and aminopyrazine as starting materials in 3 hours reaction time.

¹H-NMR (400 MHz, CDCl₃): 1.08 (s, 3H), 1.30-2.65 (m, 16H), 2.85-3.08 (m, 2H), 6.74-6.95 (m, 2H), 7.20-7.26 (m, 1H), 7.80 (s, 1H), 8.25 (d, 1H), 8.37 (d, 1H), 9.53 (s, 1H).

### Compound 119

### 3-((13S,15S,Z)-3-fluoro-16-(hydroxymethylene)-13-methyl-17-oxo-7,8,9,11,12,13,14,15,16,17-decahydro-6H-cyclopenta[a]phenanthren-15-yl)-N-(pyrazin-2-yl)propanamide

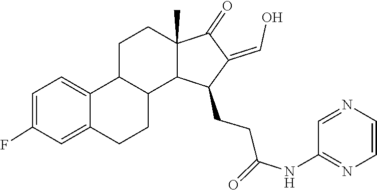 [SMILES: C[C@]12CCC3c4ccc(F)cc4CCC3C1[C@H](CCC(=O)Nc1cnccn1)/C(=C/O)C2=O]

The compound 119 was synthesized in 99% yield from the compound 118 by the Method B in overnight reaction time.

¹H-NMR (400 MHz, DMSO-d₆): 1.00 (s, 3H), 1.30-2.68 (m, 13H), 2.80-3.08 (m, 3H), 6.80-6.96 (m, 2H), 7.25-7.35 (m, 1H), 7.60 (s, 1H), 8.32 (d, 1H), 8.37 (d, 1H), 9.33 (s, 1H), 10.86 (br s, 1H), 10.96 (br s, 1H).

### Compound 120

### 3-((8aS,12S)-4-fluoro-8a-methyl-1,2,6b,7,8,8a,10,12,12a,12b-decahydronaphtho[2′,1′:4,5]indeno[1,2-c]pyrazol-12-yl)-N-(pyrazin-2-yl)propanamide

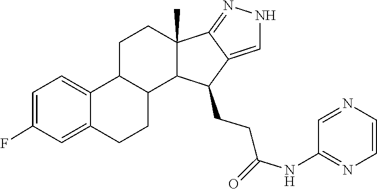 [SMILES: C[C@]12CCC3c4ccc(F)cc4CCC3C1[C@H](CCC(=O)Nc1cnccn1)c1c[nH]nc12]

The compound 120 was synthesized in quantitative yield from the compound 119 by the Method C in one hour reaction time.

¹H-NMR (400 MHz, DMSO-d₆): 1.11 (s, 3H), 1.30-2.60 (m, 13H), 2.79-3.01 (m, 3H), 6.85-6.99 (m, 2H), 7.25-7.35 (m, 1H), 7.39 (s, 1H), 8.34 (d, 1H), 8.39 (d, 1H), 9.35 (s, 1H), 10.84 (s, 1H), 12.13 (br s, 1H).

### Compound 121

### 3-((13S,15R)-3-chloro-13-methyl-17-oxo-7,8,9,11,12,13,14,15,16,17-decahydro-6H-cyclopenta[a]phenanthren-15-yl)-N-(2-oxo-1,2,5,6,7,8-hexahydroquinolin-3-yl)propanamide

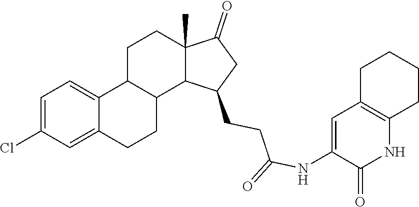 [SMILES: C[C@]12CCC3c4ccc(Cl)cc4CCC3C1[C@H](CCC(=O)Nc1cc3c([nH]c1=O)CCCC3)CC2=O]

The compound 121 was synthesized in 79% yield by the Method A1 in THE by using acid SM-XVII and 3-amino-1,2,5,6,7,8-hexahydroquinolin-2-one as starting materials in overnight reaction time.

¹H-NMR (400 MHz, DMSO-d₆): 0.97 (s, 3H), 1.30-2.47 (m, 24H), 2.80-2.95 (m, 2H), 7.14-7.17 (m, 2H), 7.28-7.31 (m, 1H), 8.01 (s, 1H), 9.14 (s, 1H), 11.68 (s, 1H).

### Compound 122

### 3-((13S,15S,Z)-3-chloro-16-(hydroxymethylene)-13-methyl-17-oxo-7,8,9,11,12,13,14,15,16,17-decahydro-6H-cyclopenta[a]phenanthren-15-yl)-N-(2-oxo-1,2,5,6,7,8-hexahydroquinolin-3-yl)propanamide

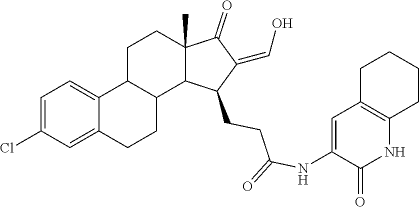 [SMILES: C[C@]12CCC3c4ccc(Cl)cc4CCC3C1[C@H](CCC(=O)Nc1cc3c([nH]c1=O)CCCC3)/C(=C/O)C2=O]

The compound 122 was synthesized in 76% yield from the compound 121 by the Method B in overnight reaction time.

¹H-NMR (400 MHz, DMSO-d₆): 0.98 (s, 3H), 1.30-2.60 (m, 22H), 2.80-2.95 (m, 2H), 7.14-7.17 (m, 2H), 7.28-7.31 (m, 1H), 7.54 (s, 1H), 7.99 (s, 1H), 8.99 (s, 1H), 11.09 (br s, 1H), 11.66 (s, 1H).

### Compound 123

### 3-((8aS,12S)-4-chloro-8a-methyl-1,2,6b,7,8,8a,10,12,12a,12b-decahydronaphtho[2′,1′:4,5]indeno[1,2-c]pyrazol-12-yl)-N-(2-oxo-1,2,5,6,7,8-hexahydroquinolin-3-yl) propanamide

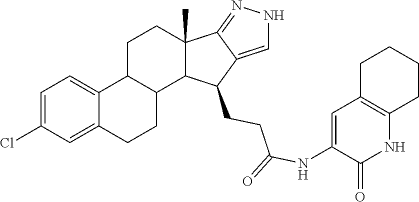 [SMILES: C[C@]12CCC3c4ccc(Cl)cc4CCC3C1[C@H](CCC(=O)Nc1cc3c([nH]c1=O)CCCC3)c1c[nH]nc12]

The compound 123 was synthesized in 46% yield from the compound 122 by the Method C in one hour reaction time.

¹H-NMR (400 MHz, DMSO-d₆): 1.09 (s, 3H), 1.30-2.59 (m, 21H), 2.75-2.96 (m, 3H), 7.14-7.19 (m, 2H), 7.28-7.33 (m, 1H), 7.41 (s, 1H), 8.00 (s, 1H), 9.14 (s, 1H), 11.68 (s, 1H), 12.11 (br s, 1H).

### Compound 124

### 6-(3-((13S,15R)-3-chloro-13-methyl-17-oxo-7,8,9,11,12,13,14,15,16,17-decahydro-6H-cyclopenta[a]phenanthren-15-yl)propanamido)-N,N-dimethylnicotinamide

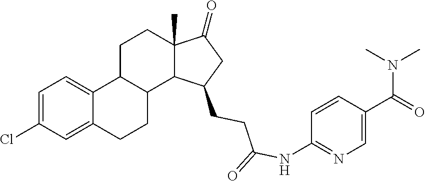 [SMILES: CN(C)C(=O)c1ccc(NC(=O)CC[C@@H]2CC(=O)[C@@]3(C)CCC4c5ccc(Cl)cc5CCC4C23)nc1]

The compound 124 was synthesized in 85% yield by the Method A1 in THE by using acid SM-XVII and 6-amino-N,N-dimethylpyridine-3-carboxamide as starting materials in overnight reaction time.

¹H-NMR (400 MHz, DMSO-d₆): 0.98 (s, 3H), 1.30-2.47 (m, 16H), 2.80-2.95 (m, 2H), 2.97 (s, 6H), 7.14-7.17 (m, 2H), 7.28-7.31 (m, 1H), 7.85 (dd, 1H), 8.14 (d, 1H), 8.38 (d, 1H), 10.71 (s, 1H).

### Compound 125

### 6-(3-((13S,15S,Z)-3-chloro-16-(hydroxymethylene)-13-methyl-17-oxo-7,8,9,11,12,13,14,15,16,17-decahydro-6H-cyclopenta[a]phenanthren-15-yl)propanamido)-N,N-dimethylnicotinamide

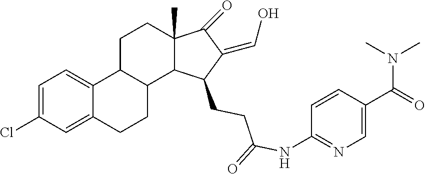 [SMILES: CN(C)C(=O)c1ccc(NC(=O)CC[C@@H]2/C(=C/O)C(=O)[C@@]3(C)CCC4c5ccc(Cl)cc5CCC4C23)nc1]

The compound 125 was synthesized in quantitative yield from the compound 124 by the Method B in overnight reaction time.

¹H-NMR (400 MHz, DMSO-d₆): 0.99 (s, 3H), 1.30-2.40 (m, 12H), 2.50-2.65 (m, 2H), 2.80-2.95 (m, 2H), 2.97 (s, 6H), 7.13-7.17 (m, 2H), 7.26-7.30 (m, 1H), 7.54 (s, 1H), 7.84 (dd, 1H), 8.13 (d, 1H), 8.37 (d, 1H), 10.64 (s, 1H), 10.98 (br s, 1H).

### Compound 126

### 6-(3-((8aS,12S)-4-chloro-8a-methyl-1,2,6b,7,8,8a,10,12,12a,12b-decahydronaphtho[2′,1′:4,5]indeno[1,2-c]pyrazol-12-yl)propanamido)-N,N-dimethylnicotinamide

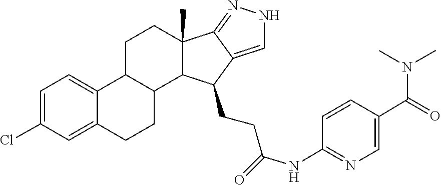 [SMILES: CN(C)C(=O)c1ccc(NC(=O)CC[C@@H]2c3c[nH]nc3[C@@]3(C)CCC4c5ccc(Cl)cc5CCC4C23)nc1]

The compound 126 was synthesized in 63% yield from the compound 125 by the Method C in one hour reaction time.

¹H-NMR (400 MHz, DMSO-d₆): 1.10 (s, 3H), 1.30-2.60 (m, 13H), 2.70-2.97 (m, 3H), 2.98 (s, 6H), 7.14-7.19 (m, 2H), 7.28-7.32 (m, 1H), 7.41 (s, 1H), 7.85 (dd, 1H), 8.15 (d, 1H), 8.39 (d, 1H), 10.76 (s, 1H), 12.12 (br s, 1H).

### Compound 127

### 3-((13S,15R)-3-chloro-13-methyl-17-oxo-7,8,9,11,12,13,14,15,16,17-decahydro-6H-cyclopenta[a]phenanthren-15-yl)-N-(4-fluoropyridin-2-yl)propanamide

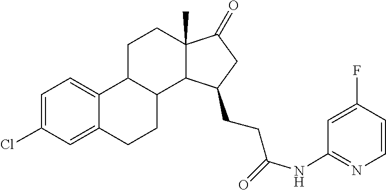 [SMILES: C[C@]12CCC3c4ccc(Cl)cc4CCC3C1[C@H](CCC(=O)Nc1cc(F)ccn1)CC2=O]

The compound 127 was synthesized in 90% yield by the Method A1 in THE by using acid SM-XVII and 2-amino-4-fluoropyridine as starting materials in 4 hours reaction time.

¹H-NMR (400 MHz, DMSO-d₆): 0.98 (s, 3H), 1.30-2.47 (m, 16H), 2.81-2.96 (m, 2H), 7.00-7.06 (m, 1H), 7.14-7.17 (m, 2H), 7.27-7.31 (m, 1H), 7.92 (dd, 1H), 8.30-8.37 (m, 1H), 10.82 (s, 1H).

### Compound 128

### 3-((13S,15S,Z)-3-chloro-16-(hydroxymethylene)-13-methyl-17-oxo-7,8,9,11,12,13,14,15,16,17-decahydro-6H-cyclopenta[a]phenanthren-15-yl)-N-(4-fluoropyridin-2-yl)propanamide

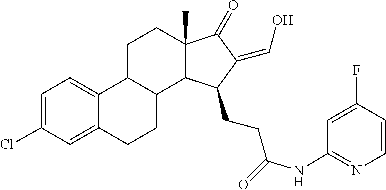 [SMILES: C[C@]12CCC3c4ccc(Cl)cc4CCC3C1[C@H](CCC(=O)Nc1cc(F)ccn1)/C(=C/O)C2=O]

The compound 128 was synthesized in quantitative yield from the compound 127 by the Method B in 4 hours reaction time.

¹H-NMR (400 MHz, DMSO-d₆): 0.99 (s, 3H), 1.30-2.65 (m, 13H), 2.81-2.99 (m, 3H), 6.98-7.06 (m, 1H), 7.09-7.20 (m, 2H), 7.26-7.31 (m, 1H), 7.55 (s, 1H), 7.92 (dd, 1H), 8.30-8.37 (m, 1H), 10.76 (s, 1H), 10.96 (br s, 1H).

### Compound 129

### 3-((8aS,12S)-4-chloro-8a-methyl-1,2,6b,7,8,8a,10,12,12a,12b-decahydronaphtho[2′1′:4,5]indeno[1,2-c]pyrazol-12-yl)-N-(4-fluoropyridin-2-yl)propanamide

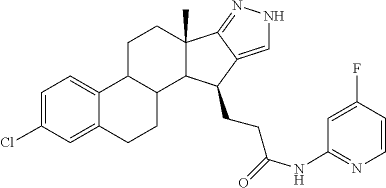 [SMILES: C[C@]12CCC3c4ccc(Cl)cc4CCC3C1[C@H](CCC(=O)Nc1cc(F)ccn1)c1c[nH]nc12]

The compound 129 was synthesized in 72% yield from the compound 128 by the Method C in one hour reaction time.

¹H-NMR (400 MHz, DMSO-d₆): 1.10 (s, 3H), 1.30-2.53 (m, 13H), 2.76-2.99 (m, 3H), 6.99-7.06 (m, 1H), 7.10-7.20 (m, 2H), 7.27-7.32 (m, 1H), 7.40 (s, 1H), 7.93 (dd, 1H), 8.30-8.38 (m, 1H), 10.86 (s, 1H), 12.12 (br s, 1H).

### Compound 130

### 3-((13S,15R)-3-chloro-13-methyl-17-oxo-7,8,9,11,12,13,14,15,16,17-decahydro-6H-cyclopenta[a]phenanthren-15-yl)-N-(3,5-difluoropyridin-2-yl)propanamide

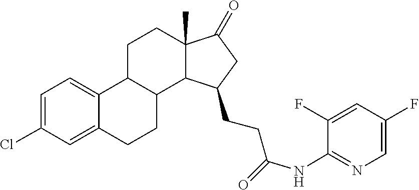 [SMILES: C[C@]12CCC3c4ccc(Cl)cc4CCC3C1[C@H](CCC(=O)Nc1ncc(F)cc1F)CC2=O]

The compound 130 was synthesized in 90% yield by the Method A1 in THE by using acid SM-XVII and 2-amino-3,5-difluoropyridine as starting materials in 5 hours reaction time.

¹H-NMR (400 MHz, DMSO-d₆): 0.98 (s, 3H), 1.30-2.47 (m, 16H), 2.80-2.94 (m, 2H), 7.15-7.16 (m, 2H), 7.28-7.30 (m, 1H), 7.98-8.03 (m, 1H), 8.34-8.35 (m, 1H), 10.31 (s, 1H).

### Compound 131

### 3-((13S,15S,Z)-3-chloro-16-(hydroxymethylene)-13-methyl-17-oxo-7,8,9,11,12,13,14,15,16,17-decahydro-6H-cyclopenta[a]phenanthren-15-yl)-N-(3,5-difluoropyridin-2-yl propanamide

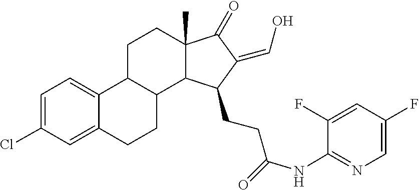 [SMILES: C[C@]12CCC3c4ccc(Cl)cc4CCC3C1[C@H](CCC(=O)Nc1ncc(F)cc1F)/C(=C/O)C2=O]

The compound 131 was synthesized in quantitative yield from the compound 130 by the Method B in 3 hours reaction time.

¹H-NMR (400 MHz, DMSO-d₆): 0.99 (s, 3H), 1.30-2.62 (m, 13H), 2.80-3.00 (m, 3H), 7.13-7.19 (m, 2H), 7.25-7.31 (m, 1H), 7.59 (s, 1H), 7.93-8.03 (m, 1H), 8.31-8.34 (m, 1H), 10.37 (s, 1H), 10.99 (br s, 1H).

### Compound 132

### 3-((8aS,12S)-4-chloro-8a-methyl-1,2,6b,7,8,8a,10,12,12a,12b-decahydronaphtho[2′,1′:4,5]indeno[1,2-c]pyrazol-12-yl)-N-(3,5-difluoropyridin-2-yl)propanamide

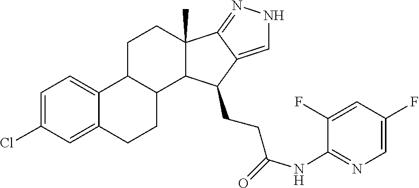 [SMILES: C[C@]12CCC3c4ccc(Cl)cc4CCC3C1[C@H](CCC(=O)Nc1ncc(F)cc1F)c1c[nH]nc12]

The compound 132 was synthesized in 67% yield from the compound 131 by the Method C in 1.5 hours reaction time.

¹H-NMR (400 MHz, DMSO-d₆): 1.10 (s, 3H), 1.30-2.49 (m, 13H), 2.80-3.00 (m, 3H), 7.13-7.19 (m, 2H), 7.27-7.31 (m, 1H), 7.43 (s, 1H), 7.96-8.04 (m, 1H), 8.34 (s, 1H), 10.34 (s, 1H), 12.13 (br s, 1H).

### Compound 133

### 3-((13S,15R)-3-chloro-13-methyl-17-oxo-7,8,9,11,12,13,14,15,16,17-decahydro-6H-cyclopenta[a]phenanthren-15-yl)-N-(6-fluoropyridin-2-yl)propanamide

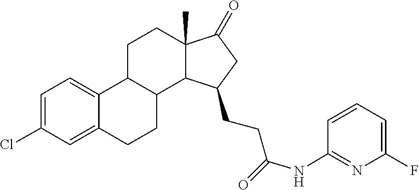 [SMILES: C[C@]12CCC3c4ccc(Cl)cc4CCC3C1[C@H](CCC(=O)Nc1cccc(F)n1)CC2=O]

The compound 133 was synthesized in 71% yield by the Method A1 in THE by using acid SM-XVII and 2-amino-6-fluoropyridine as starting materials in overnight reaction time.

¹H-NMR (400 MHz, DMSO-d₆): 0.97 (s, 3H), 1.30-2.47 (m, 16H), 2.80-2.95 (m, 2H), 6.83 (dd, 1H), 7.14-7.17 (m, 2H), 7.28-7.31 (m, 1H), 7.91-7.97 (m, 1H), 8.00-8.03 (m, 1H), 10.68 (s, 1H).

### Compound 134

### 3-((13S,15S,Z)-3-chloro-16-(hydroxymethylene)-13-methyl-17-oxo-7,8,9,11,12,13,14,15,16,17-decahydro-6H-cyclopenta[a]phenanthren-15-yl)-N-(6-fluoropyridin-2-yl)propanamide

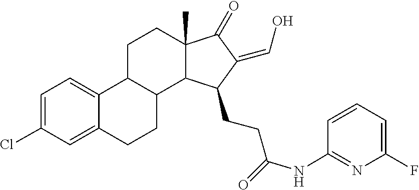 [SMILES: C[C@]12CCC3c4ccc(Cl)cc4CCC3C1[C@H](CCC(=O)Nc1cccc(F)n1)/C(=C/O)C2=O]

The compound 134 was synthesized in 69% yield from the compound 133 by the Method B in 6 hours reaction time.

¹H-NMR (400 MHz, DMSO-d₆): 0.99 (s, 3H), 1.30-2.60 (m, 13H), 2.80-2.99 (m, 3H), 6.81 (dd, 1H), 7.12-7.20 (m, 2H), 7.25-7.31 (m, 1H), 7.55 (s, 1H), 7.88-7.97 (m, 1H), 7.98-8.02 (m, 1H), 10.66 (br s, 1H), 10.95 (br s, 1H).

### Compound 135

### 3-((8aS,12S)-4-chloro-8a-methyl-1,2,6b,7,8,8a,10,12,12a,12b-decahydronaphtho[2,1′:4,5]indeno[1,2-c]pyrazol-12-yl)-N-(6-fluoropyridin-2-yl)propanamide

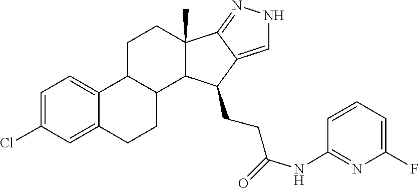 [SMILES: C[C@]12CCC3c4ccc(Cl)cc4CCC3C1[C@H](CCC(=O)Nc1cccc(F)n1)c1c[nH]nc12]

The compound 135 was synthesized in 49% yield from the compound 134 by the Method C in one hour reaction time.

¹H-NMR (400 MHz, DMSO-d₆): 1.10 (s, 3H), 1.30-2.55 (m, 13H), 2.75-2.99 (m, 3H), 6.83 (dd, 1H), 7.11-7.20 (m, 2H), 7.27-7.33 (m, 1H), 7.39 (s, 1H), 7.89-7.98 (m, 1H), 7.99-8.04 (m, 1H), 10.72 (s, 1H), 12.12 (br s, 1H).

### Compound 136

### N-(5-isopropylpyridin-2-yl)-3-((13S,15R)-13-methyl-17-oxo-7,8,9,11,12,13,14,15,16,17-decahydro-6H-cyclopenta[a]phenanthren-15-yl)propanamide

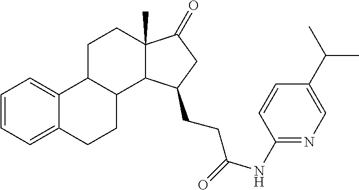 [SMILES: CC(C)c1ccc(NC(=O)CC[C@@H]2CC(=O)[C@@]3(C)CCC4c5ccccc5CCC4C23)nc1]

The compound 136 was prepared by the Method A1 from Acid SM-XXVI and 5-(1-Methyl-ethyl)-2-pyridinamine stirring overnight at room temperature. The yield after chromatographic purification was 75%.

¹H-NMR (200 MHz, CDCl₃): 1.07 (s, 3H), 1.24 & 1.28 (2×s, 6H), 1.45-2.58 (m, 16H), 2.95 (m, 3H), 7.13-7.18 (m, 3H), 7.29-7.31 (m, 1H), 7.58 (dd, 1H), 8.07 (d, 1H), 8.14 (br s, 2H).

### Compound 137

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(5-isopropylpyridin-2-yl)-propanamide

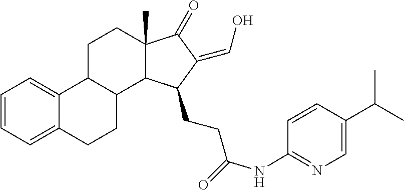 [SMILES: CC(C)c1ccc(NC(=O)CC[C@@H]2/C(=C/O)C(=O)[C@@]3(C)CCC4c5ccccc5CCC4C23)nc1]

The compound 137 was prepared from the compound 136 by the Method B in 97% yield.

¹H NMR (400 MHz, DMSO-d₆): 1.00 (s, 3H), 1.19 & 1.21 (2×s, 6H), 1.38-1.78 (m, 8H), 1.90-1.97 (m, 1H), 2.15 (m, 1H), 2.33 (m, 2H), 2.55 (m, 1H), 2.80-3.00 (m, 4H), 7.05-7.12 (m, 3H), 7.26 (m, 1H), 7.56 (s, 1H), 7.65 (dd, 1H), 8.00 (d, 1H), 8.17 (d, 1H), 10.39 (s, 1H), 11.0 (br s, 1H).

### Compound 138

### N-(5-isopropylpyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide

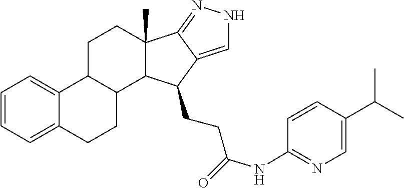 [SMILES: CC(C)c1ccc(NC(=O)CC[C@@H]2c3c[nH]nc3[C@@]3(C)CCC4c5ccccc5CCC4C23)nc1]

The compound 138 was prepared from the compound 137 by the Method C in 93% yield.

¹H NMR (400 MHz, DMSO-d₆): 1.11 (s, 3H), 1.20 & 1.21 (2×s, 6H), 1.37-2.45 (m, 13H), 2.79-2.94 (m, 4H), 7.06-7.14 (m, 3H), 7.28 (m, 1H), 7.40 (s, 1H), 7.66 (dd, 1H), 8.02 (d, 1H), 8.19 (d, 1H), 10.46 (s, 1H), 12.11 (br s, 1H).

### Compound 139

### N-(5-methoxypyridin-2-yl)-3-((13S,15R)-13-methyl-17-oxo-7,8,9,11,12,13,14,15,16,17-decahydro-6H-cyclopenta[a]phenanthren-15-yl)propanamide

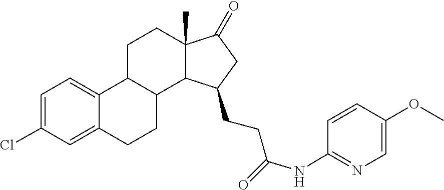 [SMILES: COc1ccc(NC(=O)CC[C@@H]2CC(=O)[C@@]3(C)CCC4c5ccc(Cl)cc5CCC4C23)nc1]

The compound 139 was synthesized in 99% yield from acid Acid SM-XXVI and 5-methoxy-2-aminopyridine by the Method A1 refluxing for two hours.

¹H-NMR (200 MHz, CDCl₃): 1.07 (s, 3H), 1.40-2.57 (m, 17H), 2.96 (m, 2H), 3.85 (s, 3H), 7.13-7.18 (m, 3H), 7.24-7.30 (m, 2H), 7.96 (d, 1H), 8.15 (d, 1H).

### Compound 140

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(5-methoxypyridin-2-yl-propanamide

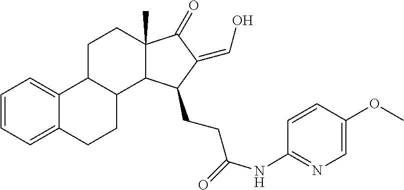 [SMILES: COc1ccc(NC(=O)CC[C@@H]2/C(=C/O)C(=O)[C@@]3(C)CCC4c5ccccc5CCC4C23)nc1]

The compound 140 was prepared from the compound 139 by the Method B in quantitative yield.

¹H NMR (400 MHz, DMSO-d₆): 1.00 (s, 3H), 1.36-2.55 (m, 13H), 2.81-2.95 (m, 3H), 3.82 (s, 3H), 7.05-7.13 (m, 3H), 7.27 (m, 1H), 7.40 (dd, 1H), 7.55 (s, 1H), 8.01 (dd, 2H), 10.33 (s, 1H), 11.01 (br s, 1H).

### Compound 141

### N-(5-methoxypyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide

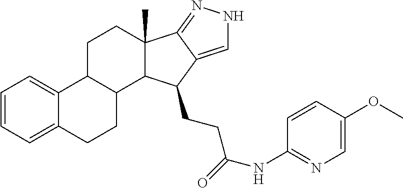 [SMILES: COc1ccc(NC(=O)CC[C@@H]2c3c[nH]nc3[C@@]3(C)CCC4c5ccccc5CCC4C23)nc1]

The compound 141 was prepared from the compound 140 by the Method C in 86% yield.

¹H NMR (400 MHz, DMSO-d₆): 1.11 (s, 3H), 1.41-1.81 (m, 5H), 2.02-2.67 (m, 8H), 2.79-2.98 (m, 3H), 3.80 (s, 3H), 7.07-7.14 (m, 3H), 7.28 (m, 1H), 7.40 (dd, 2H), 8.02 (d, 1H), 8.05 (d, 1H), 10.39 (s, 1H), 12.09 (br s, 1H).

### Compound 142

### 4-(3-((13S,15R)-13-methyl-17-oxo-7,8,9,11,12,13,14,15,16,17-decahydro-6H-cyclopenta[a]phenanthren-15-yl)propanoyl)piperazin-2-one

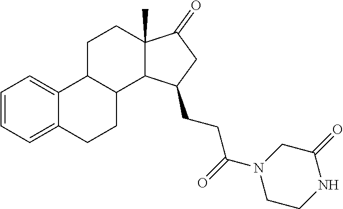 [SMILES: C[C@]12CCC3c4ccccc4CCC3C1[C@H](CCC(=O)N1CCNC(=O)C1)CC2=O]

The compound 142 was synthesized in 86% yield from acid Acid SM-XXVI and piperazin-2-one by the Method A1 stirring at room temperature for two hours.

¹H-NMR (400 MHz, DMSO-d₆): 0.97 (s, 3H), 1.37-2.45 (m, 14H), 2.87 (m, 2H), 3.16-3.24 (m, 3H), 3.63 (m, 3H), 3.93 (s, 1H), 4.04 (s, 1H), 7.10 (m, 3H), 7.27 (d, 1H), 8.08 (d, 1H).

### Compound 143

### 4-(3-((13S,15S,Z)-16-(hydroxymethylene)-13-methyl-17-oxo-7,8,9,11,12,13,14,15,16,17-decahydro-6H-cyclopenta[a]phenanthren-15-yl)propanoyl)piperazin-2-one

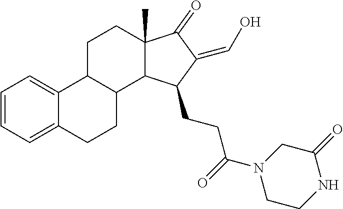 [SMILES: C[C@]12CCC3c4ccccc4CCC3C1[C@H](CCC(=O)N1CCNC(=O)C1)/C(=C/O)C2=O]

The compound 143 was prepared in 80% yield from the compound 142 by the Method B stirring overnight at room temperature.

¹H NMR (400 MHz, DMSO-d₆): 0.98 (s, 3H), 1.38-2.35 (m, 12H), 2.56 (m, 1H), 2.88 (m, 3H), 3.16 (m, 3H), 3.54-3.66 (m, 2H), 3.93 (d, 1H), 3.99 (s, 1H), 7.09 (m, 3H), 7.26 (m, 1H), 7.53 (s, 1H), 8.08 (d, 1H).

### Compound 144

### 4-(3-((8aS,12S)-8a-methyl-1,2,6b,7,8,8a,10,12,12a,12b-decahydronaphtho[2′,1′:4,5]indeno[1,2-c]pyrazol-12-yl)propanoyl)piperazin-2-one

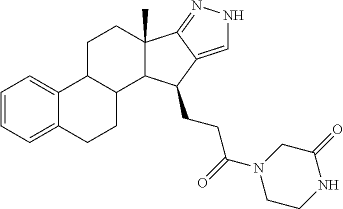 [SMILES: C[C@]12CCC3c4ccccc4CCC3C1[C@H](CCC(=O)N1CCNC(=O)C1)c1c[nH]nc12]

The compound 144 was prepared from the compound 143 by the Method C in 36% yield.

¹H NMR (400 MHz, DMSO-d₆): 1.10 (s, 3H), 1.35-2.46 (m, 15H), 2.90 (m, 2H), 3.15-3.25 (m, 2H), 3.51-3.65 (m, 2H), 3.92 (d, 1H), 7.11 (m, 3H), 7.28 (m, 1H), 7.44 (d, 1H), 8.10 (d, 1H), 12.11 (br s, 1H).

### Compound 145

### (13S,15R)-13-methyl-15-(3-oxo-3-(pyrrolidin-1-yl)propyl)-6,7,8,9,11,12,13,14,15,16-decahydro-17H-cyclopenta[a]phenanthren-17-one

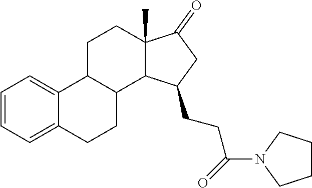 [SMILES: C[C@]12CCC3c4ccccc4CCC3C1[C@H](CCC(=O)N1CCCC1)CC2=O]

The compound 145 was synthesized in 93% yield from acid Acid SM-XXVI and pyrrolidine by the Method A1 in two hours reaction time.

¹H-NMR (400 MHz, DMSO-d₆): 0.97 (s, 3H), 1.37-2.43 (m, 20H), 2.87 (m, 2H), 3.27 (t, 2H), 3.40 (t, 2H), 7.09 (m, 3H), 7.27 (m, 1H).

### Compound 146

### (13S,15S,Z)-16-(hydroxymethylene)-13-methyl-15-(3-oxo-3-(pyrrolidin-1-yl)propyl)-6,7,8,9,11,12,13,14,15,16-decahydro-17H-cyclopenta[a]phenanthren-17-one

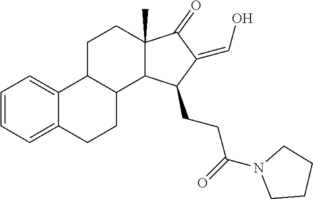 [SMILES: C[C@]12CCC3c4ccccc4CCC3C1[C@H](CCC(=O)N1CCCC1)/C(=C/O)C2=O]

The compound 146 was prepared from the compound 145 by the Method B in 58% yield.

¹H NMR (400 MHz, DMSO-d₆): 0.97 (s, 3H), 1.35-2.40 (m, 16H), 2.88 (m, 3H), 3.26-3.45 (m, 4H), 7.09 (m, 3H), 7.27 (m, 1H), 7.50 (s, 1H), 11.42 (br s, 1H).

### Compound 147

### 3-((8aS,12S)-8a-methyl-1,2,6b,7,8,8a,10,12,12a,12b-decahydronaphtho[2′,1′:4,5]indeno[1,2-c]pyrazol-12-yl)-1-(pyrrolidin-1-yl)propan-1-one

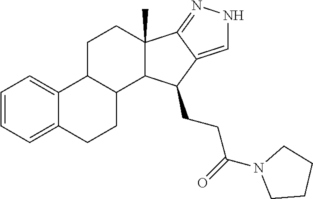 [SMILES: C[C@]12CCC3c4ccccc4CCC3C1[C@H](CCC(=O)N1CCCC1)c1c[nH]nc12]

The compound 147 was prepared from the compound 146 by the Method C in 86% yield.

¹H NMR (400 MHz, DMSO-d₆): 1.10 (s, 3H), 1.39-2.43 (m, 17H), 2.81-2.97 (m, 3H), 3.25-3.30 (m, 2H), 3.38-3.48 (m, 2H), 7.10 (m, 3H), 7.29 (m, 1H), 7.41 (s, 1H), 12.11 (br s, 1H).

### Compound 148

### 3-((13S,15R)-13-methyl-17-oxo-7,8,9,11,12,13,14,15,16,17-decahydro-6H-cyclopenta[a]phenanthren-15-yl)-N-(2-oxo-1,2,5,6,7,8-hexahydroquinolin-3-yl)propanamide

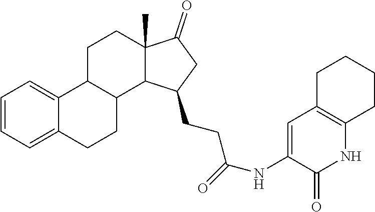 [SMILES: C[C@]12CCC3c4ccccc4CCC3C1[C@H](CCC(=O)Nc1cc3c([nH]c1=O)CCCC3)CC2=O]

Compound 148 was synthesized in 59% yield by the Method A1 in THE by using acid SM-XXVI and 3-amino-1,2,5,6,7,8-hexahydroquinolin-2-one as starting materials in overnight reaction time.

¹H-NMR (400 MHz, DMSO-d₆): 0.97 (s, 3H), 1.30-2.60 (m, 24H), 2.79-2.99 (m, 2H), 7.00-7.15 (m, 3H), 7.23-7.30 (m, 1H), 8.01 (s, 1H), 9.13 (s, 1H), 11.67 (s, 1H).

### Compound 149

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(2-oxo-1,2,5,6,7,8-hexahydroquinolin-3-yl)propanamide

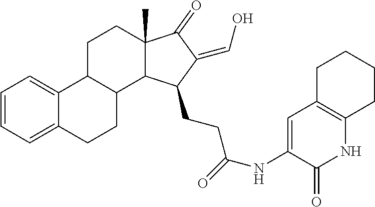 [SMILES: C[C@]12CCC3c4ccccc4CCC3C1[C@H](CCC(=O)Nc1cc3c([nH]c1=O)CCCC3)/C(=C/O)C2=O]

Compound 149 was synthesized in quantitative yield from the compound 148 by the Method B in 2 days reaction time by using 900 mol-% of ethyl formate and 800 mol-% of NaH.

¹H-NMR (400 MHz, DMSO-d₆): 0.98 (s, 3H), 1.30-2.60 (m, 21H), 2.70-2.99 (m, 3H), 7.00-7.15 (m, 3H), 7.23-7.30 (m, 1H), 7.52 (s, 1H), 8.00 (s, 1H), 8.97 (s, 1H), 11.08 (br s, 1H), 11.66 (s, 1H).

### Compound 150

### 3-((8aS,12S)-8a-methyl-1,2,6b,7,8,8a,10,12,12a,12b-decahydronaphtho[2′,1′:4,5]indeno[1,2-c]pyrazol-12-yl)-N-(2-oxo-1,2,5,6,7,8-hexahydroquinolin-3-yl)propanamide

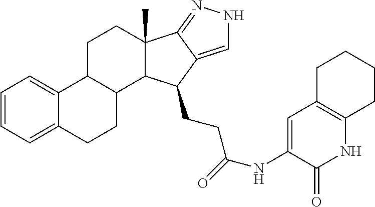 [SMILES: C[C@]12CCC3c4ccccc4CCC3C1[C@H](CCC(=O)Nc1cc3c([nH]c1=O)CCCC3)c1c[nH]nc12]

Compound 150 was synthesized in 47% yield after chromatographic purification from the compound 149 by the Method C in one hour reaction time.

¹H-NMR (400 MHz, DMSO-d₆): 1.10 (s, 3H), 1.30-2.60 (m, 21H), 2.75-2.99 (m, 3H), 7.00-7.15 (m, 3H), 7.23-7.30 (m, 1H), 7.41 (s, 1H), 8.00 (s, 1H), 9.15 (s, 1H), 11.68 (br s, 1H), 12.10 (br s, 1H).

### Compound 151

### 3-((13S,15R)-13-methyl-17-oxo-7,8,9,11,12,13,14,15,16,17-decahydro-6H-cyclopenta[a]phenanthren-15-yl)-N-(tetrahydro-2H-pyran-4-yl)propanamide

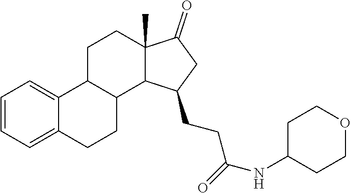 [SMILES: C[C@]12CCC3c4ccccc4CCC3C1[C@H](CCC(=O)NC1CCOCC1)CC2=O]

The compound 151 was synthesized in 99% yield from acid Acid SM-XXVI and 4-aminotetrahydropyran by the Method A1 in THE stirring at room temperature for two hours.

¹H-NMR (400 MHz, DMSO-d₆): 0.96 (s, 3H), 1.33-2.40 (m, 20H), 2.87 (m, 2H), 3.31 (m, 1H), 3.36 (m, 1H), 3.70-3.83 (m, 3H), 7.08 (m, 3H), 7.27 (d, 1H), 7.82 (d, 1H).

### Compound 152

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(tetrahydro-2H-pyran-4-yl)propanamide

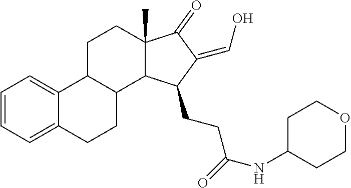 [SMILES: C[C@]12CCC3c4ccccc4CCC3C1[C@H](CCC(=O)NC1CCOCC1)/C(=C/O)C2=O]

The compound 152 was prepared from the compound 151 by the Method B in 89% yield.

¹H NMR (400 MHz, DMSO-d₆): 0.98 (s, 3H), 1.13-2.40 (m, 20H), 2.88 (m, 3H), 3.74-3.84 (m, 2H), 7.09 (m, 3H), 7.27 (m, 1H), 7.48 (br s, 1H), 8.14 (d, 1H), 11.57 (br s, 1H).

### Compound 153

### 3-((8aS,12S)-8a-methyl-1,2,6b,7,8,8a,10,12,12a,12b-decahydronaphtho[2′,1′:4,5]indeno[1,2-c]pyrazol-12-yl)-N-(tetrahydro-2H-pyran-4-yl)propanamide

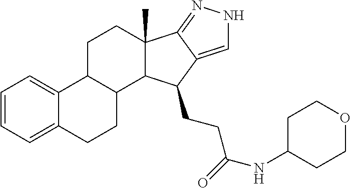 [SMILES: C[C@]12CCC3c4ccccc4CCC3C1[C@H](CCC(=O)NC1CCOCC1)c1c[nH]nc12]

The compound 153 was prepared from the compound 152 by the Method C in 59% yield.

¹H NMR (400 MHz, DMSO-d₆): 1.09 (s, 3H), 1.30-2.39 (m, 17H), 2.77 (m, 1H), 2.89 (m, 2H), 3.36 (m, 2H), 3.80 (m, 3H), 7.09 (m, 3H), 7.28 (m, 1H), 7.38 (br s, 1H), 7.85 (d, 1H), 12.10 (br s, 1H).

### Compound 154

### N-(6-methoxypyridazin-3-yl)-3-((13S,15R)-13-methyl-17-oxo-7,8,9,11,12,13,14,15,16,17-decahydro-6H-cyclopenta[a]phenanthren-15-yl)propanamide

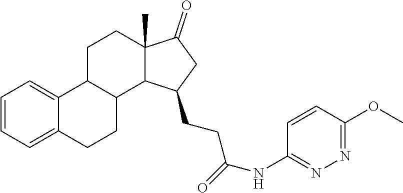 [SMILES: COc1ccc(NC(=O)CC[C@@H]2CC(=O)[C@@]3(C)CCC4c5ccccc5CCC4C23)nn1]

Compound 154 was synthesized in 91% yield by the Method A1 in THE by using acid SM-XXVI and 3-amino-6-methoxypyridazine as starting materials in overnight reaction time.

¹H-NMR (400 MHz, DMSO-d₆): 0.98 (s, 3H), 1.30-2.55 (m, 16H), 2.81-2.96 (m, 2H), 3.98 (s, 3H), 7.00-7.15 (m, 3H), 7.22-7.30 (m, 2H), 8.25 (d, 1H), 10.93 (s, 1H).

### Compound 155

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(6-methoxypyridazin-3-yl)propanamide

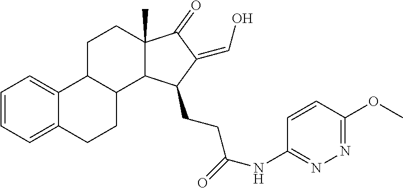 [SMILES: COc1ccc(NC(=O)CC[C@@H]2/C(=C/O)C(=O)[C@@]3(C)CCC4c5ccccc5CCC4C23)nn1]

Compound 155 was synthesized in 96% yield from the compound 154 by the Method B in 3 hours reaction time.

¹H-NMR (400 MHz, DMSO-d₆): 1.00 (s, 3H), 1.30-2.65 (m, 13H), 2.81-3.05 (m, 3H), 3.98 (s, 3H), 7.00-7.15 (m, 3H), 7.20-7.30 (m, 2H), 7.60 (s, 1H), 8.24 (d, 1H), 10.98 (s, 1H).

### Compound 156

### N-(6-methoxypyridazin-3-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide

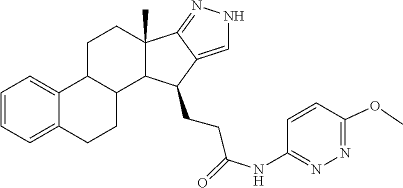 [SMILES: COc1ccc(NC(=O)CC[C@@H]2c3c[nH]nc3[C@@]3(C)CCC4c5ccccc5CCC4C23)nn1]

Compound 156 was synthesized in 74% yield from the compound 155 by the Method C in one hour reaction time.

¹H-NMR (400 MHz, DMSO-d₆): 1.11 (s, 3H), 1.40-2.60 (m, 13H), 2.80-3.02 (m, 3H), 3.98 (s, 3H), 7.00-7.15 (m, 3H), 7.23 (d, 1H), 7.25-7.32 (m, 1H), 7.41 (s, 1H), 8.25 (d, 1H), 10.96 (s, 1H), 12.11 (br s, 1H).

### Compound 157

### N-(5-fluoropyridin-2-yl)-3-((13S,15R)-13-methyl-17-oxo-7,8,9,11,12,13,14,15,16,17-decahydro-6H-cyclopenta[a]phenanthren-15-yl)propanamide

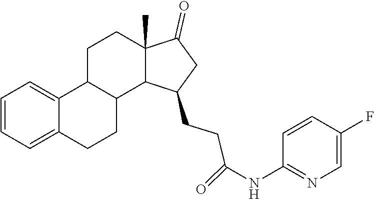 [SMILES: C[C@]12CCC3c4ccccc4CCC3C1[C@H](CCC(=O)Nc1ccc(F)cn1)CC2=O]

Compound 157 was synthesized in quantitative yield by the Method A1 in THE by using acid SM-XXVI and 2-amino-5-fluoropyridine as starting materials in 6 hours reaction time.

¹H-NMR (400 MHz, DMSO-d₆): 0.98 (s, 3H), 1.30-2.55 (m, 16H), 2.81-2.96 (m, 2H), 7.00-7.15 (m, 3H), 7.25-7.29 (m, 1H), 7.69-7.76 (m, 1H), 8.11-8.17 (m, 1H), 8.31 (d, 1H), 10.62 (s, 1H).

### Compound 158

### N-(5-fluoropyridin-2-yl)-3-((13S,15S,Z)-16-(hydroxymethylene)-13-methyl-17-oxo-7,8,9,11,12,13,14,15,16,17-decahydro-6H-cyclopenta[a]phenanthren-15-yl)propanamide

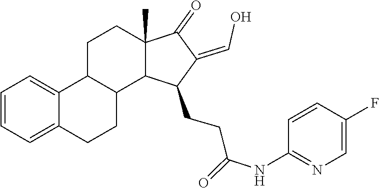 [SMILES: C[C@]12CCC3c4ccccc4CCC3C1[C@H](CCC(=O)Nc1ccc(F)cn1)/C(=C/O)C2=O]

Compound 158 was synthesized in quantitative yield from the compound 157 by the Method B in 3 hours reaction time.

¹H-NMR (400 MHz, DMSO-d₆): 1.00 (s, 3H), 1.30-2.65 (m, 13H), 2.80-2.98 (m, 3H), 7.00-7.15 (m, 3H), 7.23-7.29 (m, 1H), 7.53 (s, 1H), 7.65-7.69 (m, 1H), 8.10-8.17 (m, 1H), 8.30 (d, 1H), 10.54 (s, 1H), 10.96 (s, 1H).

### Compound 159

### N-(5-fluoropyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide

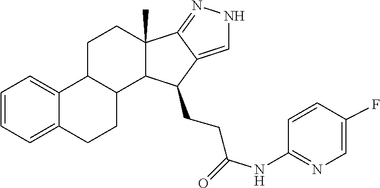 [SMILES: C[C@]12CCC3c4ccccc4CCC3C1[C@H](CCC(=O)Nc1ccc(F)cn1)c1c[nH]nc12]

Compound 159 was synthesized in 84% yield from the compound 158 by the Method C in one hour reaction time.

¹H-NMR (400 MHz, DMSO-d₆): 1.11 (s, 3H), 1.35-2.55 (m, 13H), 2.78-2.99 (m, 3H), 7.00-7.15 (m, 3H), 7.25-7.30 (m, 1H), 7.40 (s, 1H), 7.68-7.76 (m, 1H), 8.12-8.18 (m, 1H), 8.30 (d, 1H), 10.66 (s, 1H), 12.15 (br s, 1H).

### Compound 160

### N-(4-fluoropyridin-2-yl)-3-((13S,15R)-13-methyl-17-oxo-7,8,9,11,12,13,14,15,16,17-decahydro-6H-cyclopenta[a]phenanthren-15-yl)propanamide

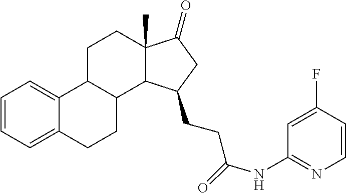 [SMILES: C[C@]12CCC3c4ccccc4CCC3C1[C@H](CCC(=O)Nc1cc(F)ccn1)CC2=O]

Compound 160 was synthesized in 78% yield by the Method A1 in THE by using acid SM-XXVI and 290 mol-% of 2-amino-4-fluoropyridine as starting materials in overnight reaction time.

¹H-NMR (400 MHz, DMSO-d₆): 0.98 (s, 3H), 1.30-2.47 (m, 16H), 2.81-2.96 (m, 2H), 7.00-7.06 (m, 1H), 7.07-7.16 (m, 3H), 7.25-7.30 (m, 1H), 7.92 (dd, 1H), 8.30-8.37 (m, 1H), 10.82 (s, 1H).

### Compound 161

### N-(4-fluoropyridin-2-yl)-3-((13S,15S,Z)-16-(hydroxymethylene)-13-methyl-17-oxo-7,8,9,11,12,13,14,15,16,17-decahydro-6H-cyclopenta[a]phenanthren-15-yl)propanamide

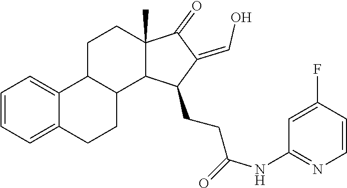 [SMILES: C[C@]12CCC3c4ccccc4CCC3C1[C@H](CCC(=O)Nc1cc(F)ccn1)/C(=C/O)C2=O]

Compound 161 was synthesized in quantitative yield from the compound 160 by the Method B in overnight reaction time and using 1200 mol-% of ethyl formate and 800 mol-% on NaH.

¹H-NMR (400 MHz, DMSO-d₆): 1.00 (s, 3H), 1.30-2.70 (m, 13H), 2.80-2.99 (m, 3H), 6.98-7.04 (m, 1H), 7.05-7.16 (m, 3H), 7.25-7.30 (m, 1H), 7.53 (s, 1H), 7.91 (dd, 1H), 8.30-8.37 (m, 1H), 10.74 (s, 1H), 10.95 (br s, 1H).

### Compound 162

### N-(4-fluoropyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide

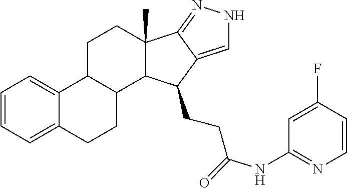 [SMILES: C[C@]12CCC3c4ccccc4CCC3C1[C@H](CCC(=O)Nc1cc(F)ccn1)c1c[nH]nc12]

Compound 162 was synthesized in 47% yield from the compound 161 by the Method C in one hour reaction time.

¹H-NMR (400 MHz, DMSO-d₆): 1.11 (s, 3H), 1.40-2.50 (m, 13H), 2.78-2.99 (m, 3H), 6.98-7.06 (m, 1H), 7.07-7.16 (m, 3H), 7.25-7.30 (m, 1H), 7.39 (s, 1H), 7.93 (dd, 1H), 8.32-8.38 (m, 1H), 10.86 (s, 1H), 12.12 (br s, 1H).

### Compound 163

### N-(4-methoxypyridin-2-yl)-3-((13S,15R)-13-methyl-17-oxo-7,8,9,11,12,13,14,15,16,17-decahydro-6H-cyclopenta[a]phenanthren-15-yl)propanamide

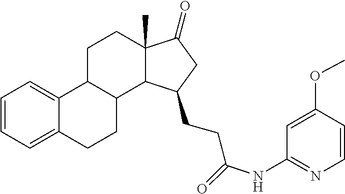 [SMILES: COc1ccnc(NC(=O)CC[C@@H]2CC(=O)[C@@]3(C)CCC4c5ccccc5CCC4C23)c1]

Compound 163 was synthesized in 53% yield by the Method A1 in THE by using acid SM-XXVI and 2-amino-4-methoxypyridine as starting materials in 6 hours reaction time.

¹H-NMR (400 MHz, DMSO-d₆): 0.98 (s, 3H), 1.30-2.47 (m, 16H), 2.81-2.96 (m, 2H), 3.80 (s, 3H), 6.69 (dd, 1H), 7.05-7.16 (m, 3H), 7.25-7.30 (m, 1H), 7.72 (d, 1H), 8.11 (d, 1H), 10.46 (s, 1H).

### Compound 164

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(4-methoxypyridin-2-yl)propanamide

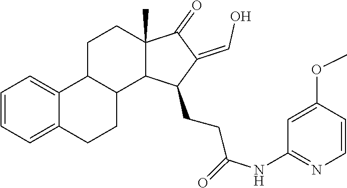 [SMILES: COc1ccnc(NC(=O)CC[C@@H]2/C(=C/O)C(=O)[C@@]3(C)CCC4c5ccccc5CCC4C23)c1]

Compound 164 was synthesized in 94% yield from the compound 163 by the Method B in 4 hours reaction time.

¹H-NMR (400 MHz, DMSO-d₆): 1.00 (s, 3H), 1.30-2.60 (m, 13H), 2.81-3.00 (m, 3H), 3.80 (s, 3H), 6.68 (dd, 1H), 7.05-7.14 (m, 3H), 7.25-7.30 (m, 1H), 7.54 (s, 1H), 7.72 (d, 1H), 8.10 (d, 1H), 10.39 (s, 1H), 10.97 (br s, 1H).

### Compound 165

### N-(4-methoxypyridin-2-yl)-3-((8aS,12S)-8a-methyl-1,2,6b,7,8,8a,10,12,12a,12b-decahydronaphtho[2′1′:4,5]indeno[1,2-c]pyrazol-12-yl)propanamide

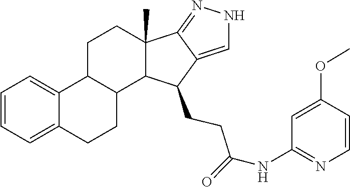 [SMILES: COc1ccnc(NC(=O)CC[C@@H]2c3c[nH]nc3[C@@]3(C)CCC4c5ccccc5CCC4C23)c1]

Compound 165 was synthesized in 76% yield from the compound 164 by the Method C in one hour reaction time.

¹H-NMR (400 MHz, DMSO-d₆): 1.11 (s, 3H), 1.35-2.49 (m, 13H), 2.79-3.00 (m, 3H), 3.80 (s, 3H), 6.68 (dd, 1H), 7.05-7.14 (m, 3H), 7.25-7.30 (m, 1H), 7.39 (s, 1H), 7.73 (d, 1H), 8.12 (d, 1H), 10.49 (s, 1H), 12.12 (br s, 1H).

### Compound 166

### 3-((13S,15R)-13-methyl-17-oxo-7,8,9,11,12,13,14,15,16,17-decahydro-6H-cyclopenta[a]phenanthren-15-yl)-N-4-methylpyridin-2-yl)propanamide

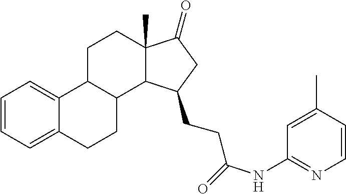 [SMILES: Cc1ccnc(NC(=O)CC[C@@H]2CC(=O)[C@@]3(C)CCC4c5ccccc5CCC4C23)c1]

Compound 166 was synthesized in 82% yield by the Method A1 in THE by using acid SM-XXVI and 2-amino-4-methylpyridine as starting materials in 6 hours reaction time.

¹H-NMR (400 MHz, DMSO-d₆): 0.98 (s, 3H), 1.30-2.48 (m, 19H), 2.80-2.95 (m, 2H), 6.92 (d, 1H), 7.02-7.19 (m, 3H), 7.25-7.29 (m, 1H), 7.95 (s, 1H), 8.15 (d, 1H), 10.41 (s, 1H).

### Compound 167

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(4-methylpyridin-2-yl) propanamide

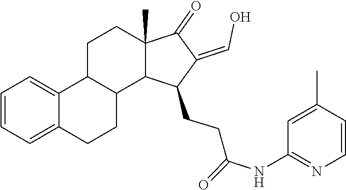 [SMILES: Cc1ccnc(NC(=O)CC[C@@H]2/C(=C/O)C(=O)[C@@]3(C)CCC4c5ccccc5CCC4C23)c1]

Compound 167 was synthesized in quantitative yield from the compound 166 by the Method B in 3 hours reaction time.

¹H-NMR (400 MHz, DMSO-d₆): 1.00 (s, 3H), 1.30-2.65 (m, 16H), 2.80-3.05 (m, 3H), 6.91 (d, 1H), 7.02-7.15 (m, 3H), 7.25-7.29 (m, 1H), 7.54 (s, 1H), 7.94 (s, 1H), 8.14 (d, 1H), 10.33 (s, 1H), 10.97 (br s, 1H).

### Compound 168

### 3-((8aS,12S)-8a-methyl-1,2,6b,7,8,8a,10,12,12a,12b-decahydronaphtho[2′,1′:4,5]indeno[1,2-c]pyrazol-12-yl)-N-(4-methylpyridin-2-yl)propanamide

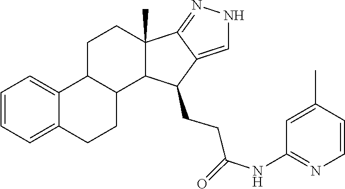 [SMILES: Cc1ccnc(NC(=O)CC[C@@H]2c3c[nH]nc3[C@@]3(C)CCC4c5ccccc5CCC4C23)c1]

Compound 168 was synthesized in 63% yield from the compound 167 by the Method C in one hour reaction time.

¹H-NMR (400 MHz, DMSO-d₆): 1.11 (s, 3H), 1.30-2.49 (m, 16H), 2.79-3.03 (m, 3H), 6.92 (d, 1H), 7.05-7.15 (m, 3H), 7.27-7.31 (m, 1H), 7.41 (s, 1H), 7.96 (s, 1H), 8.15 (d, 1H), 10.45 (s, 1H), 12.11 (br s, 1H).

### Compound 169

### 3-((13S,15R)-13-methyl-17-oxo-7,8,9,11,12,13,14,15,16,17-decahydro-6H-cyclopenta[a]phenanthren-15-yl)-N-(pyridin-2-yl)propanamide

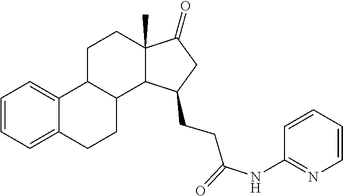 [SMILES: C[C@]12CCC3c4ccccc4CCC3C1[C@H](CCC(=O)Nc1ccccn1)CC2=O]

Compound 169 was synthesized in 75% yield by the Method A1 in THE by using acid SM-XXVI and 2-aminopyridine as starting materials in overnight reaction time.

¹H-NMR (400 MHz, DMSO-d₆): 0.99 (s, 3H), 1.30-2.45 (m, 16H), 2.80-2.96 (m, 2H), 7.00-7.15 (m, 4H), 7.21-7.30 (m, 1H), 7.70-7.82 (m, 1H), 8.09 (d, 1H), 8.28-8.33 (m, 1H), 10.50 (s, 1H).

### Compound 170

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(pyridin-2-yl)propanamide

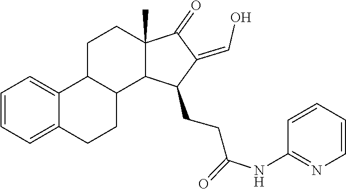 [SMILES: C[C@]12CCC3c4ccccc4CCC3C1[C@H](CCC(=O)Nc1ccccn1)/C(=C/O)C2=O]

Compound 170 was synthesized in quantitative yield from the compound 169 by the Method B in 4 hours reaction time.

¹H-NMR (400 MHz, DMSO-d₆): 1.00 (s, 3H), 1.30-2.65 (m, 13H), 2.80-3.05 (m, 3H), 7.00-7.15 (m, 4H), 7.24-7.30 (m, 1H), 7.56 (s, 1H), 7.70-7.80 (m, 1H), 8.08 (d, 1H), 8.28-8.32 (m, 1H), 10.46 (s, 1H), 10.98 (br s, 1H).

### Compound 171

### 3-((8aS,12S)-8a-methyl-1,2,6b,7,8,8a,10,12,12a,12b-decahydronaphtho[2′,1′:4,5]indeno[1,2-c]pyrazol-12-yl)-N-(pyridin-2-yl)propanamide

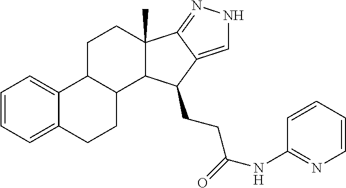 [SMILES: C[C@]12CCC3c4ccccc4CCC3C1[C@H](CCC(=O)Nc1ccccn1)c1c[nH]nc12]

Compound 171 was synthesized in 87% yield from the compound 170 by the Method C in one hour reaction time.

¹H-NMR (400 MHz, DMSO-d₆): 1.11 (s, 3H), 1.35-2.60 (m, 13H), 2.78-3.02 (m, 3H), 7.03-7.15 (m, 4H), 7.26-7.30 (m, 1H), 7.41 (s, 1H), 7.70-7.80 (m, 1H), 8.10 (d, 1H), 8.28-8.32 (m, 1H), 10.53 (s, 1H), 12.12 (br s, 1H).

### Compound 172

### N-(cyclopropylmethyl)-N-methyl-3-((8aS,12S)-8a-methyl-1,2,6b,7,8,8a,10,12,12a,12b-decahydronaphtho[2′,1′:4,5]indeno[1,2-c]pyrazol-12-yl)propanamide

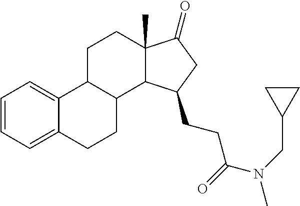 [SMILES: CN(CC1CC1)C(=O)CC[C@@H]1CC(=O)[C@@]2(C)CCC3c4ccccc4CCC3C12]

Compound 172 was synthesized in 80% yield by the Method A1 in THE by using acid SM-XXVI and 400 mol-% of (cyclopropylmethyl)(methyl)amine, 600 mol-% of pyridine and 400 mol-% of T3P in overnight reaction time at rt and 4 hours at 40° C.

¹H-NMR (400 MHz, CDCl₃): 0.21-0.27 (m, 2H), 0.45-0.55 (m, 1H), 0.55-0.65 (m, 1H), 0.87-1.02 (m, 1H), 1.07 (s, 3H), 1.35-2.65 (m, 16H), 2.85-3.05 (m, 2H), 3.01/3.07 (2×s, 3H, isomers), 3.13-3.36 (m, 2H), 7.00-7.20 (m, 3H), 7.25-7.33 (m, 1H).

### Compound 173

### N-(cyclopropylmethyl)-3-((13S,15S,Z)-16-(hydroxymethylene)-13-methyl-17-oxo-7,8,9,11,12,13,14,15,16,17-decahydro-6H-cyclopenta[a]phenanthren-15-yl)-N-methylpropanamide

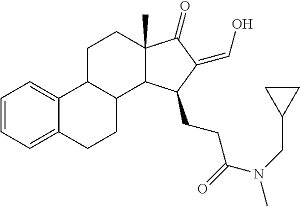 [SMILES: CN(CC1CC1)C(=O)CC[C@@H]1/C(=C/O)C(=O)[C@@]2(C)CCC3c4ccccc4CCC3C12]

Compound 173 was synthesized in 96% yield from the compound 172 by the Method B in 5 hours reaction time.

¹H-NMR (400 MHz, DMSO-d₆): 0.17-0.27 (m, 2H), 0.39-0.55 (m, 2H), 0.72-0.90 (m, 1H), 0.97/0.98 (2×s, 3H, isomers), 1.30-2.70 (m, 13H), 2.80-3.03 (m, 3H), 2.88/3.04 (2×s, 3H, isomers), 3.05-3.25 (m, 2H), 7.00-7.15 (m, 3H), 7.21-7.30 (m, 1H), 7.49/7.50 (2×s, 1H, isomers), 11.44 (br s, 1H).

### Compound 174

### N-(cyclopropylmethyl)-N-methyl-3-((8aS,12S)-8a-methyl-1,2,6b,7,8,8a,10,12,12a,12b-decahydronaphtho[2′,1′:4,5]indeno[1,2-c]pyrazol-12-yl)propanamide

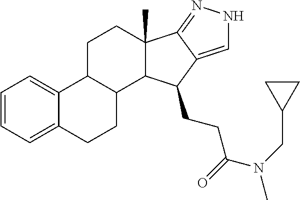 [SMILES: CN(CC1CC1)C(=O)CC[C@@H]1c2c[nH]nc2[C@@]2(C)CCC3c4ccccc4CCC3C12]

Compound 174 was synthesized in 46% yield from the compound 173 by the Method C in 1.5 hours reaction time.

¹H-NMR (400 MHz, DMSO-d₆): 0.17-0.27 (m, 2H), 0.35-0.55 (m, 2H), 0.85-1.00 (m, 1H), 1.10 (s, 3H), 1.25-2.49 (m, 13H), 2.80-3.00 (m, 3H), 2.85/3.04 (2×s, 3H, isomers), 3.05-3.25 (m, 2H), 7.00-7.15 (m, 3H), 7.21-7.30 (m, 1H), 7.38/7.42 (2×br s, 1H, isomers), 12.11 (br s, 1H).

### Compound 175

### (13S,15R)-13-methyl-15-(3-oxo-3-(8-oxa-2-azaspiro[4.5]decan-2-yl)propyl)-6,7,8,9,11,12,13,14,15,16-decahydro-17H-cyclopenta[a]phenanthren-17-one

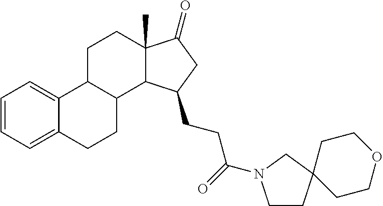 [SMILES: C[C@]12CCC3c4ccccc4CCC3C1[C@H](CCC(=O)N1CCC3(CCOCC3)C1)CC2=O]

Compound 175 was synthesized in 98% yield by the Method A1 in THE by using acid SM-XXVI and 8-oxa-2-aza-spiro(4,5)decane hydrochloride, 800 mol-% of pyridine and 400 mol-% of T3P in overnight reaction time.

¹H-NMR (400 MHz, DMSO-d₆): 0.97 (s, 3H), 1.30-2.46 (m, 22H), 2.84-2.95 (m, 2H), 3.17-3.25 (m, 1H), 3.29-3.40 (m, 2H), 3.46-3.65 (m, 5H), 7.00-7.15 (m, 3H), 7.24-7.29 (m, 1H).

### Compound 176

### (13S,15S,Z)-16-(hydroxymethylene)-13-methyl-15-(3-oxo-3-(8-oxa-2-azaspiro[4.5]decan-2-yl)propyl)-6,7,8,9,11,12,13,14,15,16-decahydro-17H-cyclopenta[a]phenanthren-17-one

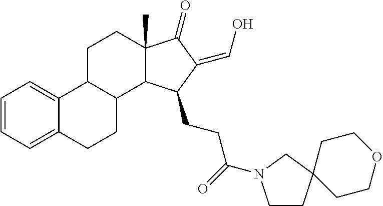 [SMILES: C[C@]12CCC3c4ccccc4CCC3C1[C@H](CCC(=O)N1CCC3(CCOCC3)C1)/C(=C/O)C2=O]

Compound 176 was synthesized in 97% yield from the compound 175 by the Method B in overnight reaction time.

¹H-NMR (400 MHz, DMSO-d₆): 0.98 (s, 3H), 1.30-2.46 (m, 19H), 2.80-2.99 (m, 3H), 3.17-3.29 (m, 1H), 3.30-3.40 (m, 2H), 3.41-3.69 (m, 5H), 7.00-7.15 (m, 3H), 7.24-7.29 (m, 1H), 7.49 (s, 1H), 11.46 (br s, 1H).

### Compound 177

### 3-((8aS,12S)-8a-methyl-1,2,6b,7,8,8a,10,12,12a,12b-decahydronaphtho[2′,1′:4,5]indeno[1,2-c]pyrazol-12-yl)-1-(8-oxa-2-azaspiro[4.5]decan-2-yl)propan-1-one

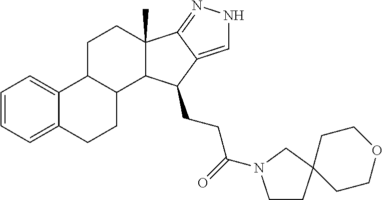 [SMILES: C[C@]12CCC3c4ccccc4CCC3C1[C@H](CCC(=O)N1CCC3(CCOCC3)C1)c1c[nH]nc12]

Compound 177 was synthesized in 68% yield from the compound 176 by the Method C in 0.5 hours reaction time.

¹H-NMR (400 MHz, DMSO-d₆): 1.10 (s, 3H), 1.30-2.46 (m, 19H), 2.80-2.99 (m, 3H), 3.20 (s, 1H), 3.27-3.40 (m, 2H), 3.41-3.69 (m, 5H), 7.00-7.16 (m, 3H), 7.24-7.29 (m, 1H), 7.41/7.45 (2×br s, 1H), 12.11/12.48 (2×br s, 1H).

### Compound 178

### (13S,15R)-15-(3-(isoindolin-2-yl)-3-oxopropyl)-13-methyl-6,7,8,9,11,12,13,14,15,16-decahydro-17H-cyclopenta[a]phenanthren-17-one

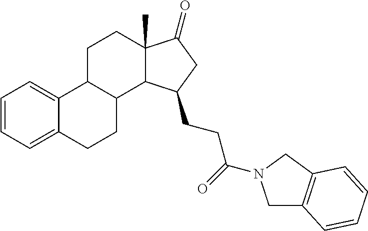 [SMILES: C[C@]12CCC3c4ccccc4CCC3C1[C@H](CCC(=O)N1Cc3ccccc3C1)CC2=O]

Compound 178 was synthesized in 56% yield by the Method A1 in THE by using acid SM-XXVI and isoindoline as starting materials in overnight reaction time.

¹H-NMR (400 MHz, CDCl₃): 1.09 (s, 3H), 1.39-2.60 (m, 16H), 2.85-3.08 (m, 2H), 4.82 (s, 4H), 7.05-7.20 (m, 3H), 7.25-7.34 (m, 5H).

### Compound 179

### (13 S,15 S,Z)-16-(hydroxymethylene)-15-(3-(isoindolin-2-yl)-3-oxopropyl)-13-methyl-6,7,8,9,11,12,13,14,15,16-decahydro-17H-cyclopenta[a]phenanthren-17-one

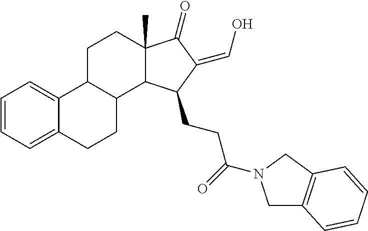 [SMILES: C[C@]12CCC3c4ccccc4CCC3C1[C@H](CCC(=O)N1Cc3ccccc3C1)/C(=C/O)C2=O]

Compound 179 was synthesized in 81% yield after chromatographic purification from the compound 178 by the Method B in overnight reaction time by using 1200 mol-% of ethyl formate and 800 mol-% of NaH.

¹H-NMR (400 MHz, DMSO-d₆): 1.00 (s, 3H), 1.30-2.66 (m, 13H), 2.79-3.05 (m, 3H), 4.65 (s, 2H), 4.75-4.88 (m, 2H), 7.05-7.15 (m, 3H), 7.25-7.40 (m, 5H). 7.54 (s, 1H), 11.09 (br s, 1H).

### Compound 180

### 1-(isoindolin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propan-1-one

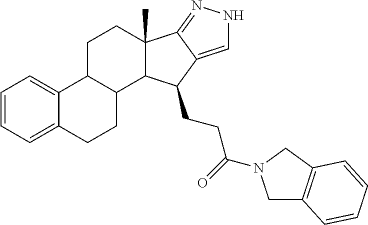 [SMILES: C[C@]12CCC3c4ccccc4CCC3C1[C@H](CCC(=O)N1Cc3ccccc3C1)c1c[nH]nc12]

Compound 180 was synthesized in 92% yield from the compound 179 by the Method C in one hour reaction time.

¹H-NMR (400 MHz, DMSO-d₆): 1.12 (s, 3H), 1.35-2.49 (m, 13H), 2.85-3.01 (m, 3H), 4.64 (s, 2H), 4.80-4.95 (m, 2H), 7.05-7.15 (m, 3H), 7.25-7.40 (m, 5H). 7.47 (s, 1H), 12.12 (br s, 1H).

### Compound 181

### N,N-dimethyl-6-(3-((13S,15R)-13-methyl-17-oxo-7,8,9,11,12,13,14,15,16,17-decahydro-6H-cyclopenta[a]phenanthren-15-yl)propanamido)nicotinamide

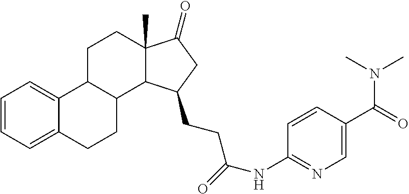 [SMILES: CN(C)C(=O)c1ccc(NC(=O)CC[C@@H]2CC(=O)[C@@]3(C)CCC4c5ccccc5CCC4C23)nc1]

Compound 181 was synthesized in 76% yield by the Method A1 in THE by using acid SM-XXVI and 6-amino-N,N-dimethylpyridine-3-carboxamide as starting materials in overnight reaction time.

¹H-NMR (400 MHz, DMSO-d₆): 0.99 (s, 3H), 1.30-2.56 (m, 16H), 2.79-2.96 (m, 2H), 2.97 (s, 6H), 7.00-7.15 (m, 3H), 7.21-7.30 (m, 1H), 7.85 (dd, 1H), 8.14 (d, 1H), 8.38 (d, 1H), 10.71 (s, 1H).

### Compound 182

### 6-(3-((13S,15S,Z)-16-(hydroxymethylene)-13-methyl-17-oxo-7,8,9,11,12,13,14,15,16,17-decahydro-6H-cyclopenta[a]phenanthren-15-yl)propanamido)-N,N-dimethylnicotinamide

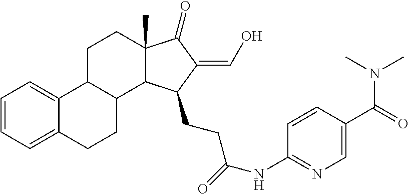 [SMILES: CN(C)C(=O)c1ccc(NC(=O)CC[C@@H]2/C(=C/O)C(=O)[C@@]3(C)CCC4c5ccccc5CCC4C23)nc1]

Compound 182 was synthesized in quantitative yield from the compound 181 by the Method B in overnight reaction time by using 1200 mol-% of ethyl formate and 1000 mol-% of NaH.

¹H-NMR (400 MHz, DMSO-d₆): 1.00 (s, 3H), 1.30-2.66 (m, 13H), 2.79-3.03 (m, 3H), 2.97 (s, 6H), 7.00-7.15 (m, 3H), 7.21-7.30 (m, 1H), 7.54 (s, 1H), 7.84 (dd, 1H), 8.13 (d, 1H), 8.37 (d, 1H), 10.64 (s, 1H), 10.97 (br s, 1H).

### Compound 183

### N,N-dimethyl-6-(3-((8aS,12S)-8a-methyl-1,2,6b,7,8,8a,10,12,12a,12b-decahydronaphtho[2′,1′:4,5]indeno[1,2-c]pyrazol-12-yl)propanamido)nicotinamide

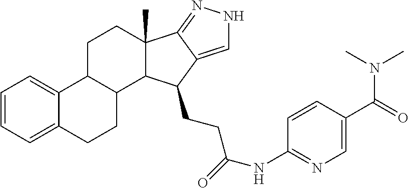 [SMILES: CN(C)C(=O)c1ccc(NC(=O)CC[C@@H]2c3c[nH]nc3[C@@]3(C)CCC4c5ccccc5CCC4C23)nc1]

Compound 183 was synthesized in 80% yield from the compound 182 by the Method C in one hour reaction time.

¹H-NMR (400 MHz, DMSO-d₆): 1.11 (s, 3H), 1.30-2.60 (m, 13H), 2.79-3.03 (m, 3H), 2.98 (s, 6H), 7.00-7.15 (m, 3H), 7.21-7.30 (m, 1H), 7.41 (s, 1H), 7.85 (dd, 1H), 8.15 (d, 1H), 8.39 (d, 1H), 10.76 (s, 1H), 12.14 (br s, 1H).

### Compound 184

### 3-((13S,15R)-13-methyl-17-oxo-7,8,9,11,12,13,14,15,16,17-decahydro-6H-cyclopenta[a]phenanthren-15-yl)-N-(1-methyl-1H-pyrazol-3-yl)propanamide

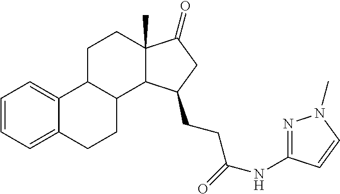 [SMILES: Cn1ccc(NC(=O)CC[C@@H]2CC(=O)[C@@]3(C)CCC4c5ccccc5CCC4C23)n1]

Compound 184 was synthesized in 85% yield by the Method A1 in THE by using acid SM-XXVI and 1-methyl-1H-pyrazol-3-amine as starting materials and 400 mol-% of T3P in 2 days reaction time and warming at 40° C. for 4 hours.

¹H-NMR (400 MHz, DMSO-d₆): 0.97 (s, 3H), 1.30-2.47 (m, 16H), 2.79-2.96 (m, 2H), 3.72 (s, 3H), 6.42 (d, 1H), 7.00-7.15 (m, 3H), 7.23-7.30 (m, 1H), 7.51 (d, 1H), 10.36 (s, 1H).

### Compound 185

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(1-methyl-1H-pyrazol-3-yl)propanamide

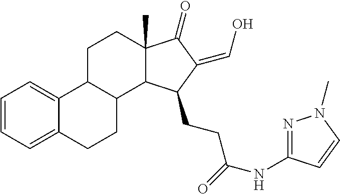 [SMILES: Cn1ccc(NC(=O)CC[C@@H]2/C(=C/O)C(=O)[C@@]3(C)CCC4c5ccccc5CCC4C23)n1]

Compound 185 was synthesized in quantitative yield from the compound 184 by the Method B in 5 hours reaction time.

¹H-NMR (400 MHz, DMSO-d₆): 0.99 (s, 3H), 1.30-2.47 (m, 13H), 2.79-2.96 (m, 3H), 3.72 (s, 3H), 6.42 (d, 1H), 7.00-7.15 (m, 3H), 7.23-7.30 (m, 1H), 7.51 (d, 1H), 7.54 (s, 1H), 10.38 (s, 1H), 11.06 (br s, 1H).

### Compound 186

### 3-((8aS,12S)-8a-methyl-1,2,6b,7,8,8a,10,12,12a,12b-decahydronaphtho[2′,1′:4,5]indeno[1,2-c]pyrazol-12-yl)-N-(1-methyl-1H-pyrazol-3-yl)propanamide

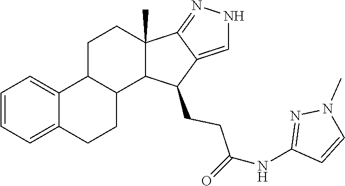 [SMILES: Cn1ccc(NC(=O)CC[C@@H]2c3c[nH]nc3[C@@]3(C)CCC4c5ccccc5CCC4C23)n1]

Compound 186 was synthesized in 62% yield from the compound 185 by the Method C in one hour reaction time.

¹H-NMR (400 MHz, DMSO-d₆): 1.10 (s, 3H), 1.30-2.47 (m, 13H), 2.75-2.96 (m, 3H), 3.72 (s, 3H), 6.43 (d, 1H), 7.00-7.15 (m, 3H), 7.23-7.30 (m, 1H), 7.40 (s, 1H), 7.52 (d, 1H), 10.40 (s, 1H), 12.11 (br s, 1H).

### Compound 187

### 3-((13S,15R)-13-methyl-17-oxo-7,8,9,11,12,13,14,15,16,17-decahydro-6H-cyclopenta[a]phenanthren-15-yl)-N-(pyrazin-2-yl)propanamide

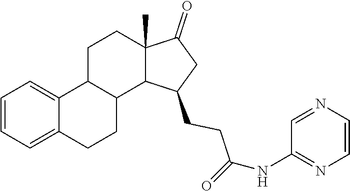 [SMILES: C[C@]12CCC3c4ccccc4CCC3C1[C@H](CCC(=O)Nc1cnccn1)CC2=O]

Compound 187 was synthesized in 66% yield by the Method A1 in THE by using acid SM-XXVI and aminopyrazine as starting materials in overnight reaction time.

¹H-NMR (400 MHz, DMSO-d₆): 0.99 (s, 3H), 1.30-2.60 (m, 16H), 2.79-2.97 (m, 2H), 7.00-7.15 (m, 3H), 7.23-7.30 (m, 1H), 8.34 (d, 1H), 8.39 (d, 1H), 9.35 (s, 1H), 10.81 (s, 1H).

### Compound 188

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(pyrazin-2-yl)propanamide

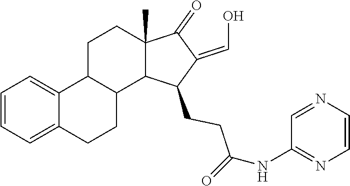 [SMILES: C[C@]12CCC3c4ccccc4CCC3C1[C@H](CCC(=O)Nc1cnccn1)/C(=C/O)C2=O]

Compound 188 was synthesized in quantitative yield from the compound 187 by the Method B in 4 hours reaction time.

¹H-NMR (400 MHz, DMSO-d₆): 1.00 (s, 3H), 1.30-2.70 (m, 13H), 2.79-3.05 (m, 3H), 7.00-7.15 (m, 3H), 7.23-7.30 (m, 1H), 7.54 (s, 1H), 8.33 (d, 1H), 8.37 (d, 1H), 9.33 (s, 1H), 10.73 (s, 1H), 10.95 (br s, 1H).

### Compound 189

### 3-((8aS,12S)-8a-methyl-1,2,6b,7,8,8a,10,12,12a,12b-decahydronaphtho[2′,1′:4,5]indeno[1,2-c]pyrazol-12-yl)-N-(pyrazin-2-yl)propanamide

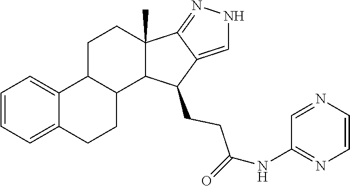 [SMILES: C[C@]12CCC3c4ccccc4CCC3C1[C@H](CCC(=O)Nc1cnccn1)c1c[nH]nc12]

Compound 189 was synthesized in 43% yield after chromatographic purification from the compound 188 by the Method C in one hour reaction time.

¹H-NMR (400 MHz, DMSO-d₆): 1.12 (s, 3H), 1.39-2.60 (m, 13H), 2.79-3.05 (m, 3H), 7.00-7.15 (m, 3H), 7.23-7.30 (m, 1H), 7.40 (s, 1H), 8.34 (d, 1H), 8.40 (d, 1H), 9.35 (s, 1H), 10.84 (s, 1H), 12.15 (br s, 1H).

### Compound 190

### N-cyclobutyl-3-((13S,15R)-13-methyl-17-oxo-7,8,9,11,12,13,14,15,16,17-decahydro-6H-cyclopenta[a]phenanthren-15-yl)propanamide

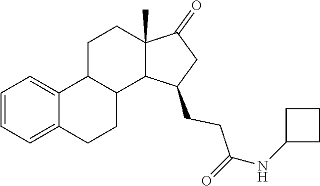 [SMILES: C[C@]12CCC3c4ccccc4CCC3C1[C@H](CCC(=O)NC1CCC1)CC2=O]

Compound 190 was synthesized in 91% yield by the Method A1 in THE by using acid SM-XXVI and cyclobutylamine as starting materials in 5 hours reaction time.

¹H-NMR (400 MHz, DMSO-d₆): 0.95 (s, 3H), 1.30-2.43 (m, 22H), 2.79-2.97 (m, 2H), 4.10-4.25 (m, 1H), 7.00-7.15 (m, 3H), 7.23-7.30 (m, 1H), 8.07 (d, 1H).

### Compound 191

### N-cyclobutyl-3-((13S,15S,Z)-16-(hydroxymethylene)-13-methyl-17-oxo-7,8,9,11,12,13,14,15,16,17-decahydro-6H-cyclopenta[a]phenanthren-15-yl)propanamide

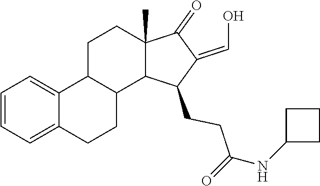 [SMILES: C[C@]12CCC3c4ccccc4CCC3C1[C@H](CCC(=O)NC1CCC1)/C(=C/O)C2=O]

Compound 191 was synthesized in quantitative yield from the compound 190 by the Method B in 4 hours reaction time.

¹H-NMR (400 MHz, DMSO-d₆): 0.97 (s, 3H), 1.30-2.43 (m, 19H), 2.79-2.97 (m, 3H), 4.10-4.25 (m, 1H), 7.00-7.15 (m, 3H), 7.23-7.30 (m, 1H), 7.53 (s, 1H), 8.39 (d, 1H).

### Compound 192

### N-cyclobutyl-3-((8aS,12S)-8a-methyl-1,2,6b,7,8,8a,10,12,12a,12b-decahydronaphtho[2′,1′:4,5]indeno[1,2-c]pyrazol-12-yl)propanamide

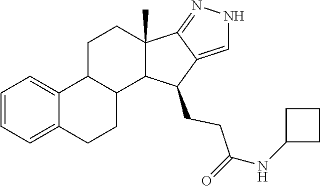 [SMILES: C[C@]12CCC3c4ccccc4CCC3C1[C@H](CCC(=O)NC1CCC1)c1c[nH]nc12]

Compound 192 was synthesized in 80% yield from the compound 191 by the Method C in one hour reaction time.

¹H-NMR (400 MHz, DMSO-d₆): 1.09 (s, 3H), 1.30-2.43 (m, 19H), 2.70-2.98 (m, 3H), 4.10-4.25 (m, 1H), 7.00-7.15 (m, 3H), 7.25-7.30 (m, 1H), 7.37 (s, 1H), 8.10 (d, 1H), 12.12 (br s, 1H).

### Compound 193

### 3-((13S,15R)-13-methyl-17-oxo-7,8,9,11,12,13,14,15,16,17-decahydro-6H-cyclopenta[a]phenanthren-15-yl)-N-(6-methylpyridazin-3-yl)propanamide

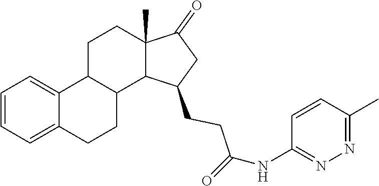 [SMILES: Cc1ccc(NC(=O)CC[C@@H]2CC(=O)[C@@]3(C)CCC4c5ccccc5CCC4C23)nn1]

Compound 193 was synthesized in 62% yield by the Method A1 in THE by using acid SM-XXVI and 3-amino-6-methylpyridazine as starting materials in 2 days reaction time.

¹H-NMR (400 MHz, DMSO-d₆): 0.99 (s, 3H), 1.30-2.49 (m, 16H), 2.55 (s, 3H), 2.79-2.97 (m, 2H), 7.00-7.15 (m, 3H), 7.23-7.30 (m, 1H), 7.54 (d, 1H), 8.22 (d, 1H), 11.04 (s, 1H).

### Compound 194

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(6-methylpyridazin-3-yl)propanamide

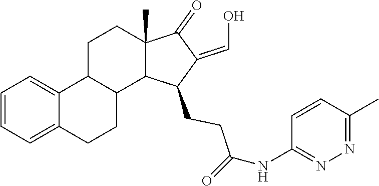 [SMILES: Cc1ccc(NC(=O)CC[C@@H]2/C(=C/O)C(=O)[C@@]3(C)CCC4c5ccccc5CCC4C23)nn1]

Compound 194 was synthesized in quantitative yield from the compound 193 by the Method B in overnight reaction time and then warming at 30° C. for 5 hours by using 1200 mol-% of ethyl formate and 800 mol-% of NaH.

¹H-NMR (400 MHz, DMSO-d₆): 1.00 (s, 3H), 1.30-2.70 (m, 13H), 2.55 (s, 3H), 2.80-3.05 (m, 3H), 7.00-7.15 (m, 3H), 7.23-7.30 (m, 1H), 7.52 (d, 1H), 7.54 (s, 1H), 8.21 (d, 1H), 10.93 (br s, 1H).

### Compound 195

### 3-((8aS,12S)-8a-methyl-1,2,6b,7,8,8a,10,12,12a,12b-decahydronaphtho[2′,1′:4,5]indeno[1,2-c]pyrazol-12-yl)-N-(6-methylpyridazin-3-yl)propanamide

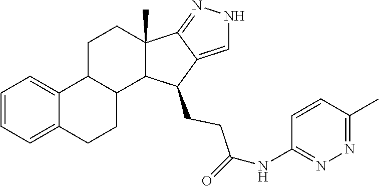 [SMILES: Cc1ccc(NC(=O)CC[C@@H]2c3c[nH]nc3[C@@]3(C)CCC4c5ccccc5CCC4C23)nn1]

Compound 195 was synthesized in 53% yield from the compound 194 by the Method C in one hour reaction time.

¹H-NMR (400 MHz, DMSO-d₆): 1.11 (s, 3H), 1.30-2.61 (m, 13H), 2.55 (s, 3H), 2.78-3.05 (m, 3H), 7.00-7.15 (m, 3H), 7.23-7.30 (m, 1H), 7.41 (s, 1H), 7.53 (d, 1H), 8.22 (d, 1H), 11.06 (s, 1H), 12.12 (br s, 1H).

### Compound 196

### 3-((13S,15R)-13-methyl-17-oxo-7,8,9,11,12,13,14,15,16,17-decahydro-6H-cyclopenta[a]phenanthren-15-yl)-N-(5-(4-methylpiperazin-1-yl)pyridin-2-yl)propanamide

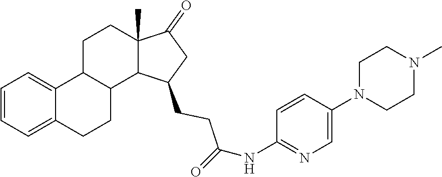 [SMILES: CN1CCN(c2ccc(NC(=O)CC[C@@H]3CC(=O)[C@@]4(C)CCC5c6ccccc6CCC5C34)nc2)CC1]

Compound 196 was synthesized in 39% yield by the Method A1 in THE by using acid SM-XXVI and 1-methyl-4-(6-aminopyridin-3-yl)piperazine as starting materials in 5 hours reaction time.

¹H-NMR (400 MHz, DMSO-d₆): 0.98 (s, 3H), 1.30-2.49 (m, 16H), 2.21 (s, 3H), 2.40-2.49 (m, 4H), 2.79-2.98 (m, 2H), 3.05-3.15 (m, 4H), 7.00-7.15 (m, 3H), 7.23-7.30 (m, 1H), 7.35-7.41 (m, 1H), 7.90-8.00 (m, 2H), 10.26 (s, 1H).

### Compound 197

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(5-(4-methylpiperazin-1-yl)pyridin-2-yl)propanamide

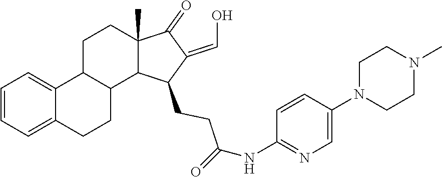 [SMILES: CN1CCN(c2ccc(NC(=O)CC[C@@H]3/C(=C/O)C(=O)[C@@]4(C)CCC5c6ccccc6CCC5C34)nc2)CC1]

Compound 197 was synthesized in 84% yield from the compound 196 by the Method B in 5 hours reaction time.

¹H-NMR (400 MHz, DMSO-d₆): 0.99 (s, 3H), 1.30-2.49 (m, 13H), 2.24 (s, 3H), 2.43-2.55 (m, 4H), 2.79-3.05 (m, 3H), 3.05-3.15 (m, 4H), 7.00-7.15 (m, 3H), 7.23-7.30 (m, 1H), 7.38 (dd, 1H), 7.59 (s, 1H), 7.93 (d, 1H), 7.98 (d, 1H), 10.31 (s, 1H).

### Compound 198

### 3-((8aS,12S)-8a-methyl-1,2,6b,7,8,8a,10,12,12a,12b-decahydronaphtho[2′,1′:4,5]indeno[1,2-c]pyrazol-12-yl)-N-(5-(4-methylpiperazin-1-yl)pyridin-2-yl)propanamide

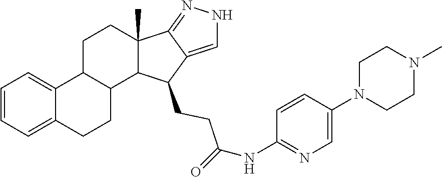 [SMILES: CN1CCN(c2ccc(NC(=O)CC[C@@H]3c4c[nH]nc4[C@@]4(C)CCC5c6ccccc6CCC5C34)nc2)CC1]

Compound 198 was synthesized in 81% yield from the compound 197 by the Method C in one hour reaction time.

¹H-NMR (400 MHz, DMSO-d₆): 1.10 (s, 3H), 1.30-2.49 (m, 13H), 2.22 (s, 3H), 2.43-2.49 (m, 4H), 2.77-3.05 (m, 3H), 3.05-3.15 (m, 4H), 7.00-7.15 (m, 3H), 7.23-7.30 (m, 1H), 7.38 (dd, 1H), 7.40 (s, 1H), 7.95 (d, 1H), 7.98 (d, 1H), 10.29 (s, 1H), 12.10 (br s, 1H).

### Compound 199

### N-(tert-butyl)-3-((13S,15R)-13-methyl-17-oxo-7,8,9,11,12,13,14,15,16,17-decahydro-6H-cyclopenta[a]phenanthren-15-yl)propanamide

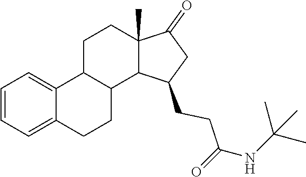 [SMILES: CC(C)(C)NC(=O)CC[C@@H]1CC(=O)[C@@]2(C)CCC3c4ccccc4CCC3C12]

Compound 199 was synthesized in 68% yield by the Method A1 in THE by using acid SM-XXVI and tert-butylamine as starting materials in 2 days reaction time.

¹H-NMR (400 MHz, DMSO-d₆): 0.96 (s, 3H), 1.24 (s, 9H), 1.30-2.46 (m, 16H), 2.82-2.97 (m, 2H), 7.00-7.15 (m, 3H), 7.24-7.29 (m, 1H), 7.44 (s, 1H).

### Compound 200

### N-(tert-butyl)-3-((13S,15S,Z)-16-(hydroxymethylene)-13-methyl-17-oxo-7,8,9,11,12,13,14,15,16,17-decahydro-6H-cyclopenta[a]phenanthren-15-yl)propanamide

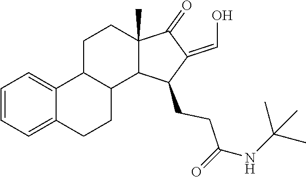 [SMILES: CC(C)(C)NC(=O)CC[C@@H]1/C(=C/O)C(=O)[C@@]2(C)CCC3c4ccccc4CCC3C12]

Compound 200 was synthesized in quantitative yield from the compound 199 by the Method B in overnight reaction time by using 1200 mol-% of ethyl formate and 800 mol-% of NaH.

¹H-NMR (400 MHz, DMSO-d₆): 0.98 (s, 3H), 1.28 (s, 9H), 1.30-2.47 (m, 13H), 2.82-2.97 (m, 3H), 7.01-7.15 (m, 3H), 7.24-7.29 (m, 1H), 7.46 (s, 1H), 7.87 (br s, 1H), 11.88 (br s, 1H).

### Compound 201

### N-(tert-butyl)-3-((8aS,12S)-8a-methyl-1,2,6b,7,8,8a,10,12,12a,12b-decahydronaphtho[2′,1′:4,5]indeno[1,2-c]pyrazol-12-yl)propanamide

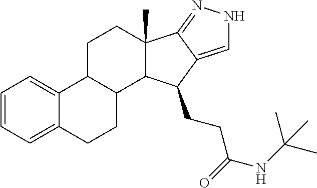 [SMILES: CC(C)(C)NC(=O)CC[C@@H]1c2c[nH]nc2[C@@]2(C)CCC3c4ccccc4CCC3C12]

Compound 201 was synthesized in 30% yield after chromatographic purification from the compound 200 by the Method C in one hour reaction time.

¹H-NMR (400 MHz, DMSO-d₆): 1.09 (s, 3H), 1.25 (s, 9H), 1.13-2.47 (m, 13H), 2.70-2.97 (m, 3H), 7.01-7.16 (m, 3H), 7.24-7.33 (m, 1H), 7.37 (s, 1H), 7.45 (s, 1H), 12.10 (br s, 1H).

### Compound 202

### 3-((13S,15R)-4-fluoro-13-methyl-17-oxo-7,8,9,11,12,13,14,15,16,17-decahydro-6H-cyclopenta[a]phenanthren-15-yl)-N-(6-fuoropyridin-2-yl)propanamide

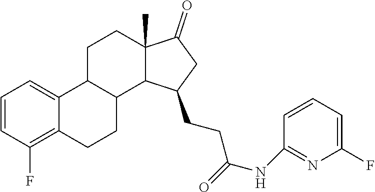 [SMILES: C[C@]12CCC3c4cccc(F)c4CCC3C1[C@H](CCC(=O)Nc1cccc(F)n1)CC2=O]

Compound 202 was synthesized in 88% yield by Method A1 in THE by using acid SM-IX and 2-amino-6-fluoropyridine as starting materials in two hours reaction time.

¹H-NMR (400 MHz, DMSO-d₆): 0.98 (s, 3H), 1.35-1.46 (m, 3H), 1.57-1.77 (m, 4H), 1.93 (m, 1H), 2.16-2.47 (m, 8H), 2.68-2.90 (m, 2H), 6.83 (dd, 1H), 6.97 (dd, 1H), 7.12-7.20 (m, 2H), 7.95 (dd, 1H), 8.01 (d, 1H), 10.69 (s, 1H).

### Compound 203

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(6-fluoropyridin-2-yl)propanamide

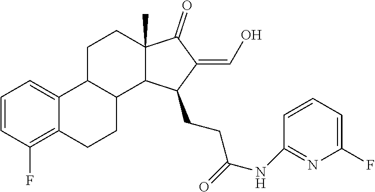 [SMILES: C[C@]12CCC3c4cccc(F)c4CCC3C1[C@H](CCC(=O)Nc1cccc(F)n1)/C(=C/O)C2=O]

The compound 203 was prepared in 90% yield from the compound 202 by Method B stirring overnight at room temperature.

¹H NMR (400 MHz, DMSO-d₆): 0.99 (s, 3H), 1.18-2.37 (m, 13H), 2.45-2.96 (m, 4H), 6.81 (dd, 1H), 6.96 (t, 1H), 7.12-7.19 (m, 2H), 7.55 (s, 1H), 7.92-8.02 (m, 2H), 10.64 (s, 1H).

### Compound 204

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(6-fluoropyridin-2-yl)propanamide

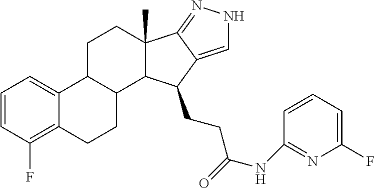 [SMILES: C[C@]12CCC3c4cccc(F)c4CCC3C1[C@H](CCC(=O)Nc1cccc(F)n1)c1c[nH]nc12]

The compound 204 was prepared in 16% yield from the compound 203 by the Method C and purified by crystallization from acetonitrile.

¹H NMR (400 MHz, DMSO-d₆): 1.11 (s, 3H), 1.41-2.42 (m, 13H), 2.72-2.93 (m, 3H), 6.83 (dd, 1H), 6.98 (t, 1H), 7.16 (m, 2H), 7.40 (s, 1H), 7.94 (m, 1H), 8.02 (d, 1H), 10.72 (s, 1H), 12.13 (br s, 1H).

### Compound 205

### N-cyclohexyl-3-((13S,15R)-4-fluoro-13-methyl-17-oxo-7,8,9,11,12,13,14,15,16,17-decahydro-6H-cyclopenta[a]phenanthren-15-yl)propanamide

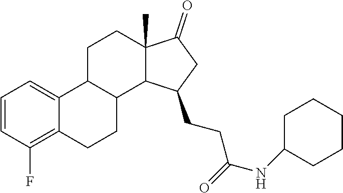 [SMILES: C[C@]12CCC3c4cccc(F)c4CCC3C1[C@H](CCC(=O)NC1CCCCC1)CC2=O]

Compound 205 was synthesized in 64% yield by Method A1 using acid SM-IX and cyclohexylamine as starting materials in 2 hours reaction time. The crude product was purified by chromatography.

¹H-NMR (400 MHz, DMSO-d₆): 0.95 (s, 3H), 1.10-2.41 (m, 26H), 2.67-2.76 (m, 1H), 2.84-2.91 (m, 1H), 3.50-3.53 (m, 1H), 6.94-7.00 (m, 1H), 7.10-7.22 (m, 2H), 7.71 (br d, 1H).

### Compound 206

### N-cyclohexyl-3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)propanamide

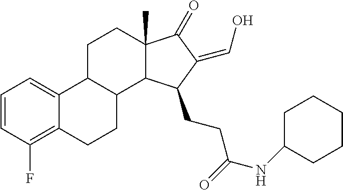 [SMILES: C[C@]12CCC3c4cccc(F)c4CCC3C1[C@H](CCC(=O)NC1CCCCC1)/C(=C/O)C2=O]

Compound 206 was synthesized in 67% yield from the compound 205 by the Method B in 2 hours reaction time.

¹H-NMR (400 MHz, DMSO-d₆): 0.97 (s, 3H), 1.09-1.90 (m, 18H), 2.03-2.16 (m, 2H), 2.30-2.45 (m, 3H), 2.65-2.76 (m, 1H), 2.80-2.95 (m, 2H), 3.50-3.65 (m, 1H), 6.90-7.02 (m, 1H), 7.08-7.25 (m, 2H), 7.50 (s, 1H), 8.14 (br s, 1H) 11.84 (br s, 1H).

### Compound 207

### N-cyclohexyl-3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamide

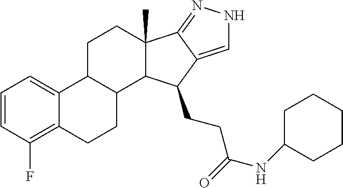 [SMILES: C[C@]12CCC3c4cccc(F)c4CCC3C1[C@H](CCC(=O)NC1CCCCC1)c1c[nH]nc12]

Compound 207 was synthesized in 49% yield from the compound 206 by the Method C in one hour reaction time.

¹H-NMR (400 MHz, DMSO-d₆): 1.08 (s, 3H), 1.09-1.80 (m, 16H), 1.90-2.00 (m, 1H), 2.00-2.25 (m, 4H), 2.30-2.45 (m, 2H), 2.65-2.80 (m, 2H), 2.82-2.95 (m, 1H), 3.45-3.60 (m, 1H), 6.90-7.02 (m, 1H), 7.08-7.25 (m, 2H), 7.39 (s, 1H), 7.73 (d, 1H) 12.12 (br s, 1H).

### Compound 208

### (13S,15R)-4-fluoro-13-methyl-15-(3-oxo-3-(pyrrolidin-1-yl)propyl)-6,7,8,9,11,12,13,14,15,16-decahydro-17H-cyclopenta[a]phenanthren-17-one

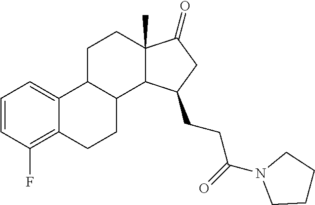 [SMILES: C[C@]12CCC3c4cccc(F)c4CCC3C1[C@H](CCC(=O)N1CCCC1)CC2=O]

Compound 208 was synthesized in 90% yield by Method A1 using acid SM-IX and pyrrolidine as starting materials in 4 hours reaction time.

¹H-NMR (400 MHz, DMSO-d₆): 0.97 (s, 3H), 1.43-2.45 (m, 20H), 2.71-2.91 (m, 2H), 3.39-3.47 (m, 4H), 6.97 (dd, 1H), 7.10-7.20 (m, 2H).

### Compound 209

### (13S,15S,Z)-4-fluoro-16-(hydroxymethylene)-13-methyl-15-(3-oxo-3-(pyrrolidin-1-yl)propyl)-6,7,8,9,11,12,13,14,15,16-decahydro-17H-cyclopenta[a]phenanthren-17-one

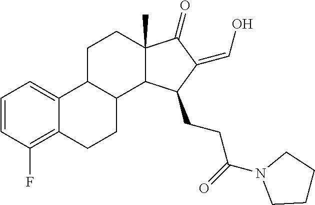 [SMILES: C[C@]12CCC3c4cccc(F)c4CCC3C1[C@H](CCC(=O)N1CCCC1)/C(=C/O)C2=O]

The compound 209 was prepared in 96% yield from the compound 208 by Method B stirring two hours at room temperature.

¹H NMR (400 MHz, CDCl₃): 1.08 (s, 3H), 1.48-2.99 (m, 19H), 3.16 (m, 1H), 3.43-3.66 (m, 4H), 6.88 (dd, 1H), 7.09 (m, 2H), 7.66 (d, 1H), 12.41 (d, 1H).

### Compound 210

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-1-(pyrrolidin-1-yl)propan-1-one

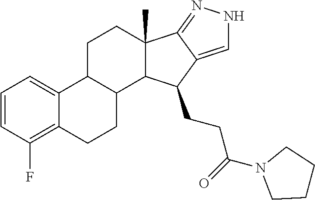 [SMILES: C[C@]12CCC3c4cccc(F)c4CCC3C1[C@H](CCC(=O)N1CCCC1)c1c[nH]nc12]

The compound 210 was prepared in 90% yield from the compound 209 by the Method C.

¹H NMR (400 MHz, DMSO-d₆): 1.10 (s, 3H), 1.36-2.42 (m, 17H), 2.67-2.91 (m, 3H), 3.27-3.47 (m, 4H), 6.98 (dd, 1H), 7.16 (m, 2H), 7.41 (s, 1H), 12.14 (br s, 1H). MS m/z (TOF ES+): 422 (M+1)

### Compound 211

### N-(5-(tert-butyl)isoxazol-3-yl)-3-((13S,15R)-4-fluoro-13-methyl-17-oxo-7,8,9,11,12,13,14,15,16,17-decahydro-6H-cyclopenta[a]phenanthren-15-yl)propanamide

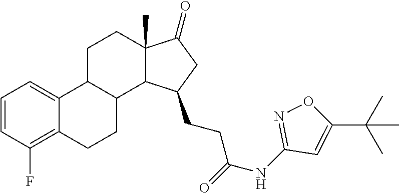 [SMILES: CC(C)(C)c1cc(NC(=O)CC[C@@H]2CC(=O)[C@@]3(C)CCC4c5cccc(F)c5CCC4C23)no1]

Compound 211 was synthesized in 83% yield by the Method A1 by using acid SM-IX and 3-Amino-5-tert-butylisoxazole as starting materials.

¹H NMR (400 MHz, CDCl₃): 1.06 (s, 3H), 1.35 (s, 9H), 1.44-1.95 (m, 8H), 2.09-2.61 (m, 8H), 2.75-3.00 (m, 2H), 6.73 (s, 1H), 6.88 (t, 1H), 7.08 (m, 2H), 9.54 (s, 1H).

### Compound 212

### N-(5-(tert-butyl)isoxazol-3-yl)-3-((13S,15 S,Z)-4-fluoro-16-(hydroxymethylene)-13-methyl-17-oxo-7,8,9,11,12,13,14,15,16,17-decahydro-6H-cyclopenta[a]phenanthren-15-yl)propanamide

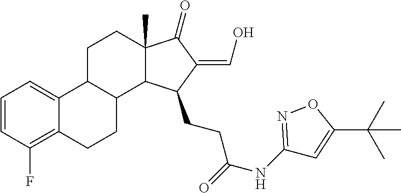 [SMILES: CC(C)(C)c1cc(NC(=O)CC[C@@H]2/C(=C/O)C(=O)[C@@]3(C)CCC4c5cccc(F)c5CCC4C23)no1]

The compound 212 was prepared in 91% yield from the compound 211 by Method B stirring two hours at 0° C.

¹H NMR (400 MHz, DMSO-d₆): 0.99 (s, 3H), 1.28 (s, 9H), 1.38-2.30 (m, 14H), 2.68-2.95 (m, 3H), 6.59 (dd, 1H), 6.97 (t, 1H), 7.14 (m, 2H), 7.56 (s, 1H), 10.92 (s, 1H).

### Compound 213

### N-(5-(tert-butyl)isoxazol-3-yl)-3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamide

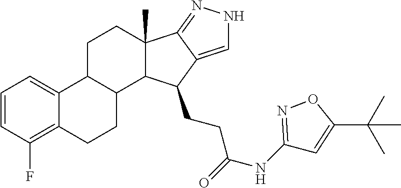 [SMILES: CC(C)(C)c1cc(NC(=O)CC[C@@H]2c3c[nH]nc3[C@@]3(C)CCC4c5cccc(F)c5CCC4C23)no1]

The compound 213 was prepared in 90% yield from the compound 212 by the Method C.

¹H NMR (400 MHz, DMSO-d₆): 1.10 (s, 3H), 1.29 (s, 9H), 1.40-2.44 (m, 13H), 2.67-2.93 (m, 3H), 6.61 (s, 1H), 6.98 (t, 1H), 7.16 (m, 2H), 7.39 (s, 1H), 10.97 (s, 1H), 12.13 (br s, 1H). MS m/z (TOF ES+): 491 (M+1)

### Compound 214

### N,N-diethyl-3-((13S,15R)-4-fluoro-13-methyl-17-oxo-7,8,9,11,12,13,14,15,16,17-decahydro-6H-cyclopenta[a]phenanthren-15-yl)propanamide

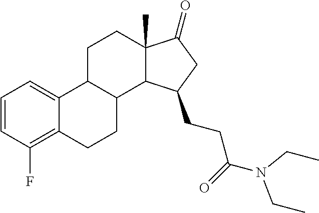 [SMILES: CCN(CC)C(=O)CC[C@@H]1CC(=O)[C@@]2(C)CCC3c4cccc(F)c4CCC3C12]

Compound 214 was synthesized in 97% yield by the Method A2 in THE using acid SM-IX and diethylamine as starting materials and 200 mol-% of EDCI and HOBT in 2 hours reaction time.

¹H-NMR (200 MHz, DMSO-d₆): 0.97 (s, 3H), 1.01 (t, 3H), 1.11 (t, 3H), 1.20-2.47 (m, 16H), 2.60-2.99 (m, 2H), 3.15-3.40 (m, 4H), 6.90-7.06 (m, 1H), 7.08-7.25 (m, 2H).

### Compound 215

### N,N-diethyl-3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)propanamide

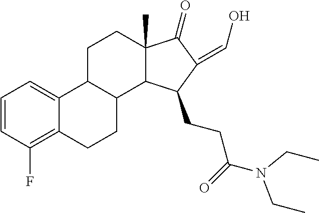 [SMILES: CCN(CC)C(=O)CC[C@@H]1/C(=C/O)C(=O)[C@@]2(C)CCC3c4cccc(F)c4CCC3C12]

Compound 215 was synthesized in quantitative yield from the compound 214 by the Method B in overnight reaction time.

¹H-NMR (400 MHz, DMSO-d₆): 0.97 (s, 3H), 1.03 (t, 3H), 1.12 (t, 3H), 1.30-2.45 (m, 13H), 2.55-2.99 (m, 3H), 3.15-4.00 (m, 4H), 6.90-7.02 (m, 1H), 7.08-7.25 (m, 2H), 7.49 (s, 1H), 11.46 (br s, 1H).

### Compound 216

### N,N-diethyl-3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamide

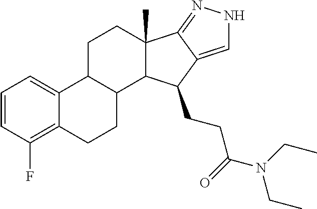 [SMILES: CCN(CC)C(=O)CC[C@@H]1c2c[nH]nc2[C@@]2(C)CCC3c4cccc(F)c4CCC3C12]

Compound 216 was synthesized in 86% yield from the compound 215 by the Method C in 0.5 hours reaction time.

¹H-NMR (400 MHz, DMSO-d₆): 1.00 (t, 3H), 1.10 (s, 3H), 1.12 (t, 3H), 1.30-2.45 (m, 13H), 2.69-2.95 (m, 3H), 3.15-3.40 (m, 4H), 6.90-7.02 (m, 1H), 7.08-7.25 (m, 2H), 7.39 (s, 1H), 12.18 (br s, 1H).

### Compound 217

### (13S,15R)-4-fluoro-13-methyl-15-(3-oxo-3-(8-oxa-2-azaspiro[4.5]decan-2-yl)propyl)-6,7,8,9,11,12,13,14,15,16-decahydro-17H-cyclopenta[a]phenanthren-17-one

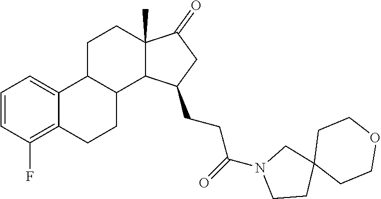 [SMILES: C[C@]12CCC3c4cccc(F)c4CCC3C1[C@H](CCC(=O)N1CCC3(CCOCC3)C1)CC2=O]

Compound 217 was synthesized in 93% yield by the Method A3 by using acid SM-IX and 8-oxa-2-aza-spiro(4,5)decane hydrochloride as starting materials in 4 hours reaction time.

¹H-NMR (400 MHz, DMSO-d₆): 0.97 (s, 3H), 1.30-2.45 (m, 22H), 2.65-2.76 (m, 1H), 2.84-2.91 (m, 1H), 3.19-3.22 (m, 1H), 3.29-3.42 (m, 2H), 3.46-3.65 (m, 5H), 6.94-7.00 (m, 1H), 7.10-7.22 (m, 2H).

### Compound 218

### (13S,15S,Z)-4-fluoro-16-(hydroxymethylene)-13-methyl-15-(3-oxo-3-(8-oxa-2-azaspiro[4.5]decan-2-yl)propyl)-6,7,8,9,11,12,13,14,15,16-decahydro-17H-cyclopenta[a]phenanthren-17-one

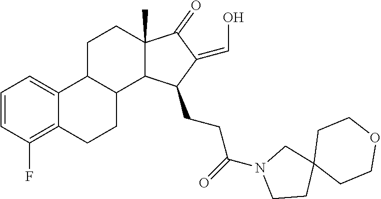 [SMILES: C[C@]12CCC3c4cccc(F)c4CCC3C1[C@H](CCC(=O)N1CCC3(CCOCC3)C1)/C(=C/O)C2=O]

Compound 218 was synthesized in 98% yield from the compound 217 by the Method B in overnight reaction time by using 1000 mol-% of ethyl formate and 600 mol-% of NaH.

¹H-NMR (400 MHz, DMSO-d₆): 0.97 (s, 3H), 1.30-2.46 (m, 19H), 2.65-2.99 (m, 3H), 3.18-3.66 (m, 8H), 6.94-7.00 (m, 1H), 7.10-7.22 (m, 2H), 7.52 (s, 1H).

### Compound 219

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-1-(8-oxa-2-azaspiro[4.5]decan-2-yl)propan-1-one

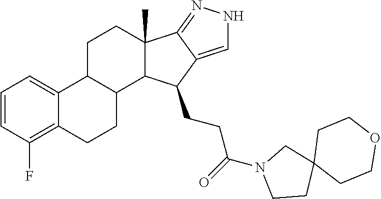 [SMILES: C[C@]12CCC3c4cccc(F)c4CCC3C1[C@H](CCC(=O)N1CCC3(CCOCC3)C1)c1c[nH]nc12]

Compound 219 was synthesized in 29% yield from the compound 218 by the Method C in one hour reaction time after chromatographic purification.

¹H-NMR (400 MHz, DMSO-d₆): 1.09 (s, 3H), 1.30-2.45 (m, 19H), 2.65-2.99 (m, 3H), 3.19-3.65 (m, 8H), 6.94-7.00 (m, 1H), 7.13-7.20 (m, 2H), 7.40-7.44 (m, 1H, isom.), 12.12 (br s, 1H).

### Compound 220

### 3-((13S,15R)-4-fluoro-13-methyl-17-oxo-7,8,9,11,12,13,14,15,16,17-decahydro-6H-cyclopenta[a]phenanthren-15-yl)-N,N-dimethylpropanamide

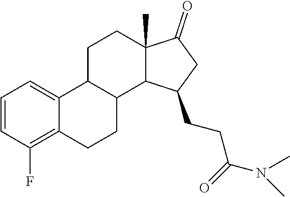 [SMILES: CN(C)C(=O)CC[C@@H]1CC(=O)[C@@]2(C)CCC3c4cccc(F)c4CCC3C12]

Compound 220 was synthesized in 85% yield by the Method A3 in DCM by using acid SM-IX and dimethylamine hydrochloride as starting materials in 2 hours reaction time.

¹H-NMR (200 MHz, DMSO-d₆): 0.97 (s, 3H), 1.28-2.40 (m, 16H), 2.62-2.94 (m, 2H), 2.82 (s, 3H), 2.97 (s, 3H), 6.90-7.03 (m, 1H), 7.10-7.25 (m, 2H).

### Compound 221

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N,N-dimethylpropanamide

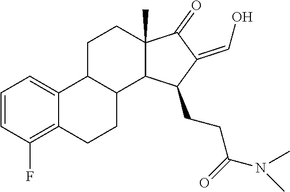 [SMILES: CN(C)C(=O)CC[C@@H]1/C(=C/O)C(=O)[C@@]2(C)CCC3c4cccc(F)c4CCC3C12]

Compound 221 was synthesized in quantitative yield from the compound 220 by the Method B in 4 hours reaction time by using 500 mol-% of ethyl formate and 200 mol-% of NaH.

¹H-NMR (200 MHz, DMSO-d₆): 0.97 (s, 3H), 1.28-2.48 (m, 13H), 2.62-2.94 (m, 3H), 2.83 (s, 3H), 2.97 (s, 3H), 6.90-7.03 (m, 1H), 7.10-7.25 (m, 2H), 7.57 (br s, 1H).

### Compound 222

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N,N-dimethylpropanamide

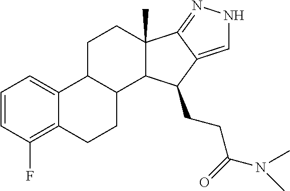 [SMILES: CN(C)C(=O)CC[C@@H]1c2c[nH]nc2[C@@]2(C)CCC3c4cccc(F)c4CCC3C12]

Compound 222 was synthesized in 30% yield after chromatographic purification from the compound 221 by the Method C by refluxing for one hour.

¹H-NMR (400 MHz, DMSO-d₆): 1.10 (s, 3H), 1.28-2.48 (m, 13H), 2.62-2.94 (m, 3H), 2.81 (s, 3H), 2.99 (s, 3H), 6.92-7.03 (m, 1H), 7.10-7.25 (m, 2H), 7.42 (s, 1H), 12.13 (br s, 1H).

### Compound 223

### 3-((13S,15R)-4-fluoro-13-methyl-17-oxo-7,8,9,11,12,13,14,15,16,17-decahydro-6H-cyclopenta[a]phenanthren-15-yl)-N-(tetrahydrofuran-3-yl)propanamide

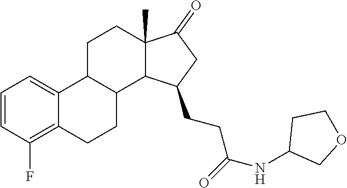 [SMILES: C[C@]12CCC3c4cccc(F)c4CCC3C1[C@H](CCC(=O)NC1CCOC1)CC2=O]

Compound 223 was synthesized in 73% yield by the Method A2 by using acid SM-IX and 3-aminotetrahydrofuran as starting materials in two hours reaction time.

¹H NMR (400 MHz, DMSO-d₆): 0.95 (s, 3H), 1.34-2.41 (m, 18H), 2.70-2.90 (m, 2H), 344 (m, 1H), 3.64-3.79 (m, 3H), 4.22 (m, 1H), 6.97 (m, 1H), 7.14 (m, 2H), 8.09 (d, 1H).

### Compound 224

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(tetrahydrofuran-3-yl)propanamide

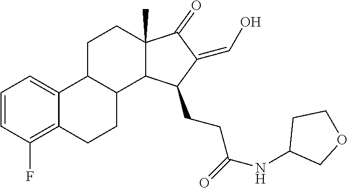 [SMILES: C[C@]12CCC3c4cccc(F)c4CCC3C1[C@H](CCC(=O)NC1CCOC1)/C(=C/O)C2=O]

The compound 224 was prepared in 98% yield from the compound 223 by Method B stirring 1.5 hours at room temperature.

¹H NMR (400 MHz, DMSO-d₆): 0.97 (s, 3H), 1.45-2.36 (m, 15H), 2.69-2.90 (m, 3H), 3.48 (m, 3H), 3.51-3.81 (m, 3H), 6.97 (t, 1H), 7.14 (m, 2H), 7.52 (s, 1H), 8.35 (br s, 1H).

### Compound 225

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(tetrahydrofuran-3-yl)propanamide

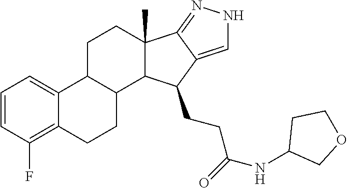 [SMILES: C[C@]12CCC3c4cccc(F)c4CCC3C1[C@H](CCC(=O)NC1CCOC1)c1c[nH]nc12]

The compound 225 was prepared in 96% yield from the compound 224 by the Method C and purified by chromatography.

¹H NMR (400 MHz, DMSO-d₆): 1.09 (s, 3H), 1.36-2.41 (m, 15H), 2.75-2.92 (m, 3H), 3.45 (m, 1H), 3.66-3.81 (m, 3H), 4.23 (m, 1H), 6.98 (t, 1H), 7.15 (m, 2H), 7.39 (s, 1H), 8.12 (d, 1H), 12.12 (br s, 1H).

### Compound 226

### 3-((13S,15R)-4-fluoro-13-methyl-17-oxo-7,8,9,11,12,13,14,15,16,17-decahydro-6H-cyclopenta[a]phenanthren-15-yl)-N-(6-methylpyridazin-3-yl)propanamide

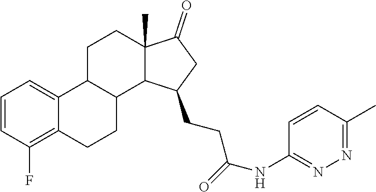 [SMILES: Cc1ccc(NC(=O)CC[C@@H]2CC(=O)[C@@]3(C)CCC4c5cccc(F)c5CCC4C23)nn1]

Compound 226 was synthesized in 83% yield by the Method A2 in THE by using 200 mol-% of EDCI and HOBT and acid SM-IX and 3-amino-6-methylpyridazine as starting materials in 4 hours reaction time.

¹H-NMR (200 MHz, DMSO-d₆): 0.98 (s, 3H), 1.20-2.47 (m, 16H), 2.55 (s, 3H), 2.70-2.95 (m, 2H), 6.89-7.06 (m, 1H), 7.08-7.25 (m, 2H), 7.54 (d, 1H), 8.23 (d, 1H), 11.05 (s, 1H).

### Compound 227

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(6-methylpyridazin-3-yl)propanamide

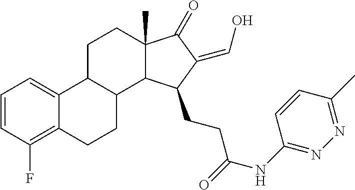 [SMILES: Cc1ccc(NC(=O)CC[C@@H]2/C(=C/O)C(=O)[C@@]3(C)CCC4c5cccc(F)c5CCC4C23)nn1]

Compound 227 was synthesized in quantitative yield from the compound 226 by the Method B in overnight reaction time.

¹H-NMR (400 MHz, DMSO-d₆): 1.00 (s, 3H), 1.30-2.47 (m, 13H), 2.55 (s, 3H), 2.60-3.05 (m, 3H), 6.90-7.05 (m, 1H), 7.08-7.25 (m, 2H), 7.53 (d, 1H), 7.57 (s, 1H), 8.22 (d, 1H), 11.00 (s, 1H), 11.01 (br s, 1H).

### Compound 228

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(6-methylpyridazin-3-yl)propanamide

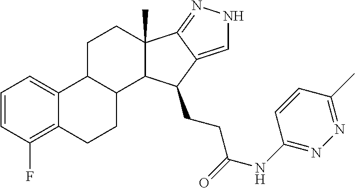 [SMILES: Cc1ccc(NC(=O)CC[C@@H]2c3c[nH]nc3[C@@]3(C)CCC4c5cccc(F)c5CCC4C23)nn1]

Compound 228 was synthesized in 47% yield from the compound 227 by the Method C in 0.5 hours reaction time.

¹H-NMR (400 MHz, DMSO-d₆): 1.11 (s, 3H), 1.30-2.47 (m, 13H), 2.55 (s, 3H), 2.69-2.95 (m, 3H), 6.90-7.05 (m, 1H), 7.08-7.25 (m, 2H), 7.41 (s, 1H), 7.54 (d, 1H), 8.23 (d, 1H), 11.07 (s, 1H), 12.16 (br s, 1H).

### Compound 229

### 3-((13S,15R)-4-fluoro-13-methyl-17-oxo-7,8,9,11,12,13,14,15,16,17-decahydro-6H-cyclopenta[a]phenanthren-15-yl)-N-(3-fluoropyridin-2-yl)propanamide

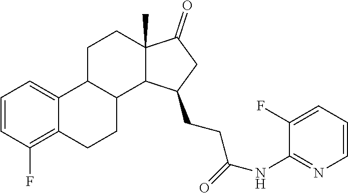 [SMILES: C[C@]12CCC3c4cccc(F)c4CCC3C1[C@H](CCC(=O)Nc1ncccc1F)CC2=O]

Compound 229 was synthesized in 96% yield by the Method A1 using acid SM-IX and 2-amino-3-fluoropyridine as starting materials in overnight reaction time.

¹H-NMR (400 MHz, DMSO-d₆): 0.98 (s, 3H), 1.34-1.98 (m, 8H), 2.18-2.47 (m, 8H), 2.68-2.77 (m, 1H), 2.84-2.90 (m, 1H), 6.97 (m, 1H), 7.10-7.20 (m, 2H), 7.34 (m, 1H), 7.77 (dd, 1H), 8.24 (d, 1H), 10.28 (s, 1H).

### Compound 230

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(3-fluoropyridin-2-yl)propanamide

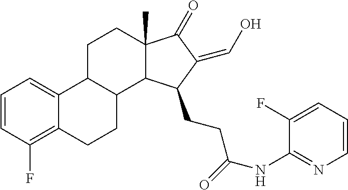 [SMILES: C[C@]12CCC3c4cccc(F)c4CCC3C1[C@H](CCC(=O)Nc1ncccc1F)/C(=C/O)C2=O]

The compound 230 was prepared in 88% yield from the compound 229 by Method B.

¹H NMR (400 MHz, DMSO-d₆): 0.99 (s, 3H), 1.36-1.83 (m, 7H), 1.94 (m, 1H), 2.20-2.45 (m, 3H), 2.55-2.97 (m, 5H), 6.97 (dd, 1H), 7.14 (m, 2H), 7.35 (m, 1H), 7.55 (s, 1H), 7.75 (m, 1H), 8.23 (d, 1H), 10.24 (s, 1H), 11.01 (br s, 1H).

### Compound 231

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(3-fluoropyridin-2-yl)propanamide

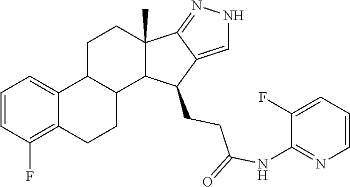 [SMILES: C[C@]12CCC3c4cccc(F)c4CCC3C1[C@H](CCC(=O)Nc1ncccc1F)c1c[nH]nc12]

The compound 231 was prepared in 43% yield from the compound 230 by the Method C.

¹H NMR (400 MHz, DMSO-d₆): 1.11 (s, 3H), 1.41-2.39 (m, 13H), 2.67-2.92 (m, 3H), 6.97 (dd, 1H), 7.16 (m, 2H), 7.34 (m, 1H), 7.44 (s, 1H), 7.76 (m, 1H), 8.25 (d, 1H), 10.32 (s, 1H), 12.15 (br s, 1H).

### Compound 232

### (13S,15R)-4-fluoro-13-methyl-15-(3-morpholino-3-oxopropyl)-6,7,8,9,11,12,13,14,15,16-decahydro-17H-cyclopenta[a]phenanthren-17-one

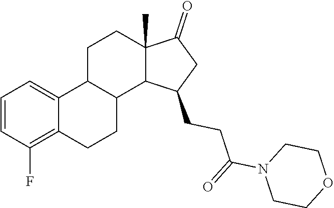 [SMILES: C[C@]12CCC3c4cccc(F)c4CCC3C1[C@H](CCC(=O)N1CCOCC1)CC2=O]

Compound 232 was synthesized in 83% yield by Method A2 in DMF using acid SM-IX and morpholine as starting materials in two hours reaction time.

¹H-NMR (200 MHz, DMSO-d₆): 0.97 (s, 3H), 1.35-2.37 (m, 15H), 2.76-2.92 (m, 3H), 3.45 (br s, 4H), 3.55 (br s 4H), 6.93-7.02 (m, 1H), 7.16-7.23 (m, 2H).

### Compound 233

### (13S,15S,Z)-4-fluoro-16-(hydroxymethylene)-13-methyl-15-(3-morpholino-3-oxopropyl)-6,7,8,9,11,12,13,14,15,16-decahydro-17H-cyclopenta[a]phenanthren-17-one

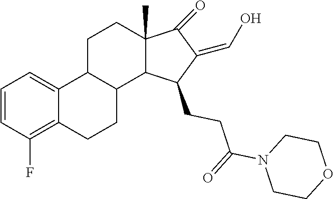 [SMILES: C[C@]12CCC3c4cccc(F)c4CCC3C1[C@H](CCC(=O)N1CCOCC1)/C(=C/O)C2=O]

The compound 233 was prepared in quantitative yield from the compound 232 by Method B stirring 2.5 hours at room temperature.

¹H NMR (400 MHz, DMSO-d₆): 0.97 (s, 3H), 1.38-2.40 (m, 14H), 2.73-3.00 (m, 5H), 3.55 (m, 6H), 6.97 (m, 1H), 7.14 (m, 2H), 7.55 (br s, 1H).

### Compound 234

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-1-morpholinopropan-1-one

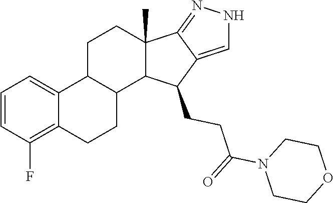 [SMILES: C[C@]12CCC3c4cccc(F)c4CCC3C1[C@H](CCC(=O)N1CCOCC1)c1c[nH]nc12]

The compound 234 was prepared in 62% yield from the compound 233 by the Method C.

¹H NMR (400 MHz, DMSO-d₆): 1.09 (s, 3H), 1.34-2.43 (m, 14H), 2.74-2.94 (m, 3H), 3.45-3.56 (m, 7H), 6.98 (m, 1H), 7.17 (m, 2H), 7.42 (s, 1H), 12.15 (br s, 1H). MS m/z (TOF ES+): 438 (M+1)

### Compound 235

### 3-((13S,15R)-4-fluoro-13-methyl-17-oxo-7,8,9,11,12,13,14,15,16,17-decahydro-6H-cyclopenta[a]phenanthren-15-yl)-N-(pyridazin-3-yl)propanamide

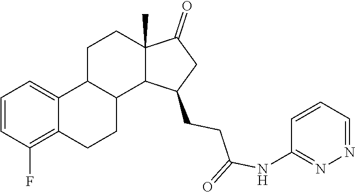 [SMILES: C[C@]12CCC3c4cccc(F)c4CCC3C1[C@H](CCC(=O)Nc1cccnn1)CC2=O]

Compound 235 was synthesized in 42% yield by the Method A2 in DMF by using acid SM-IX and 3-aminopyridazine as starting materials in 2 hours reaction time, crystallized from ethanol.

¹H-NMR (200 MHz, DMSO-d₆): 0.99 (s, 3H), 1.36-2.45 (m, 16H), 2.78-2.91 (m, 2H), 6.92-6.97 (m, 1H), 7.15-7.23 (m, 2H), 7.67 (dd, 1H), 8.33 (d, 1H), 8.95 (d, 1H), 11.14 (s, 1H).

### Compound 236

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(pyridazin-3-yl)propanamide

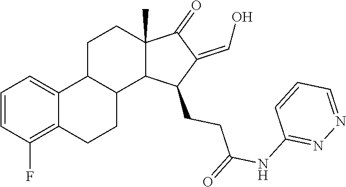 [SMILES: C[C@]12CCC3c4cccc(F)c4CCC3C1[C@H](CCC(=O)Nc1cccnn1)/C(=C/O)C2=O]

Compound 236 was synthesized in quantitative yield from the compound 235 by the Method B in overnight reaction time.

¹H-NMR (400 MHz, DMSO-d₆): 0.99 (s, 3H), 1.30-2.47 (m, 13H), 2.57-2.95 (m, 3H), 6.90-7.00 (m, 1H), 7.10-7.20 (m, 2H), 7.64 (dd, 1H), 7.73 (br s, 1H), 8.32 (d, 1H), 8.92 (d, 1H), 11.04 (br s, 1H).

### Compound 237

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(pyridazin-3-yl)propanamide

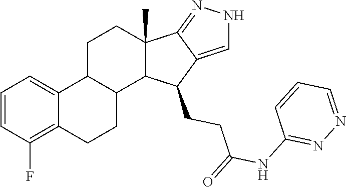 [SMILES: C[C@]12CCC3c4cccc(F)c4CCC3C1[C@H](CCC(=O)Nc1cccnn1)c1c[nH]nc12]

Compound 237 was synthesized in quantitative yield from the compound 236 by the Method C in 0.5 hours reaction time.

¹H-NMR (400 MHz, DMSO-d₆): 1.11 (s, 3H), 1.30-2.47 (m, 13H), 2.60-2.95 (m, 3H), 6.90-7.05 (m, 1H), 7.10-7.20 (m, 2H), 7.42 (s, 1H), 7.66 (dd, 1H), 8.33 (d, 1H), 8.95 (d, 1H), 11.17 (s, 1H), 12.15 (br s, 1H). MS m/z (TOF ES+): 446 (M+1).

### Compound 238

### 3-((13S,15R)-4-fluoro-13-methyl-17-oxo-7,8,9,11,12,13,14,15,16,17-decahydro-6H-cyclopenta[a]phenanthren-15-yl)-N-(4-morpholinopyridin-2-yl)propanamide

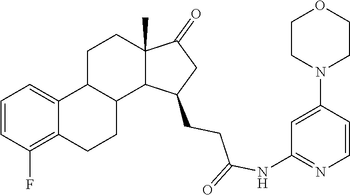 [SMILES: C[C@]12CCC3c4cccc(F)c4CCC3C1[C@H](CCC(=O)Nc1cc(N3CCOCC3)ccn1)CC2=O]

Compound 238 was synthesized in 40% yield by the Method A3 in DMF using acid SM-IX and 4-morpholinopyridin-2-amine as starting materials in overnight reaction time. The crude product was purified by chromatography.

¹H-NMR (400 MHz, DMSO-d₆): 0.97 (s, 3H), 1.34-2.48 (m, 16H), 2.68-2.90 (m, 2H), 3.22 (m, 4H), 3.71 (m, 4H), 6.60 (dd, 1H), 6.97 (m, 1H), 7.14 (m, 2H), 7.67 (s, 1H), 7.94 (d, 1H), 10.22 (s, 1H).

### Compound 239

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(4-morpholinopyridin-2-yl)propanamide

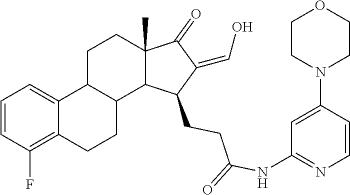 [SMILES: C[C@]12CCC3c4cccc(F)c4CCC3C1[C@H](CCC(=O)Nc1cc(N3CCOCC3)ccn1)/C(=C/O)C2=O]

The compound 239 was prepared in 68% yield from the compound 238 by Method B stirring three hours at room temperature. The product was obtained by neutralizing the acidic phase during work-up process.

¹H NMR (400 MHz, DMSO-d₆): 0.99 (s, 3H), 1.18-2.37 (m, 13H), 2.68-2.94 (m, 4H), 3.23 (m, 4H), 3.71 (m, 4H), 6.60 (d, 1H), 6.96 (m, 1H), 7.14 (m, 2H), 7.55 (s, 1H), 7.63 (s, 1H), 7.93 (d, 1H), 10.18 (br s, 1H).

### Compound 240

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(4-morpholinopyridin-2-yl)propanamide

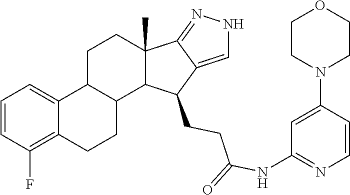 [SMILES: C[C@]12CCC3c4cccc(F)c4CCC3C1[C@H](CCC(=O)Nc1cc(N3CCOCC3)ccn1)c1c[nH]nc12]

The compound 240 was prepared in 27% yield from the compound 239 by the Method C and purified by heptane trituration.

¹H NMR (400 MHz, DMSO-d₆): 1.10 (s, 3H), 1.42-2.42 (m, 12H), 2.67 (m, 1H), 2.76-2.92 (m, 3H), 3.22 (m, 4H), 3.71 (m, 4H), 6.60 (dd, 1H), 6.98 (m, 1H), 7.16 (m, 2H), 7.39 (s, 1H), 7.67 (s, 1H), 7.95 (d, 1H), 10.25 (s, 1H), 12.12 (br s, 1H).

### Compound 241

### 3-((13S,15R)-4-fluoro-13-methyl-17-oxo-7,8,9,11,12,13,14,15,16,17-decahydro-6H-cyclopenta[a]phenanthren-15-yl)-N-(2-hydroxy-2-methylpropyl)-N-methylpropanamide

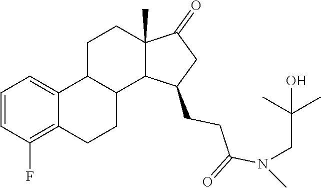 [SMILES: CN(CC(C)(C)O)C(=O)CC[C@@H]1CC(=O)[C@@]2(C)CCC3c4cccc(F)c4CCC3C12]

Compound 241 was synthesized in 83% yield as an isomeric mixture 60:40 by the Method A2 in DMF by using acid SM-IX and 2-methyl-1-(methylamino)-2-propanol as starting materials in 2 hours reaction time.

¹H-NMR (400 MHz, DMSO-d₆): 0.97 (s, 3H), 1.04 (s, 3H), 1.12 (s, 3H), 1.35-1.90 (m, 8H), 2.26-2.41 (m, 8H), 2.68-2.87 (m, 3H), 3.98 (s, 2H), 3.28 (m, 2H), 4.50 (s, 0.6H, isomer), 4.57 (s, 0.4, isomer), 6.97 (m, 1H), 7.15 (m, 2H).

### Compound 242

### 3-((13S,15S,Z)-4-fluoro-16-(hydroxymethylene)-13-methyl-17-oxo-7,8,9,11,12,13,14,15,16,17-decahydro-6H-cyclopenta[a]phenanthren-15-yl)-N-(2-hydroxy-2-methylpropyl)-N-methylpropanamide

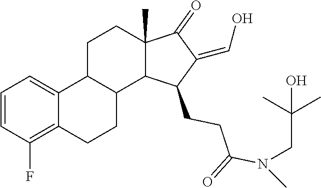 [SMILES: CN(CC(C)(C)O)C(=O)CC[C@@H]1/C(=C/O)C(=O)[C@@]2(C)CCC3c4cccc(F)c4CCC3C12]

The compound 242 was prepared in 88% yield from the compound 241 by Method B stirring two hours at room temperature.

¹H NMR (400 MHz, DMSO-d₆): 0.9 (s, 3H), 1.05 (s, 3H), 1.11 (s, 3H), 1.23-2.34 (m, 15H), 2.56-2.91 (m, 5H), 3.08 (s, 2H), 4.51 (br s, 1H), 6.97 (m, 1H), 7.14 (m, 2H), 7.55 (s, 1H).

### Compound 243

### 3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(2-hydroxy-2-methylpropyl)-N-methylpropanamide

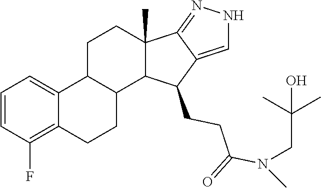 [SMILES: CN(CC(C)(C)O)C(=O)CC[C@@H]1c2c[nH]nc2[C@@]2(C)CCC3c4cccc(F)c4CCC3C12]

The compound 243 was prepared in 89% yield from the compound 242 by the Method C as an isomeric mixture 60:40.

¹H NMR (400 MHz, DMSO-d₆): 1.03 (d, 4H), 1.10 (s, 4H), 1.13 (s, 1H), 1.38-2.38 (m, 13H), 2.70-2.99 (m, 4H), 3.10 (s, 2H), 3.28 (m, 2H), 4.50 (s, 0.6H, isomer), 4.59 (s, 0.4H, isomer), 6.98 (dd, 1H), 7.16 (m, 2H), 7.40 (m, 1H), 12.12 (br s, 1H).

### Compound 244

### 3-((13S,15R,E)-13-methyl-17-oxo-7,8,9,11,12,13,14,15,16,17-decahydro-6H-cyclopenta[a]phenanthren-15-yl)-N-(5-methylisoxazol-3-yl)propanamide

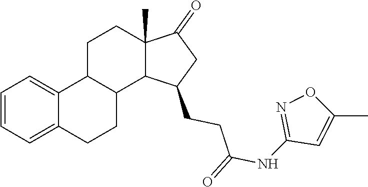 [SMILES: Cc1cc(NC(=O)CC[C@@H]2CC(=O)[C@@]3(C)CCC4c5ccccc5CCC4C23)no1]

Compound 244 was synthesized in 90% yield by the Method A1 in THE by using acid SM-XXVI and 3-amino-5-methylisoxazole as starting materials in 5 hours reaction time.

¹H-NMR (400 MHz, DMSO-d₆): 0.97 (s, 3H), 1.29-2.49 (m, 16H), 2.36 (s, 3H), 2.80-2.95 (m, 2H), 6.64 (s, 1H), 7.05-7.15 (m, 3H), 7.24-7.28 (m, 1H), 10.88 (s, 1H). MS m/z (TOF ES+): 407 (M+1), 429 (M+Na).

### Compound 245

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(5-methylisoxazol-3-yl)propanamide

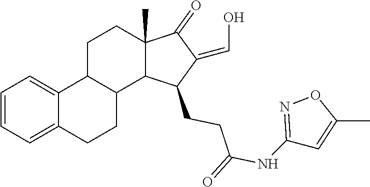 [SMILES: Cc1cc(NC(=O)CC[C@@H]2/C(=C/O)C(=O)[C@@]3(C)CCC4c5ccccc5CCC4C23)no1]

Compound 245 was synthesized in 59% yield from the compound 244 by the Method B in overnight reaction time.

¹H-NMR (400 MHz, DMSO-d₆): 0.99 (s, 3H), 1.34-2.60 (m, 13H), 2.36 (s, 3H), 2.80-2.99 (m, 3H), 6.62 (s, 1H), 7.03-7.15 (m, 3H), 7.23-7.30 (m, 1H), 7.56 (s, 1H), 10.88 (s, 1H), 10.95 (br s, 1H).

### Compound 246

### 3-((8aS,12S)-8a-methyl-1,2,6b,7,8,8a,10,12,12a,12b-decahydronaphtho[2′,1′:4,5]indeno[1,2-c]pyrazol-12-yl)-N-(5-methylisoxazol-3-yl)propanamide

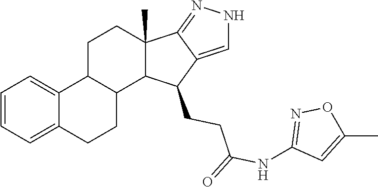 [SMILES: Cc1cc(NC(=O)CC[C@@H]2c3c[nH]nc3[C@@]3(C)CCC4c5ccccc5CCC4C23)no1]

Compound 246 was synthesized in 42% yield after chromatographic purification from the compound 245 by the Method C in 1.5 hours reaction time.

¹H-NMR (400 MHz, DMSO-d₆): 1.10 (s, 3H), 1.34-2.50 (m, 13H), 2.36 (s, 3H), 2.80-2.99 (m, 3H), 6.64 (s, 1H), 7.05-7.15 (m, 3H), 7.25-7.31 (m, 1H), 7.37 (s, 1H), 10.90 (s, 1H), 12.11 (br s, 1H).

### Compound 247

### (13S,15R)-13-methyl-15-(3-morpholino-3-oxopropyl)-6,7,8,9,11,12,13,14,15,16-decahydro-17H-cyclopenta[a]phenanthren-17-one

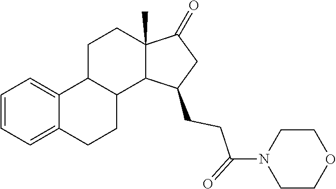 [SMILES: C[C@]12CCC3c4ccccc4CCC3C1[C@H](CCC(=O)N1CCOCC1)CC2=O]

Compound 247 was synthesized in 89% yield by the Method A1 in THE by using acid SM-XXVI and pre-dried morpholino as starting materials in 1.5 hours reaction time.

¹H-NMR (400 MHz, DMSO-d₆): 0.97 (s, 3H), 1.33-2.44 (m, 16H), 2.87 (m, 2H), 3.44 (m, 4H), 3.56 (m, 4H), 7.06-7.14 (m, 3H), 7.27 (m, 1H).

### Compound 248

### (13S,15S,Z)-16-(hydroxymethylene)-13-methyl-15-(3-morpholino-3-oxopropyl)-6,7,8,9,11,12,13,14,15,16-decahydro-17H-cyclopenta[a]phenanthren-17-one

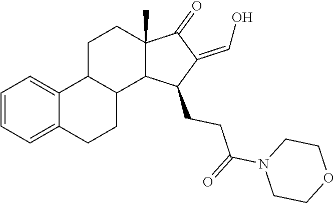 [SMILES: C[C@]12CCC3c4ccccc4CCC3C1[C@H](CCC(=O)N1CCOCC1)/C(=C/O)C2=O]

Compound 248 was synthesized in quantitative yield from the compound 247 by the Method B in overnight reaction time.

¹H-NMR (400 MHz, DMSO-d₆): 0.97 (s, 3H), 1.35-2.47 (m, 13H), 2.91 (m, 3H), 3.44 (m, 4H), 3.54 (m, 4H), 7.10 (m, 3H), 7.26 (d, 1H), 7.50 (s, 1H), 11.18 (br s, 1H).

### Compound 249

### 3-((8aS,12S)-8a-methyl-1,2,6b,7,8,8a,10,12,12a,12b-decahydronaphtho[2′,1′:4,5]indeno[1,2-c]pyrazol-12-yl)-1-morpholinopropan-1-one

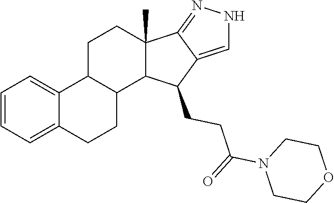 [SMILES: C[C@]12CCC3c4ccccc4CCC3C1[C@H](CCC(=O)N1CCOCC1)c1c[nH]nc12]

Compound 249 was synthesized in 66% yield from the compound 248 by the Method C in 30 minutes reaction time.

¹H-NMR (400 MHz, DMSO-d₆): 1.10 (s, 3H), 1.41-2.43 (m, 13H), 2.89 (s, 3H), 3.43-3.58 (m, 8H), 7.10 (m, 3H), 7.29 (m, 1H), 7.41 (s, 1H), 12.11 (br s, 1H).

### Compound 250

### 3-((13S,15R)-13-methyl-17-oxo-7,8,9,11,12,13,14,15,16,17-decahydro-6H-cyclopenta[a]phenanthren-15-yl)-N-(5-morpholinopyridin-2-yl)propanamide

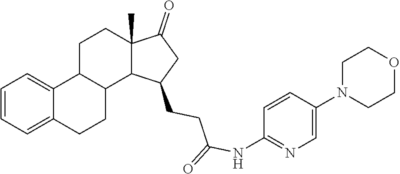 [SMILES: C[C@]12CCC3c4ccccc4CCC3C1[C@H](CCC(=O)Nc1ccc(N3CCOCC3)cn1)CC2=O]

Compound 250 was synthesized in 89% yield by the Method A1 in THE by using acid SM-XXVI and 5-morpholinopyridin-2-amine as starting materials in overnight reaction time.

¹H-NMR (400 MHz, DMSO-d₆): 0.98 (s, 3H), 1.30-2.48 (m, 16H), 2.80-2.95 (m, 2H), 3.06-3.11 (m, 4H), 3.70-3.78 (m, 4H), 7.03-7.15 (m, 3H), 7.25-7.29 (m, 1H), 7.40 (dd, 1H), 7.96 (d, 1H), 8.00 (d, 1H), 10.28 (s, 1H).

### Compound 251

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(5-morpholinopyridin-2-yl)propanamide

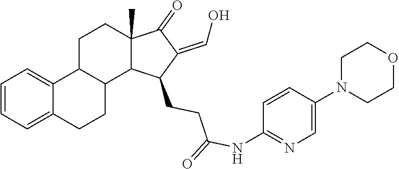 [SMILES: C[C@]12CCC3c4ccccc4CCC3C1[C@H](CCC(=O)Nc1ccc(N3CCOCC3)cn1)/C(=C/O)C2=O]

Compound 251 was synthesized in 83% yield from the compound 250 by the Method B in overnight reaction time.

¹H-NMR (400 MHz, DMSO-d₆): 0.99 (s, 3H), 1.34-2.60 (m, 13H), 2.80-2.95 (m, 3H), 3.06-3.11 (m, 4H), 3.70-3.78 (m, 4H), 7.03-7.15 (m, 3H), 7.25-7.29 (m, 1H), 7.40 (dd, 1H), 7.54 (s, 1H), 7.95 (d, 1H), 8.00 (d, 1H), 10.24 (s, 1H), 11.02 (br s, 1H).

### Compound 252

### 3-((8aS,12S)-8a-methyl-1,2,6b,7,8,8a,10,12,12a,12b-decahydronaphtho[2′,1′:4,5]indeno[1,2-c]pyrazol-12-yl)-N-(5-morpholinopyridin-2-yl)propanamide

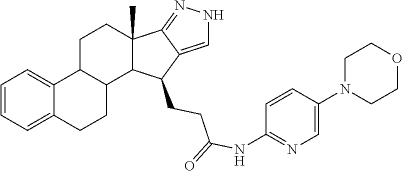 [SMILES: C[C@]12CCC3c4ccccc4CCC3C1[C@H](CCC(=O)Nc1ccc(N3CCOCC3)cn1)c1c[nH]nc12]

Compound 252 was synthesized in 73% yield from the compound 251 by the Method C in one hour reaction time.

¹H-NMR (400 MHz, DMSO-d₆): 1.11 (s, 3H), 1.34-2.50 (m, 13H), 2.78-2.95 (m, 3H), 3.06-3.11 (m, 4H), 3.70-3.78 (m, 4H), 7.03-7.15 (m, 3H), 7.25-7.29 (m, 1H), 7.39 (dd, 1H), 7.40 (s, 1H), 7.95 (d, 1H), 8.00 (d, 1H), 10.30 (s, 1H), 12.09 (br s, 1H).

### Compound 253

### 3-((13S,15R)-13-methyl-17-oxo-7,8,9,11,12,13,14,15,16,17-decahydro-6H-cyclopenta[a]phenanthren-15-yl)-N-(pyridazin-3-yl)propanamide

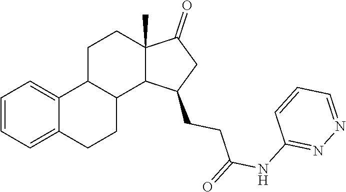 [SMILES: C[C@]12CCC3c4ccccc4CCC3C1[C@H](CCC(=O)Nc1cccnn1)CC2=O]

Compound 253 was synthesized in 43% yield by the Method A1 in THE by using acid SM-XXVI and 3-aminopyridazine as starting materials in overnight reaction time.

¹H-NMR (400 MHz, DMSO-d₆): 0.99 (s, 3H), 1.30-2.62 (m, 16H), 2.80-2.96 (m, 2H), 7.05-7.15 (m, 3H), 7.25-7.29 (m, 1H), 7.66 (dd, 1H), 8.32 (d, 1H), 8.95 (d, 1H), 11.13 (s, 1H).

### Compound 254

### 3-((13S,15S,Z)-16-(hydroxymethylene)-13-methyl-17-oxo-7,8,9,11,12,13,14,15,16,17-decahydro-6H-cyclopenta[a]phenanthren-15-yl)-N-(pyridazin-3-yl)propanamide

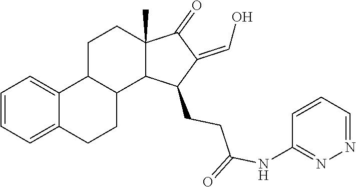 [SMILES: C[C@]12CCC3c4ccccc4CCC3C1[C@H](CCC(=O)Nc1cccnn1)/C(=C/O)C2=O]

Compound 254 was synthesized in quantitative yield from the compound 253 by the Method B in 6 hours reaction time.

¹H-NMR (400 MHz, DMSO-d₆): 1.00 (s, 3H), 1.34-2.70 (m, 13H), 2.80-2.99 (m, 3H), 7.03-7.15 (m, 3H), 7.23-7.30 (m, 1H), 7.56 (s, 1H), 7.65 (dd, 1H), 8.31 (d, 1H), 8.93 (d, 1H), 10.95 (br s, 1H), 11.07 (s, 1H).

### Compound 255

### 3-((8aS,12S)-8a-methyl-1,2,6b,7,8,8a,10,12,12a,12b-decahydronaphtho[2′,1′:4,5]indeno[1,2-c]pyrazol-12-yl)-N-(pyridazin-3-yl)propanamide

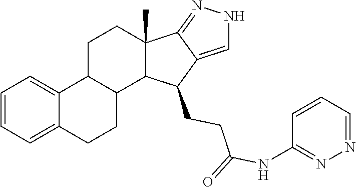 [SMILES: C[C@]12CCC3c4ccccc4CCC3C1[C@H](CCC(=O)Nc1cccnn1)c1c[nH]nc12]

Compound 255 was synthesized in 57% yield from the compound 254 by the Method C in one hour reaction time.

¹H-NMR (400 MHz, DMSO-d₆): 1.12 (s, 3H), 1.38-2.63 (m, 13H), 2.80-2.99 (m, 3H), 7.03-7.15 (m, 3H), 7.23-7.30 (m, 1H), 7.41 (s, 1H), 7.66 (dd, 1H), 8.33 (d, 1H), 8.95 (d, 1H), 11.16 (s, 1H), 12.13 (br s, 1H).

### Compound 256

### N-(6-fluoropyridin-2-yl)-3-((13S,15R)-13-methyl-17-oxo-7,8,9,11,12,13,14,15,16,17-decahydro-6H-cyclopenta[a]phenanthren-15-yl)propanamide

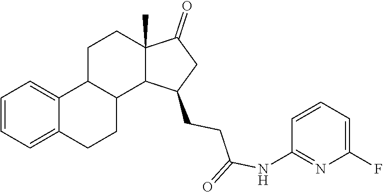 [SMILES: C[C@]12CCC3c4ccccc4CCC3C1[C@H](CCC(=O)Nc1cccc(F)n1)CC2=O]

Compound 256 was synthesized in 92% yield by the Method A1 in THE by using acid SM-XXVI and 2-amino-6-fluoropyridine as starting materials in 4 hours reaction time.

¹H-NMR (400 MHz, DMSO-d₆): 0.98 (s, 3H), 1.30-2.47 (m, 16H), 2.80-2.95 (m, 2H), 6.83 (dd, 1H), 7.05-7.15 (m, 3H), 7.26-7.28 (m, 1H), 7.91-7.97 (m, 1H), 8.00-8.03 (m, 1H), 10.69 (s, 1H).

### Compound 257

### N-(6-fluoropyridin-2-yl)-3-((13S,15S,Z)-16-(hydroxymethylene)-13-methyl-17-oxo-7,8,9,11,12,13,14,15,16,17-decahydro-6H-cyclopenta[a]phenanthren-15-yl)propanamide

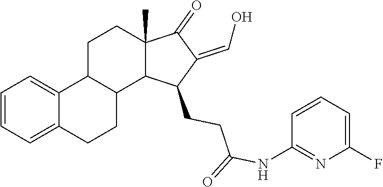 [SMILES: C[C@]12CCC3c4ccccc4CCC3C1[C@H](CCC(=O)Nc1cccc(F)n1)/C(=C/O)C2=O]

Compound 257 was synthesized in 46% yield after chromatographic purification from the compound 256 by the Method B in 2 hours reaction time.

¹H-NMR (400 MHz, DMSO-d₆): 1.00 (s, 3H), 1.34-2.60 (m, 13H), 2.80-2.99 (m, 3H), 6.81 (dd, 1H), 7.03-7.15 (m, 3H), 7.23-7.30 (m, 1H), 7.58 (s, 1H), 7.88-7.97 (m, 1H), 7.98-8.03 (m, 1H), 10.69 (s, 1H), 10.96 (br s, 1H).

### Compound 258

### N-(6-fluoropyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide

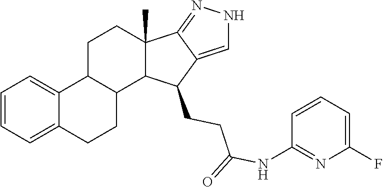 [SMILES: C[C@]12CCC3c4ccccc4CCC3C1[C@H](CCC(=O)Nc1cccc(F)n1)c1c[nH]nc12]

Compound 258 was synthesized in 29% yield after chromatographic purification from the compound 257 by the Method C in 1.5 hours reaction time.

¹H-NMR (400 MHz, DMSO-d₆): 1.11 (s, 3H), 1.36-2.49 (m, 13H), 2.75-2.99 (m, 3H), 6.83 (dd, 1H), 7.03-7.19 (m, 3H), 7.25-7.33 (m, 1H), 7.39 (s, 1H), 7.90-7.99 (m, 1H), 8.00-8.04 (m, 1H), 10.72 (s, 1H), 12.12 (br s, 1H).

### Compound 259

### N-(3,5-difluoropyridin-2-yl)-3-((13S,15R)-13-methyl-17-oxo-7,8,9,11,12,13,14,15,16,17-decahydro-6H-cyclopenta[a]phenanthren-15-yl)propanamide

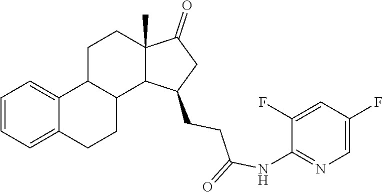 [SMILES: C[C@]12CCC3c4ccccc4CCC3C1[C@H](CCC(=O)Nc1ncc(F)cc1F)CC2=O]

Compound 259 was synthesized in 90% yield by the Method A1 in THE by using acid SM-XXVI and 2-amino-3,5-difluoropyridine as starting materials in overnight reaction time.

¹H-NMR (400 MHz, DMSO-d₆): 0.98 (s, 3H), 1.30-2.47 (m, 16H), 2.80-2.94 (m, 2H), 7.05-7.15 (m, 3H), 7.26-7.28 (m, 1H), 7.98-8.03 (m, 1H), 8.34-8.35 (m, 1H), 10.31 (s, 1H).

### Compound 260

### N-(3,5-difluoropyridin-2-yl)-3-((13S,15S,Z)-16-(hydroxymethylene)-13-methyl-17-oxo-7,8,9,11,12,13,14,15,16,17-decahydro-6H-cyclopenta[a]phenanthren-15-yl)propanamide

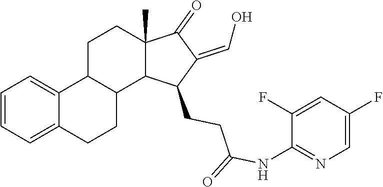 [SMILES: C[C@]12CCC3c4ccccc4CCC3C1[C@H](CCC(=O)Nc1ncc(F)cc1F)/C(=C/O)C2=O]

Compound 260 was synthesized in quantitative yield from the compound 259 by the Method B in 4 hours reaction time.

¹H-NMR (400 MHz, DMSO-d₆): 0.99 (s, 3H), 1.30-2.62 (m, 13H), 2.80-2.99 (m, 3H), 7.03-7.15 (m, 3H), 7.23-7.30 (m, 1H), 7.55 (s, 1H), 7.95-8.04 (m, 1H), 8.32 (d, 1H), 10.26 (s, 1H), 10.98 (br s, 1H).

### Compound 261

### N-(3,5-difluoropyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide

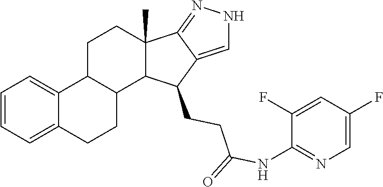 [SMILES: C[C@]12CCC3c4ccccc4CCC3C1[C@H](CCC(=O)Nc1ncc(F)cc1F)c1c[nH]nc12]

Compound 261 was synthesized in 66% yield from the compound 260 by the Method C in one hour reaction time.

¹H-NMR (400 MHz, DMSO-d₆): 1.11 (s, 3H), 1.36-2.50 (m, 13H), 2.80-2.99 (m, 3H), 7.03-7.15 (m, 3H), 7.25-7.30 (m, 1H), 7.44 (s, 1H), 7.95-8.05 (m, 1H), 8.34 (d, 1H), 10.35 (s, 1H), 12.14 (br s, 1H).

### Compound 262

### N-(3-fluoropyridin-2-yl)-3-((13S,15R)-13-methyl-17-oxo-7,8,9,11,12,13,14,15,16,17-decahydro-6H-cyclopenta[a]phenanthren-15-yl)propanamide

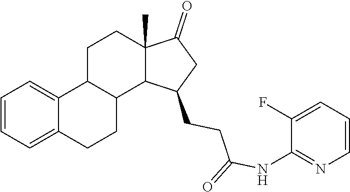 [SMILES: C[C@]12CCC3c4ccccc4CCC3C1[C@H](CCC(=O)Nc1ncccc1F)CC2=O]

Compound 262 was synthesized in 88% yield by the Method A1 in THE by using acid SM-XXVI and 2-amino-3-fluoropyridine as starting materials in overnight reaction time.

¹H-NMR (400 MHz, DMSO-d₆): 0.99 (s, 3H), 1.30-2.47 (m, 16H), 2.81-2.96 (m, 2H), 7.05-7.16 (m, 3H), 7.26-7.28 (m, 1H), 7.30-7.37 (m, 1H), 7.73-7.79 (m, 1H), 8.23-8.25 (m, 1H), 10.27 (s, 1H).

### Compound 263

### N-(3-fluoropyridin-2-yl)-3-((13S,15S,Z)-16-(hydroxymethylene)-13-methyl-17-oxo-7,8,9,11,12,13,14,15,16,17-decahydro-6H-cyclopenta[a]phenanthren-15-yl)propanamide

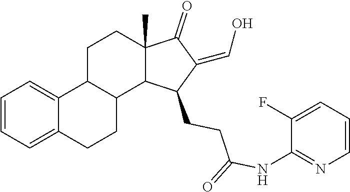 [SMILES: C[C@]12CCC3c4ccccc4CCC3C1[C@H](CCC(=O)Nc1ncccc1F)/C(=C/O)C2=O]

Compound 263 was synthesized in quantitative yield from the compound 262 by the Method B in 5 hours reaction time.

¹H-NMR (400 MHz, DMSO-d₆): 1.00 (s, 3H), 1.34-2.65 (m, 13H), 2.80-2.99 (m, 3H), 7.03-7.15 (m, 3H), 7.20-7.30 (m, 1H), 7.31-7.36 (m, 1H), 7.57 (s, 1H), 7.70-7.78 (m, 1H), 8.20-8.24 (m, 1H), 10.30 (s, 1H), 11.01 (br s, 1H).

### Compound 264

### N-(3-fluoropyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide

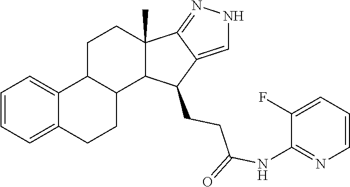 [SMILES: C[C@]12CCC3c4ccccc4CCC3C1[C@H](CCC(=O)Nc1ncccc1F)c1c[nH]nc12]

Compound 264 was synthesized in 40% yield from the compound 263 by the Method C in one hour reaction time.

¹H-NMR (400 MHz, DMSO-d₆): 1.11 (s, 3H), 1.39-2.50 (m, 13H), 2.80-2.99 (m, 3H), 7.03-7.15 (m, 3H), 7.25-7.30 (m, 1H), 7.31-7.36 (m, 1H), 7.45 (s, 1H), 7.73-7.79 (m, 1H), 8.22-8.26 (m, 1H), 10.32 (s, 1H), 12.13 (br s, 1H).

### Compound 265

### 3-((13S,15R)-3-chloro-13-methyl-17-oxo-7,8,9,11,12,13,14,15,16,17-decahydro-6H-cyclopenta[a]phenanthren-15-yl)-N-(3-fluoropyridin-2-yl)propanamide

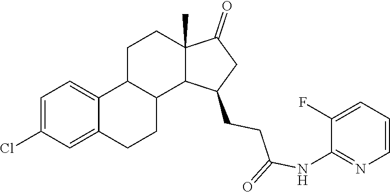 [SMILES: C[C@]12CCC3c4ccc(Cl)cc4CCC3C1[C@H](CCC(=O)Nc1ncccc1F)CC2=O]

Compound 265 was synthesized in 81% yield by the Method A1 in THE by using acid SM-XVII and 2-amino-3-fluoropyridine as starting materials in overnight reaction time.

¹H-NMR (400 MHz, DMSO-d₆): 0.98 (s, 3H), 1.30-2.47 (m, 16H), 2.81-2.94 (m, 2H), 7.15-7.16 (m, 2H), 7.28-7.30 (m, 1H), 7.32-7.36 (m, 1H), 7.73-7.79 (m, 1H), 8.23-8.25 (d, 1H), 10.27 (s, 1H).

### Compound 266

### 3-((13S,15S,Z)-3-chloro-16-(hydroxymethylene)-13-methyl-17-oxo-7,8,9,11,12,13,14,15,16,17-decahydro-6H-cyclopenta[a]phenanthren-15-yl)-N-(3-fluoropyridin-2-yl)propanamide

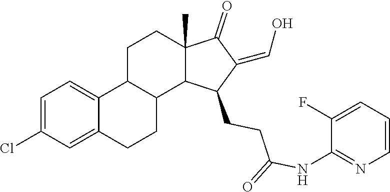 [SMILES: C[C@]12CCC3c4ccc(Cl)cc4CCC3C1[C@H](CCC(=O)Nc1ncccc1F)/C(=C/O)C2=O]

Compound 266 was synthesized in quantitative yield from the compound 265 by the Method B in 4 hours reaction time.

¹H-NMR (400 MHz, DMSO-d₆): 0.99 (s, 3H), 1.34-2.60 (m, 13H), 2.80-2.99 (m, 3H), 7.10-7.23 (m, 2H), 7.25-7.35 (m, 2H), 7.59 (s, 1H), 7.70-7.79 (m, 1H), 8.20-8.25 (m, 1H), 10.34 (br s, 1H), 11.01 (br s, 1H).

### Compound 267

### 3-((8aS,12S)-4-chloro-8a-methyl-1,2,6b,7,8,8a,10,12,12a,12b-decahydronaphtho[2′,1′:4,5]indeno[1,2-c]pyrazol-12-yl)-N-(3-fluoropyridin-2-yl)propanamide

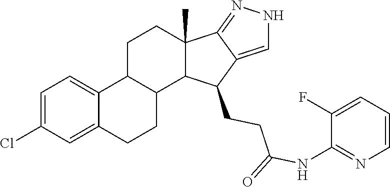 [SMILES: C[C@]12CCC3c4ccc(Cl)cc4CCC3C1[C@H](CCC(=O)Nc1ncccc1F)c1c[nH]nc12]

Compound 267 was synthesized in 58% yield from the compound 266 by the Method C in one hour reaction time.

¹H-NMR (400 MHz, DMSO-d₆): 1.11 (s, 3H), 1.34-2.50 (m, 13H), 2.80-2.99 (m, 3H), 7.11-7.22 (m, 2H), 7.25-7.37 (m, 2H), 7.44 (s, 1H), 7.70-7.79 (m, 1H), 8.23-8.26 (m, 1H), 10.32 (s, 1H), 12.13 (br s, 1H).

### Pharmacological Tests

The following tests are provided to demonstrate the present invention in illustrative way and should not be considered as limiting in the scope of invention. Further, the concentrations of the compound in the assays are exemplary and should not be taken as limiting. A person skilled in the art may define pharmaceutically relevant concentrations with method known in the art.

### Inhibition of 17β-Hydroxysteroid Dehydrogenase Type 1 Enzyme

17β-HSD1 production and isolation: Recombinant baculovirus was generated by the “Bac to Bac Expression System” (Invitrogen). Recombinant bacmid was transfected to Sd9 insect cells using “Cellfectin Reagent” (Invitrogen). 60 h later cells were harvested; the microsomal fraction was isolated as described by Puranen, T. J., Poutanen, M. H., Peltoketo, H. E., Vihko, P. T. and Vihko, R. K. (1994) Site-directed mutagenesis of the putative active site of human 17 β-hydroxysteroid dehydrogenase type 1. Biochem. J. 304: 289-293. Aliquots were stored frozen until determination of enzymatic activity.

Assay—Inhibition of recombinant human 17β-HSD1: Recombinant protein (1 μg/ml) was incubated in 20 mM KH2PO4 pH 7.4 with 30 nM estrone (including 800 000 cpm/ml of ³H-estrone) and 1 mM NADPH for 30 min at RT, in the presence of the potential inhibitor at concentrations 1 μM or 0.1 μM. Inhibitor stock solutions were prepared in DMSO. Final concentration of DMSO was adjusted to 1% in all samples. The enzyme reaction was stopped by addition of 10% trichloroacetic acid (final concentration). Samples were centrifuged in a microtiter plate at 4000 rpm for 10 min. Supernatants were applied to reverse phase HPLC on a Waters Symmetry C18 column, equipped with a Waters Sentry Guard column. Isocratic HPLC runs were performed at RT at a flow rate of 1 ml/min in acetonitrile:water 48:52 as running solvent. Radioactivity was monitored in the eluate by a Scintillation Analyzer. Total radioactivity for estrone and estradiol were determined in each sample and percent conversion of estrone to estradiol was calculated according to the following formula:

% conversion=100×{(cpm estradiol in sample with inhibitor)/[(cpm estrone in sample with inhibitor)+(cpm estradiol in sample with inhibitor)]}/{[(cpm estradiol in sample without inhibitor)/[(cpm estrone in sample without inhibitor)+(cpm estradiol in sample without inhibitor)]}.

Percent inhibition was calculated followingly: % inhibition=100−% conversion.

The values % inhibition were determined for exemplified compounds and the results are summarized in Table 2.

### Inhibition of the 17β-Hydroxysteroid Dehydrogenase Type 2 Enzyme

17β-HSD2 production and isolation: Similarly to 173-HSD1 the Recombinant baculovirus was generated by the “Bac to Bac Expression System” (Invitrogen). Recombinant bacmid was transfected to Sd9 insect cells using “Cellfectin Reagent” (Invitrogen). 60 h later cells were harvested and supernatant were fractionated by the following protocol:
cells were dissolved into 40 ml of A-buffer (40 mM TRIS, pH8.0, 20% glycerol, 20 μM NAD, 0.4 mM PMSF, 150 mM NaCl, 0.5% dodecyl-β-maltoside+protease inhibitor cocktail)
cells were sonicated
lysate was incubated on ice for 15 min
lysate was centrifuged 5000 rpm 15 min, +4° C.
centrifugation of the supernatant 180 000 g 30 min, +4° C.
pellet was dissolved into 8 ml of A-buffer
not resuspended material was removed by centrifugation 5000 rpm 15 min, +4° C.
the clear supernatant was divided into 100 μl aliquots and were stored frozen until determination of enzymatic activity.

The amount of 173-HSD2 was analysed by immunoblotting and total protein concentration of each extract batch was determined.

Assay—Inhibition of recombinant human 17β-HSD2: Recombinant protein (4 μg/ml) was incubated in 20 mM KH2PO4 pH 8.5 with 50 nM estradiol (including 800 000 cpm/ml of ³H-estradiol) and 1 mM NADH for 30 min at RT, in the presence of the potential inhibitor at concentrations 1 μM or 0.1 μM. Inhibitor stock solutions were prepared in DMSO. Final concentration of DMSO was adjusted to 1% in all samples. The enzyme reaction was stopped by addition of 10% trichloroacetic acid (final concentration). Samples were centrifuged in a microtiter plate at 4000 rpm for 10 min. Supernatants were applied to reverse phase HPLC on a Waters Symmetry C18 column, equipped with a Waters Sentry Guard column. Isocratic HPLC runs were performed at RT at a flow rate of1 ml/min in acetonitrile:water 48:52 as running solvent. Radioactivity was monitored in the eluate by a Scintillation Analyzer. Total radioactivity for estrone and estradiol were determined in each sample and percent conversion of estradiol to estrone was calculated according to the following formula:

% conversion=100×{(cpm estrone in sample with inhibitor)/[(cpm estradiol in sample with inhibitor)+(cpm estrone in sample with inhibitor)]}/{[(cpm estrone in sample without inhibitor)/[(cpm estradiol in sample without inhibitor)+(cpm estrone in sample without inhibitor)]}.

Percent inhibition was calculated followingly: % inhibition=100−% conversion.

The values % inhibition were determined for exemplified compounds and the results are summarized in Table 2.

### Inhibition of the Estrone to Estradiol Conversion in a Rabbit Tissue Homogenate

The assay is based on an enzymatic reaction where HSD1 enzyme that is expressed in rabbit placenta tissue converts its natural substance estrone (E1) to estradiol (E2) in the presence of a co-factor β-NADPH.

Homogenization of rabbit placenta tissue: Weight a piece of the frozen tissue into a Precellys ck28 bead tube. Add buffer solution (20 mM KH2PO4 with 1 mM EDTA, pH 7.4) in 1:2 ratio (e.g. 300 mg of tissue:600 μl reaction buffer solution). Insert the bead tubes to homogenizer and homogenize 2×30 s. 6000 rpm.

Centrifugated 5 min., 2600 rpm at +4° C. and collect supernatant. Aliquots of homogenate are stored in −80° C.

Assay—Inhibition of E1 to E2 conversion in rabbit placenta tissue: The reaction takes place in a buffer solution (20 mM KH2PO4 with 1 mM EDTA, pH 7,4), including appropriate amount of rabbit placenta homogenate, co-factor (1 mM β-NADPH), Substrate (30 nM estrone), labelled substrate as tracer (5 nM [³H]-estrone), and the potential inhibitor at concentrations 1 μM or 0.1 μM. Inhibitor stock solutions were prepared in DMSO. Final concentration of DMSO was adjusted to 1% in all samples. During a 30-minute incubation part of the estrone is converted to estradiol. The reaction is stopped by lowering the pH to 1 with 10% trichloro acetic acid (TCA). The substrate and conversion products are analyzed by HPLC and a Scintillation counter analyzer. Total radioactivity for estrone and estradiol were determined in each sample and percent conversion of estrone to estradiol was calculated according to the following formula:

% conversion=100×{(cpm estradiol in sample with inhibitor)/[(cpm estrone in sample with inhibitor)+(cpm estradiol in sample with inhibitor)]}/{(cpm estradiol in sample without inhibitor)/[(cpm estrone in sample without inhibitor)+(cpm estradiol in sample without inhibitor)]}.

Percent inhibition was calculated followingly: % inhibition=100−% conversion. The values % inhibition were determined for exemplified compounds and the results are summarized in Table 2.

### Metabolic Stability Assay

Compound stock solutions of the invention were prepared in DM. Final concentration of DMSO was adjusted to 1% in all samples. The in vitro metabolic stability of the compounds of the invention was determined for exemplified compounds using human hepatocyte incubations; study compounds at concentration of 1 μM were incubated 0, 10, 20, 40 and 60 min at 37° C. Samples were collected at all time points and compounds were detected by LC-MS/MS analysis. The percent compound remaining is calculated by comparing the peak area of the parent compound at each time point to time zero. In vitro metabolic stability was determined as half life (T1/2), which was determined by regression analysis of the percent parent disappearance vs. time curve. The results are summarized in Table 2.

### Pharmacological Test Results

TABLE 2

| | | | Human | Rabbit |
|---|---|---|---|---|
| | HSD1_in- | HSD2_in- | hepatocytes | placenta |
| Com- | hibition % | hibition % | MetStab | inhibition % |
| pound | @100 nM | @1 μM | T½ min | @100 nM |
| 3 | 98 | 9 | | 54 |
| 6 | 93 | 8 | | 86 |
| 9 | 82 | 3 | 61 | 26 |
| 12 | 92 | 5 | | 46 |
| 15 | 96 | 6 | 43 | 64 |
| 18 | 91 | 1 | 41 | 44 |
| 21 | 97 | 4 | 40 | 68 |
| 24 | 83 | 1 | 33 | 27 |
| 27 | 96 | 2 | | 55 |
| 30 | 95 | 3 | 117 | 53 |
| 33 | 95 | 8 | 107 | 68 |
| 36 | 100 | 8 | 37 | 55 |
| 39 | 91 | 9 | 67 | 69 |
| 42 | 91 | 34 | 47 | 82 |
| 45 | 90 | 3 | 35 | |
| 48 | 91 | 5 | 34 | 57 |
| 51 | 90 | 1 | 31 | 59 |
| 54 | 87 | 8 | 68 | 52 |
| 57 | 92 | 16 | 58 | 48 |
| 60 | 93 | 9 | | 16 |
| 63 | 96 | 2 | | 62 |
| 66 | 74 | 3 | | 39 |
| 69 | 93 | 1 | | 10 |
| 72 | 93 | 2 | | |
| 75 | 71 | 2 | | 15 |
| 78 | 87 | 3 | | 39 |
| 81 | 81 | 3 | | 31 |
| 84 | 87 | 2 | 148 | 27 |
| 87 | 87 | 6 | 127 | 16 |
| 90 | 70 | 3 | 77 | 30 |
| 93 | 79 | 4 | 66 | 31 |
| 96 | 74 | 5 | 75 | 42 |
| 99 | 84 | 4 | 97 | 10 |
| 102 | 80 | 8 | | 19 |
| 105 | 82 | 9 | | 28 |
| 108 | 56 | −2 | | 45 |
| 111 | 48 | 1 | | 55 |
| 114 | 53 | −1 | | 43 |
| 117 | 73 | 2 | | |
| 120 | 67 | 4 | | 34 |
| 123 | 87 | 8 | 155 | 17 |
| 126 | 89 | 12 | 147 | 37 |
| 129 | 84 | 8 | | 28 |
| 135 | 86 | 2 | | 31 |
| 138 | 94 | 7 | 51 | 18 |
| 141 | 93 | −1 | 45 | 35 |
| 147 | 86 | 11 | 9 | |
| 153 | 85 | 4 | | 10 |
| 156 | 81 | −3 | 93 | 56 |
| 159 | 90 | 3 | 77 | 23 |
| 162 | 91 | 9 | 83 | 44 |
| 165 | 85 | −2 | 44 | 34 |
| 168 | 90 | 5 | 68 | 58 |
| 171 | 89 | −1 | 73 | 27 |
| 174 | 88 | 10 | | 14 |
| 177 | 89 | 4 | | 16 |
| 180 | 92 | 12 | | 14 |
| 183 | 91 | 10 | | 50 |
| 186 | 81 | 1 | | 11 |
| 189 | 90 | 9 | 30 | 46 |
| 192 | 86 | 2 | | 14 |
| 195 | 70 | 10 | | 61 |
| 198 | 88 | 18 | | 77 |
| 201 | 87 | 4 | 13 | |
| 204 | 96 | 1 | | 63 |
| 207 | 95 | 4 | 18 | 14 |
| 210 | 94 | 0 | 11 | 19 |
| 213 | 93 | 10 | | 25 |
| 216 | 93 | 6 | 8 | 10 |
| 219 | 91 | 3 | 7 | 14 |
| 222 | 91 | 0 | 12 | 11 |
| 225 | 88 | −2 | | 10 |
| 228 | 87 | 3 | | 67 |
| 231 | 86 | 4 | 29 | 35 |
| 234 | 85 | 0 | 26 | |
| 237 | 84 | 1 | | 58 |
| 240 | 84 | 2 | | 35 |
| 243 | 80 | 5 | 15 | |
| 246 | 80 | 15 | | 19 |
| 249 | 59 | 7 | | |
| 252 | 90 | 7 | | 46 |
| 255 | 66 | 0 | | 46 |
| 258 | 90 | 2 | | 43 |
| 261 | 50 | 9 | | |
| 264 | 54 | 4 | | |

### Utility of the Invention

Compounds of the invention show selective inhibitory potential of the 17β-HSD1 enzyme and little or no inhibitory activity to the 17β-HSD2 enzyme and therefore, may be useful for the treatment of a steroid hormone dependent disease or disorder, in particular for treatment and prevention of several diseases and conditions that include, but are not limited to, breast cancer, prostate carcinoma, ovarian cancer, uterine cancer, endometrial cancer, endometrial hyperplasia, endometriosis, uterine fibroids, adenomyosis, polycystic ovarian syndrome, dysmenorrhea, menorrhagia, metrorrhagia, contraception, prostadynia, benign prostatic hyperplasia, urinary dysfunction, lower urinary tract symptoms, chronic prostatitis/chronic pelvic pain syndrome (CP/CPPS), systemic lupus erythematosus (SLE), multiple sclerosis, obesity, rheumatoid arthritis, chronic obstructive pulmonary disease (COPD), lung cancer, colon cancer, tissue wounds, skin wrinkles and cataracts.

Further, compounds of the present invention may be useful for the treatment of diseases and disorders associated with increased levels of estradiol and which may be prevented, treated, and/or ameliorated by an inhibitor of 17β-HSD1 enzyme.

“Treatment or prevention” as used herein includes prophylaxis, or prevention of, as well as lowering the individual's risk of falling ill with the named disorder or condition, or alleviation, amelioration, elimination, or cure of the said disorder once it has been established.

Compounds of the present invention may be administered in an effective amount within the dosage range of about 0.1 μg/kg to about 300 mg/kg, preferably between 1.0 μg/kg to 10 mg/kg body weight. Compounds of the present invention may be administered in a single daily dose, or the total daily dosage may be administered in divided doses of two, three or four times daily.

“An effective amount” refers to an amount of a compound that confers a therapeutic effect on the treated subject. The therapeutic effect may be objective (i.e. measurable by some test or marker) or subjective (i.e. subject gives an indication of or feels an effect). Such treatment need not necessarily completely ameliorate the condition of disease. Further, such treatment or prevention can be used in conjunction with other traditional treatments for reducing the condition known to those skilled in the art.

Compounds of the invention are most preferably used alone or in combination i.e. administered simultaneously, separately or sequentially with other active ingredients. Compounds of the invention may be administered by various routes, for example, oral, parenteral, subcutaneous, intravenous, intraarticular, intrathecal, intramuscular, intraperitoneal, and by intradermal injections, and via transdermal, rectal, buccal, oromucosal, nasal, ocular routes and via inhalation and via implant.

Compounds may be formulated into a suitable composition; suitable administration forms include, for example, solutions, dispersions, suspensions, powders, capsules, tablet, pills, controlled release capsules, controlled release tablets and controlled release pills. In addition to the pharmacologically active compounds, the pharmaceutical compositions of the compounds can contain suitable pharmaceutically acceptable carriers comprising excipients and auxiliaries that facilitate processing of the active compounds into preparations that can be used pharmaceutically.

Furthermore, compounds of formula (I) can be used as synthesis intermediates for the preparation of other compounds, in particular of other pharmaceutically active ingredients, which are obtainable from compounds of formula (I), for example by introduction of substituents or modification of functional groups.

It will be obvious to a person skilled in the art that, as the technology advances, the inventive concept can be implemented in various ways. The invention and its embodiments are not limited to the examples described above but may vary within the scope of the claims.

1. A compound of formula (I) 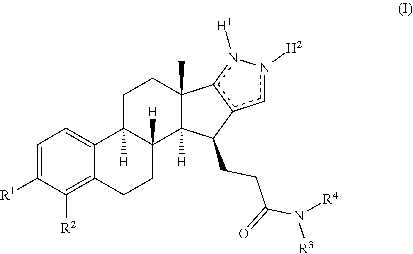
wherein
R1 and R2 are each independently selected from the group consisting of H and halogen;
(i) R3 is selected from the group consisting of H and C1-4 alkyl and
R4 is selected from the group consisting of
C1-6-alkyl optionally substituted with OH;
—(CH₂)ₙ—R5, where n is 1 to 3 and R5 is a 3 to 7 membered alicycle;
4 to 7 membered unsubstituted saturated alicycle or unsubstituted heterocycle comprising one heteroatom selected from the group consisting of nitrogen, sulfur, and oxygen;
5 membered partially unsaturated heterocycle or aromatic heterocycle comprising 1 to 3 heteroatom(s) selected from the group consisting of nitrogen and oxygen, and being optionally substituted with one or two substituents selected from the group consisting of halogen, CN, C1-4-alkyl, C1-3-(per)haloalkyl, OH, C1-3-alkoxy, C(O)N(C1-3-alkyl)₂, and 6 membered saturated heterocycle comprising 1 to 3 heteroatom(s) independently selected from the group consisting of nitrogen, oxygen and sulfur and being optionally substituted with one or two substituent(s) independently selected from the group consisting of halogen, CN, C1-4-alkyl, C1-3-(per)haloalkyl, OH, and C1-3-alkoxy;
5 membered unsubstituted unsaturated or aromatic heterocycle comprising 1 to 3 heteroatom(s) independently selected from the group consisting of nitrogen, sulfur, and oxygen;
5 membered unsaturated or aromatic heterocycle comprising 1 to 3 heteroatom(s) independently selected from the group consisting of nitrogen and oxygen, and being optionally substituted with one or two substituent(s) independently selected from the group consisting of halogen, CN, C1-4-alkyl, C1-3-(per)haloalkyl, OH, C1-3-alkoxy, C(O)N(C1-3-alkyl)₂, and 6 membered saturated heterocycle containing 1 to 3 heteroatom(s) independently selected from the group consisting of nitrogen, oxygen and sulfur and being optionally substituted with one or two substituent(s) independently selected from the group consisting of halogen, CN, C1-4-alkyl, C1-3-(per)haloalkyl, OH, and C1-3-alkoxy;
phenyl, 6 membered unsaturated, or aromatic heterocycle comprising 1 to 3 heteroatom(s) independently selected from the group consisting of nitrogen, sulfur, and oxygen, and being optionally substituted with one to five substituent(s) independently selected from the group consisting of halogen, CN, C1-4-alkyl, C1-3-(per)haloalkyl, OH, oxo, C1-3-alkoxy, morpholino, C(O)N(C1-3-alkyl)₂ and a 6 membered saturated heterocycle with 1 to 3 heteroatom(s) selected from the group consisting of nitrogen, oxygen and sulfur, optionally substituted with C1-4-alkyl; and
6 membered saturated heterocycle comprising 1 to 3 heteroatom(s) independently selected from the group consisting of nitrogen, oxygen and sulfur and being optionally substituted with one to three substituent(s) independently selected from the group consisting of halogen, CN, C1-4-alkyl, C1-3-(per)haloalkyl, OH, oxo, and C1-3-alkoxy, or two adjacent substituents may form a 5 or 6 membered saturated fused ring;
or
(ii) R3 and R4 form together with the nitrogen atom they are attached to a group selected from a 4 to 7 membered saturated heterocycle comprising said nitrogen atom and optionally one additional heteroatom selected from the group consisting of nitrogen, oxygen and sulfur including sulfonyl, and being optionally substituted with a substituent selected from the group consisting of halogen, CN, methyl, C1-3-(per)haloalkyl, OH, oxo, C1-3-alkoxy and a 4 to 7 membered alicycle or saturated heterocycle with 1 to 3 heteroatoms selected from the group consisting of nitrogen, oxygen and sulfur, optionally substituted with C1-4-alkyl;
or
(iii) R3 and R4 form together with the nitrogen atom they are attached to a group selected from a 4 to 7 membered saturated heterocycle comprising said nitrogen atom and said saturated heterocycle forms a fused or spirocycle ring with a 4 to 7 membered saturated or unsaturated alicycle or heterocycle with 1 to 3 heteroatoms selected from the group nitrogen, oxygen and sulfur;
provided that only one of hydrogens H¹ and H² is present at the same time, and the position of the double bonds in the pyrazole ring to which the hydrogens H¹ and H² are attached is determined based on which of the hydrogen H¹ and H² is present
or a pharmaceutically acceptable salt thereof.

2. The compound according to claim 1, wherein the compound has formula (Ia) 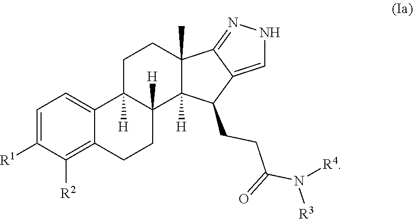
in which formula (Ia) R1, R2, R3, R4 and R5 are as defined in claim 1,
or a pharmaceutically acceptable salt thereof.

3. The compound according to claim 1, wherein
R1 is selected from the group consisting of H, F and Cl,
R2 is selected from the group consisting of H, F and Cl, and
R3, R4 and R5 are as defined in claim 1,
or a pharmaceutically acceptable salt thereof.

4. The compound according to claim 1, wherein
R1 is selected from the group consisting of H, F and Cl,
R2 is H or F, and
R3, R4 and R5 are as defined in claim 1,
or a pharmaceutically acceptable salt thereof.

5. The compound according to claim 1, wherein
R1 is H
R2 is F, and
R3, R4 and R5 are as defined in claim 1,
or a pharmaceutically acceptable salt thereof.

6. The compound according to claim 1, wherein
R3 is H,
R4 is a 6 membered unsaturated or aromatic heterocycle with 1 to 3 heteroatoms selected from the group consisting of nitrogen, oxygen and sulfur, optionally substituted with one or two substituent(s) selected from the group consisting of CN, C1-4-alkyl, C1-3-alkoxy, halogen and C(O)N(C1-3-alkyl)₂ or
R4 is 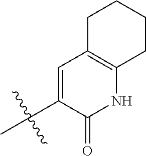 [SMILES: *c1cc2c([nH]c1=O)CCCC2]
or a pharmaceutically acceptable salt thereof.

7. The compound according to claim 1, wherein
the 1 to 3 heteroatoms of the 5 membered unsubstituted unsaturated or aromatic heterocycle are independently selected from nitrogen and oxygen,
the 1 to 3 heteroatoms of the 5 membered unsubstituted unsaturated or aromatic heterocycle are independently selected from 2 nitrogens and 1 sulphur, or
the 1 to 3 heteroatoms of the 5 membered unsubstituted unsaturated or aromatic heterocycle are independently selected from 2 nitrogens and 1 oxygen,
or a pharmaceutically acceptable salt thereof.

8. The compound according to claim 1, wherein
R3 is H,
R4 is a 5 membered unsaturated or aromatic heterocycle with 1 to 3 heteroatoms selected from the group consisting of nitrogen, and oxygen, optionally substituted with one or two substituent(s) selected from the group consisting of CN, C1-4-alkyl, C1-3-alkoxy, halogen and C(O)N(C1-3-alkyl)₂,
or a pharmaceutically acceptable salt thereof.

9. The compound according to claim 1, wherein
R1 is H, Cl or F,
R2 is H, Cl or F,
R3 is H,
R4 is a 6 membered aromatic heterocycle with 1 to 3 heteroatoms selected from the group consisting of nitrogen, oxygen and sulfur, optionally substituted with one or two substituent(s) selected from the group consisting of CN, C1-4-alkyl, C1-3-alkoxy, halogen and C(O)N(C1-3-alkyl)₂, or
R4 is 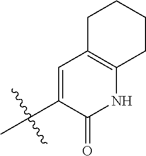 [SMILES: *c1cc2c([nH]c1=O)CCCC2]
or a pharmaceutically acceptable salt thereof.

10. The compound according to claim 1, wherein
R1 is H or F,
R2 is H or F,
R3 is H,
R4 is a 6 membered aromatic heterocycle with 1 to 3 heteroatoms selected from the group consisting of nitrogen, oxygen and sulfur, optionally substituted with one or two substituent(s) selected from the group consisting of CN, C1-4-alkyl, C1-3-alkoxy, halogen and C(O)N(C1-3-alkyl)₂, or
R4 is 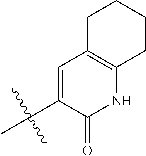 [SMILES: *c1cc2c([nH]c1=O)CCCC2]
or a pharmaceutically acceptable salt thereof.

11. The compound according to claim 1, wherein
R1 is H,
R2 is H or F,
R3 is H,
R4 is a 6 membered aromatic heterocycle with 1 to 3 heteroatoms selected from the group consisting of nitrogen, oxygen and sulfur, optionally substituted with one or two substituent(s) selected from the group consisting of CN, C1-4-alkyl, C1-3-alkoxy, halogen and C(O)N(C1-3-alkyl)₂,
or a pharmaceutically acceptable salt thereof.

12. The compound according to claim 1, wherein
R1 is H,
R2 is F,
R3 is H,
R4 is a 6 membered aromatic heterocycle with 1 to 3 heteroatoms selected from the group consisting of nitrogen, oxygen and sulfur, optionally substituted with one or two substituent(s) selected from the group consisting of CN, methyl, methoxy, F and C(O)N(methyl)₂, or
R4 is 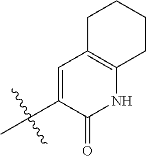 [SMILES: *c1cc2c([nH]c1=O)CCCC2]
or a pharmaceutically acceptable salt thereof.

13. The compound according to claim 1, wherein the compound is selected from the group consisting of:
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(6-methoxypyridazin-3-yl)propanamide,
N-(5-Cyanopyridin-2-yl)-3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(4-fluoropyridin-2-yl)propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(5-methoxypyridin-2-yl)propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(4-methylpyridin-2-yl)propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(2-oxo-1,2,5,6,7,8-hexahydroquinolin-3-yl)propanamide,
6-(3-((8aS,12S)-3-fluoro-8a-methyl-1,2,6b,7,8,8a,10,12,12a,12b-decahydronaphtho[2′,1′:4,5]indeno[1,2-c]pyrazol-12-yl)propanamido)-N,N-dimethylnicotinamide,
N-(6-methoxypyridazin-3-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide,
N,N-dimethyl-6-(3-((8aS,12S)-8a-methyl-1,2,6b,7,8,8a,10,12,12a,12b-decahydronaphtho[2′,1′:4,5]indeno[1,2-c]pyrazol-12-yl)propanamido)nicotinamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(1-methyl-1H-pyrazol-3-yl)-propanamide,
3-((8aS,12S)-3-fluoro-8a-methyl-1,2,6b,7,8,8a,10,12,12a,12b-decahydronaphtho[2′,1′:4,5]indeno[1,2-c]pyrazol-12-yl)-N-(1-methyl-1H-pyrazol-4-yl)-propanamide,
N-(5-(tert-butyl)isoxazol-3-yl)-3-((8aS,12S)-4-fluoro-8a-methyl-1,2,6b,7,8,8a,10,12,12a,12b-decahydronaphtho[2′,1′:4,5]indeno[1,2-c]pyrazol-12-yl)propanamide,
N-(5-fluoropyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide,
N-(4-fluoropyridin-2-yl)-3-((8aS,12S)-8a-methyl-1,2,6b,7,8,8a,10,12,12a,12b-decahydronaphtho[2′,1′:4,5]indeno[1,2-c]pyrazol-12-yl)propanamide, and
3-((8aS,12S)-8a-methyl-1,2,6b,7,8,8a,10,12,12a,12b-decahydronaphtho[2′,1′:4,5]indeno[1,2-c]pyrazol-12-yl)-N-(1-methyl-1H-pyrazol-3-yl)propanamide,
or a pharmaceutically acceptable salt thereof.

14. A method for the preparation of a compound of formula (I) as defined in claim 1,
comprising reacting a compound of formula (II) 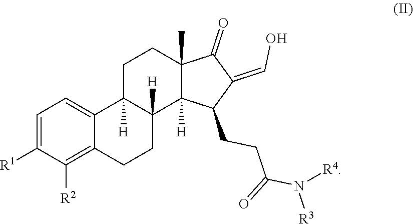
in which formula (II) R1, R2, R3, R4 and R5 are as defined in claim 1,
with hydrazine hydrate to obtain a compound of formula (I); and optionally converting the compound of formula (I) to a pharmaceutically acceptable salt thereof.

15. A compound of formula (II) 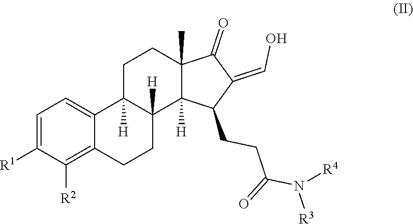
in which formula (II) R1, R2, R3, R4 and R5 are as defined in claim 1.

16. A compound according to claim 1, for use as a medicament.

17. A compound according to claim 1, for use in treatment or prevention of a disease selected from a group consisting of breast cancer, prostate carcinoma, ovarian cancer, uterine cancer, endometrial cancer, endometrial hyperplasia, endometriosis, uterine fibroids, adenomyosis, polycystic ovarian syndrome, dysmenorrhea, menorrhagia, metrorrhagia, contraception, prostadynia, benign prostatic hyperplasia, urinary dysfunction, lower urinary tract symptoms, chronic prostatitis/chronic pelvic pain syndrome (CP/CPPS), systemic lupus erythematosus (SLE), multiple sclerosis, obesity, rheumatoid arthritis, chronic obstructive pulmonary disease (COPD), lung cancer, colon cancer, tissue wounds, skin wrinkles and cataracts.

18. A compound according to claim 1, for use in treatment of a disease selected from a group consisting of breast cancer, prostate carcinoma, ovarian cancer, uterine cancer, endometrial cancer, endometrial hyperplasia, endometriosis, uterine fibroids, adenomyosis, polycystic ovarian syndrome, dysmenorrhea, menorrhagia, metrorrhagia, contraception, prostadynia, benign prostatic hyperplasia, urinary dysfunction, lower urinary tract symptoms, chronic prostatitis/chronic pelvic pain syndrome (CP/CPPS), systemic lupus erythematosus (SLE), multiple sclerosis, obesity, rheumatoid arthritis, chronic obstructive pulmonary disease (COPD), lung cancer, colon cancer, tissue wounds, skin wrinkles and cataracts.

19. A pharmaceutical composition comprising an effective amount of one or more compound(s) according to claim 1, together with one or more pharmaceutically acceptable excipient(s).

20. The pharmaceutical composition according to claim 19, in combination with one or more other active ingredient(s).
